(19) United States
(12) Patent Application Publication
(10) Pub. No.: US 2025/0042896 A1
(43) Pub. Date: Feb. 6, 2025

(54) POLYCYCLIC COMPOUND AS CBL-B INHIBITOR

(71) Applicant: HAINAN SIMCERE ZAIMING PHARMACEUTICAL CO., LTD., Hainan (CN)

(72) Inventors: Wansun MAI, Shanghai (CN); Xiaowu LIU, Shanghai (CN); Wenqiang SHI, Shanghai (CN); Zhixiong DENG, Shanghai (CN); Wei ZHU, Shanghai (CN); Zhengtao LI, Shanghai (CN); Hao ZOU, Shanghai (CN)

(21) Appl. No.: 18/705,948

(22) PCT Filed: Oct. 28, 2022
(86) PCT No.: PCT/CN2022/128426

§ 371 Date: Apr. 29, 2024

(30) Foreign Application Priority Data
Oct. 29, 2021 (CN) 202111271704.9
Jan. 27, 2022 (CN) 202210103242.8
Jul. 28, 2022 (CN) 202210899553.X
Sep. 1, 2022 (CN) 202211063507.2

(51) Int. Cl. C07D 471/04; A61K 31/4196; A61K 31/436; A61K 31/454; A61K 31/4545; A61K 31/519; C07D 405/10; C07D 405/14; C07D 491/052; C07D 513/04

(52) U.S. Cl. CPC C07D 471/04; A61K 31/4196; A61K 31/436; A61K 31/454; A61K 31/4545; A61K 31/519; C07D 405/10; C07D 405/14; C07D 491/052; C07D 513/04

(57) ABSTRACT

The present application describes a polycyclic compound represented by general formula (I) as a Cbl-b inhibitor, or a stereoisomer or pharmaceutically acceptable salt thereof, a pharmaceutical composition comprising the compound of general formula (I) or the stereoisomer or pharmaceutically acceptable salt thereof, and a use of the compound of general formula (I) or the stereoisomer or pharmaceutically acceptable salt thereof, or the pharmaceutical composition in the prevention or treatment of diseases or conditions mediated by Cbl-b.

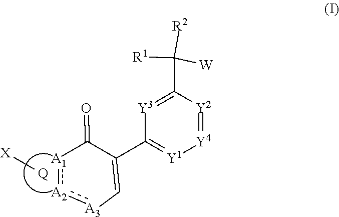

### CROSS REFERENCE TO RELATED APPLICATION

The present application claims the rights and priorities of the following four Chinese patent applications for invention, which are hereby incorporated herein by reference in their entireties:
patent application No. 202111271704.9 submitted to the China National Intellectual Property Administration on Oct. 29, 2021;
patent application No. 202210103242.8 submitted to the China National Intellectual Property Administration on Jan. 27, 2022;
patent application No. 202210899553.X submitted to the China National Intellectual Property Administration on Jul. 28, 2022; and
patent application No. 202211063507.2 submitted to the China National Intellectual Property Administration on Sep. 1, 2022.

### TECHNICAL FIELD

The present application relates to a polycyclic compound as a Cbl-b inhibitor or a stereoisomer or pharmaceutically acceptable salt thereof, a preparation method therefor, a pharmaceutical composition comprising the compound or the stereoisomer or pharmaceutically acceptable salt thereof, and a use of the compound or the stereoisomer or pharmaceutically acceptable salt thereof, or the pharmaceutical composition in the prevention or treatment of diseases or conditions mediated by Cbl-b.

### BACKGROUND

Intracellular signaling cascades are usually regulated by protein phosphorylation, and gene expression and regulation are affected by epigenetics. The proteins that regulate these signals are themselves regulated by the “production-degradation” balance of intracellular proteins, so as to maintain cell homeostasis. It is currently known that protein degradation mainly occurs through two pathways: lysosomal degradation pathway and ubiquitin-mediated proteasomal degradation pathway. The ubiquitin-mediated pathway is a specific protein degradation pathway subject to strict spatiotemporal regulation. The ubiquitin system widely exists in eukaryotes and is a precise intracellular protein degradation regulatory system. Ubiquitin (Ub) is a highly conserved small protein present in most eukaryotic cells. Its main function is to mark proteins that need to be broken down for hydrolysis. The ubiquitin system consists of ubiquitin, 26S proteasome, and various enzymes (E1, E2, E3, deubiquitinase, etc.). The ubiquitination of proteins is completed through the E1-E2-E3 cascade reaction involving Ub activating enzyme E1, Ub conjugating enzyme E2, and Ub ligase E3, and then the ubiquitinated proteins are degraded by the 26S proteasome (A Patent Review of the Ubiquitin Ligase System: 2015-201 Expert Opin Ther Pat. 2018, 28(12): 919-937).

The human genome encodes approximately 35 E2 conjugating enzymes and more than 500 E3 ligases. Casitas B-lineage lymphoma proto-oncogene-b (Cbl-b) is an E3 ligase that negatively regulates T cell activation. Cbl-b belongs to the Cbl family, which includes c-Cbl, Cbl-b and Cbl-3 (Cbl: Many Adaptations to Regulate Protein Tyrosine Kinases. Nat. Rev. Mol. Cell Biol. 2001, 2(4): 294-307). Cbl proteins mainly negatively regulate T cell activation, growth factors (such as epidermal growth factor receptor (EGFR), c-KIT, platelet-derived growth factor receptor (PDGFR)) and non-receptor tyrosine kinase (such as Src family kinases and Zap70) signals (Regulating the Regulator: Negative Regulation of Cbl Ubiquitin Ligases. Trends Biochem Sci, 2006, 31(2): 79-88; The Cbl Family Proteins: Ring Leaders in Regulation of Cell Signaling. J Cell Physiol. 2006, 209: 21-43). Studies have found that functional inactivation of Cbl proteins is related to human cancer (Germline CBL Mutations Cause Developmental Abnormalities and Predispose to Juvenile Myelomonocytic Leukemia. Nat Genet, 2010, 42(9): 794-800). Among the three Cbl proteins, Cbl-b plays a key role in establishing T cell activation threshold and controlling peripheral T cell tolerance, and increasing evidence suggests that Cbl-b also regulates innate immune responses and plays an important role in the host's defense against pathogens. Cbl-b knockout mice show severe autoimmune diseases (Negative Regulation of Lymphocyte Activation and Autoimmunity by the Molecular Adaptor Cbl-b. Nature, 2000, 403: 211-216). Therefore, Cbl-b can serve as an important immunomodulatory therapeutic target.

### SUMMARY OF THE INVENTION

The present application relates to a compound of formula (I), or a stereoisomer or pharmaceutically acceptable salt thereof,

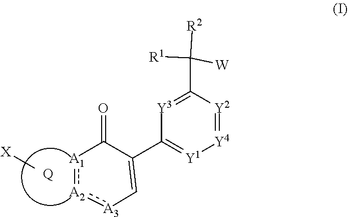

wherein

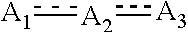

is selected from any of the following: i) C═C-A₃, wherein the A₃ is selected from CR^{11a}R^{11b}, NR¹², O or S; ii) A₁-C=A₃, wherein the A₁ is selected from C or N, and A₃ is selected from CR^{11c} or N; iii) A₁-A₂-A₃, wherein the A₁ and A₂ are independently selected from C(R¹¹)ₙ or N, and A₃ is selected from CR^{11a}R^{11b}, NR¹², O or S; n is selected from 0 or 1;
R^{11a}, R^{11b}, R^{11c}, R¹¹, and R¹² are each independently selected from H, halogen, OH, CN, NH₂, NH(C₁-C₆ alkyl), N(C₁-C₆ alkyl)₂, C₁-C₆ alkyl, C₁-C₆ alkoxy, C₃-C₆ cycloalkyl or C₃-C₆ cycloalkyloxy, wherein the C₁-C₆ alkyl, C₁-C₆ alkoxy, C₃-C₆ cycloalkyl or C₃-C₆ cycloalkyloxy is optionally substituted with R^{11d};
ring Q is selected from phenyl, 5- to 6-membered heteroaryl or 5- to 7-membered heterocyclyl, wherein the phenyl, 5- to 6-membered heteroaryl or 5- to 7-membered heterocyclyl is optionally substituted with R¹⁰;
R¹⁰ is selected from halogen, ═O, OH, NH₂, NO₂, CN, C₁-C₆ alkyl, C₂-C₄ alkenyl, C₂-C₄ alkynyl, C₁-C₆ alkoxy, C₃-C₆ cycloalkyl, C₃-C₆ cycloalkyloxy, C₃-C₆ cycloalkyl-NH—, 4- to 7-membered heterocyclyl, 4- to 7-membered heterocyclyloxy or 4- to 7-membered heterocyclyl-NH—, wherein the NH₂, C₁-C₆ alkyl, C₂-C₄ alkenyl, C₂-C₄ alkynyl, C₁-C₆ alkoxy, C₃-C₆ cycloalkyl, C₃-C₆ cycloalkyloxy, C₃-C₆ cycloalkyl-NH—, 4- to 7-membered heterocyclyl, 4- to 7-membered heterocyclyloxy or 4- to 7-membered heterocyclyl-NH— is optionally substituted with R^{10a};
Y¹, Y², Y³ and Y⁴ are independently selected from CR^{b} or N;
X is selected from halogen, CN, OH, COOH, CONH₂, C₁-C₆ alkyl, C₁-C₆ alkoxy,

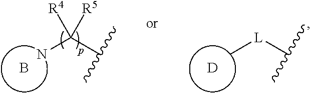

wherein the C₁-C₆ alkyl or C₁-C₆ alkoxy is optionally substituted with R^{e}, ring B is selected from the following groups optionally substituted with R³: 4- to 10-membered nitrogen-containing heterocyclyl or 5- to 10-membered nitrogen-containing heteroaryl, ring D is selected from the following groups optionally substituted with R⁶: C₃-C₁₀ cycloalkyl, 4- to 10-membered heterocyclyl, phenyl or 5- to 10-membered heteroaryl, and ring D is connected to L via a non-N atom, with L being selected from a bond, —NR⁷—, —NR⁷CR⁸R⁹—, —O—, —C(═O)—, —C(═O)NH— or —CR⁸R⁹—;
R^{b} is selected from H, halogen, OH, CN, C₁-C₆ alkyl, C₁-C₆ alkoxy, C₁-C₆ alkylthio, NH₂, NH(C₁-C₆ alkyl), N(C₁-C₆ alkyl)₂, NHC(O)(C₁-C₆ alkyl), NHC(O)—O(C₁-C₆ alkyl), N(C₁-C₆ alkyl)C(O)—O(C₁-C₆ alkyl), NHS(O)₂(C₁-C₆ alkyl), C₃-C₆ cycloalkyl, C₃-C₆ cycloalkyl-O—, C₃-C₆ cycloalkyl-NH—, N(C₃-C₆ cycloalkyl)₂, NHC(O)—C₃-C₆ cycloalkyl, NHS(O)₂—C₃-C₆ cycloalkyl, 4- to 7-membered heterocyclyl, 4- to 7-membered heterocyclyloxy, 4- to 7-membered heterocyclyl-NH—, N(4- to 7-membered heterocyclyl)₂, NHC(O)-4- to 7-membered heterocyclyl, NHS(O)₂-4- to 7-membered heterocyclyl, C₆-C₁₀ aryl, C₆-C₁₀ aryloxy, C₆-C₁₀ aryl-NH—, N(C₆-C₁₀ aryl)₂, NHC(O)—C₆-C₁₀ aryl, NHS(O)₂—C₆-C₁₀ aryl, 5- to 10-membered heteroaryl, 5- to 10-membered heteroaryloxy or 5- to 10-membered heteroaryl-NH—, N(5- to 10-membered heteroaryl)₂, NHC(O)-5- to 10-membered heteroaryl, or NHS(O)₂-5- to 10-membered heteroaryl, wherein the C₁-C₆ alkyl, C₁-C₆ alkoxy, C₁-C₆ alkylthio, C₃-C₆ cycloalkyl, 4- to 7-membered heterocyclyl, C₆-C₁₀ aryl or 5- to 10-membered heteroaryl is optionally substituted with R^{2a};
or two R^{b} together with the C atom to which they are attached form C₃-C₆ cycloalkenyl, phenyl, 4- to 7-membered heterocyclyl or 5- to 6-membered heteroaryl, wherein the C₃-C₆ cycloalkenyl, phenyl, 4- to 7-membered heterocyclyl or 5- to 6-membered heteroaryl is optionally substituted with R^{2a};
R⁴, R⁵, R⁷, R⁸ and R⁹ are each independently selected from H, halogen, OH, C₁-C₆ alkyl or C₁-C₆ alkoxy, wherein the C₁-C₆ alkyl or C₁-C₆ alkoxy is optionally substituted with R^{4a};
or R⁸ and R⁹ together with the atom to which they are attached form C₃-C₆ cycloalkyl or 4- to 7-membered heterocyclyl, wherein the C₃-C₆ cycloalkyl or 4- to 7-membered heterocyclyl is optionally further substituted with R^{8a};
or R⁴ and R⁵ together with the atom to which they are attached form C₃-C₆ cycloalkyl or 4- to 7-membered heterocyclyl, wherein the C₃-C₆ cycloalkyl or 4- to 7-membered heterocyclyl is optionally further substituted with R^{8a}; or when p is selected from 2, two R⁴ together with the atom to which they are attached form C₃-C₆ cycloalkyl or 4- to 7-membered heterocyclyl, wherein the C₃-C₆ cycloalkyl or 4- to 7-membered heterocyclyl is optionally further substituted with R^{8a};
or R⁴ and R⁵ together form ═O;
R³ and R⁶ are independently selected from halogen, CN, ═O, OH, NO₂, C₁-C₆ alkyl, OR^{6a}, SR^{6a}, N(R^{6a})₂, S(O)₂R^{6a}, S(O)₂N(R^{6a})₂, S(O)R^{6a}, S(O)N(R^{6a})₂, C(O)R^{6a}, C(O)OR^{6a}, C(O)N(R^{6a})₂, C(O)N(R^{6a})OR^{6a}, OC(O)R^{6a}, OC(O)N(R^{6a})₂, N(R^{6a})C(O)OR^{6a}, N(R^{6a})C(O)R^{6a}, N(R^{6a})C(O)N(R^{6a})₂, N(R^{6a})C(NR^{6a})N(R^{6a})₂, N(R^{6a})S(O)₂N(R^{6a})₂, N(R^{6a})S(O)₂R^{6a}, C₃-C₁₀ cycloalkyl, 4- to 7-membered heterocyclyl, 6- to 10-membered aryl or 5- to 10-membered heteroaryl, wherein the C₁-C₆ alkyl, C₃-C₁₀ cycloalkyl, 4- to 7-membered heterocyclyl, C₆-C₁₀ aryl or 5- to 10-membered heteroaryl is optionally further substituted with R^{3a};
each R^{6a} is independently selected from H, C₁-C₆ alkyl, phenyl, 4- to 7-membered heterocyclyl or 5- to 6-membered heteroaryl, wherein the C₁-C₆ alkyl, phenyl, 4- to 7-membered heterocyclyl or 5- to 6-membered heteroaryl is optionally further substituted with R^{6b}, or two R^{6a} on one N atom together with the N atom to which they are attached form 4- to 7-membered heterocyclyl or 5- to 6-membered heteroaryl, wherein the 4- to 7-membered heterocyclyl or 5- to 6-membered heteroaryl is optionally further substituted with R^{6b};
R^{3a}, R^{4a}, R^{6b} and R^{e} are independently selected from halogen, OH, CN, ═O, C₁-C₆ alkyl, NH₂, NH(C₁-C₆ alkyl), N(C₁-C₆ alkyl)₂, COOH or C₁-C₆ alkoxy, wherein the C₁-C₆ alkyl or C₁-C₆ alkoxy is optionally further substituted with R^{f};
R^{f} is selected from halogen, OH, ═O, NH₂, NH(C₁-C₆ alkyl) or N(C₁-C₆ alkyl)₂;
R^{11d}, R^{2a} and R^{10a} are independently selected from halogen, OH, CN, ═O, NH₂, NH(C₁-C₆ alkyl), N(C₁-C₆ alkyl)₂, C₁-C₆ alkyl, halo C₁-C₆ alkyl, C₁-C₆ alkoxy or halo C₁-C₆ alkoxy;
R¹ and R² are independently selected from H, halogen, CN, NH₂, NH(C₁-C₆ alkyl), N(C₁-C₆ alkyl)₂, C₁-C₆ alkyl, C₁-C₆ alkoxy, C₃-C₁₀ cycloalkyl or 4- to 10-membered heterocyclyl, wherein the C₁-C₆ alkyl, C₁-C₆ alkoxy, C₃-C₁₀ cycloalkyl or 4- to 10-membered heterocyclyl is optionally substituted with R^{1a},
or R¹ and R² together with the atom to which they are attached form C₃-C₁₀ cycloalkyl or 4- to 10-membered heterocyclyl, wherein the C₃-C₁₀ cycloalkyl or 4- to 10-membered heterocyclyl is optionally substituted with R^{1b};
or R¹ and R^{b} together with the atom and bond to which they are respectively attached form C₃-C₆ cycloalkenyl or 4- to 7-membered heterocyclyl, wherein the C₃-C₆ cycloalkenyl or 4- to 7-membered heterocyclyl is optionally substituted with R^{1d};
R^{1a} and R^{1b} are independently selected from halogen, OH, CN, ═O, NH₂, NH(C₁-C₆ alkyl), N(C₁-C₆ alkyl)₂, C₁-C₆ alkyl or C₁-C₆ alkoxy, wherein the C₁-C₆ alkyl or C₁-C₆ alkoxy is optionally further substituted with R^{1c};
R^{1c} is selected from halogen, OH, CN, ═O, NH₂ or COOH;
W is selected from (CR¹³R¹⁴)ₖW₁; the W₁ is selected from 5- to 10-membered heteroaryl or 4- to 10-membered heterocyclyl, wherein the 5- to 10-membered heteroaryl or 4- to 10-membered heterocyclyl is optionally substituted with R¹⁵; R¹³ and R¹⁴ are independently selected from H, halogen, OH, C₁-C₆ alkyl or C₁-C₆ alkoxy; R¹⁵ is selected from halogen, OH, NH₂, NH(C₁-C₆ alkyl), N(C₁-C₆ alkyl)₂, C₁-C₆ alkyl, C₃-C₁₀ cycloalkyl or 4- to 7-membered heterocyclyl, wherein the C₁-C₆ alkyl, C₃-C₁₀ cycloalkyl or 4- to 7-membered heterocyclyl is optionally substituted with R^{15a};
or R¹ and R¹³ together with the atom and bond to which they are respectively attached form C₃-C₆ cycloalkyl or 4- to 7-membered heterocyclyl, wherein the C₃-C₆ cycloalkyl or 4- to 7-membered heterocyclyl is optionally substituted with R^{13a};
R^{8a}, R^{13a} and R^{1d} are independently selected from halogen, OH, CN, C₁-C₆ alkyl or C₁-C₆ alkoxy, wherein the C₁-C₆ alkyl or C₁-C₆ alkoxy is optionally further substituted with halogen; R^{15a} is selected from halogen, ═O, OH, CN or C₁-C₆ alkyl;
k is selected from 0 or 1;
p is selected from 0, 1 or 2.

In some embodiments, ring D is selected from the following groups optionally substituted with R⁶: C₃-C₁₀ cycloalkyl, 4- to 10-membered heterocyclyl, phenyl or 5- to 10-membered heteroaryl, and ring D is connected to L via a non-N atom, with L being selected from a bond, —NR⁷—, —NR⁷CH₂—, —O—, —C(═O)—, —C(═O)NH— or —CR⁸R⁹—.

In some embodiments, R^{b} is selected from H, halogen, OH, CN, C₁-C₆ alkyl, C₁-C₆ alkoxy, NH₂, NH(C₁-C₆ alkyl), N(C₁-C₆ alkyl)₂, NHC(O)(C₁-C₆ alkyl), NHC(O)—O(C₁-C₆ alkyl), N(C₁-C₆ alkyl)C(O)—O(C₁-C₆ alkyl), NHS(O)₂(C₁-C₆ alkyl), C₃-C₆ cycloalkyl, C₃-C₆ cycloalkyl-O—, C₃-C₆ cycloalkyl-NH—, N(C₃-C₆ cycloalkyl)₂, NHC(O)—C₃-C₆ cycloalkyl, NHS(O)₂—C₃-C₆ cycloalkyl, 4- to 7-membered heterocyclyl, 4- to 7-membered heterocyclyloxy, 4- to 7-membered heterocyclyl-NH—, N(4- to 7-membered heterocyclyl)₂, NHC(O)-4- to 7-membered heterocyclyl, NHS(O)₂-4- to 7-membered heterocyclyl, C₆-C₁₀ aryl, C₆-C₁₀ aryloxy, C₆-C₁₀ aryl-NH—, N(C₆-C₁₀ aryl)₂, NHC(O)—C₆-C₁₀ aryl, NHS(O)₂—C₆-C₁₀ aryl, 5- to 10-membered heteroaryl, 5- to 10-membered heteroaryloxy or 5- to 10-membered heteroaryl-NH—, N(5- to 10-membered heteroaryl)₂, NHC(O)-5- to 10-membered heteroaryl, NHS(O)₂-5- to 10-membered heteroaryl, wherein the C₁-C₆ alkyl, C₁-C₆ alkoxy, C₃-C₆ cycloalkyl, 4- to 7-membered heterocyclyl, C₆-C₁₀ aryl or 5- to 10-membered heteroaryl is optionally substituted with R^{2a}; or two R^{b} together with the C atom to which they are attached form C₃-C₆ cycloalkenyl, phenyl, 4- to 7-membered heterocyclyl or 5- to 6-membered heteroaryl, wherein the C₃-C₆ cycloalkenyl, phenyl, 4- to 7-membered heterocyclyl or 5- to 6-membered heteroaryl is optionally substituted with R^{2a}.

In some embodiments,

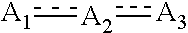

is selected from: i) C═C-A₃, wherein the A₃ is selected from CR^{11a}R^{11b}, NR¹², O or S; or ii) A₁-C=A₃, wherein the A₁ is selected from C or N, and A₃ is selected from CR^{11c} or N.

In some embodiments,

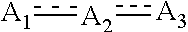

is selected from A₁-C=A₃, wherein the A₁ is selected from C or N, and A₃ is selected from CR^{11c} or N.

In some embodiments,

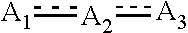

is selected from A₁-C=A₃, wherein the A₁ is selected from N, and A₃ is selected from CR^{11c} or N.

In some embodiments,

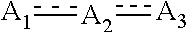

is selected from C═C-A₃, wherein the A₃ is selected from CR^{11a}R^{11b}, NR¹², O or S.

In some embodiments,

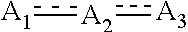

is selected from C═C-A₃, wherein the A₃ is selected from NR¹², O or S.

In some embodiments,

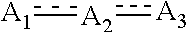

is selected from C═C-A₃, wherein the A₃ is selected from O.

In some embodiments,

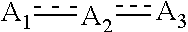

is selected from A₁-A₂-A₃, wherein the A₁ and A₂ are independently selected from C(R¹¹)ₙ or N, and A₃ is selected from NR¹², O or S.

In some embodiments,

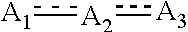

is selected from A₁-A₂-A₃, wherein the A₁ is selected from N, A₂ is selected from C(R¹¹)ₙ, and A₃ is selected from NR¹².

In some embodiments, R^{11a}, R^{11b}, R^{11c}, R¹¹, and R¹² are each independently selected from H, halogen, OH, NH₂, NH(C₁-C₆ alkyl), N(C₁-C₆ alkyl)₂, C₁-C₆ alkyl, C₁-C₆ alkoxy, C₃-C₆ cycloalkyl or C₃-C₆ cycloalkyloxy, wherein the C₁-C₆ alkyl, C₁-C₆ alkoxy, C₃-C₆ cycloalkyl or C₃-C₆ cycloalkyloxy is optionally substituted with R^{11d}.

In some embodiments, R^{11a}, R^{11b}, R^{11c}, R¹¹, and R¹² are each independently selected from H, halogen, OH, NH₂, C₁-C₆ alkyl or C₃-C₆ cycloalkyl, wherein the C₁-C₆ alkyl or C₃-C₆ cycloalkyl is optionally substituted with R^{11d}. In some embodiments, R^{11a}, R^{11b}, R^{11c}, R¹¹, and R¹² are each independently selected from H, halogen or C₁-C₆ alkyl. In some embodiments, R^{11a}, R^{11b}, R^{11e}, R¹¹, and R¹² are each independently selected from H, F or methyl.

In some embodiments, R^{11d} is selected from halogen, OH, NH₂, C₁-C₆ alkyl or halo C₁-C₆ alkyl.

In some embodiments, n is selected from 0.

In some embodiments, R^{11c} is selected from H, halogen or C₁-C₆ alkyl, wherein the C₁-C₆ alkyl is optionally substituted with R^{11d}.

In some embodiments, R^{11c} is selected from H or halogen.

In some embodiments, R^{11c} is selected from F.

In some embodiments, R¹² is selected from H, halogen, OH, C₁-C₆ alkyl or C₃-C₆ cycloalkyl, wherein the C₁-C₆ alkyl or C₃-C₆ cycloalkyl is optionally substituted with R^{11d}.

In some embodiments, R¹² is selected from H or C₁-C₃ alkyl.

In some embodiments, R¹² is selected from H or methyl.

In some embodiments, R¹² is selected from methyl.

In some embodiments,

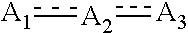

is selected from any of the following: i) C═C-A₃, wherein the A₃ is selected from NH, N—CH₃, O or S; ii) A₁-C=A₃, wherein the A₁ is selected from N, and A₃ is selected from N or C—F; iii) A₁-A₂-A₃, wherein the A₁ is selected from N, A₂ is selected from C, and A₃ is selected from N—CH₃.

In some embodiments,

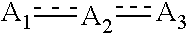

is selected from any of the following: i) C═C-A₃, wherein the A₃ is selected from N—CH₃, O or S; ii) A₁-C=A₃, wherein the A₁ is selected from N, and A₃ is selected from N or C—F; iii) A₁-A₂-A₃, wherein the A₁ is selected from N, A₂ is selected from C, and A₃ is selected from N—CH₃.

In some embodiments,

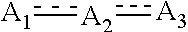

is selected from A₁-C=A₃, wherein the A₁ is selected from N, and A₃ is selected from N.

In some embodiments, ring Q is selected from the following groups optionally substituted with R¹⁰: phenyl, pyridyl, pyrimidyl, pyrazolyl, imidazolyl, oxazolyl, isoxazolyl, thiazolyl, isothiazolyl or 5- to 7-membered heterocyclyl, wherein the 5- to 7-membered heterocyclyl comprises 1 or 2 N atoms as heteroatoms.

In some embodiments, ring Q is selected from the following groups optionally substituted with R¹⁰: phenyl,

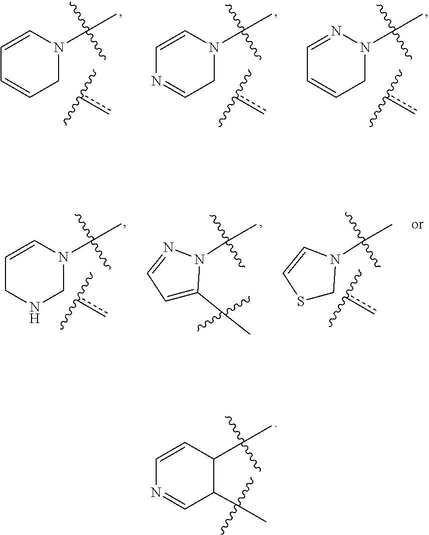

In some embodiments, ring Q is selected from the following groups optionally substituted with R¹⁰: phenyl,

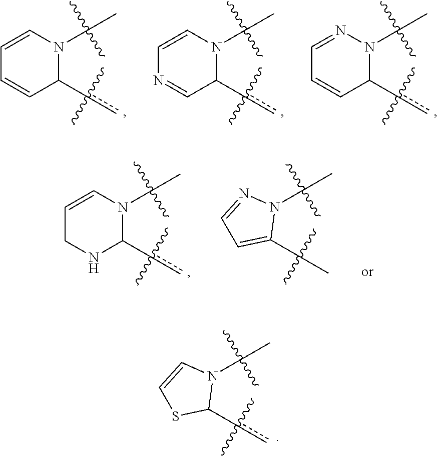

In some embodiments, ring Q is selected from the following groups optionally substituted with R¹⁰: phenyl,

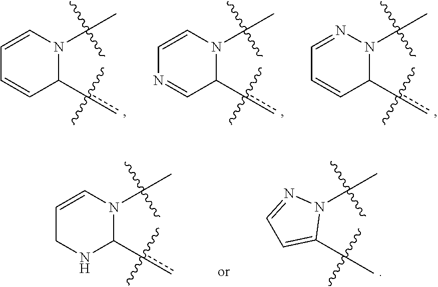

In some embodiments, ring Q is selected from

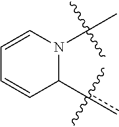

optionally substituted with R¹⁰.

In some embodiments, ring Q is selected from phenyl optionally substituted with R¹⁰.

In some embodiments, R¹⁰ is selected from halogen, ═O, OH, NH₂, CN, C₁-C₆ alkyl, C₂-C₄ alkenyl, C₂-C₄ alkynyl, C₁-C₆ alkoxy, C₃-C₆ cycloalkyl, C₃-C₆ cycloalkyl-O—, C₃-C₆ cycloalkyl-NH—, 4- to 7-membered heterocyclyl, 4- to 7-membered heterocyclyloxy or 4- to 7-membered heterocyclyl-NH—, wherein the NH₂, C₁-C₆ alkyl, C₂-C₄ alkenyl, C₂-C₄ alkynyl, C₁-C₆ alkoxy or C₃-C₆ cycloalkyl is optionally substituted with R^{10a}.

In some embodiments, R¹⁰ is selected from halogen, ═O, NH₂, CN, C₁-C₆ alkyl, C₂-C₄ alkynyl, C₁-C₆ alkoxy, C₃-C₆ cycloalkyl, C₃-C₆ cycloalkyl-O— or C₃-C₆ cycloalkyl-NH—, wherein the NH₂, C₁-C₆ alkyl, C₂-C₄ alkynyl, C₁-C₆ alkoxy or C₃-C₆ cycloalkyl is optionally substituted with R^{10a}. In some embodiments, R¹⁰ is selected from halogen, ═O, NH₂, C₁-C₆ alkyl, C₂-C₄ alkynyl, C₁-C₆ alkoxy, C₃-C₆ cycloalkyl, C₃-C₆ cycloalkyl-O— or C₃-C₆ cycloalkyl-NH—, wherein the NH₂, C₁-C₆ alkyl, C₂-C₄ alkynyl, C₁-C₆ alkoxy or C₃-C₆ cycloalkyl is optionally substituted with R^{10a}.

In some embodiments, R¹⁰ is selected from halogen, ═O, NH₂, C₁-C₆ alkyl, C₂-C₄ alkynyl, C₁-C₆ alkoxy, or C₃-C₆ cycloalkyl, wherein the NH₂, C₁-C₆ alkyl, C₂-C₄ alkynyl, C₁-C₆ alkoxy or C₃-C₆ cycloalkyl is optionally substituted with R^{10a}.

In some embodiments, R¹⁰ is selected from halogen, NH₂, CN, C₁-C₆ alkyl, C₁-C₆ alkoxy or C₃-C₆ cycloalkyl, wherein the NH₂ or C₁-C₆ alkyl is optionally substituted with R^{10a}. In some embodiments, R¹⁰ is selected from halogen, C₁-C₆ alkyl optionally substituted with halogen, C₁-C₆ alkoxy or C₃-C₆ cycloalkyl.

In some embodiments, R^{10a} is selected from halogen, OH, NH₂, C₁-C₆ alkyl, halo C₁-C₆ alkyl, C₁-C₆ alkoxy or halo C₁-C₆ alkoxy.

In some embodiments, R^{10a} is selected from halogen, OH, C₁-C₆ alkyl or halo C₁-C₆ alkyl. In some embodiments, R^{10a} is selected from halogen or C₁-C₆ alkyl.

In some embodiments, R^{10a} is selected from F or CH₃.

In some embodiments, R¹⁰ is selected from ═O, F, Cl, Br, CN, methyl, ethyl, ethynyl, CF₃, CHF₂, methoxy, N(CH₃)₂, cyclopropyl, cyclopropyl-O— or cyclopropyl-NH—.

In some embodiments, R¹⁰ is selected from ═O, F, Cl, ethynyl, CF₃, CHF₂, methoxy, N(CH3)₂, cyclopropyl, cyclopropyl-O— or cyclopropyl-NH—.

In some embodiments, R¹⁰ is selected from ═O, F, Cl, Br, CN, methyl, ethyl, ethynyl, CF₃, methoxy, N(CH₃)₂ or cyclopropyl.

In some embodiments, R¹⁰ is selected from ═O, F, Cl, ethynyl, CF₃, methoxy, N(CH₃)₂ or cyclopropyl.

In some embodiments, R¹⁰ is selected from F, Cl, methyl, Br, ethyl, CF₃, methoxy or cyclopropyl.

In some embodiments,

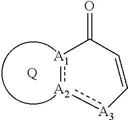

is selected from

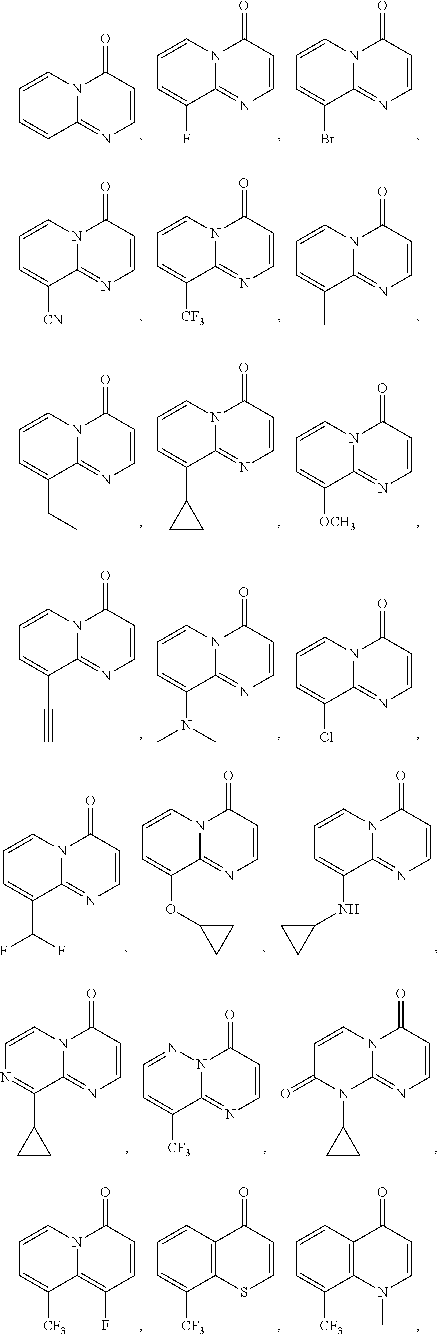

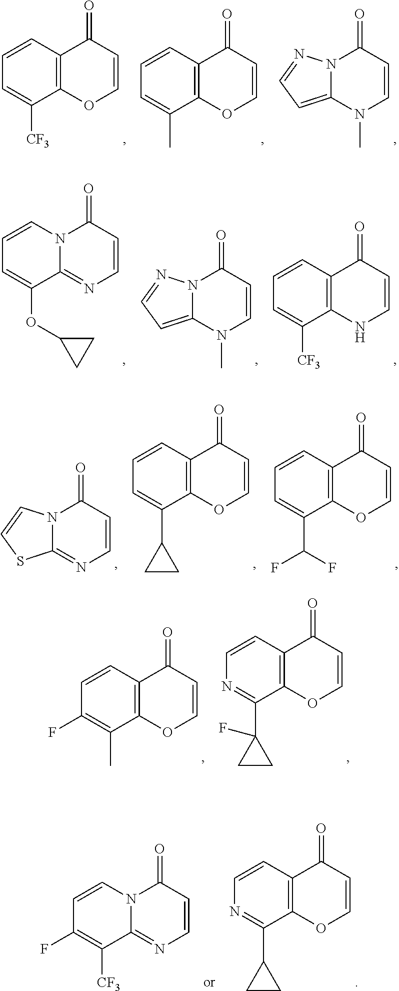

In some embodiments,

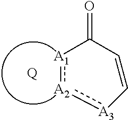

is selected from

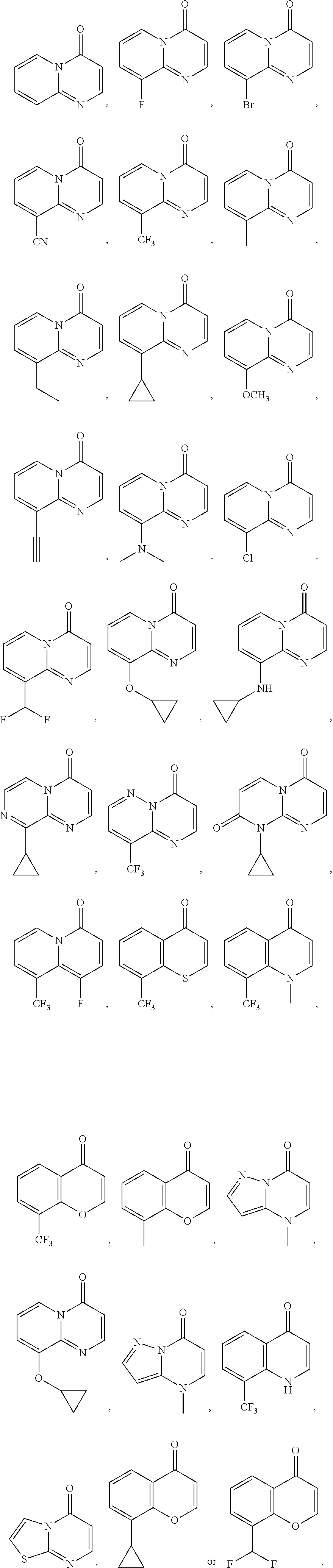

In some embodiments,

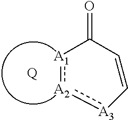

is selected from

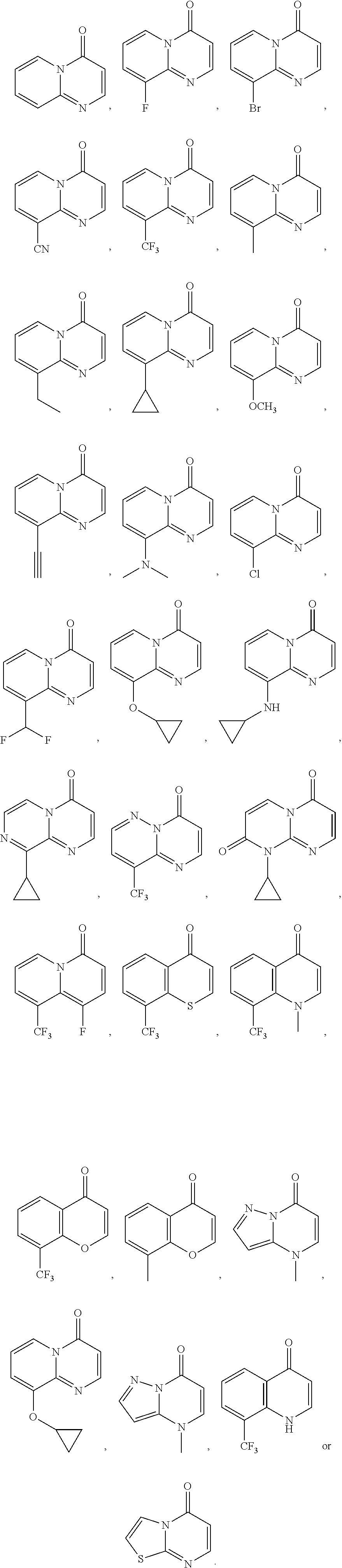

In some embodiments,

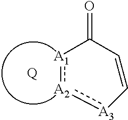

is selected from

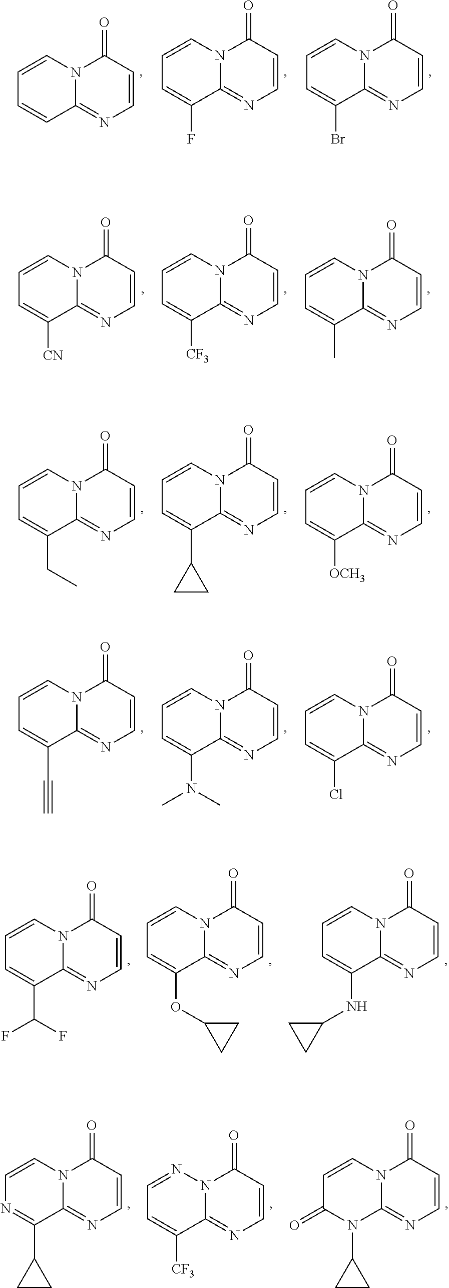

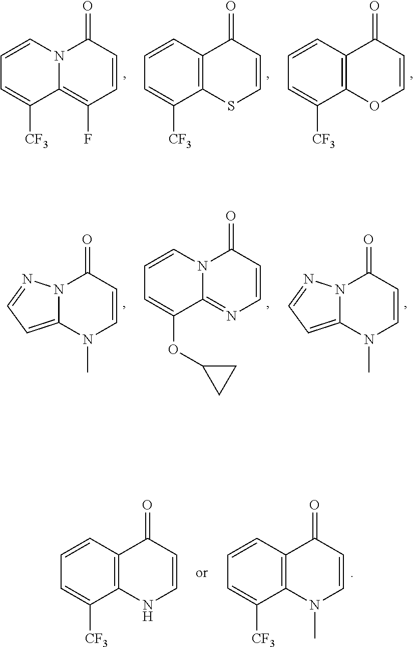

In some embodiments,

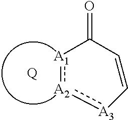

is selected from

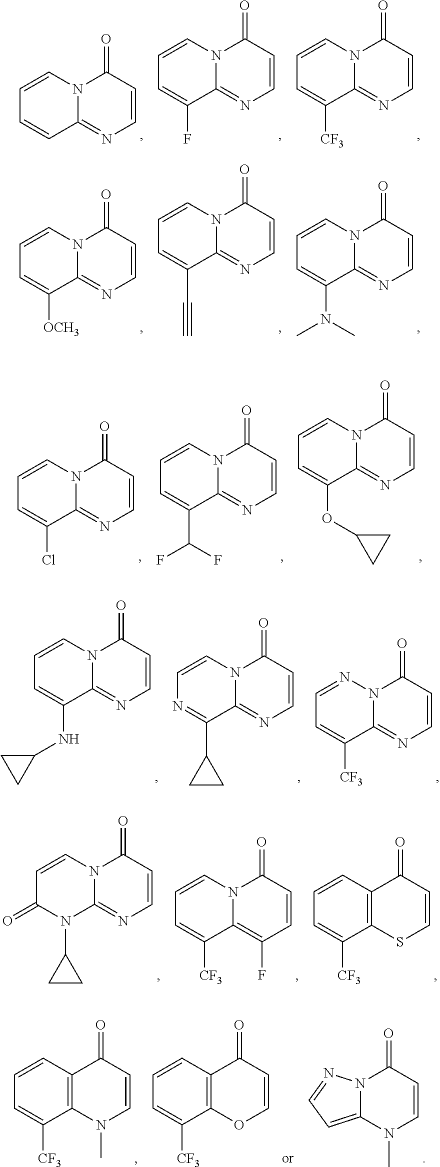

In some embodiments,

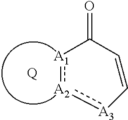

is selected from

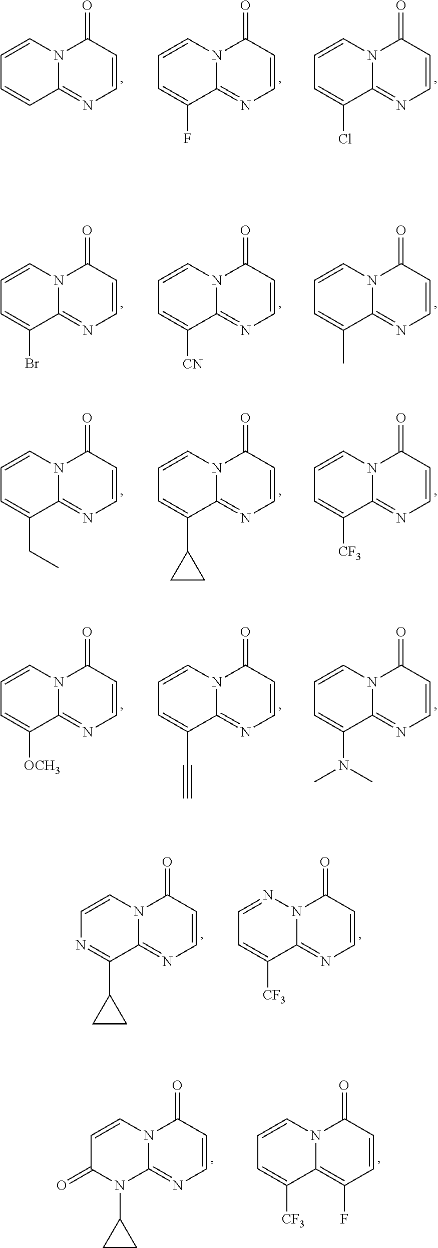

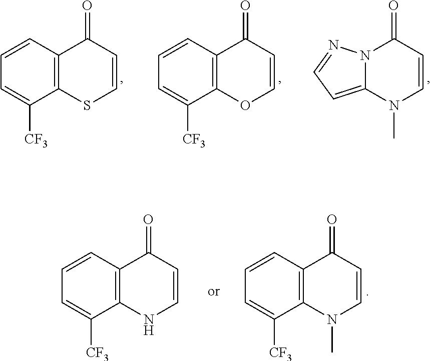

In some embodiments,

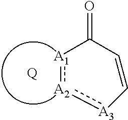

is selected from

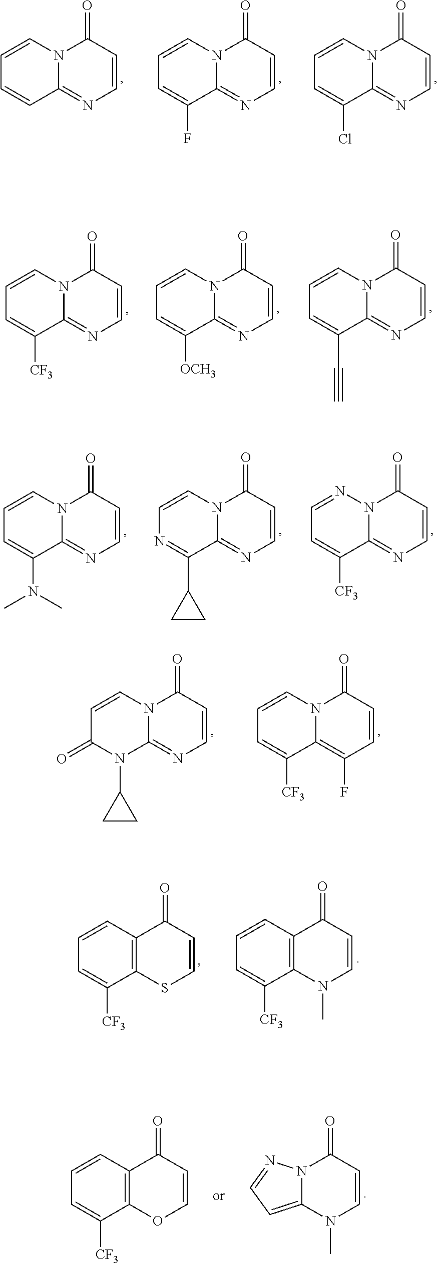

In some embodiments,

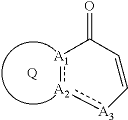

is selected from

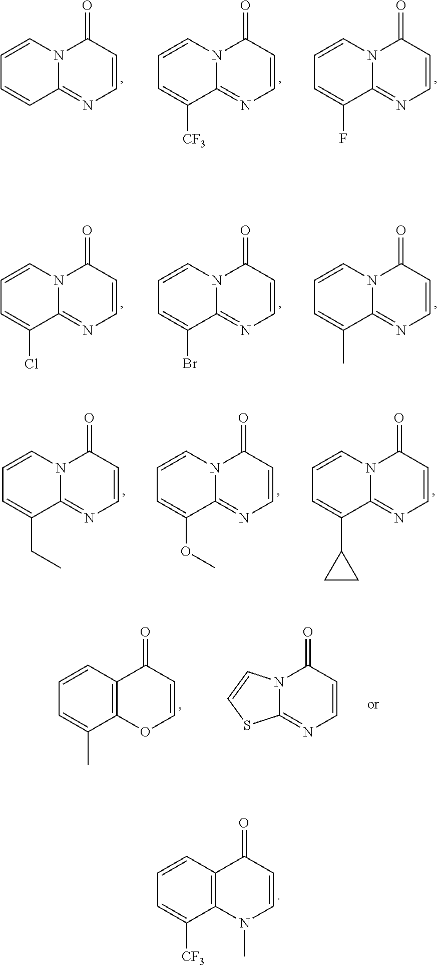

In some embodiments,

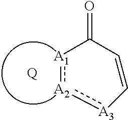

is selected from

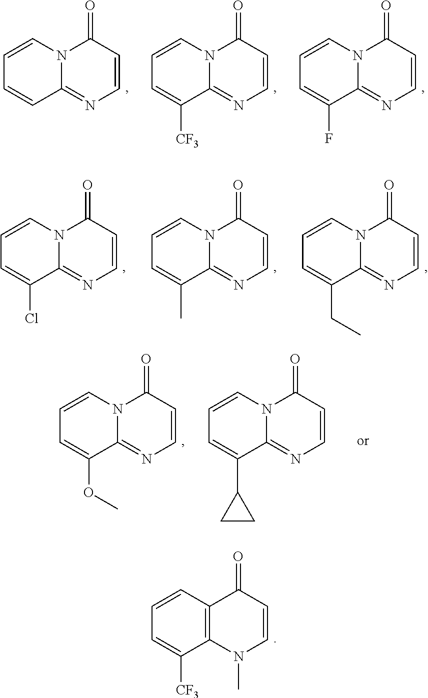

In some embodiments,

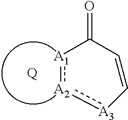

is selected from

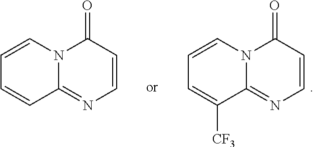 [SMILES: O=c1ccnc2ccccn12 ; O=c1ccnc2c(C(F)(F)F)cccn12]

In some embodiments,

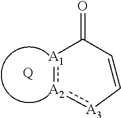

is selected from

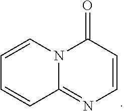 [SMILES: O=c1ccnc2ccccn12]

In some embodiments,

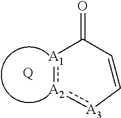

is selected from

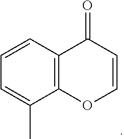

In some embodiments, Y₁, Y₂ and Y₄ are independently selected from CR^{b} or N, and Y₃ is selected from CR^{b}.

In some embodiments, Y¹ and Y² are independently selected from CR^{b} or N, and Y³ and Y⁴ are independently selected from CR^{b}.

In some embodiments, Y¹, Y², Y³ and Y⁴ are all CR^{b}.

In some embodiments, Y¹ and Y² are both N, and Y³ and Y⁴ are independently selected from CR^{b}.

In some embodiments, Y¹ is N, and Y², Y³ and Y⁴ are independently selected from CR^{b}.

In some embodiments, Y¹, Y² and Y³ are all CR^{b}, and Y⁴ is N.

In some embodiments, Y¹, Y² and Y³ are all CH, and Y⁴ is CR^{b}; or, Y¹ is N, Y² and Y³ are both CH, and Y⁴ is CR^{b}; or, Y¹, Y² and Y³ are all CH, and Y⁴ is N.

In some embodiments, R^{b} is selected from H, halogen, CN, C₁-C₆ alkyl, C₁-C₆ alkoxy, C₁-C₆ alkylthio, NH₂, NH(C₁-C₆ alkyl), C₃-C₆ cycloalkyl, C₃-C₆ cycloalkyl-O—, C₃-C₆ cycloalkyl-NH—, 4- to 7-membered heterocyclyl, 4- to 7-membered heterocyclyloxy, 4- to 7-membered heterocyclyl-NH—, 5- to 10-membered heteroaryl, 5- to 10-membered heteroaryloxy or 5- to 10-membered heteroaryl-NH—, wherein the C₁-C₆ alkyl, C₁-C₆ alkoxy, C₁-C₆ alkylthio, C₃-C₆ cycloalkyl, 4- to 7-membered heterocyclyl or 5- to 10-membered heteroaryl is optionally substituted with R^{2a}.

In some embodiments, R^{b} is selected from H, halogen, OH, C₁-C₆ alkyl, C₁-C₆ alkoxy, C₁-C₆ alkylthio, NH₂, NH(C₁-C₆ alkyl), N(C₁-C₆ alkyl)₂, C₃-C₆ cycloalkyl-O—, C₃-C₆ cycloalkyl-NH—, 4- to 7-membered heterocyclyl-O—, 4- to 7-membered heterocyclyl-NH— or 5- to 10-membered heteroaryl, wherein the C₁-C₆ alkyl, C₁-C₆ alkoxy, C₁-C₆ alkylthio, C₃-C₆ cycloalkyl, 4- to 7-membered heterocyclyl or 5- to 10-membered heteroaryl is optionally substituted with R^{2a}.

In some embodiments, R^{b} is selected from H, halogen, OH, C₁-C₆ alkyl, C₁-C₆ alkoxy, NH₂, NH(C₁-C₆ alkyl), N(C₁-C₆ alkyl)₂, C₃-C₆ cycloalkyl-O—, C₃-C₆ cycloalkyl-NH—, 4- to 7-membered heterocyclyl-O—, 4- to 7-membered heterocyclyl-NH— or 5- to 10-membered heteroaryl, wherein the C₁-C₆ alkyl, C₁-C₆ alkoxy, C₃-C₆ cycloalkyl, 4- to 7-membered heterocyclyl or 5- to 10-membered heteroaryl is optionally substituted with R^{2a}.

In some embodiments, R^{b} is selected from H, halogen, CN, or the following groups optionally substituted with R^{2a}: methyl, ethoxy, NHCH₃, NHEt, NH(i-Pr), pyrazolyl, cyclopropyl-O—, cyclobutyl-NH—, oxetanyl-O—, cyclopropyl, SCH₃.

In some embodiments, R^{b} is selected from H, halogen, or the following groups optionally substituted with R^{2a}: methyl, ethoxy, NHCH₃, NHEt, NH(i-Pr), pyrazolyl, cyclopropyl-O—, cyclobutyl-NH— or oxetanyl-O—.

In some embodiments, R^{2a} is selected from halogen, OH, ═O, C₁-C₃ alkyl or C₁-C₃ alkoxy.

In some embodiments, R^{2a} is selected from halogen, OH or C₁-C₃ alkyl.

In some embodiments, R^{2a} is selected from F, OH or methyl.

In some embodiments, R^{b} is selected from H, F, CN, CH₃, CF₃, OEt,

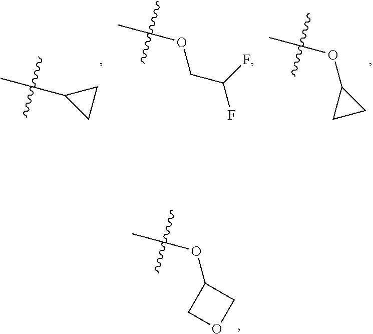

NHCH₃, NHEt, NH(i-Pr), SCH₃,

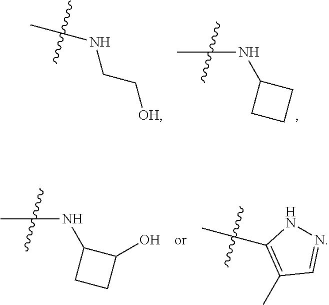

In some embodiments, R^{b} is selected from H, F, CF₃, OEt,

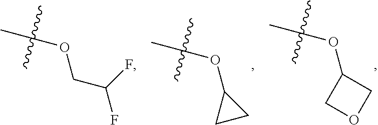 [SMILES: *OCC(F)F ; *OC1CC1 ; *OC1COC1]

### NHCH₃, NHEt, NH(i-Pr),

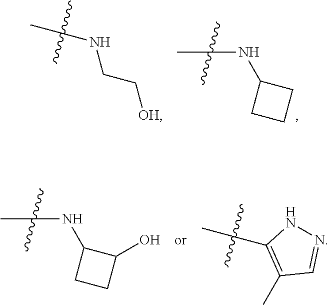

In some embodiments, R^{b} is selected from H, F, CF₃, CN, CH₃,

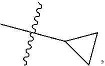 [SMILES: *C1CC1]

OEt, NHEt or SCH₃. In some embodiments, R^{b} is selected from H or CF₃.

In some embodiments, R^{b} and R¹ together with the atom and bond to which they are respectively attached form C₃-C₆ cycloalkenyl or 4- to 7-membered heterocyclyl, wherein the C₃-C₆ cycloalkyl or 4- to 7-membered heterocyclyl is optionally substituted with R^{1d}.

In some embodiments, R^{b} and R¹ together with the atom and bond to which they are respectively attached form C₃-C₆ cycloalkenyl optionally substituted with R^{1d}.

In some embodiments, R^{1d} is selected from halogen, OH, C₁-C₃ alkyl or halo C₁-C₃ alkyl.

In some embodiments, R^{b} and R¹ together with the atom and bond to which they are respectively attached form cyclopentenyl.

In some embodiments,

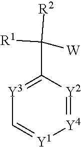

is selected from

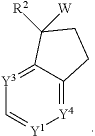

In some embodiments,

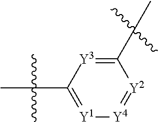

is selected from

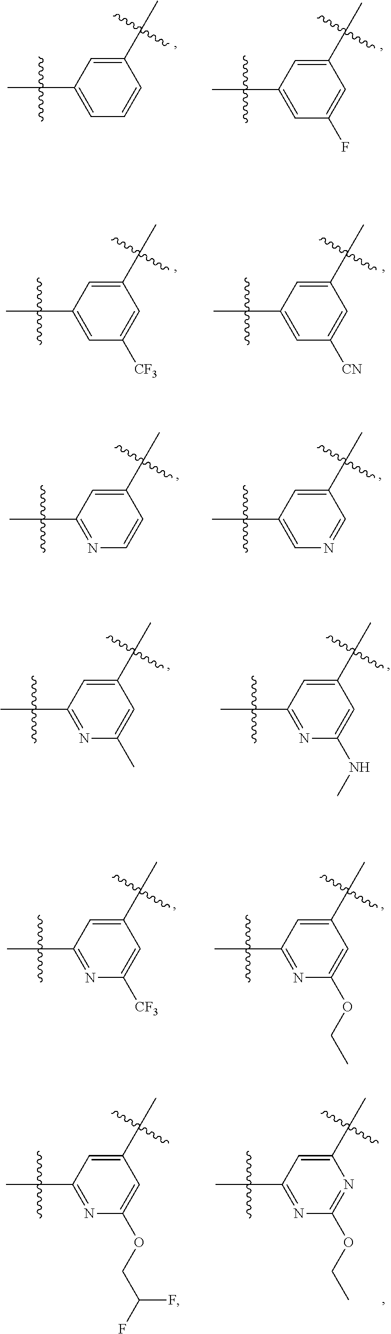

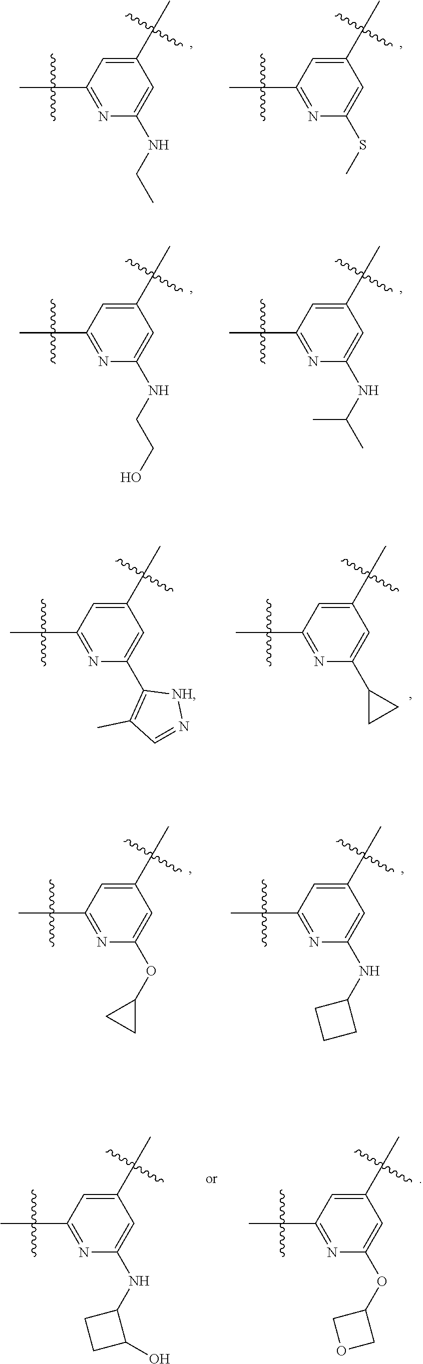

In some embodiments,

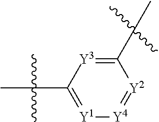

is selected from

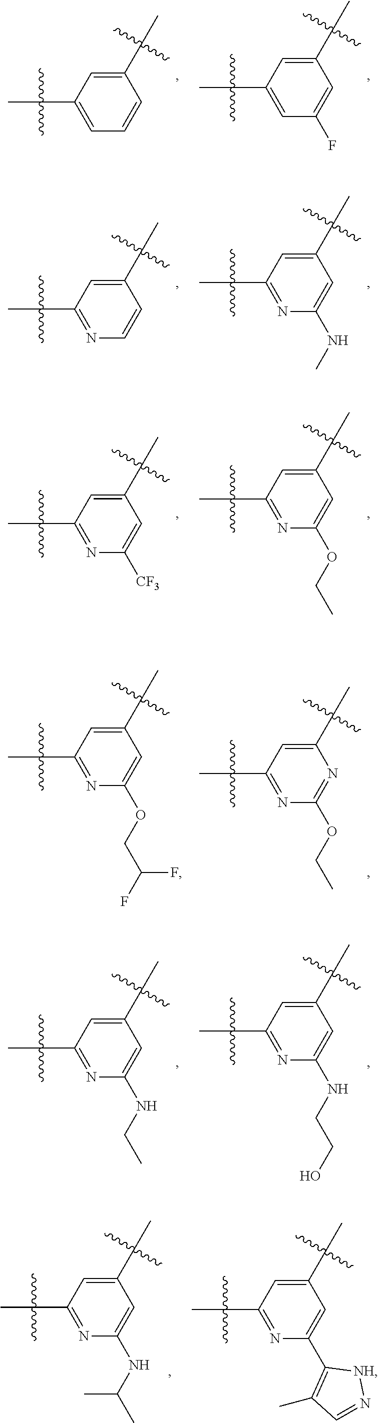

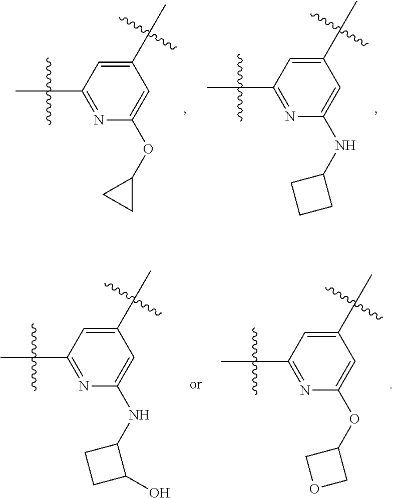 [SMILES: *c1cc(*)nc(OC2CC2)c1 ; *c1cc(*)nc(NC2CCC2)c1 ; *c1cc(*)nc(NC2CCC2O)c1 ; *c1cc(*)nc(OC2COC2)c1]

In some embodiments,

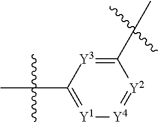

is selected from

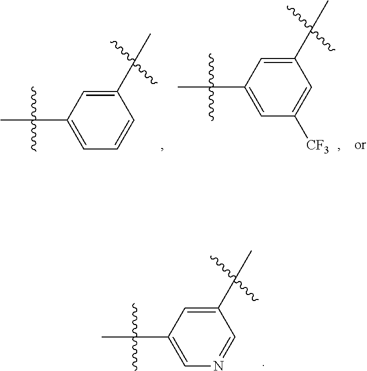 [SMILES: *c1cccc(*)c1 ; *c1cc(*)cc(C(F)(F)F)c1 ; *c1cncc(*)c1]

In some embodiments, X is selected from

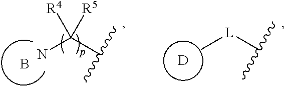

or C₁-C₆ alkyl optionally substituted with R^{e}.

In some embodiments, X is selected from

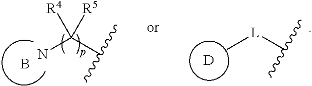

In some embodiments, X is selected from

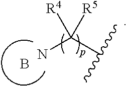

In some embodiments, ring B is selected from 5- to 10-membered nitrogen-containing heteroaryl, 4- to 7-membered monocyclic nitrogen-containing heterocyclyl or 6- to 10-membered nitrogen-containing heterocyclyl which is optionally substituted with R³.

In some embodiments, ring B is selected from the following groups optionally substituted with R³: tetrahydropyrrolyl, piperidyl, piperazinyl, morpholinyl,

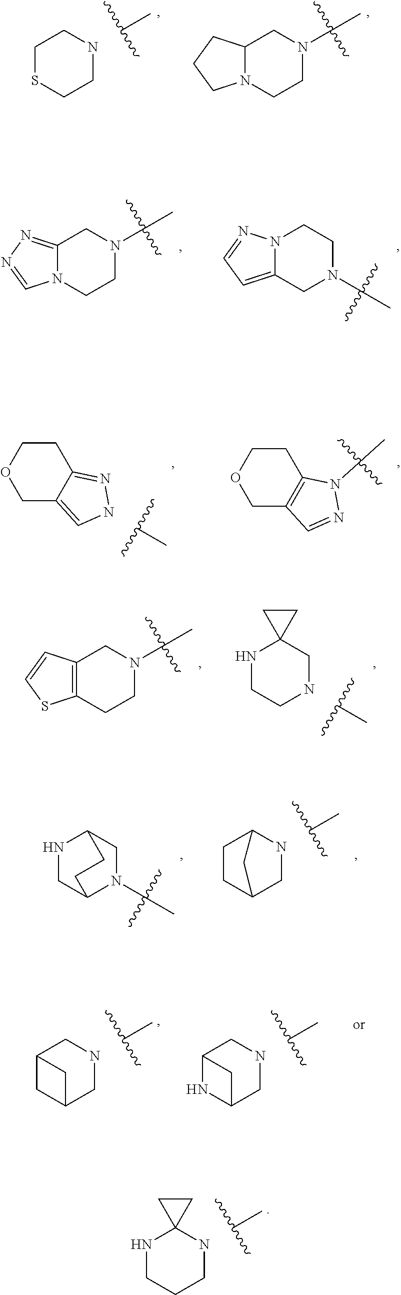

In some embodiments, ring B is selected from the following groups optionally substituted with R³: tetrahydropyrrolyl, piperidyl, piperazinyl, morpholinyl,

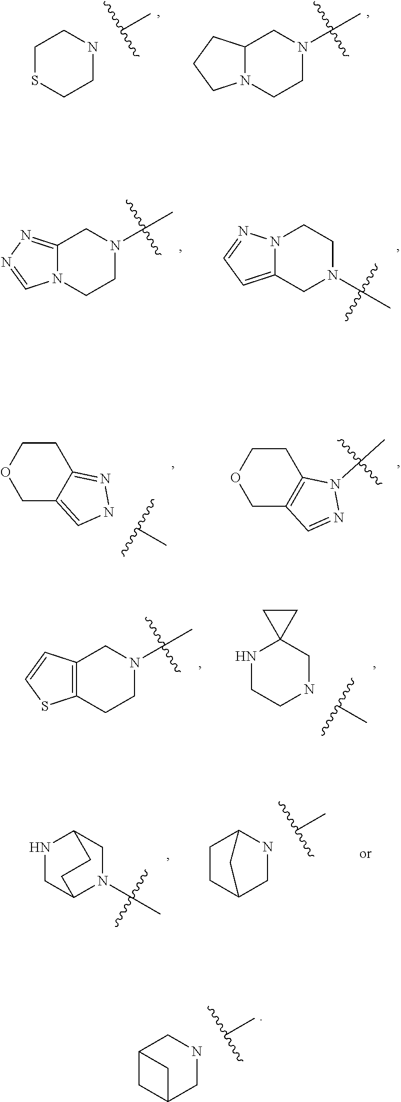

In some embodiments, ring B is selected from the following groups optionally substituted with R³: tetrahydropyrrolyl, piperidyl, piperazinyl, morpholinyl,

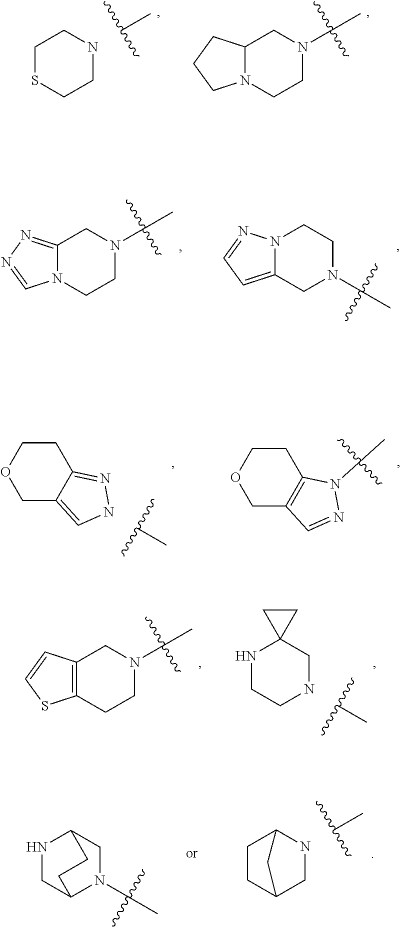

In some embodiments, ring B is selected from the following groups optionally substituted with R³: tetrahydropyrrolyl, piperidyl, piperazinyl, morpholinyl,

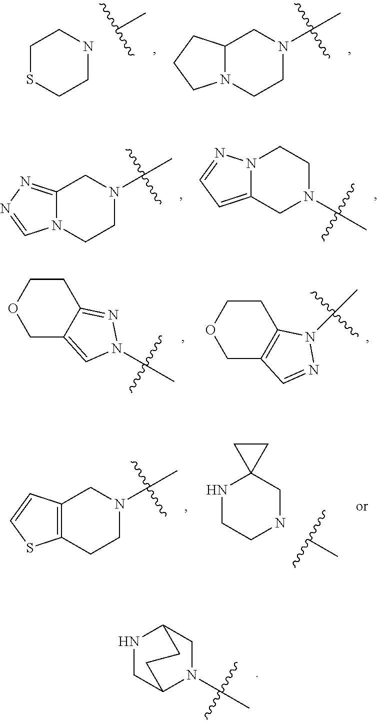

In some embodiments, R³ is selected from halogen, OH, ═O, CN, C₁-C₆ alkyl, C₁-C₆ alkoxy, C₃-C₆ cycloalkyl, C₆-C₁₀ aryl or 5- to 10-membered heteroaryl, wherein the C₁-C₆ alkyl, C₁-C₆ alkoxy, C₃-C₆ cycloalkyl, C₆-C₁₀ aryl or 5- to 10-membered heteroaryl is optionally further substituted with R^{3a}.

In some embodiments, R³ is selected from halogen, OH, ═O, C₁-C₃ alkyl, C₃-C₆ cycloalkyl or phenyl, wherein the C₁-C₃ alkyl, C₃-C₆ cycloalkyl or phenyl is optionally further substituted with R^{3a}.

In some embodiments, R^{3a} is selected from halogen, OH, ═O or C₁-C₃ alkoxy.

In some embodiments, R^{3a} is selected from F.

In some embodiments, R³ is selected from ═O, OH, F, methyl, isopropyl, CF₃, cyclopropyl or phenyl.

In some embodiments, R³ is selected from ═O, OH, F, methyl, CF₃, cyclopropyl or phenyl.

In some embodiments, R⁴ and R⁵ are independently selected from H, halogen, OH, or C₁-C₃ alkyl optionally substituted with R^{4a}; or R⁴ and R⁵ together with the atom to which they are attached form C₃-C₆ cycloalkyl optionally substituted with R^{8a}; or R⁴ and R⁵ together form ═O; or when p is selected from 2, two R⁴ together with the atom to which they are attached form C₃-C₆ cycloalkyl optionally substituted with R^{8a}.

In some embodiments, R⁴ and R⁵ are independently selected from H, halogen, OH, or C₁-C₃ alkyl optionally substituted with R^{4a}; or R⁴ and R⁵ together with the atom to which they are attached form C₃-C₆ cycloalkyl optionally substituted with R^{8a}; or R⁴ and R⁵ together form ═O.

In some embodiments, R^{4a} is selected from halogen, OH or C₁-C₃ alkoxy.

In some embodiments, R^{4a} is selected from F or OH.

In some embodiments, R^{8a} is selected from halogen, OH or C₁-C₃ alkyl.

In some embodiments, R⁴ and R⁵ are independently selected from H, methyl, hydroxymethyl or CF₃, or R⁴ and R⁵ together with the atom to which they are attached form cyclopropyl, or R⁴ and R⁵ together form ═O.

In some embodiments, p is selected from 1.

In some embodiments, p is selected from 0.

In some embodiments, p is selected from 2, and two R⁴ together with the atom to which they are attached form C₃-C₆ cycloalkyl optionally substituted with R^{8a}.

In some embodiments, p is selected from 2, and two R⁴ together with the atom to which they are attached form cyclopropyl or cyclobutyl.

In some embodiments,

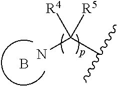

is selected from the following groups:

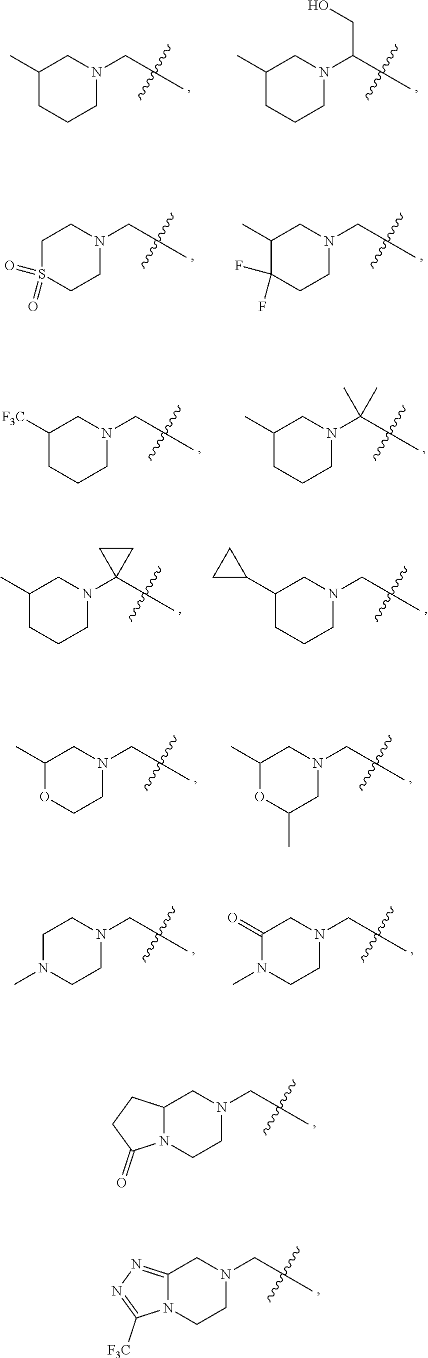

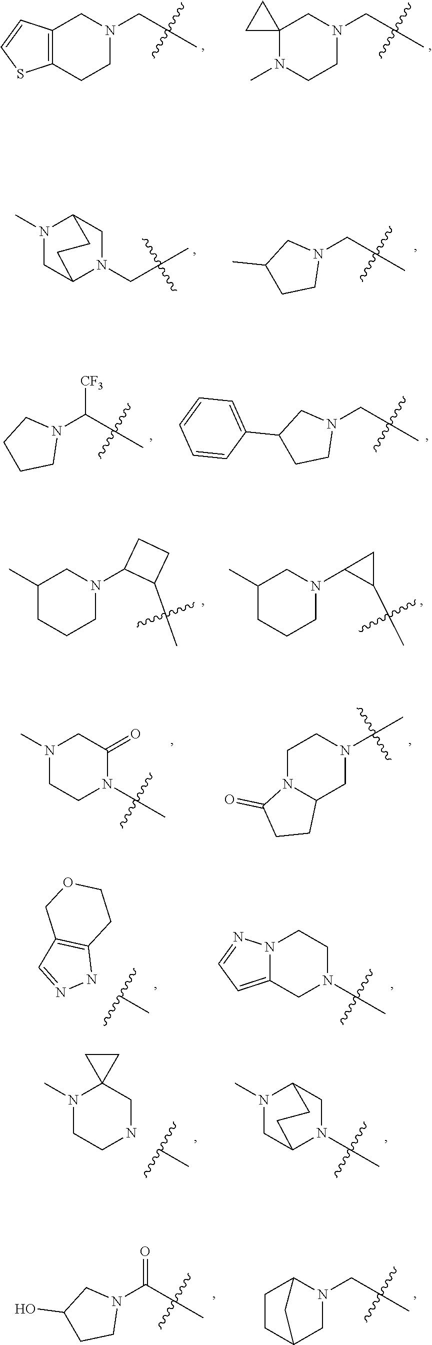

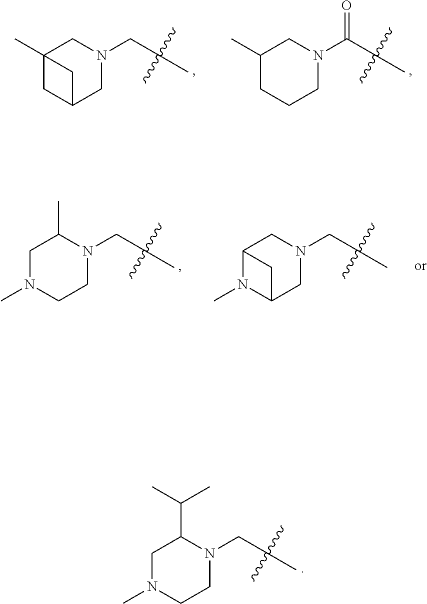

In some embodiments,

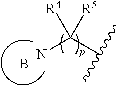

is selected from the following groups:

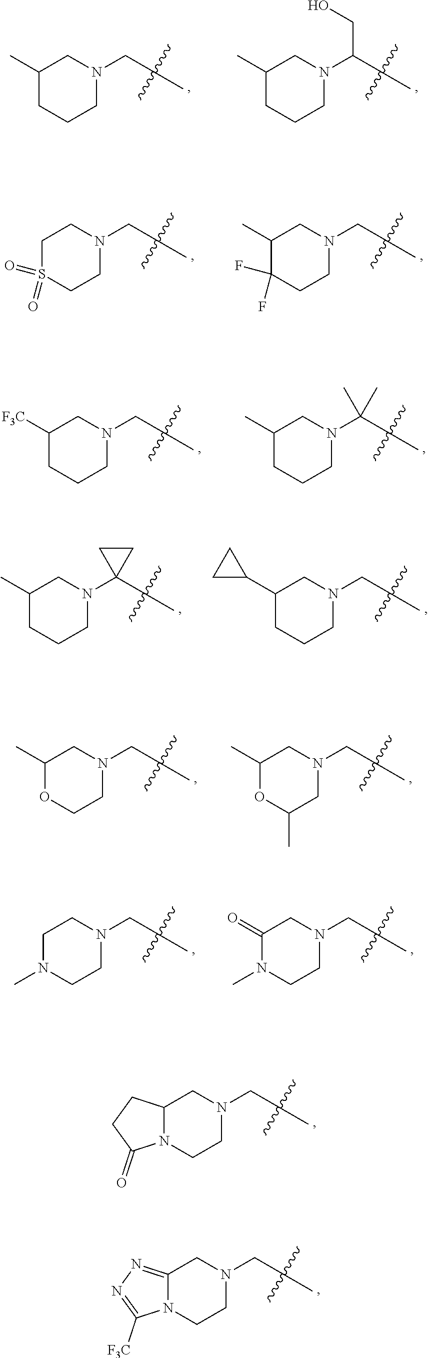

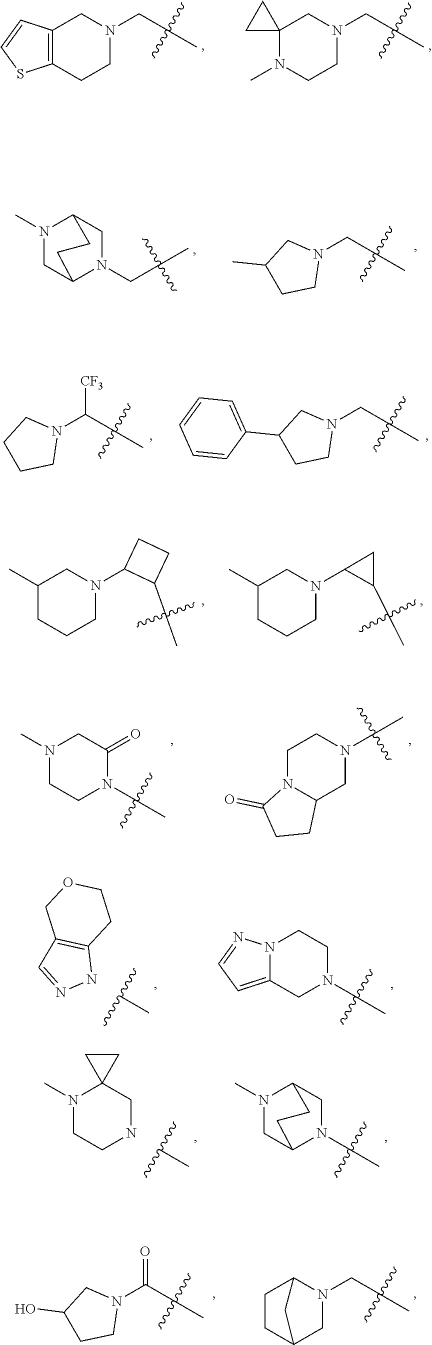

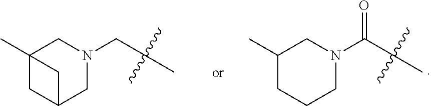

In some embodiments,

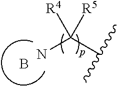

is selected from the following groups:

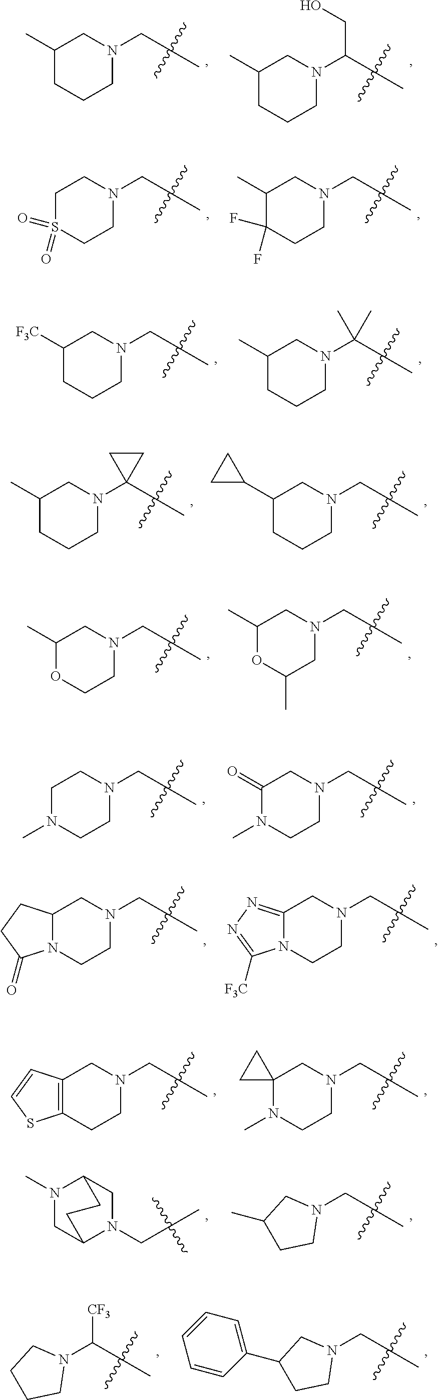

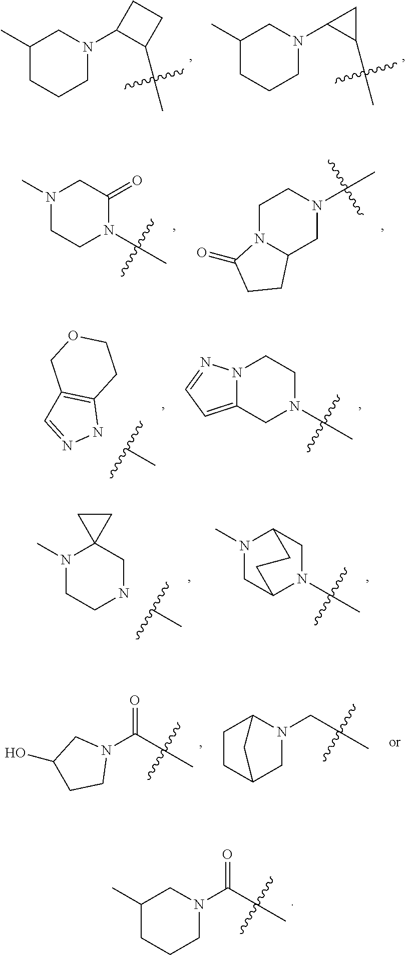

In some embodiments,

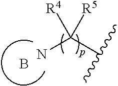

is selected from the following groups:

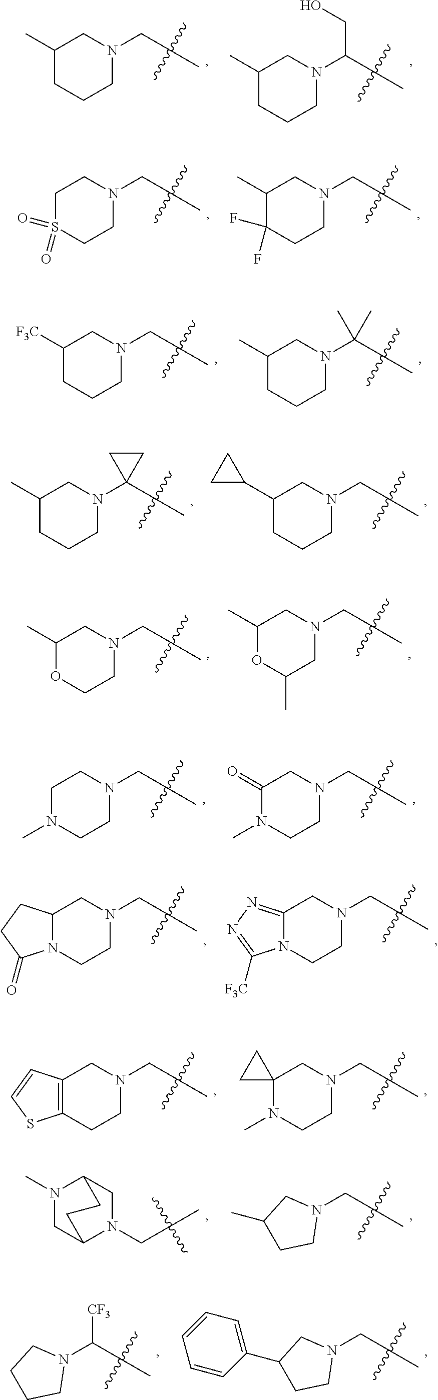

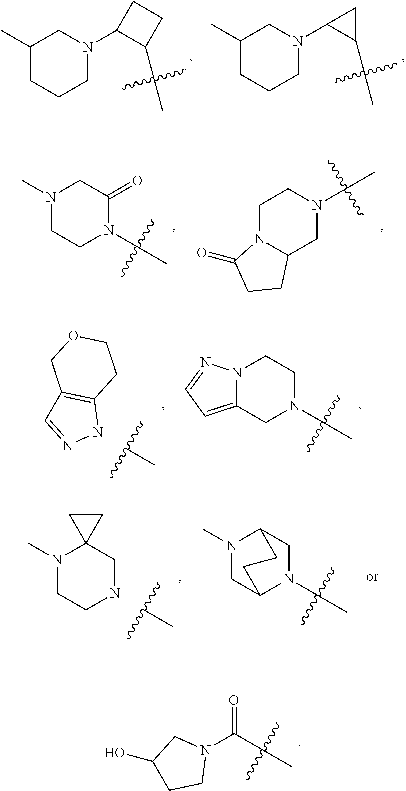

In some embodiments,

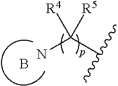

is selected from the following groups:

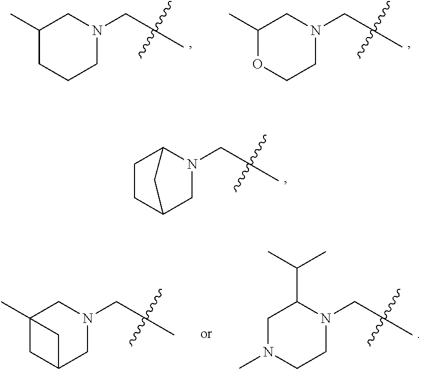

In some embodiments, ring D is selected from the following groups optionally substituted with R⁶: C₃-C₆ cycloalkyl, 5- to 6-membered heteroaryl, 4- to 7-membered monocyclic nitrogen-containing heterocyclyl or 6- to 10-membered nitrogen-containing heterocyclyl, and ring D is connected to L via a non-N atom.

In some embodiments, ring D is selected from the following groups optionally substituted with R⁶: cyclopropyl, cyclobutyl, cyclopentyl, tetrahydropyrrolyl, piperidyl, piperazinyl,

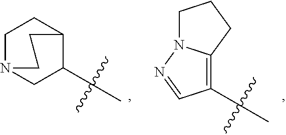 [SMILES: *C1CN2CCC1CC2 ; *c1cnn2c1CCC2]

pyrazolyl, imidazolyl, oxazolyl, isoxazolyl, triazolyl, thiazolyl, isothiazolyl or pyridyl.

In some embodiments, ring D is selected from the following groups optionally substituted with R⁶: cyclobutyl, cyclopentyl, piperidyl, pyridyl,

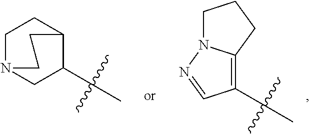 [SMILES: *C1CN2CCC1CC2 ; *c1cnn2c1CCC2]

In some embodiments, R⁶ is selected from halogen, OH, CN, ═O, or C₁-C₃ alkyl optionally substituted with R^{3a}.

In some embodiments, R⁶ is selected from halogen, ═O, OH or C₁-C₃ alkyl.

In some embodiments, R⁶ is selected from F or methyl.

In some embodiments, L is selected from a bond, —NR⁷—, —NR⁷CR⁸R⁹—, —O— or —CR⁸R⁹—.

In some embodiments, L is selected from a bond, —NR⁷—, —NR⁷CH₂—, —O— or —CR⁸R⁹—.

In some embodiments, R⁷, R⁸ and R⁹ are independently selected from H, halogen, C₁-C₃ alkyl or OH.

In some embodiments, R⁷, R⁸ and R⁹ are independently selected from H, halogen or C₁-C₃ alkyl (such as, methyl).

In some embodiments, R⁷, R⁸ and R⁹ are independently selected from H or halogen (such as, F).

In some embodiments, R⁷ is selected from H.

In some embodiments, R⁸ and R⁹ are selected from H, methyl or F.

In some embodiments, L is selected from a bond, —NH—, —NHCH₂—, —NHCH(CH₃)—, —O—, —C(F)₂— or —CH₂—.

In some embodiments, L is selected from a bond, —NH—, —NHCH₂—, —O—, —C(F)₂— or —CH₂—.

In some embodiments,

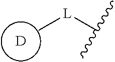

is selected from the following groups:

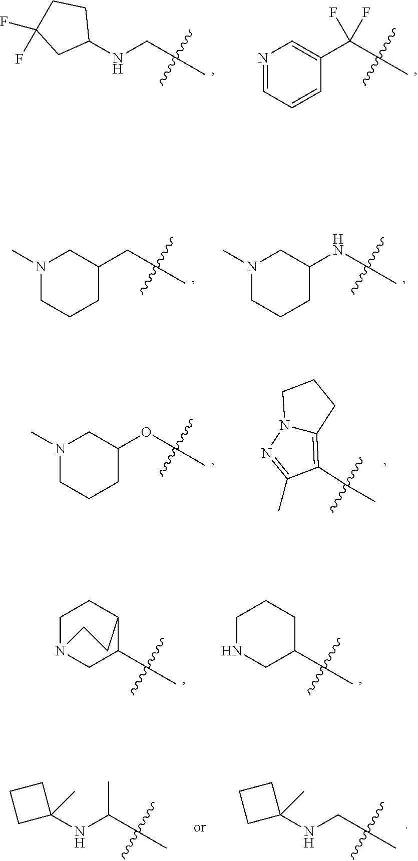

In some embodiments,

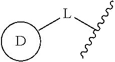

is selected from the following groups:

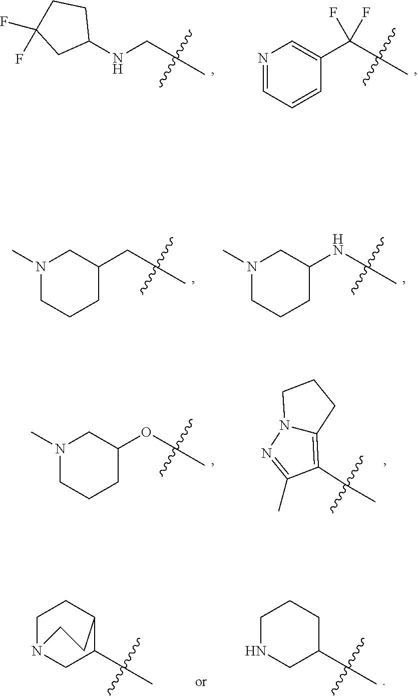

In some embodiments,

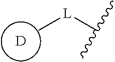

is selected from the following groups:

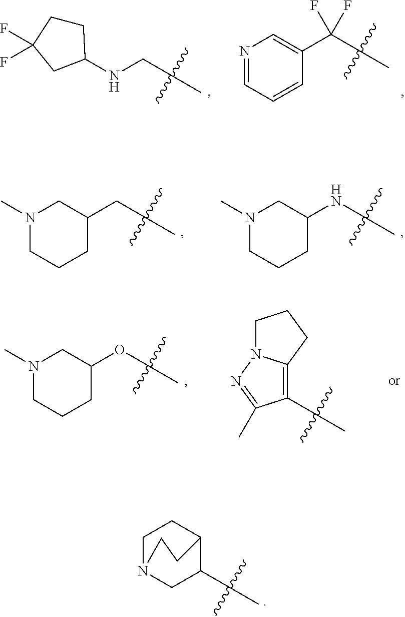

In some embodiments,

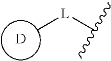

is selected from

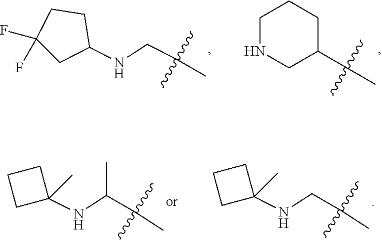

In some embodiments,

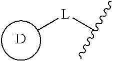

is selected from

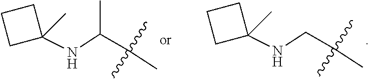

In some embodiments, X is selected from C₁-C₆ alkyl optionally substituted with R^{e}.

In some embodiments, R^{e} is selected from halogen, OH, C₁-C₃ alkyl, NH₂, NH(C₁-C₃ alkyl), N(C₁-C₃ alkyl)₂ or C₁-C₃ alkoxy, wherein the C₁-C₃ alkyl or C₁-C₃ alkoxy is further optionally substituted with R^{f}.

In some embodiments, R^{e} is selected from C₁-C₃ alkyl, N(C₁-C₃ alkyl)₂ or C₁-C₃ alkoxy, wherein the C₁-C₃ alkyl or C₁-C₃ alkoxy is further optionally substituted with R^{f}.

In some embodiments, R^{f} is selected from halogen, OH or N(C₁-C₆ alkyl)₂.

In some embodiments, R^{f} is selected from N(CH₃)₂.

In some embodiments, R^{e} is selected from N(CH₃)₂, CH₂N(CH₃)₂ or

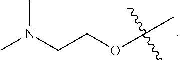 [SMILES: *OCCN(C)C]

In some embodiments, X is selected from

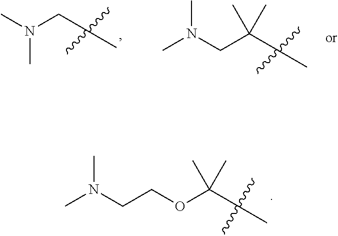

In some embodiments, X is selected from the following groups:

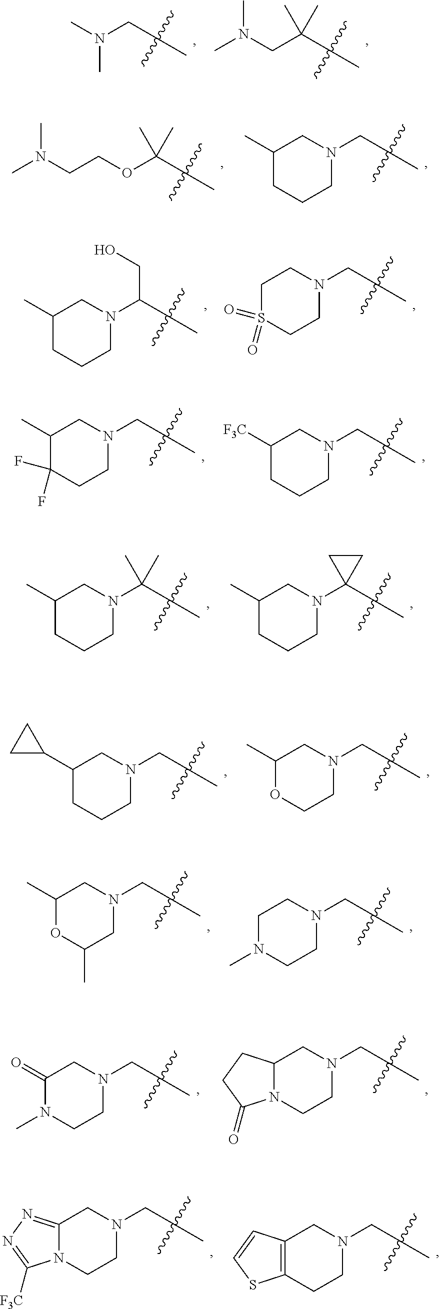

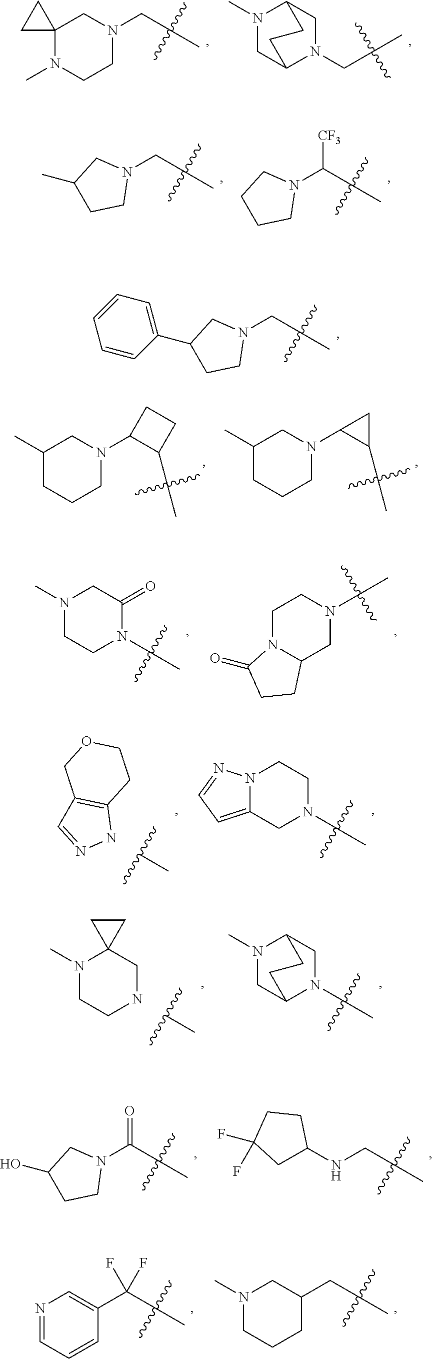

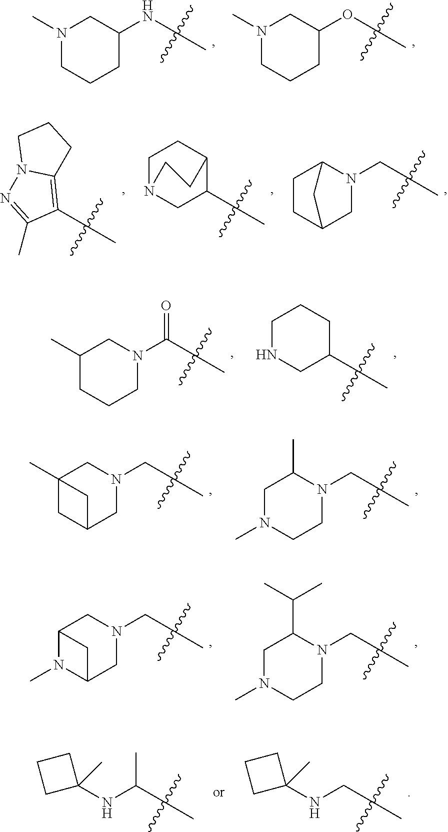

In some embodiments, X is selected from the following groups:

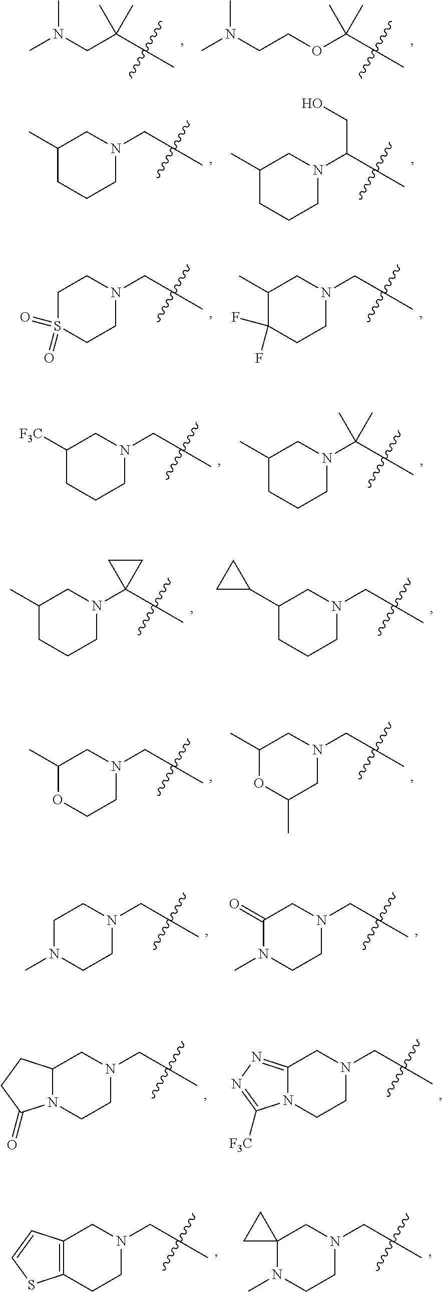

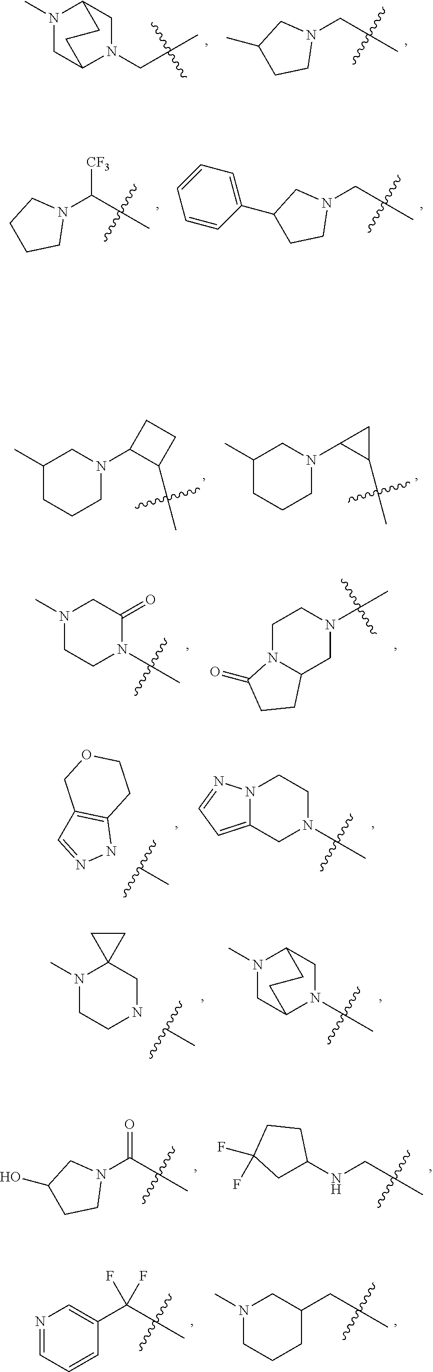

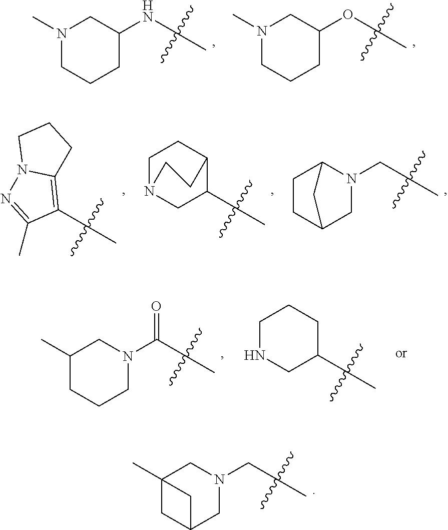

In some embodiments, X is selected from the following groups:

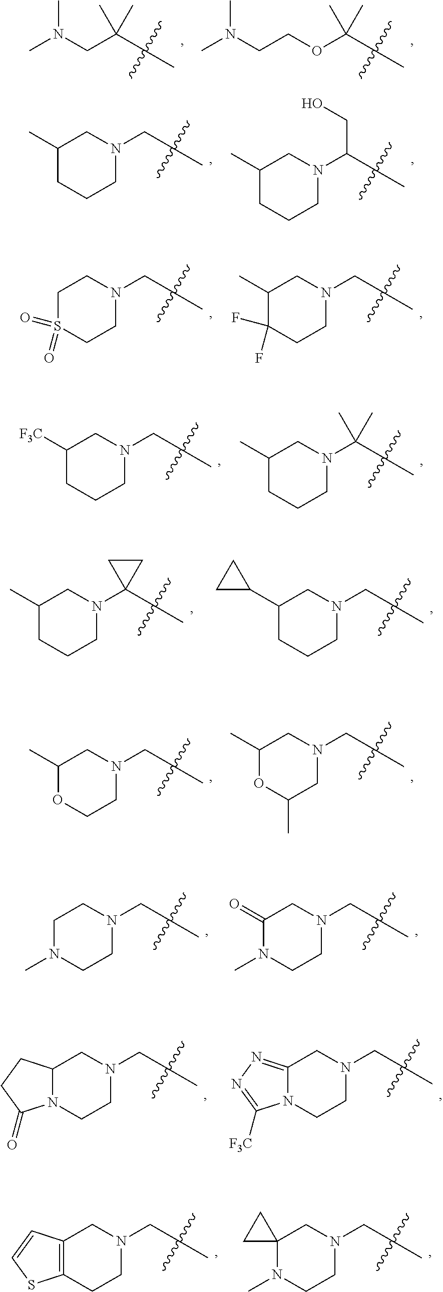

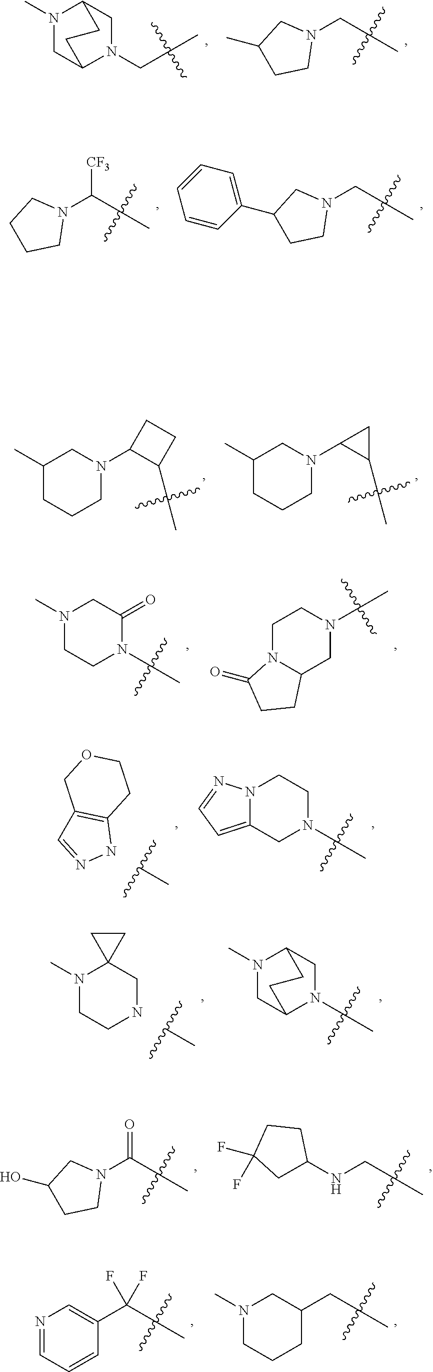

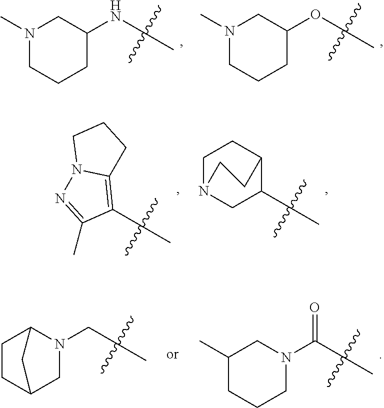

In some embodiments, X is selected from the following groups:

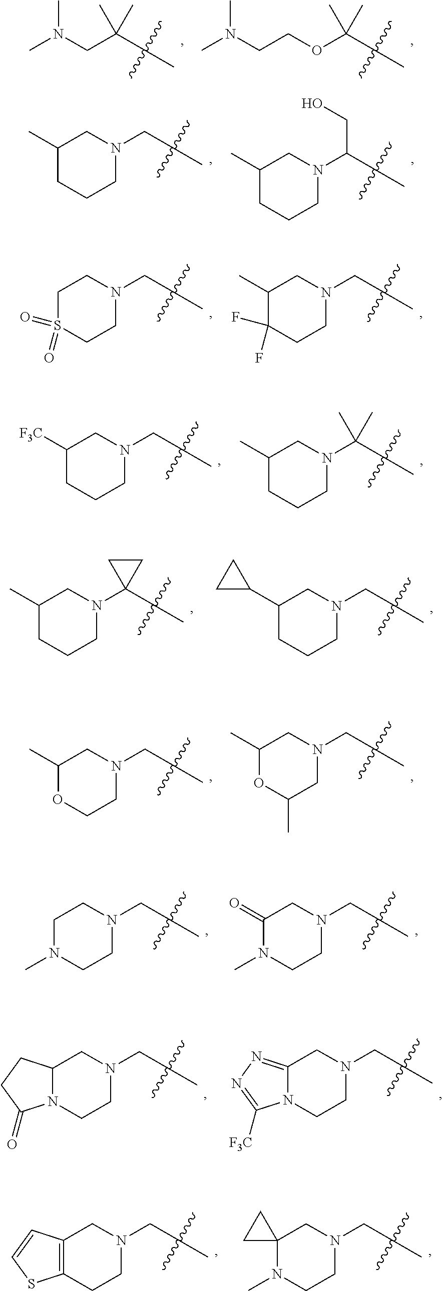

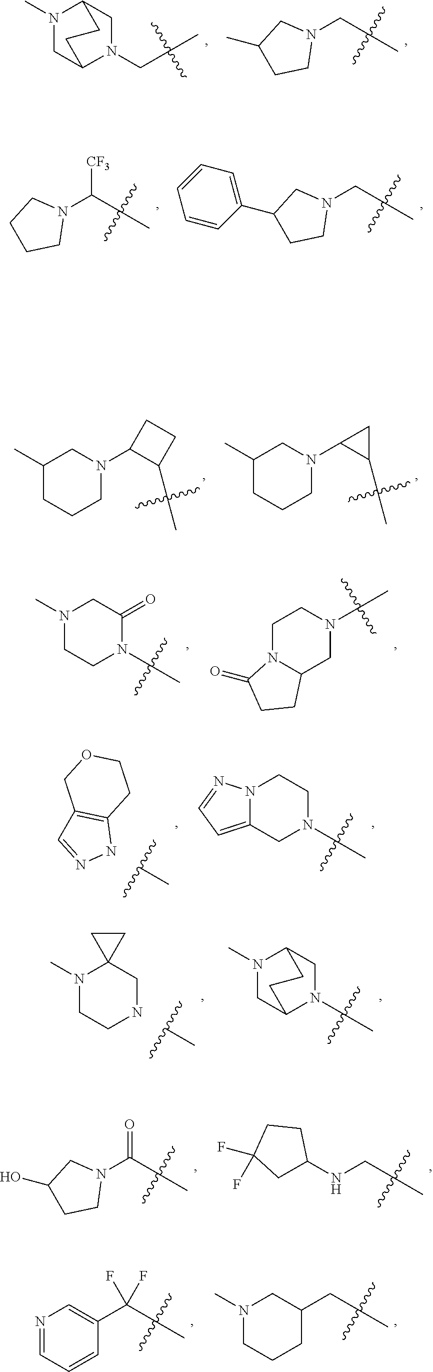

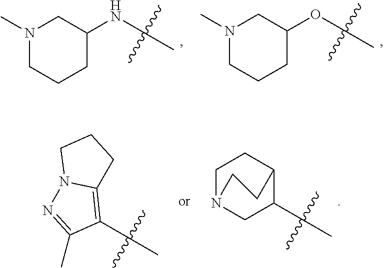

In some embodiments, X is selected from

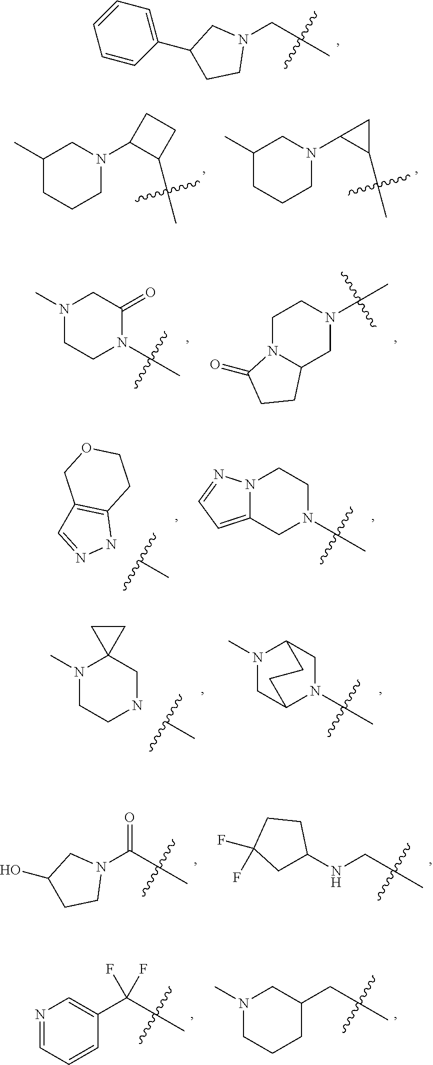

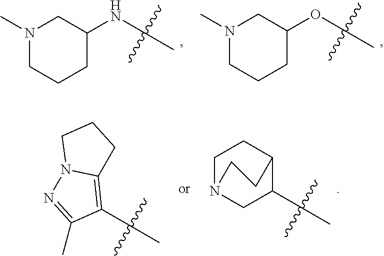

In some embodiments, X is selected from

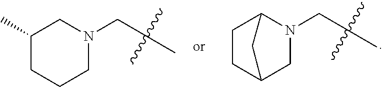

In some embodiments, X is selected from

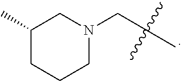 [SMILES: *CN1CCC[C@H](C)C1]

In some embodiments, R¹ and R² together with the atom to which they are attached form C₃-C₈ cycloalkyl or 4- to 10-membered heterocyclyl, wherein the C₃-C₈ cycloalkyl or 4- to 10-membered heterocyclyl is optionally substituted with R^{1b}.

In some embodiments, R¹ and R² together with the atom to which they are attached form C₃-C₆ cycloalkyl or 4- to 7-membered heterocyclyl, wherein the C₃-C₆ cycloalkyl or 4- to 7-membered heterocyclyl is optionally substituted with R^{1b}.

In some embodiments, R¹ and R² together with the atom to which they are attached form the following groups optionally substituted with R^{1b}: cyclobutyl, spiro[2,3]hexyl or oxetanyl.

In some embodiments, R^{1b} is selected from halogen, OH, CN, ═O, NH₂, C₁-C₃ alkyl or C₁-C₃ alkoxy, wherein the C₁-C₃ alkyl or C₁-C₃ alkoxy is optionally substituted with R^{1c}.

In some embodiments, R^{1b} is selected from halogen, CN, C₁-C₃ alkyl or C₁-C₃ alkoxy, wherein the C₁-C₃ alkyl or C₁-C₃ alkoxy is optionally substituted with R^{1c}.

In some embodiments, R^{1c} is selected from halogen, OH or CN.

In some embodiments, R^{1c} is selected from CN.

In some embodiments, R^{1b} is selected from F, CN, methyl, methoxy or CH₂CN.

In some embodiments, R¹ and R² are independently selected from H, halogen, CN, NH₂, NH(C₁-C₆ alkyl), N(C₁-C₆ alkyl)₂, C₁-C₆ alkyl, C₁-C₆ alkoxy, C₃-C₈ cycloalkyl or 4- to 7-membered heterocyclyl, wherein the C₁-C₆ alkyl, C₁-C₆ alkoxy, C₃-C₈ cycloalkyl or 4- to 7-membered heterocyclyl is optionally substituted with R^{1a}, and the C₃-C₈ cycloalkyl or 4- to 7-membered heterocyclyl may exist in the form of a spirocyclic ring, bridged ring or fused ring.

In some embodiments, R¹ and R² are independently selected from H, halogen, CN, C₁-C₃ alkyl, C₁-C₃ alkoxy or C₃-C₆ cycloalkyl, wherein the C₁-C₃ alkyl, C₁-C₃ alkoxy or C₃-C₆ cycloalkyl is optionally substituted with R^{1a}.

In some embodiments, R¹ and R² are independently selected from H, C₁-C₃ alkyl or C₃-C₆ cycloalkyl, wherein the C₁-C₃ alkyl or C₃-C₆ cycloalkyl is optionally substituted with R^{1a}.

In some embodiments, R¹ and R² are independently selected from H or C₁-C₃ alkyl, wherein the C₁-C₃ alkyl is optionally substituted with R^{1a}.

In some embodiments, R^{1a} is selected from halogen, OH, CN, ═O, NH₂, C₁-C₃ alkyl or C₁-C₃ alkoxy.

In some embodiments, R¹ and R² are independently selected from H, F, methyl, CF₃, cyclopropyl, cyclobutyl or

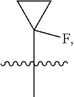 [SMILES: *C1(F)CC1]

or R¹ and R² together with the atom to which they are attached form the following groups:

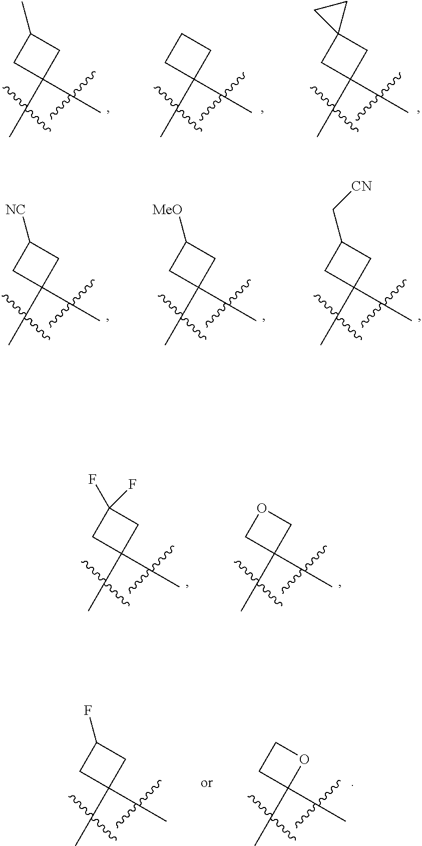

In some embodiments, R¹ and R² are independently selected from H, methyl or cyclobutyl, or R¹ and R² together with the atom to which they are attached form the following groups:

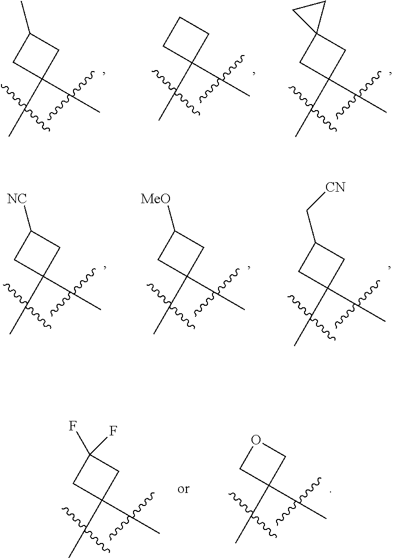

In some embodiments, R¹ and R² are independently selected from H or methyl, or R¹ and R² together with the atom to which they are attached form the following groups:

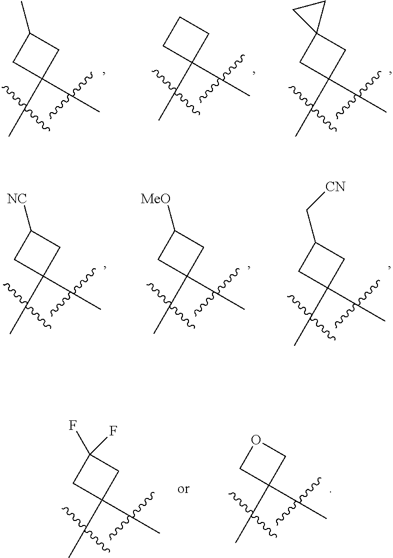

In some embodiments, R¹ and R² are independently selected from H, F, cyclopropyl, methyl, or

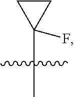 [SMILES: *C1(F)CC1]

or R¹ and R² together with the atom to which they are attached form the following groups:

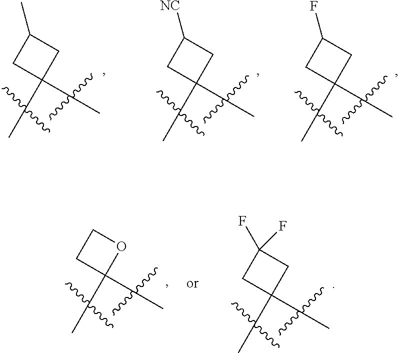

In some embodiments, R¹ and R² together with the atom to which they are attached form the following groups:

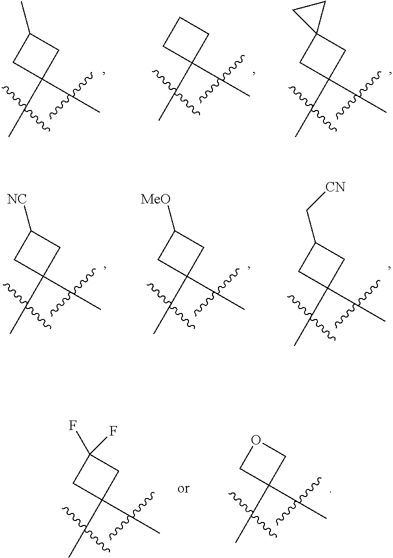

In some embodiments, R¹ and R² together with the atom to which they are attached form the following groups:

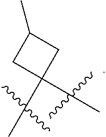 [SMILES: *C1(*)CC(C)C1]

In some embodiments, R¹ and R² are independently selected from H, methyl or cyclobutyl.

In some embodiments, R¹ and R² are independently selected from H or methyl.

In some embodiments, W is selected from —(CR¹³R¹⁴)W₁.

In some embodiments, R¹³ and R¹⁴ are independently selected from H, halogen, OH or methyl.

In some embodiments, R¹³ and R¹⁴ are both H.

In some embodiments, R¹ and R¹³ together with the atom and bond to which they are respectively attached form C₃-C₆ cycloalkyl optionally substituted with R^{13a}.

In some embodiments, R^{13a} is selected from C₁-C₃ alkyl.

In some embodiments, R^{13a} is selected from methyl.

In some embodiments, R¹ and R¹³ together with the atom and bond to which they are respectively attached form

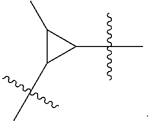 [SMILES: *C1C(*)C1C]

In some embodiments, W is selected from W₁.

In some embodiments, W₁ is selected from 5- to 10-membered heteroaryl or 6- to 10-membered heterocyclyl optionally substituted with R¹⁵.

In some embodiments, W₁ is selected from 5- to 10-membered heteroaryl optionally substituted with R¹⁵.

In some embodiments, W₁ is selected from 5-membered heteroaryl or 8-membered heterocyclyl optionally substituted with R¹⁵.

In some embodiments, W₁ is selected from 5-membered heteroaryl optionally substituted with R¹⁵.

In some embodiments, W₁ is selected from the following groups optionally substituted with R¹⁵: pyrrolyl, thienyl, furyl, pyrazolyl, imidazolyl, thiazolyl, isothiazolyl, thiadiazolyl, triazolyl, oxazolyl, isoxazolyl, oxadiazolyl or 6,7-dihydro-5H-pyrrolo[2,1-c][1,2,4]triazolyl.

In some embodiments, W₁ is selected from the following groups optionally substituted with R¹⁵: pyrrolyl, thienyl, furyl, pyrazolyl, imidazolyl, thiazolyl, isothiazolyl, thiadiazolyl, triazolyl, oxazolyl, isoxazolyl or oxadiazolyl.

In some embodiments, W₁ is selected from the following groups optionally substituted with R¹⁵: triazolyl, oxazolyl, isoxazolyl, oxadiazolyl or 6,7-dihydro-5H-pyrrolo[2,1-c][1,2,4]triazolyl.

In some embodiments, W₁ is selected from the following groups optionally substituted with R¹⁵: triazolyl, oxazolyl, isoxazolyl or oxadiazolyl.

In some embodiments, R¹⁵ is selected from halogen, OH, NH₂, C₁-C₃ alkyl or C₃-C₆ cycloalkyl, wherein the C₁-C₃ alkyl or C₃-C₆ cycloalkyl is optionally substituted with R^{15a}.

In some embodiments, R¹⁵ is selected from OH, C₁-C₃ alkyl or C₃-C₆ cycloalkyl, wherein the C₁-C₃ alkyl or C₃-C₆ cycloalkyl is optionally substituted with R^{15a}.

In some embodiments, R¹⁵ is selected from methyl or cyclopropyl, wherein the methyl or cyclopropyl is optionally substituted with R^{15a}.

In some embodiments, R^{15a} is selected from halogen, OH or CN.

In some embodiments, R^{15a} is selected from F.

In some embodiments, R¹⁵ is selected from methyl, CHF₂ or cyclopropyl.

In some embodiments, W₁ is selected from the following groups:

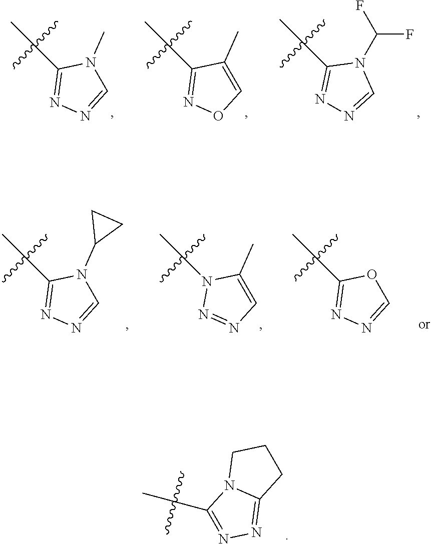

In some embodiments, W₁ is selected from the following groups:

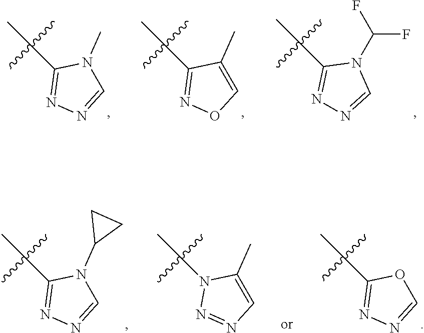

In some embodiments, W is selected from

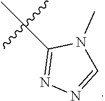 [SMILES: *c1nncn1C]

In some embodiments, the compound of formula (I), or the stereoisomer or pharmaceutically acceptable salt thereof according to the present application is selected from a compound of formula (II), or a stereoisomer or pharmaceutically acceptable salt thereof:

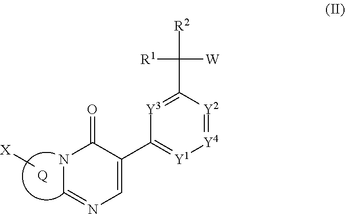

wherein, Y¹, Y², Y³, Y⁴, X, Q, W, R¹ and R² are as defined above.

In some embodiments, the compound of formula (I), or the stereoisomer or pharmaceutically acceptable salt thereof according to the present application is selected from a compound of formula (III), or a stereoisomer or pharmaceutically acceptable salt thereof:

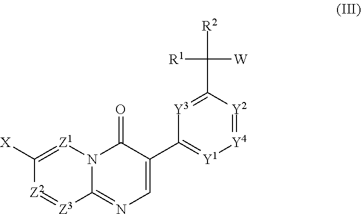

wherein, Z¹, Z² and Z³ are independently selected from CH, CR¹⁰ or N; R¹⁰, Y¹, Y², Y³, Y⁴, X, W, R¹ and R² are as defined above.

In some embodiments, the compound of formula (I), or the stereoisomer or pharmaceutically acceptable salt thereof according to the present application is selected from a compound of formula (IV), or a stereoisomer or pharmaceutically acceptable salt thereof:

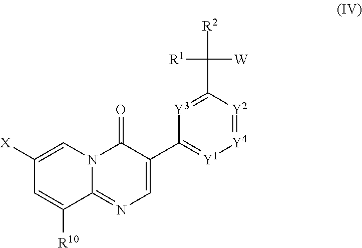

wherein R¹⁰, Y¹, Y², Y³, Y⁴, X, W, R¹ and R² are as defined above.

In some embodiments, the compound of formula (I), or the stereoisomer or pharmaceutically acceptable salt thereof according to the present application is selected from a compound of formula (V), or a stereoisomer or pharmaceutically acceptable salt thereof:

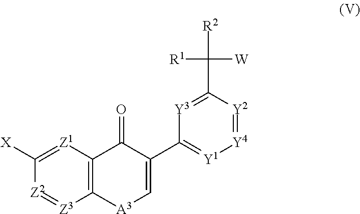

wherein, Z¹, Z² and Z³ are independently selected from CH, CR¹⁰ or N; A³ is selected from CR^{11a}R^{11b}, NR¹², O or S; R¹⁰, R^{11a}, R^{11b}, R¹², Y¹, Y², Y³, Y⁴, X, W, R¹ and R² are as defined above.

In some embodiments, the compound of formula (I), or the stereoisomer or pharmaceutically acceptable salt thereof according to the present application is selected from a compound of formula (VI), or a stereoisomer or pharmaceutically acceptable salt thereof:

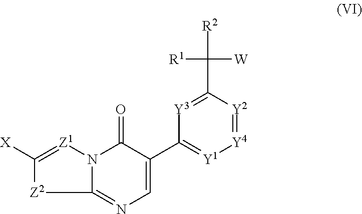

wherein, Z¹ is selected from CH, CR¹⁰ or N; Z² is selected from CH₂, CHR¹⁰, NH or NR¹⁰, O or S; R¹⁰, Y¹, Y², Y³, Y⁴, X, W, R¹ and R² are as defined above.

In some embodiments, the compound of formula (I) or the stereoisomer or pharmaceutically acceptable salt thereof according to the present application is selected from the following compound or a pharmaceutically acceptable salt thereof:

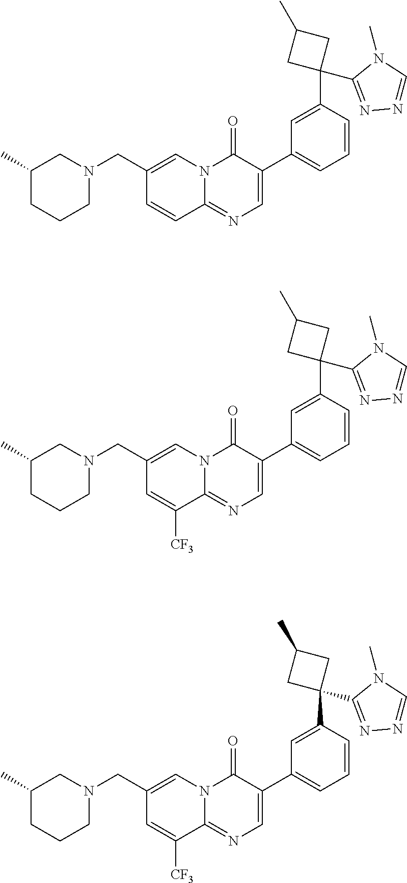 [SMILES: CC1CC(c2cccc(-c3cnc4ccc(CN5CCC[C@H](C)C5)cn4c3=O)c2)(c2nncn2C)C1 ; CC1CC(c2cccc(-c3cnc4c(C(F)(F)F)cc(CN5CCC[C@H](C)C5)cn4c3=O)c2)(c2nncn2C)C1 ; C[C@H]1CCCN(Cc2cc(C(F)(F)F)c3ncc(-c4cccc([C@]5(c6nncn6C)C[C@H](C)C5)c4)c(=O)n3c2)C1]

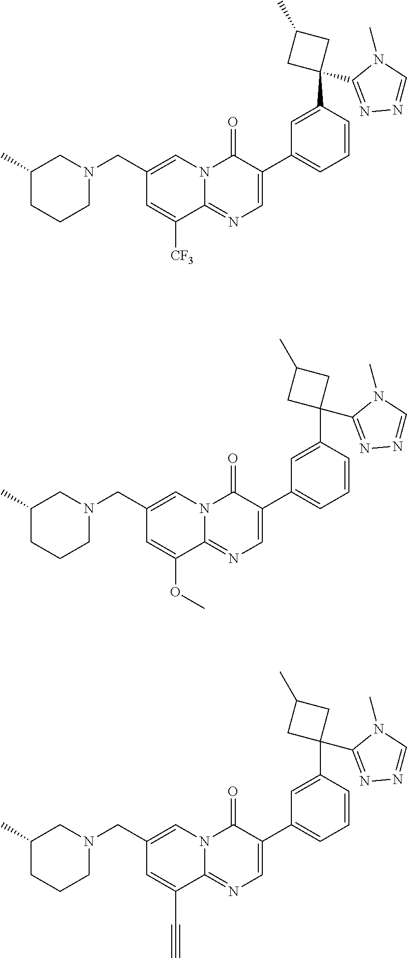 [SMILES: C[C@H]1CCCN(Cc2cc(C(F)(F)F)c3ncc(-c4cccc([C@]5(c6nncn6C)C[C@@H](C)C5)c4)c(=O)n3c2)C1 ; COc1cc(CN2CCC[C@H](C)C2)cn2c(=O)c(-c3cccc(C4(c5nncn5C)CC(C)C4)c3)cnc12 ; C#Cc1cc(CN2CCC[C@H](C)C2)cn2c(=O)c(-c3cccc(C4(c5nncn5C)CC(C)C4)c3)cnc12]

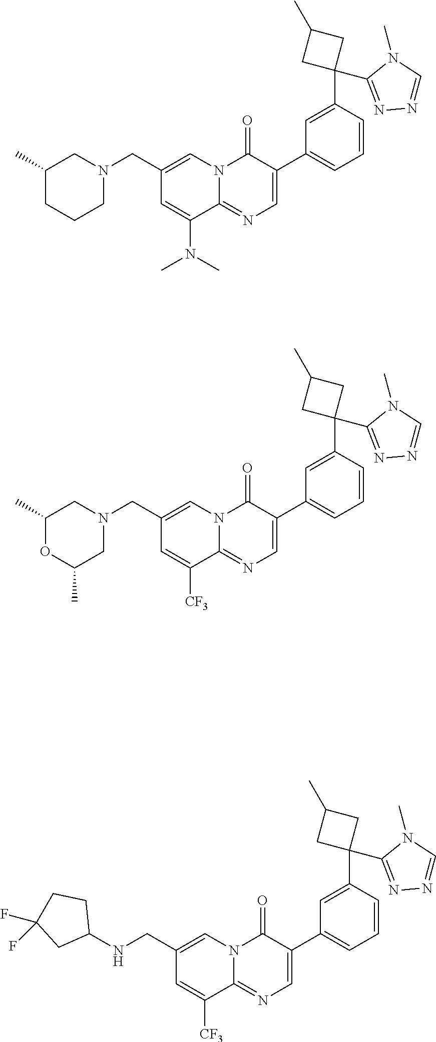 [SMILES: CC1CC(c2cccc(-c3cnc4c(N(C)C)cc(CN5CCC[C@H](C)C5)cn4c3=O)c2)(c2nncn2C)C1 ; CC1CC(c2cccc(-c3cnc4c(C(F)(F)F)cc(CN5C[C@@H](C)O[C@@H](C)C5)cn4c3=O)c2)(c2nncn2C)C1 ; CC1CC(c2cccc(-c3cnc4c(C(F)(F)F)cc(CNC5CCC(F)(F)C5)cn4c3=O)c2)(c2nncn2C)C1]

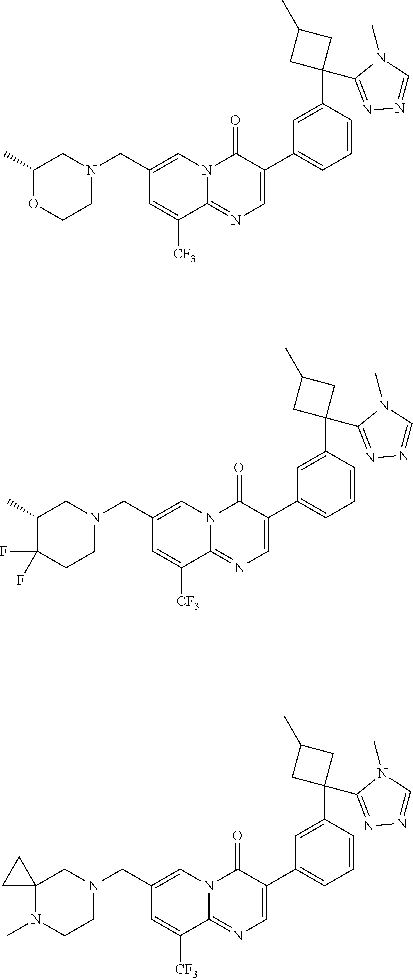 [SMILES: CC1CC(c2cccc(-c3cnc4c(C(F)(F)F)cc(CN5CCO[C@H](C)C5)cn4c3=O)c2)(c2nncn2C)C1 ; CC1CC(c2cccc(-c3cnc4c(C(F)(F)F)cc(CN5CCC(F)(F)[C@H](C)C5)cn4c3=O)c2)(c2nncn2C)C1 ; CC1CC(c2cccc(-c3cnc4c(C(F)(F)F)cc(CN5CCN(C)C6(CC6)C5)cn4c3=O)c2)(c2nncn2C)C1]

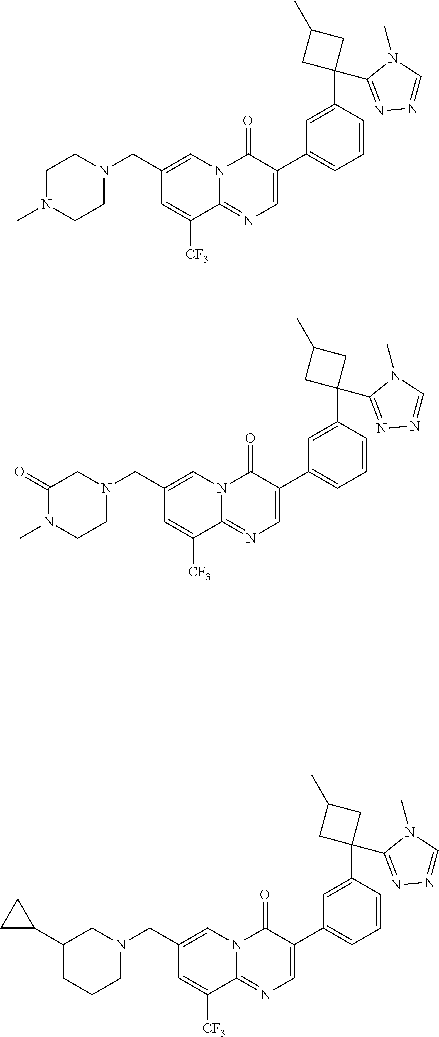 [SMILES: CC1CC(c2cccc(-c3cnc4c(C(F)(F)F)cc(CN5CCN(C)CC5)cn4c3=O)c2)(c2nncn2C)C1 ; CC1CC(c2cccc(-c3cnc4c(C(F)(F)F)cc(CN5CCN(C)C(=O)C5)cn4c3=O)c2)(c2nncn2C)C1 ; CC1CC(c2cccc(-c3cnc4c(C(F)(F)F)cc(CN5CCCC(C6CC6)C5)cn4c3=O)c2)(c2nncn2C)C1]

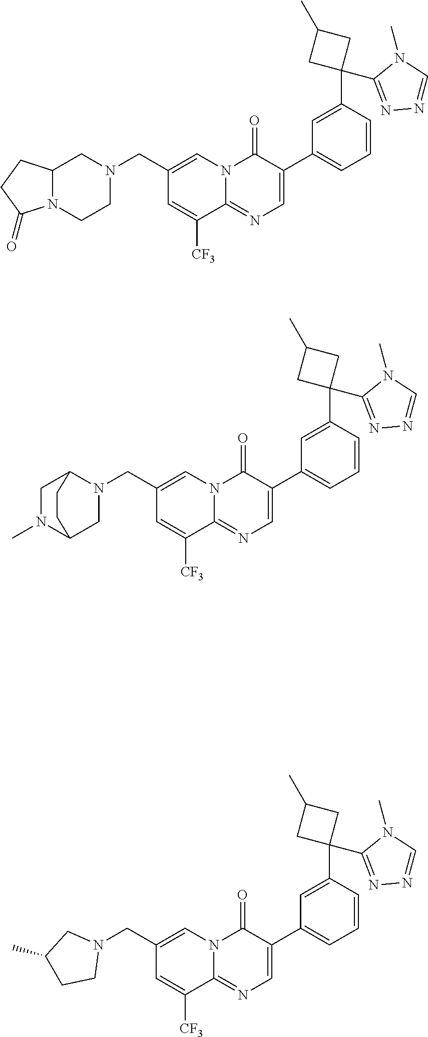 [SMILES: CC1CC(c2cccc(-c3cnc4c(C(F)(F)F)cc(CN5CCN6C(=O)CCC6C5)cn4c3=O)c2)(c2nncn2C)C1 ; CC1CC(c2cccc(-c3cnc4c(C(F)(F)F)cc(CN5CC6CCC5CN6C)cn4c3=O)c2)(c2nncn2C)C1 ; CC1CC(c2cccc(-c3cnc4c(C(F)(F)F)cc(CN5CC[C@H](C)C5)cn4c3=O)c2)(c2nncn2C)C1]

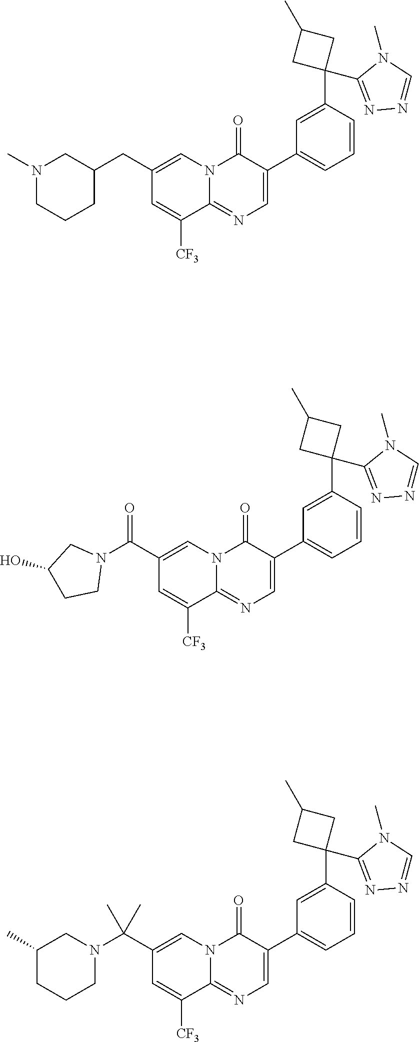 [SMILES: CC1CC(c2cccc(-c3cnc4c(C(F)(F)F)cc(CC5CCCN(C)C5)cn4c3=O)c2)(c2nncn2C)C1 ; CC1CC(c2cccc(-c3cnc4c(C(F)(F)F)cc(C(=O)N5CC[C@H](O)C5)cn4c3=O)c2)(c2nncn2C)C1 ; CC1CC(c2cccc(-c3cnc4c(C(F)(F)F)cc(C(C)(C)N5CCC[C@H](C)C5)cn4c3=O)c2)(c2nncn2C)C1]

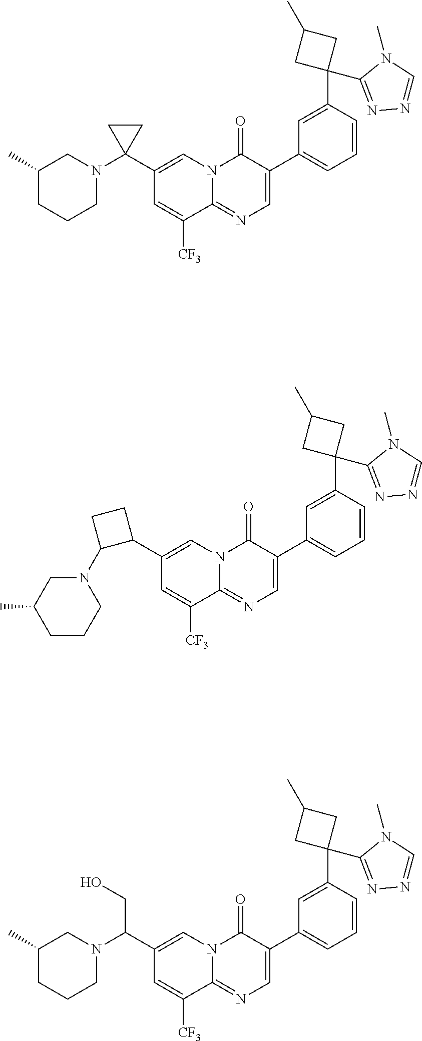 [SMILES: CC1CC(c2cccc(-c3cnc4c(C(F)(F)F)cc(C5(N6CCC[C@H](C)C6)CC5)cn4c3=O)c2)(c2nncn2C)C1 ; CC1CC(c2cccc(-c3cnc4c(C(F)(F)F)cc(C5CCC5N5CCC[C@H](C)C5)cn4c3=O)c2)(c2nncn2C)C1 ; CC1CC(c2cccc(-c3cnc4c(C(F)(F)F)cc(C(CO)N5CCC[C@H](C)C5)cn4c3=O)c2)(c2nncn2C)C1]

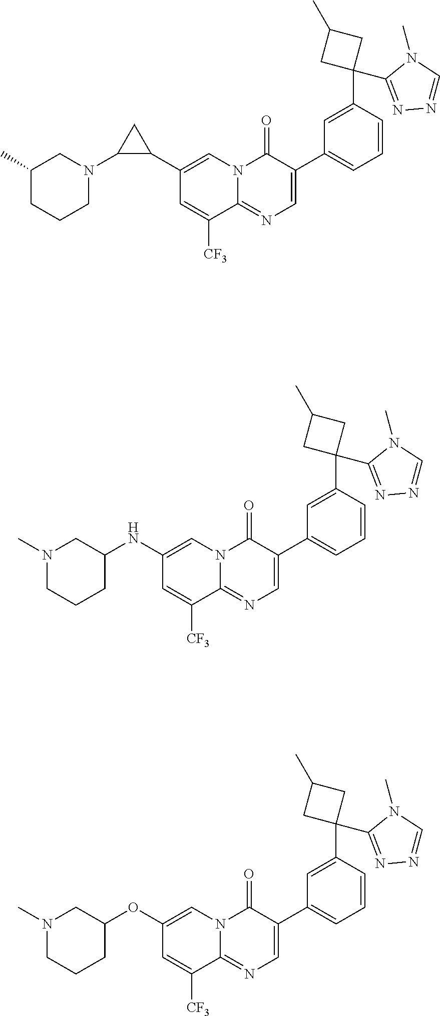 [SMILES: CC1CC(c2cccc(-c3cnc4c(C(F)(F)F)cc(C5CC5N5CCC[C@H](C)C5)cn4c3=O)c2)(c2nncn2C)C1 ; CC1CC(c2cccc(-c3cnc4c(C(F)(F)F)cc(NC5CCCN(C)C5)cn4c3=O)c2)(c2nncn2C)C1 ; CC1CC(c2cccc(-c3cnc4c(C(F)(F)F)cc(OC5CCCN(C)C5)cn4c3=O)c2)(c2nncn2C)C1]

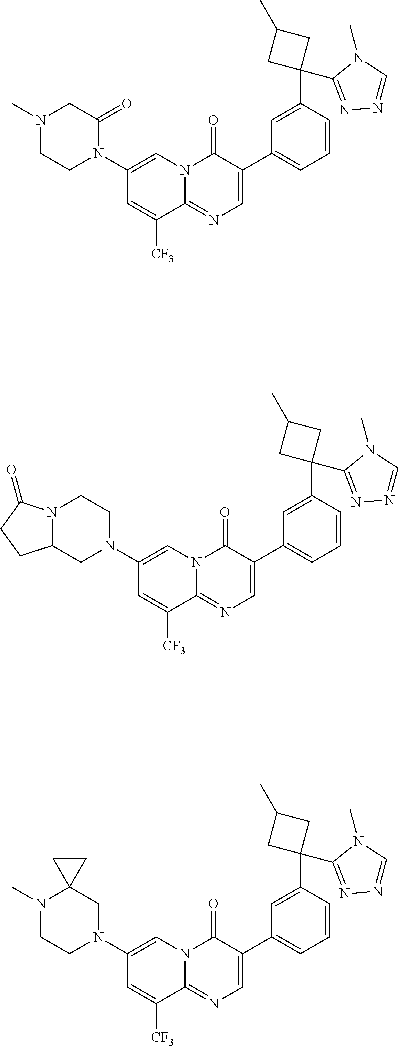 [SMILES: CC1CC(c2cccc(-c3cnc4c(C(F)(F)F)cc(N5CCN(C)CC5=O)cn4c3=O)c2)(c2nncn2C)C1 ; CC1CC(c2cccc(-c3cnc4c(C(F)(F)F)cc(N5CCN6C(=O)CCC6C5)cn4c3=O)c2)(c2nncn2C)C1 ; CC1CC(c2cccc(-c3cnc4c(C(F)(F)F)cc(N5CCN(C)C6(CC6)C5)cn4c3=O)c2)(c2nncn2C)C1]

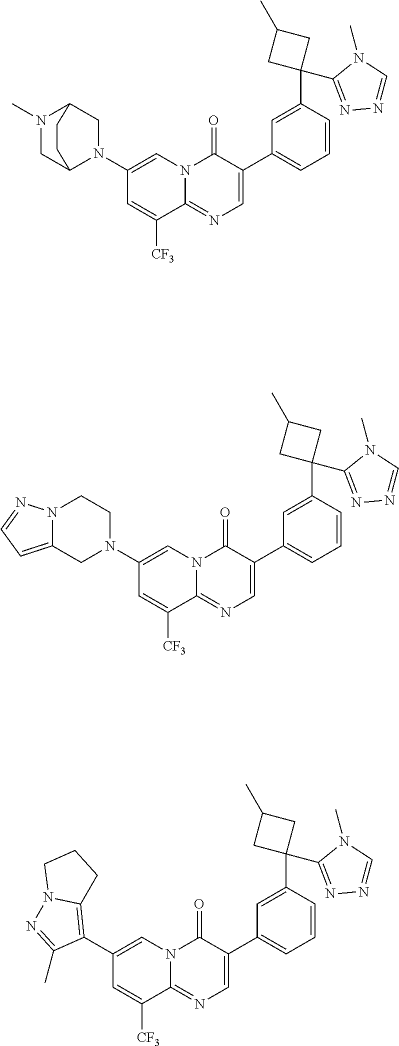 [SMILES: CC1CC(c2cccc(-c3cnc4c(C(F)(F)F)cc(N5CC6CCC5CN6C)cn4c3=O)c2)(c2nncn2C)C1 ; CC1CC(c2cccc(-c3cnc4c(C(F)(F)F)cc(N5CCn6nccc6C5)cn4c3=O)c2)(c2nncn2C)C1 ; Cc1nn2c(c1-c1cc(C(F)(F)F)c3ncc(-c4cccc(C5(c6nncn6C)CC(C)C5)c4)c(=O)n3c1)CCC2]

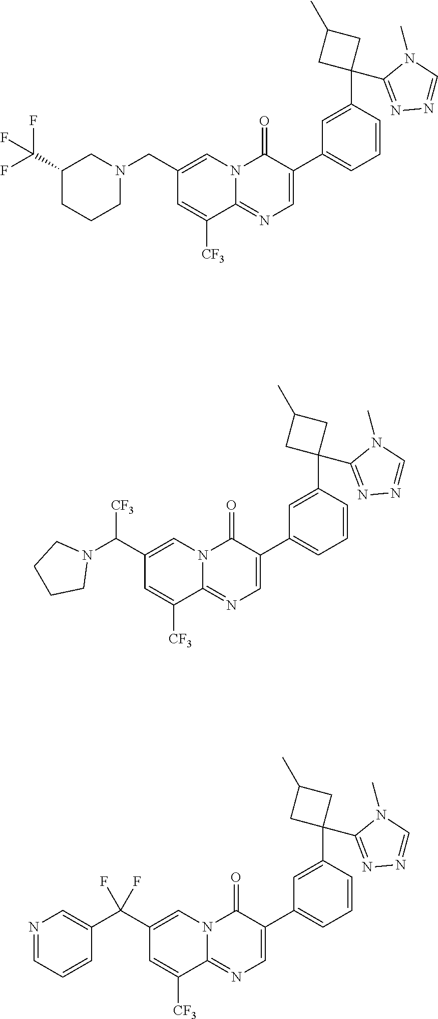 [SMILES: CC1CC(c2cccc(-c3cnc4c(C(F)(F)F)cc(CN5CCC[C@H](C(F)(F)F)C5)cn4c3=O)c2)(c2nncn2C)C1 ; CC1CC(c2cccc(-c3cnc4c(C(F)(F)F)cc(C(N5CCCC5)C(F)(F)F)cn4c3=O)c2)(c2nncn2C)C1 ; CC1CC(c2cccc(-c3cnc4c(C(F)(F)F)cc(C(F)(F)c5cccnc5)cn4c3=O)c2)(c2nncn2C)C1]

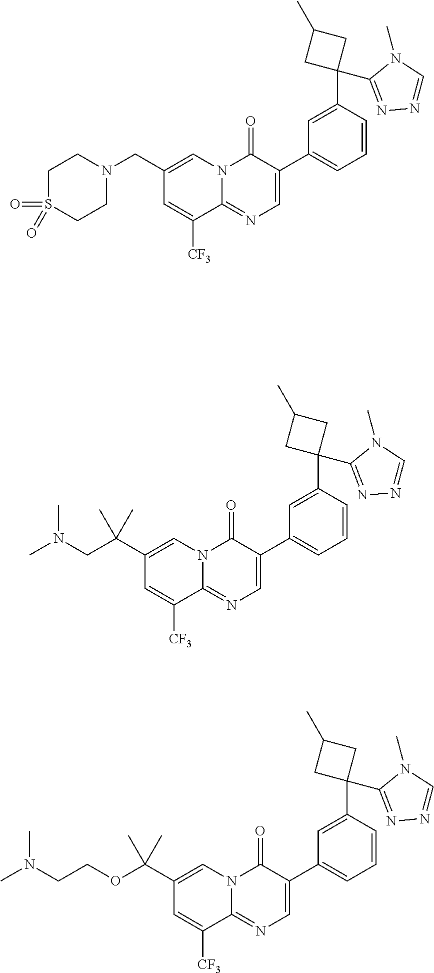 [SMILES: CC1CC(c2cccc(-c3cnc4c(C(F)(F)F)cc(CN5CCS(=O)(=O)CC5)cn4c3=O)c2)(c2nncn2C)C1 ; CC1CC(c2cccc(-c3cnc4c(C(F)(F)F)cc(C(C)(C)CN(C)C)cn4c3=O)c2)(c2nncn2C)C1 ; CC1CC(c2cccc(-c3cnc4c(C(F)(F)F)cc(C(C)(C)OCCN(C)C)cn4c3=O)c2)(c2nncn2C)C1]

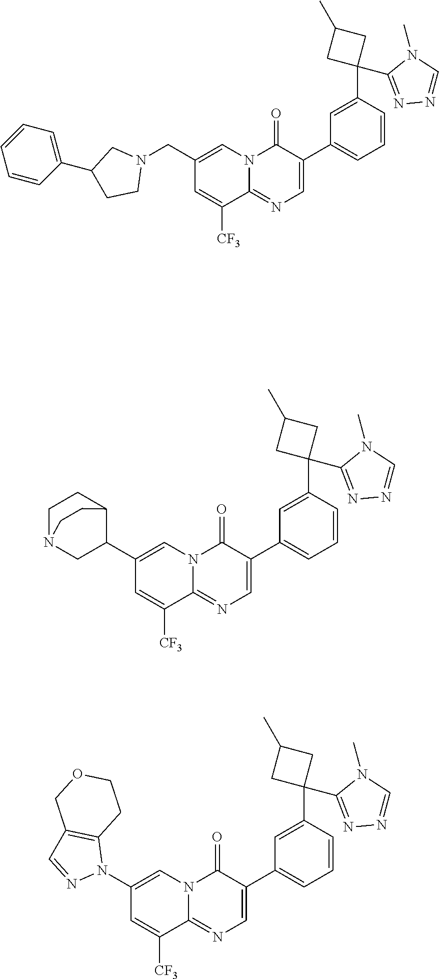 [SMILES: CC1CC(c2cccc(-c3cnc4c(C(F)(F)F)cc(CN5CCC(c6ccccc6)C5)cn4c3=O)c2)(c2nncn2C)C1 ; CC1CC(c2cccc(-c3cnc4c(C(F)(F)F)cc(C5CN6CCC5CC6)cn4c3=O)c2)(c2nncn2C)C1 ; CC1CC(c2cccc(-c3cnc4c(C(F)(F)F)cc(-n5ncc6c5CCOC6)cn4c3=O)c2)(c2nncn2C)C1]

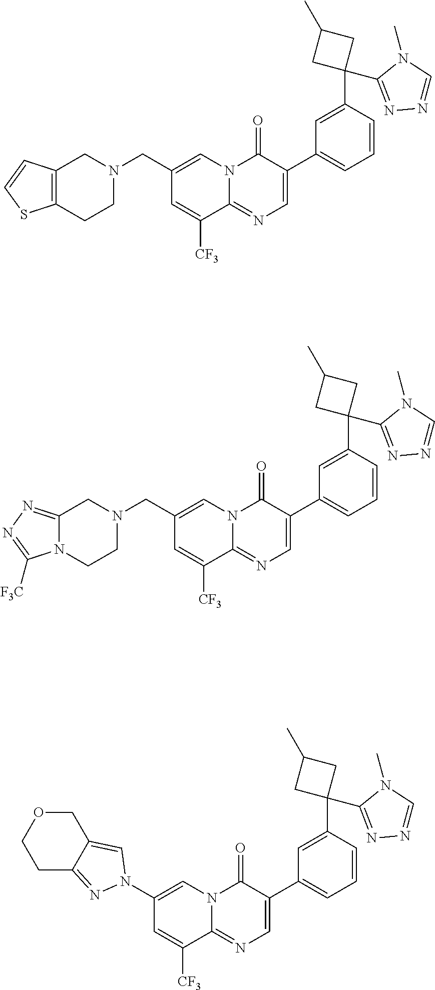 [SMILES: CC1CC(c2cccc(-c3cnc4c(C(F)(F)F)cc(CN5CCc6sccc6C5)cn4c3=O)c2)(c2nncn2C)C1 ; CC1CC(c2cccc(-c3cnc4c(C(F)(F)F)cc(CN5CCn6c(nnc6C(F)(F)F)C5)cn4c3=O)c2)(c2nncn2C)C1 ; CC1CC(c2cccc(-c3cnc4c(C(F)(F)F)cc(-n5cc6c(n5)CCOC6)cn4c3=O)c2)(c2nncn2C)C1]

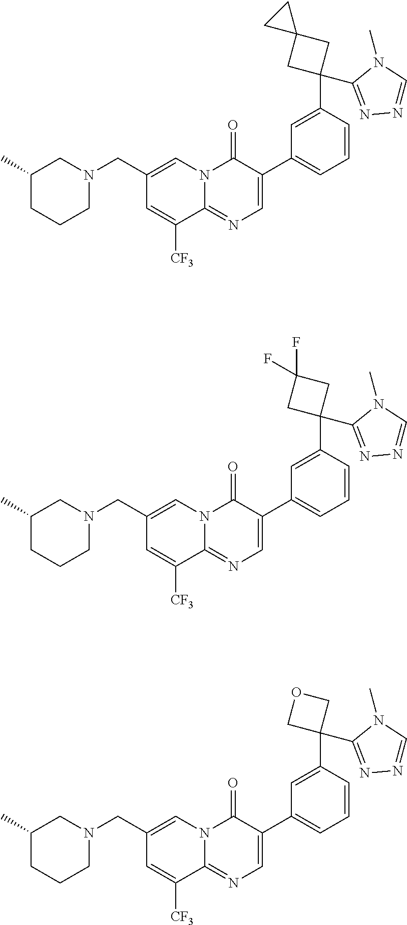 [SMILES: C[C@H]1CCCN(Cc2cc(C(F)(F)F)c3ncc(-c4cccc(C5(c6nncn6C)CC6(CC6)C5)c4)c(=O)n3c2)C1 ; C[C@H]1CCCN(Cc2cc(C(F)(F)F)c3ncc(-c4cccc(C5(c6nncn6C)CC(F)(F)C5)c4)c(=O)n3c2)C1 ; C[C@H]1CCCN(Cc2cc(C(F)(F)F)c3ncc(-c4cccc(C5(c6nncn6C)COC5)c4)c(=O)n3c2)C1]

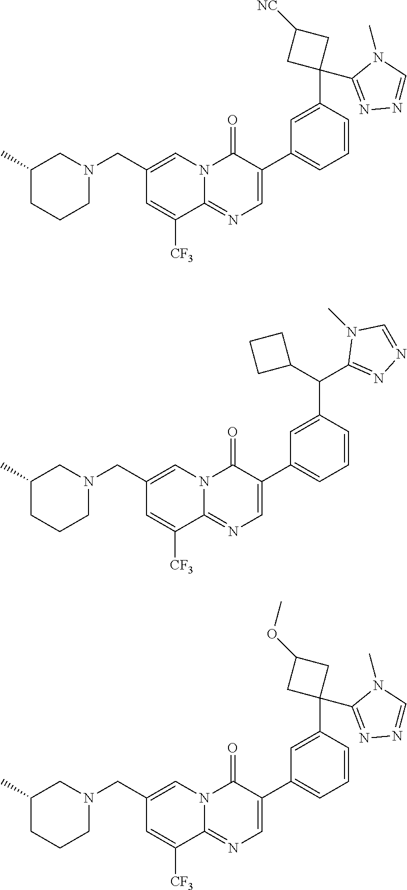 [SMILES: C[C@H]1CCCN(Cc2cc(C(F)(F)F)c3ncc(-c4cccc(C5(c6nncn6C)CC(C#N)C5)c4)c(=O)n3c2)C1 ; C[C@H]1CCCN(Cc2cc(C(F)(F)F)c3ncc(-c4cccc(C(c5nncn5C)C5CCC5)c4)c(=O)n3c2)C1 ; COC1CC(c2cccc(-c3cnc4c(C(F)(F)F)cc(CN5CCC[C@H](C)C5)cn4c3=O)c2)(c2nncn2C)C1]

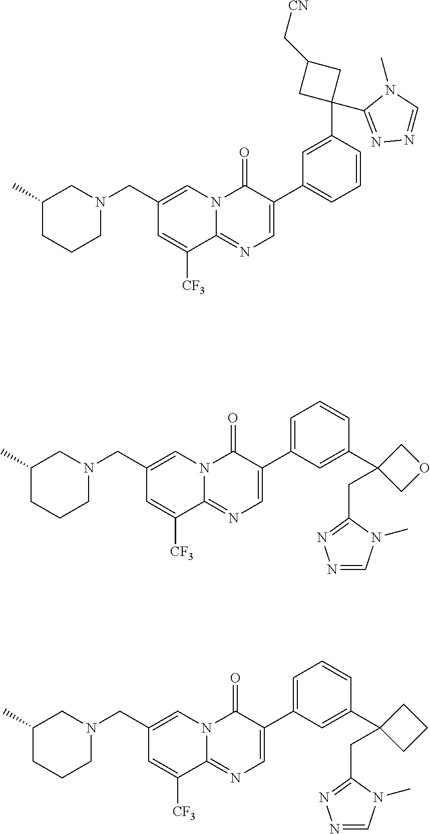 [SMILES: C[C@H]1CCCN(Cc2cc(C(F)(F)F)c3ncc(-c4cccc(C5(c6nncn6C)CC(CC#N)C5)c4)c(=O)n3c2)C1 ; C[C@H]1CCCN(Cc2cc(C(F)(F)F)c3ncc(-c4cccc(C5(Cc6nncn6C)COC5)c4)c(=O)n3c2)C1 ; C[C@H]1CCCN(Cc2cc(C(F)(F)F)c3ncc(-c4cccc(C5(Cc6nncn6C)CCC5)c4)c(=O)n3c2)C1]

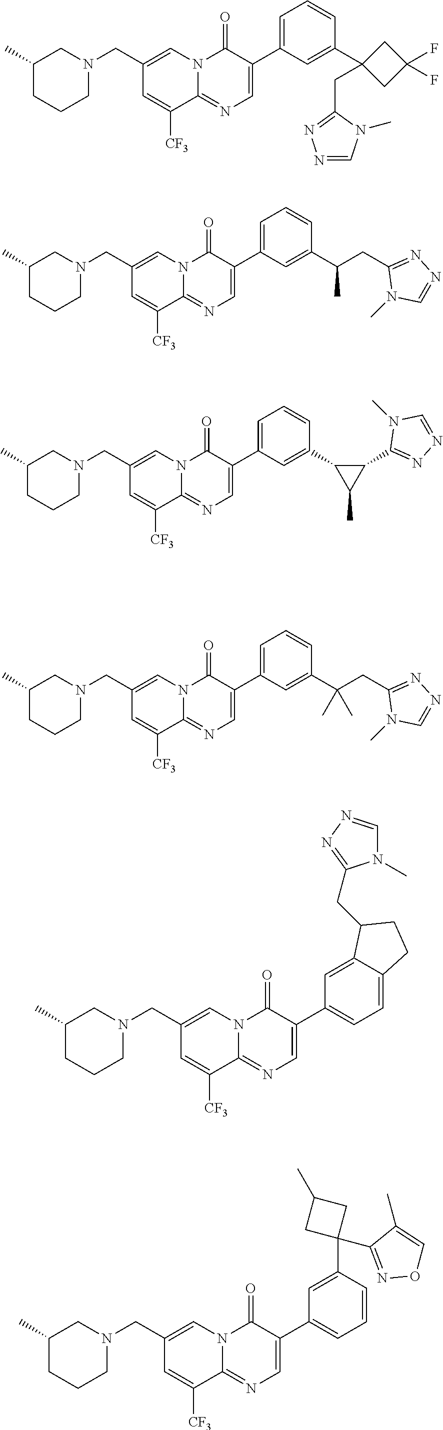 [SMILES: C[C@H]1CCCN(Cc2cc(C(F)(F)F)c3ncc(-c4cccc(C5(Cc6nncn6C)CC(F)(F)C5)c4)c(=O)n3c2)C1 ; C[C@H]1CCCN(Cc2cc(C(F)(F)F)c3ncc(-c4cccc([C@H](C)Cc5nncn5C)c4)c(=O)n3c2)C1 ; C[C@H]1CCCN(Cc2cc(C(F)(F)F)c3ncc(-c4cccc([C@H]5[C@H](C)[C@H]5c5nncn5C)c4)c(=O)n3c2)C1 ; C[C@H]1CCCN(Cc2cc(C(F)(F)F)c3ncc(-c4cccc(C(C)(C)Cc5nncn5C)c4)c(=O)n3c2)C1 ; C[C@H]1CCCN(Cc2cc(C(F)(F)F)c3ncc(-c4ccc5c(c4)C(Cc4nncn4C)CC5)c(=O)n3c2)C1 ; Cc1conc1C1(c2cccc(-c3cnc4c(C(F)(F)F)cc(CN5CCC[C@H](C)C5)cn4c3=O)c2)CC(C)C1]

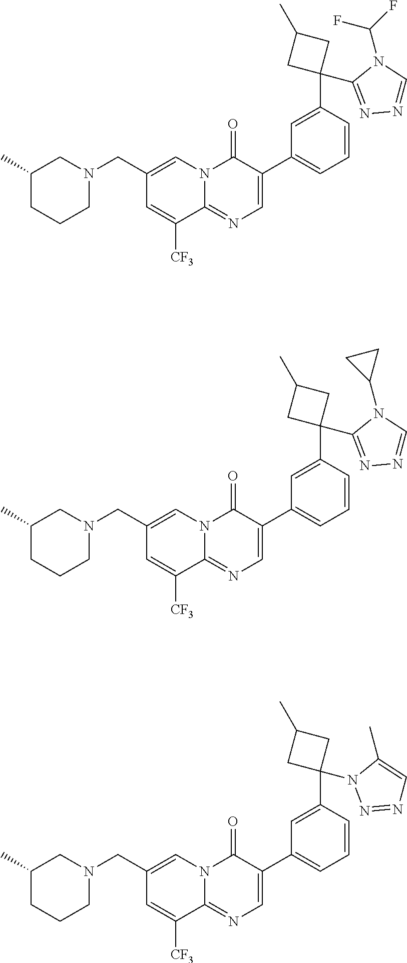 [SMILES: CC1CC(c2cccc(-c3cnc4c(C(F)(F)F)cc(CN5CCC[C@H](C)C5)cn4c3=O)c2)(c2nncn2C(F)F)C1 ; CC1CC(c2cccc(-c3cnc4c(C(F)(F)F)cc(CN5CCC[C@H](C)C5)cn4c3=O)c2)(c2nncn2C2CC2)C1 ; Cc1cnnn1C1(c2cccc(-c3cnc4c(C(F)(F)F)cc(CN5CCC[C@H](C)C5)cn4c3=O)c2)CC(C)C1]

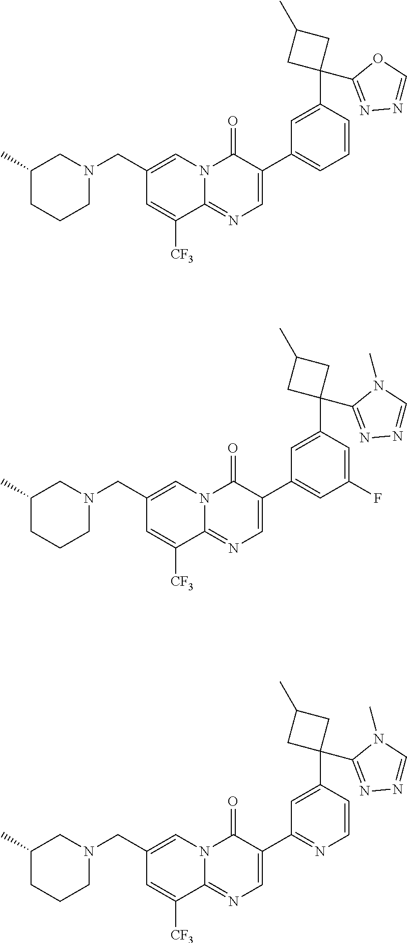 [SMILES: CC1CC(c2cccc(-c3cnc4c(C(F)(F)F)cc(CN5CCC[C@H](C)C5)cn4c3=O)c2)(c2nnco2)C1 ; CC1CC(c2cc(F)cc(-c3cnc4c(C(F)(F)F)cc(CN5CCC[C@H](C)C5)cn4c3=O)c2)(c2nncn2C)C1 ; CC1CC(c2ccnc(-c3cnc4c(C(F)(F)F)cc(CN5CCC[C@H](C)C5)cn4c3=O)c2)(c2nncn2C)C1]

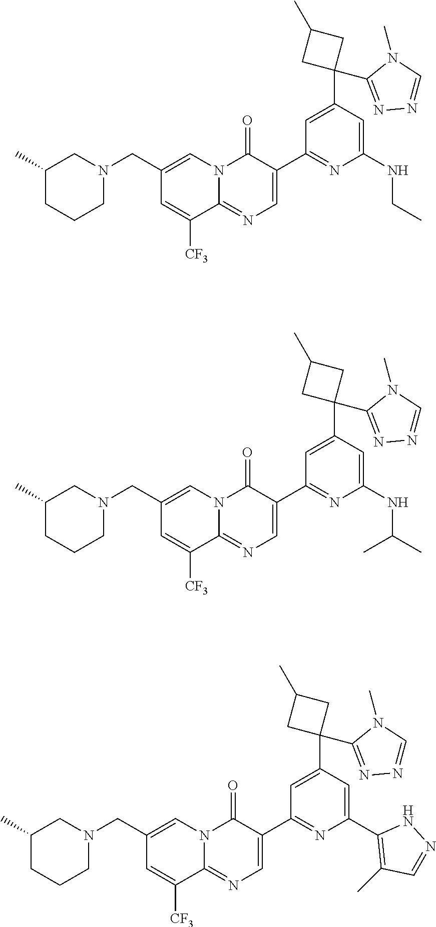

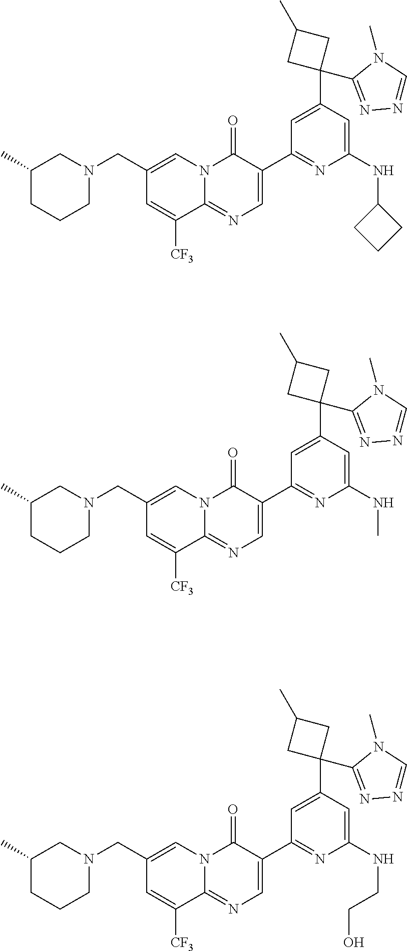 [SMILES: CC1CC(c2cc(NC3CCC3)nc(-c3cnc4c(C(F)(F)F)cc(CN5CCC[C@H](C)C5)cn4c3=O)c2)(c2nncn2C)C1 ; CNc1cc(C2(c3nncn3C)CC(C)C2)cc(-c2cnc3c(C(F)(F)F)cc(CN4CCC[C@H](C)C4)cn3c2=O)n1 ; CC1CC(c2cc(NCCO)nc(-c3cnc4c(C(F)(F)F)cc(CN5CCC[C@H](C)C5)cn4c3=O)c2)(c2nncn2C)C1]

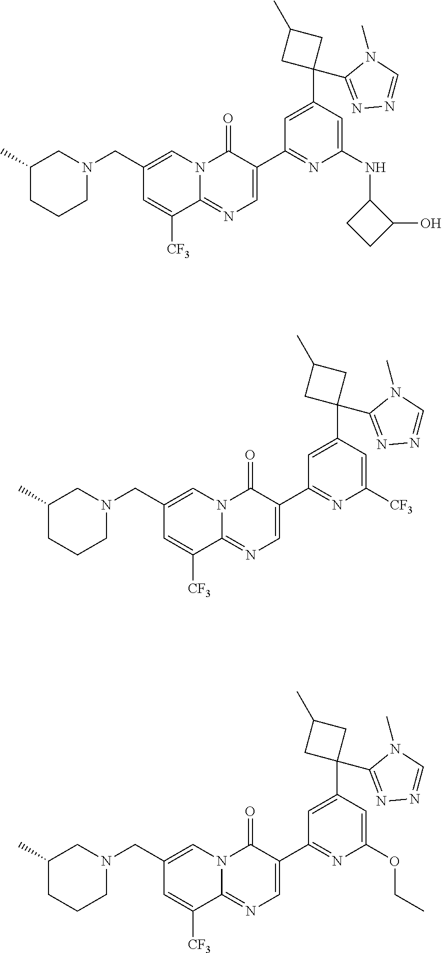 [SMILES: CC1CC(c2cc(NC3CCC3O)nc(-c3cnc4c(C(F)(F)F)cc(CN5CCC[C@H](C)C5)cn4c3=O)c2)(c2nncn2C)C1 ; CC1CC(c2cc(-c3cnc4c(C(F)(F)F)cc(CN5CCC[C@H](C)C5)cn4c3=O)nc(C(F)(F)F)c2)(c2nncn2C)C1 ; CCOc1cc(C2(c3nncn3C)CC(C)C2)cc(-c2cnc3c(C(F)(F)F)cc(CN4CCC[C@H](C)C4)cn3c2=O)n1]

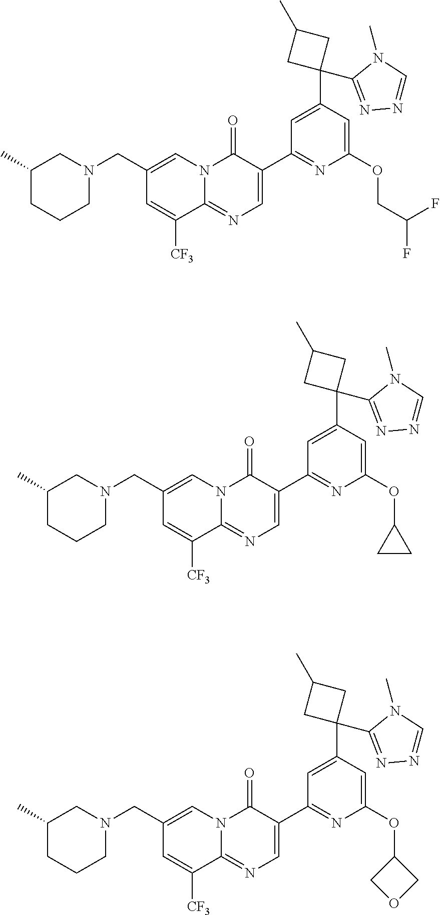 [SMILES: CC1CC(c2cc(OCC(F)F)nc(-c3cnc4c(C(F)(F)F)cc(CN5CCC[C@H](C)C5)cn4c3=O)c2)(c2nncn2C)C1 ; CC1CC(c2cc(OC3CC3)nc(-c3cnc4c(C(F)(F)F)cc(CN5CCC[C@H](C)C5)cn4c3=O)c2)(c2nncn2C)C1 ; CC1CC(c2cc(OC3COC3)nc(-c3cnc4c(C(F)(F)F)cc(CN5CCC[C@H](C)C5)cn4c3=O)c2)(c2nncn2C)C1]

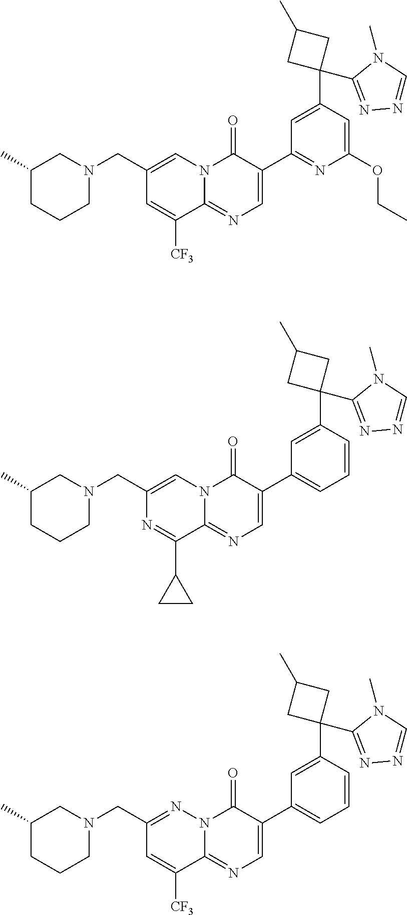

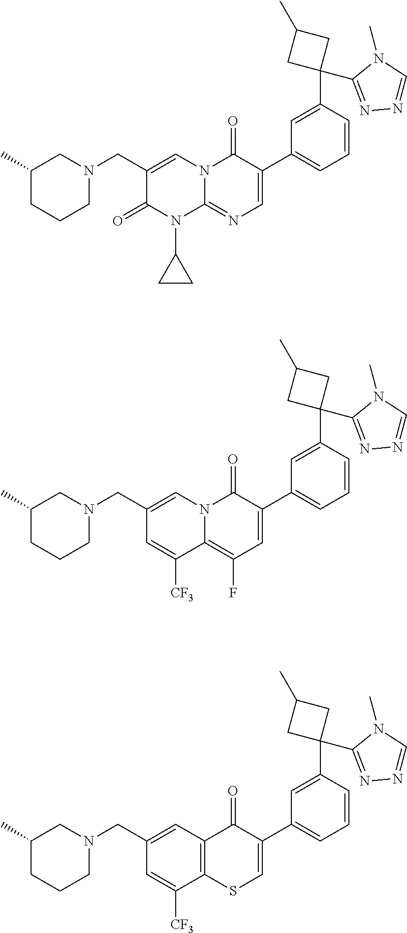 [SMILES: CC1CC(c2cccc(-c3cnc4n(C5CC5)c(=O)c(CN5CCC[C@H](C)C5)cn4c3=O)c2)(c2nncn2C)C1 ; CC1CC(c2cccc(-c3cc(F)c4c(C(F)(F)F)cc(CN5CCC[C@H](C)C5)cn4c3=O)c2)(c2nncn2C)C1 ; CC1CC(c2cccc(-c3csc4c(C(F)(F)F)cc(CN5CCC[C@H](C)C5)cc4c3=O)c2)(c2nncn2C)C1]

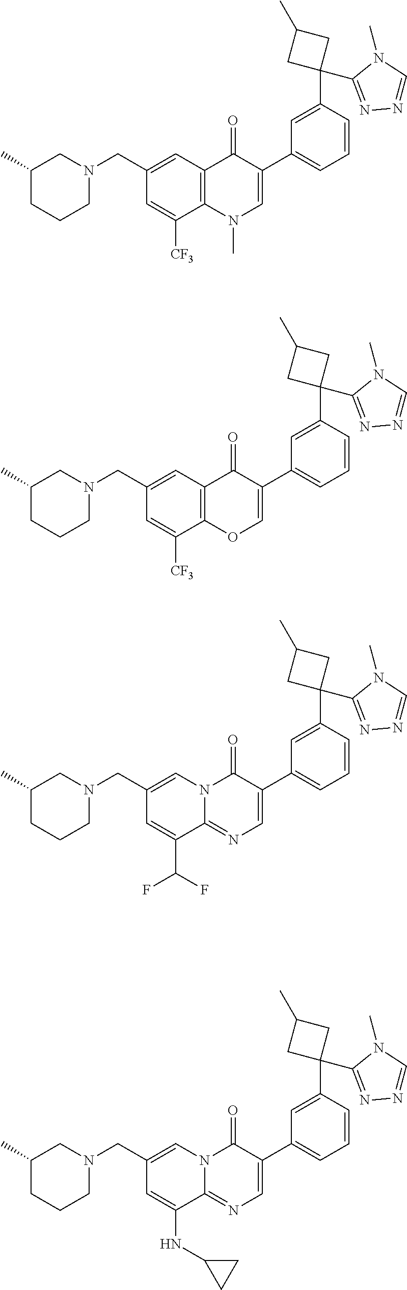 [SMILES: CC1CC(c2cccc(-c3cn(C)c4c(C(F)(F)F)cc(CN5CCC[C@H](C)C5)cc4c3=O)c2)(c2nncn2C)C1 ; CC1CC(c2cccc(-c3coc4c(C(F)(F)F)cc(CN5CCC[C@H](C)C5)cc4c3=O)c2)(c2nncn2C)C1 ; CC1CC(c2cccc(-c3cnc4c(C(F)F)cc(CN5CCC[C@H](C)C5)cn4c3=O)c2)(c2nncn2C)C1 ; CC1CC(c2cccc(-c3cnc4c(NC5CC5)cc(CN5CCC[C@H](C)C5)cn4c3=O)c2)(c2nncn2C)C1]

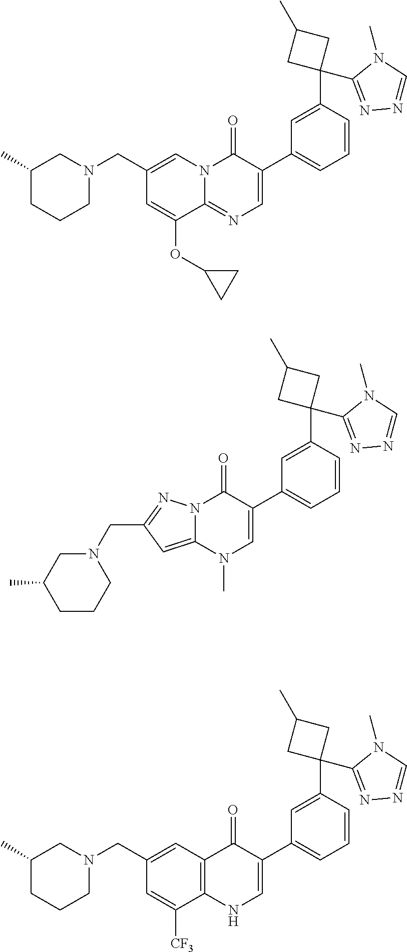 [SMILES: CC1CC(c2cccc(-c3cnc4c(OC5CC5)cc(CN5CCC[C@H](C)C5)cn4c3=O)c2)(c2nncn2C)C1 ; CC1CC(c2cccc(-c3cn(C)c4cc(CN5CCC[C@H](C)C5)nn4c3=O)c2)(c2nncn2C)C1 ; CC1CC(c2cccc(-c3c[nH]c4c(C(F)(F)F)cc(CN5CCC[C@H](C)C5)cc4c3=O)c2)(c2nncn2C)C1]

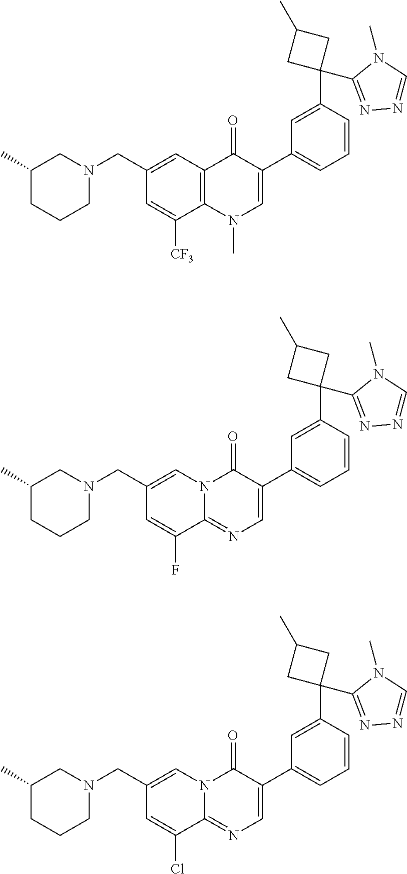 [SMILES: CC1CC(c2cccc(-c3cn(C)c4c(C(F)(F)F)cc(CN5CCC[C@H](C)C5)cc4c3=O)c2)(c2nncn2C)C1 ; CC1CC(c2cccc(-c3cnc4c(F)cc(CN5CCC[C@H](C)C5)cn4c3=O)c2)(c2nncn2C)C1 ; CC1CC(c2cccc(-c3cnc4c(Cl)cc(CN5CCC[C@H](C)C5)cn4c3=O)c2)(c2nncn2C)C1]

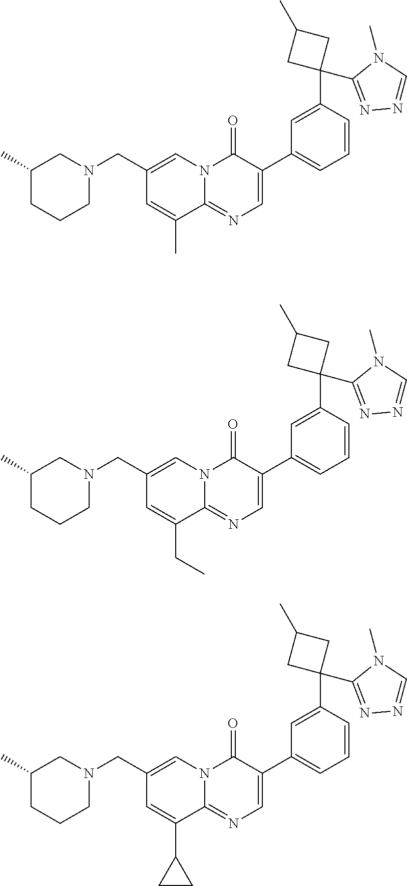 [SMILES: Cc1cc(CN2CCC[C@H](C)C2)cn2c(=O)c(-c3cccc(C4(c5nncn5C)CC(C)C4)c3)cnc12 ; CCc1cc(CN2CCC[C@H](C)C2)cn2c(=O)c(-c3cccc(C4(c5nncn5C)CC(C)C4)c3)cnc12 ; CC1CC(c2cccc(-c3cnc4c(C5CC5)cc(CN5CCC[C@H](C)C5)cn4c3=O)c2)(c2nncn2C)C1]

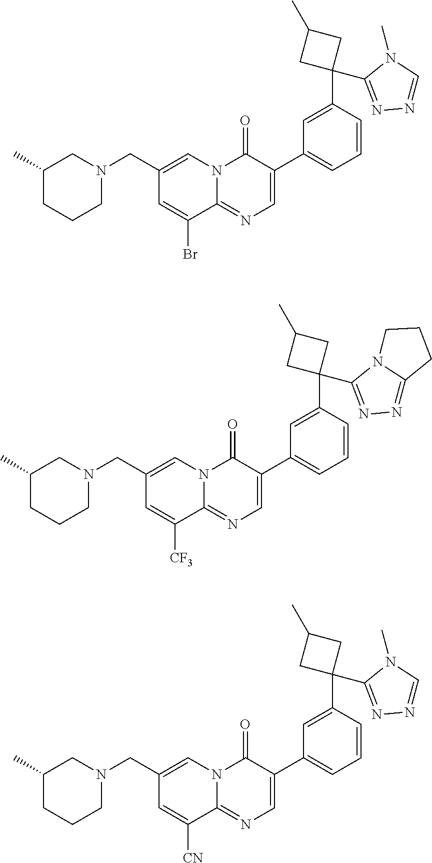 [SMILES: CC1CC(c2cccc(-c3cnc4c(Br)cc(CN5CCC[C@H](C)C5)cn4c3=O)c2)(c2nncn2C)C1 ; CC1CC(c2cccc(-c3cnc4c(C(F)(F)F)cc(CN5CCC[C@H](C)C5)cn4c3=O)c2)(c2nnc3n2CCC3)C1 ; CC1CC(c2cccc(-c3cnc4c(C#N)cc(CN5CCC[C@H](C)C5)cn4c3=O)c2)(c2nncn2C)C1]

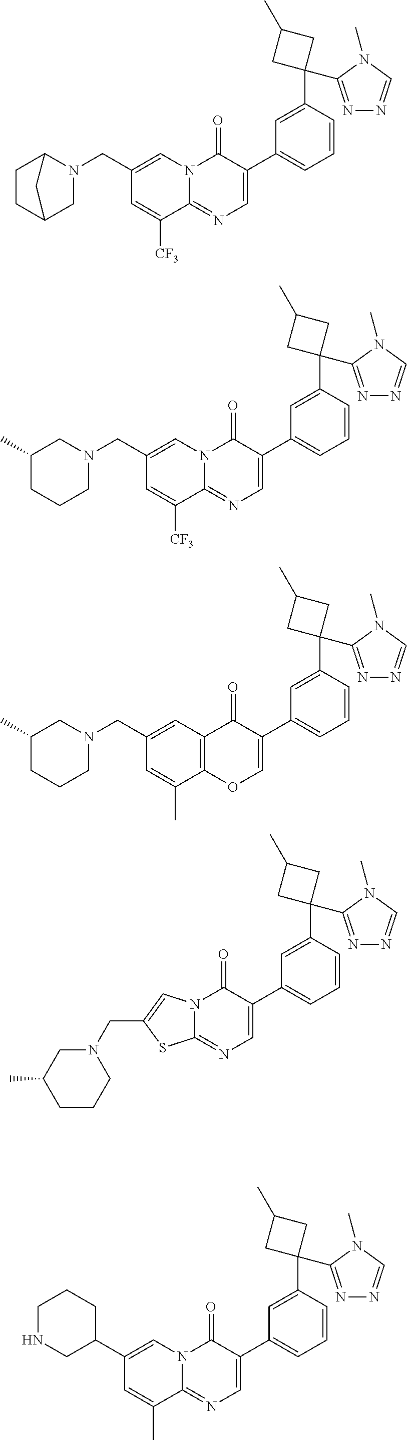 [SMILES: CC1CC(c2cccc(-c3cnc4c(C(F)(F)F)cc(CN5CC6CCC5C6)cn4c3=O)c2)(c2nncn2C)C1 ; CC1CC(c2cccc(-c3cnc4c(C(F)(F)F)cc(CN5CCC[C@H](C)C5)cn4c3=O)c2)(c2nncn2C)C1 ; Cc1cc(CN2CCC[C@H](C)C2)cc2c(=O)c(-c3cccc(C4(c5nncn5C)CC(C)C4)c3)coc12 ; CC1CC(c2cccc(-c3cnc4sc(CN5CCC[C@H](C)C5)cn4c3=O)c2)(c2nncn2C)C1 ; Cc1cc(C2CCCNC2)cn2c(=O)c(-c3cccc(C4(c5nncn5C)CC(C)C4)c3)cnc12]

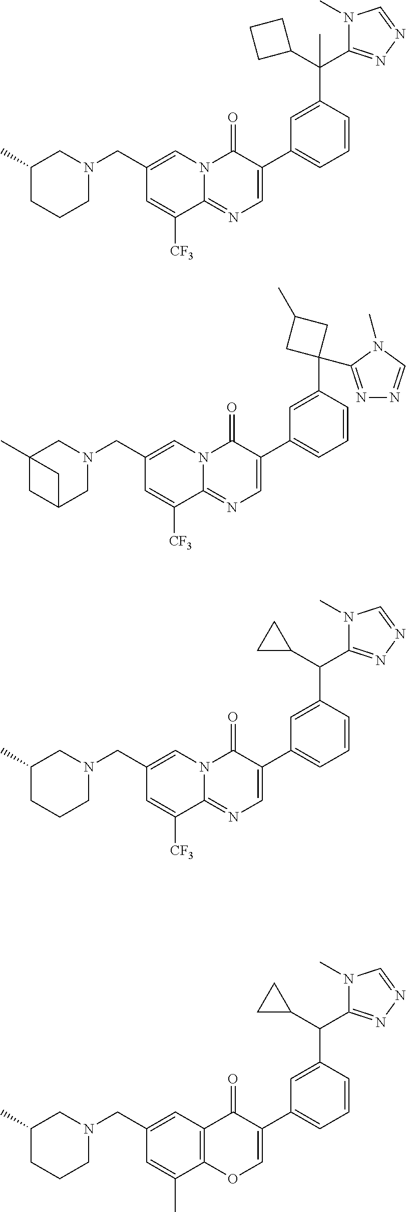 [SMILES: C[C@H]1CCCN(Cc2cc(C(F)(F)F)c3ncc(-c4cccc(C(C)(c5nncn5C)C5CCC5)c4)c(=O)n3c2)C1 ; CC1CC(c2cccc(-c3cnc4c(C(F)(F)F)cc(CN5CC6CC(C)(C6)C5)cn4c3=O)c2)(c2nncn2C)C1 ; C[C@H]1CCCN(Cc2cc(C(F)(F)F)c3ncc(-c4cccc(C(c5nncn5C)C5CC5)c4)c(=O)n3c2)C1 ; Cc1cc(CN2CCC[C@H](C)C2)cc2c(=O)c(-c3cccc(C(c4nncn4C)C4CC4)c3)coc12]

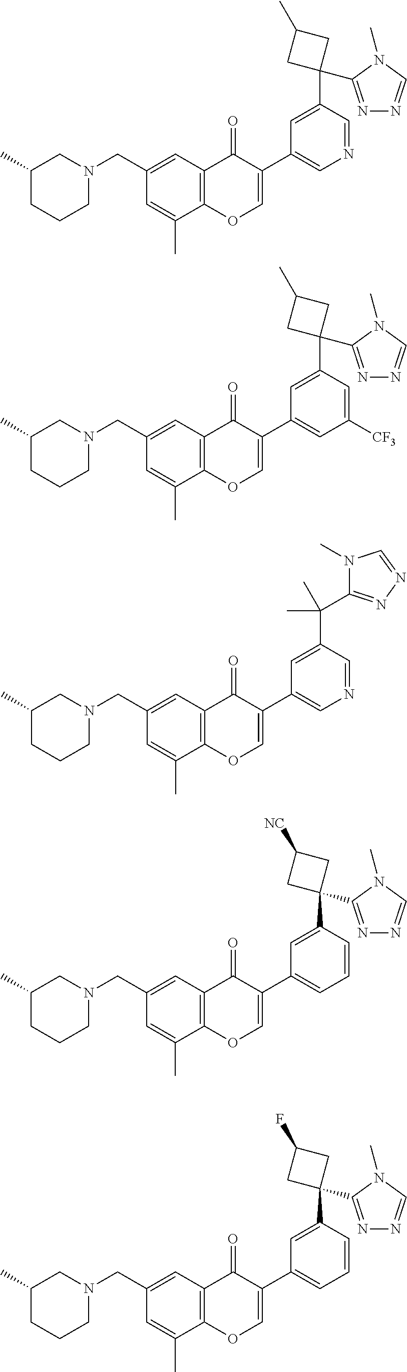 [SMILES: Cc1cc(CN2CCC[C@H](C)C2)cc2c(=O)c(-c3cncc(C4(c5nncn5C)CC(C)C4)c3)coc12 ; Cc1cc(CN2CCC[C@H](C)C2)cc2c(=O)c(-c3cc(C(F)(F)F)cc(C4(c5nncn5C)CC(C)C4)c3)coc12 ; Cc1cc(CN2CCC[C@H](C)C2)cc2c(=O)c(-c3cncc(C(C)(C)c4nncn4C)c3)coc12 ; Cc1cc(CN2CCC[C@H](C)C2)cc2c(=O)c(-c3cccc([C@]4(c5nncn5C)C[C@H](C#N)C4)c3)coc12 ; Cc1cc(CN2CCC[C@H](C)C2)cc2c(=O)c(-c3cccc([C@]4(c5nncn5C)C[C@H](F)C4)c3)coc12]

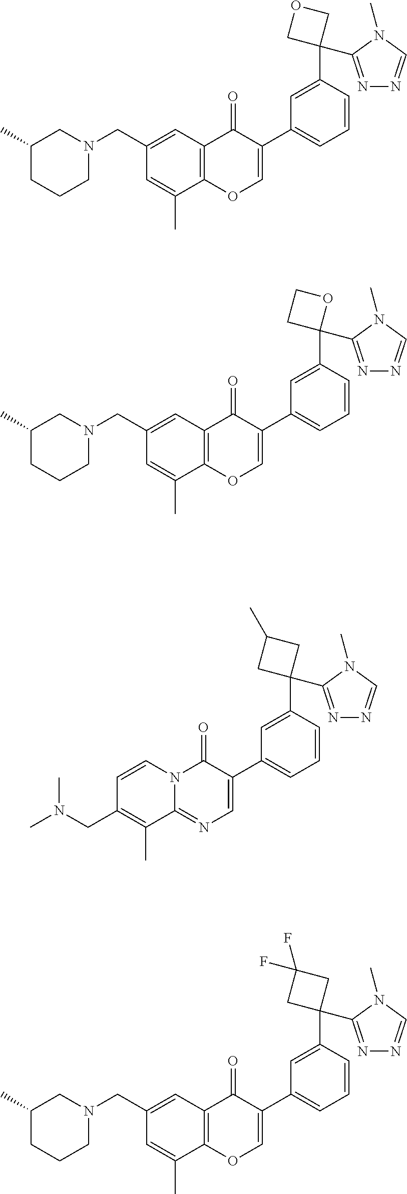 [SMILES: Cc1cc(CN2CCC[C@H](C)C2)cc2c(=O)c(-c3cccc(C4(c5nncn5C)COC4)c3)coc12 ; Cc1cc(CN2CCC[C@H](C)C2)cc2c(=O)c(-c3cccc(C4(c5nncn5C)CCO4)c3)coc12 ; Cc1c(CN(C)C)ccn2c(=O)c(-c3cccc(C4(c5nncn5C)CC(C)C4)c3)cnc12 ; Cc1cc(CN2CCC[C@H](C)C2)cc2c(=O)c(-c3cccc(C4(c5nncn5C)CC(F)(F)C4)c3)coc12]

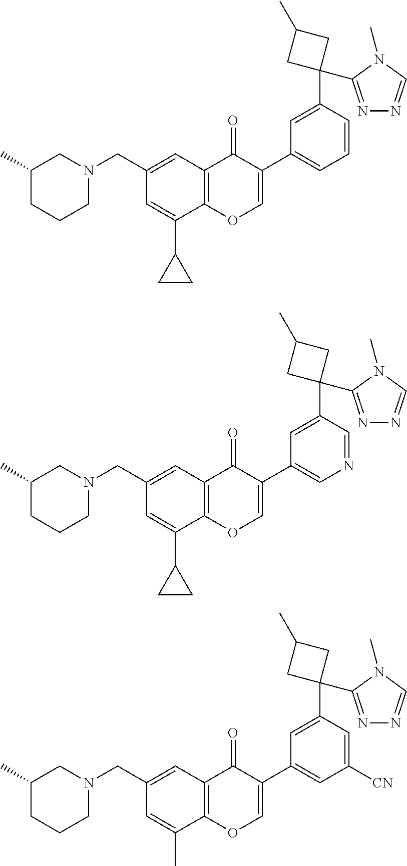 [SMILES: CC1CC(c2cccc(-c3coc4c(C5CC5)cc(CN5CCC[C@H](C)C5)cc4c3=O)c2)(c2nncn2C)C1 ; CC1CC(c2cncc(-c3coc4c(C5CC5)cc(CN5CCC[C@H](C)C5)cc4c3=O)c2)(c2nncn2C)C1 ; Cc1cc(CN2CCC[C@H](C)C2)cc2c(=O)c(-c3cc(C#N)cc(C4(c5nncn5C)CC(C)C4)c3)coc12]

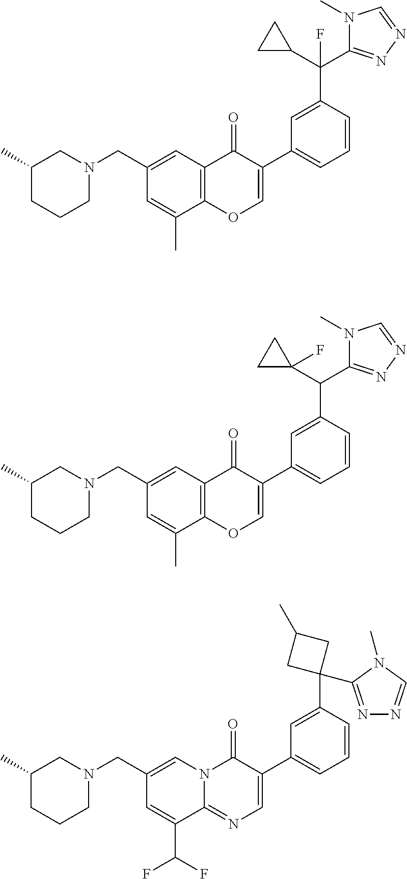 [SMILES: Cc1cc(CN2CCC[C@H](C)C2)cc2c(=O)c(-c3cccc(C(F)(c4nncn4C)C4CC4)c3)coc12 ; Cc1cc(CN2CCC[C@H](C)C2)cc2c(=O)c(-c3cccc(C(c4nncn4C)C4(F)CC4)c3)coc12 ; CC1CC(c2cccc(-c3cnc4c(C(F)F)cc(CN5CCC[C@H](C)C5)cn4c3=O)c2)(c2nncn2C)C1]

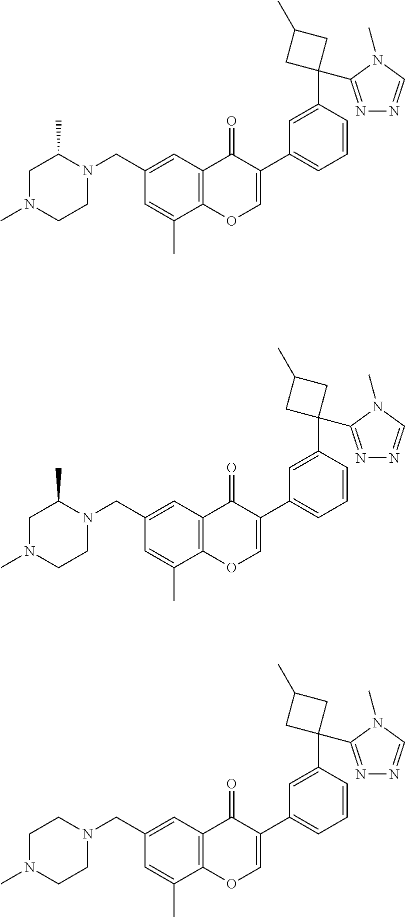 [SMILES: Cc1cc(CN2CCN(C)C[C@@H]2C)cc2c(=O)c(-c3cccc(C4(c5nncn5C)CC(C)C4)c3)coc12 ; Cc1cc(CN2CCN(C)C[C@H]2C)cc2c(=O)c(-c3cccc(C4(c5nncn5C)CC(C)C4)c3)coc12 ; Cc1cc(CN2CCN(C)CC2)cc2c(=O)c(-c3cccc(C4(c5nncn5C)CC(C)C4)c3)coc12]

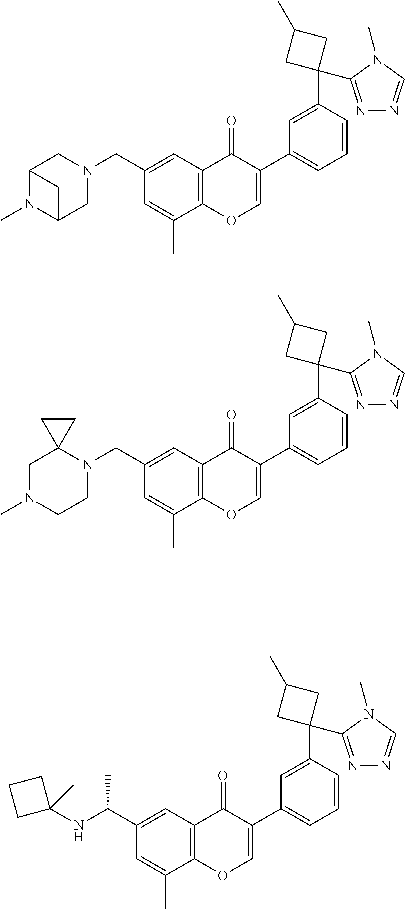 [SMILES: Cc1cc(CN2CC3CC(C2)N3C)cc2c(=O)c(-c3cccc(C4(c5nncn5C)CC(C)C4)c3)coc12 ; Cc1cc(CN2CCN(C)CC23CC3)cc2c(=O)c(-c3cccc(C4(c5nncn5C)CC(C)C4)c3)coc12 ; Cc1cc([C@@H](C)NC2(C)CCC2)cc2c(=O)c(-c3cccc(C4(c5nncn5C)CC(C)C4)c3)coc12]

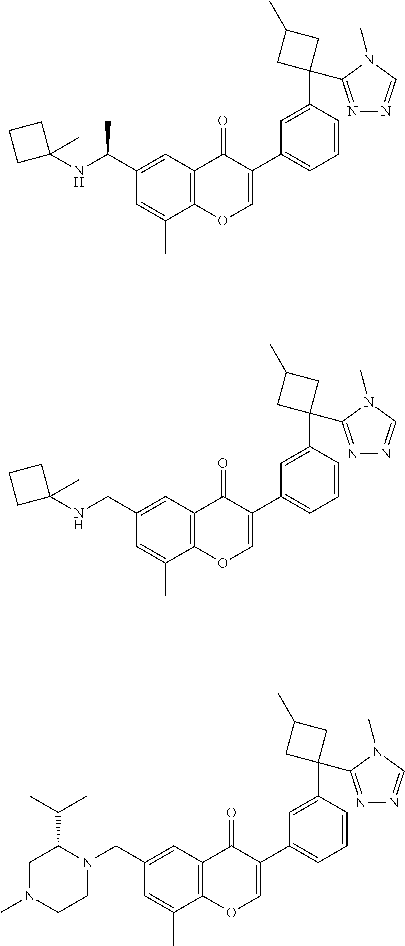 [SMILES: Cc1cc([C@H](C)NC2(C)CCC2)cc2c(=O)c(-c3cccc(C4(c5nncn5C)CC(C)C4)c3)coc12 ; Cc1cc(CNC2(C)CCC2)cc2c(=O)c(-c3cccc(C4(c5nncn5C)CC(C)C4)c3)coc12 ; Cc1cc(CN2CCN(C)C[C@@H]2C(C)C)cc2c(=O)c(-c3cccc(C4(c5nncn5C)CC(C)C4)c3)coc12]

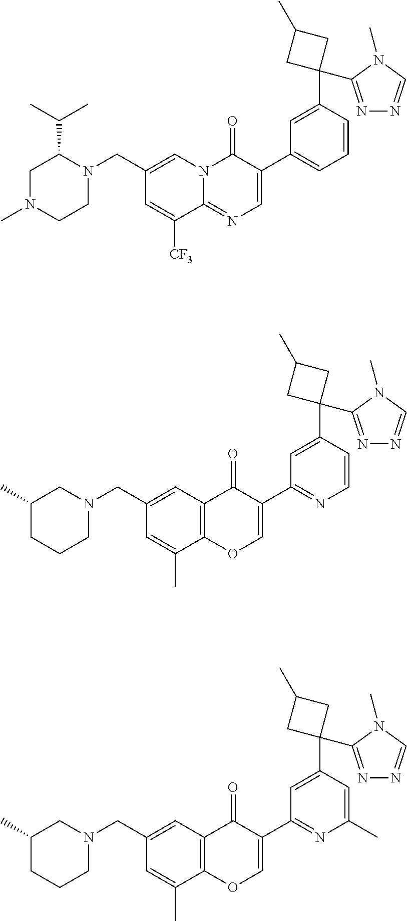 [SMILES: CC1CC(c2cccc(-c3cnc4c(C(F)(F)F)cc(CN5CCN(C)C[C@@H]5C(C)C)cn4c3=O)c2)(c2nncn2C)C1 ; Cc1cc(CN2CCC[C@H](C)C2)cc2c(=O)c(-c3cc(C4(c5nncn5C)CC(C)C4)ccn3)coc12 ; Cc1cc(C2(c3nncn3C)CC(C)C2)cc(-c2coc3c(C)cc(CN4CCC[C@H](C)C4)cc3c2=O)n1]

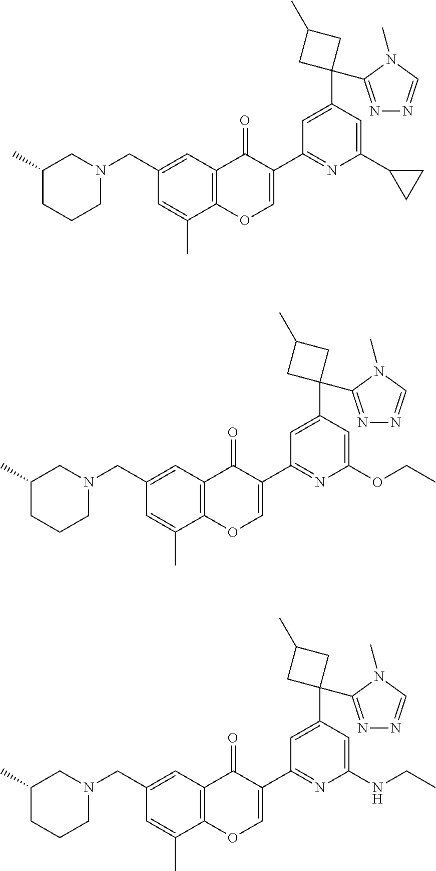

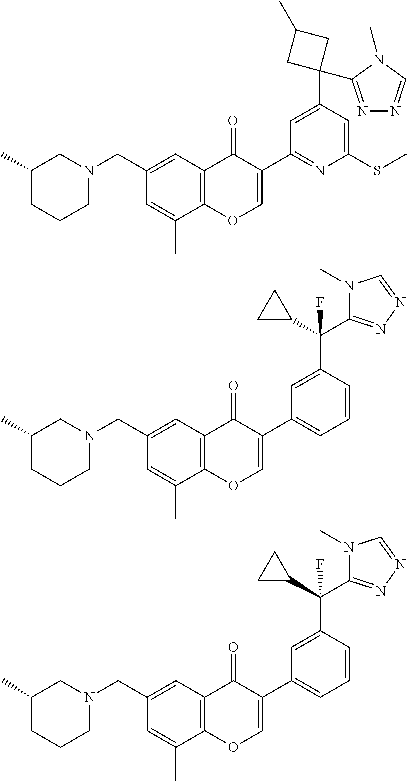

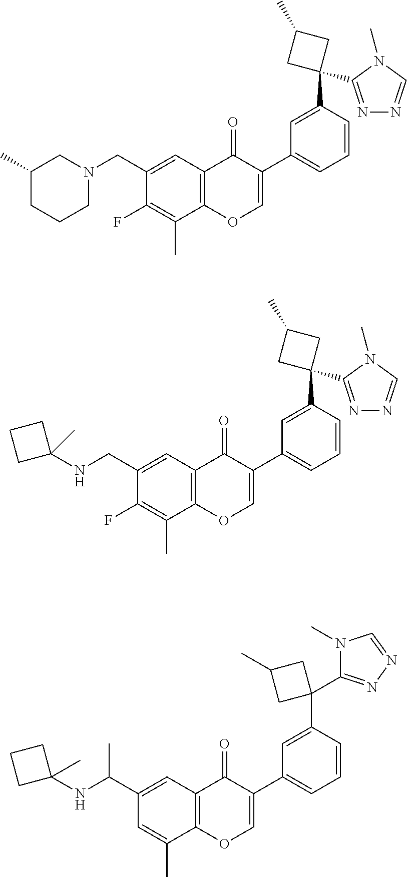 [SMILES: Cc1c(F)c(CN2CCC[C@H](C)C2)cc2c(=O)c(-c3cccc([C@]4(c5nncn5C)C[C@@H](C)C4)c3)coc12 ; Cc1c(F)c(CNC2(C)CCC2)cc2c(=O)c(-c3cccc([C@]4(c5nncn5C)C[C@@H](C)C4)c3)coc12 ; Cc1cc(C(C)NC2(C)CCC2)cc2c(=O)c(-c3cccc(C4(c5nncn5C)CC(C)C4)c3)coc12]

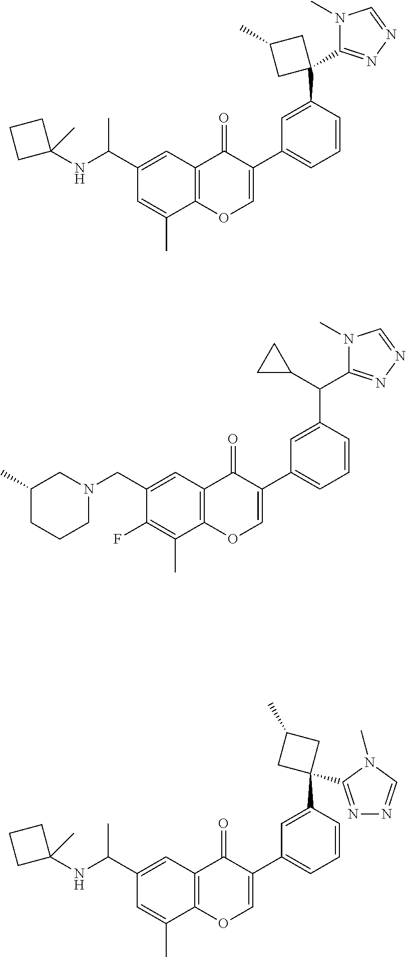

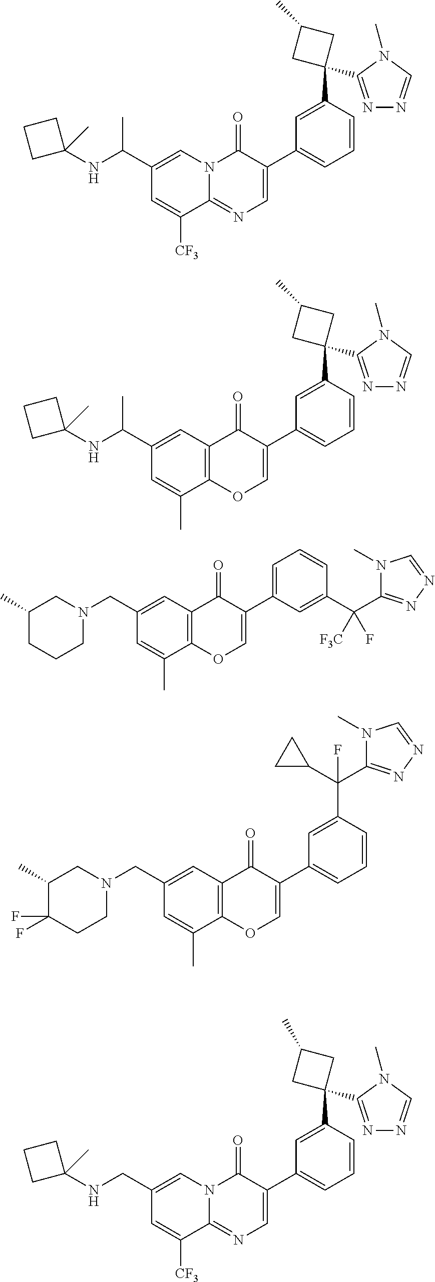 [SMILES: CC(NC1(C)CCC1)c1cc(C(F)(F)F)c2ncc(-c3cccc([C@]4(c5nncn5C)C[C@@H](C)C4)c3)c(=O)n2c1 ; Cc1cc(C(C)NC2(C)CCC2)cc2c(=O)c(-c3cccc([C@]4(c5nncn5C)C[C@@H](C)C4)c3)coc12 ; Cc1cc(CN2CCC[C@H](C)C2)cc2c(=O)c(-c3cccc(C(F)(c4nncn4C)C(F)(F)F)c3)coc12 ; Cc1cc(CN2CCC(F)(F)[C@H](C)C2)cc2c(=O)c(-c3cccc(C(F)(c4nncn4C)C4CC4)c3)coc12 ; Cn1cnnc1[C@]1(c2cccc(-c3cnc4c(C(F)(F)F)cc(CNC5(C)CCC5)cn4c3=O)c2)C[C@H](C)C1]

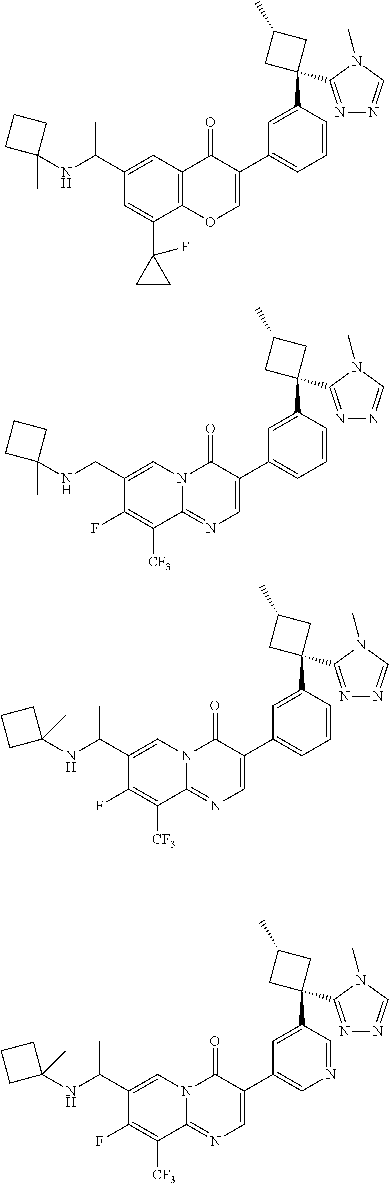 [SMILES: CC(NC1(C)CCC1)c1cc(C2(F)CC2)c2occ(-c3cccc([C@]4(c5nncn5C)C[C@@H](C)C4)c3)c(=O)c2c1 ; Cn1cnnc1[C@]1(c2cccc(-c3cnc4c(C(F)(F)F)c(F)c(CNC5(C)CCC5)cn4c3=O)c2)C[C@H](C)C1 ; CC(NC1(C)CCC1)c1cn2c(=O)c(-c3cccc([C@]4(c5nncn5C)C[C@@H](C)C4)c3)cnc2c(C(F)(F)F)c1F ; CC(NC1(C)CCC1)c1cn2c(=O)c(-c3cncc([C@]4(c5nncn5C)C[C@@H](C)C4)c3)cnc2c(C(F)(F)F)c1F]

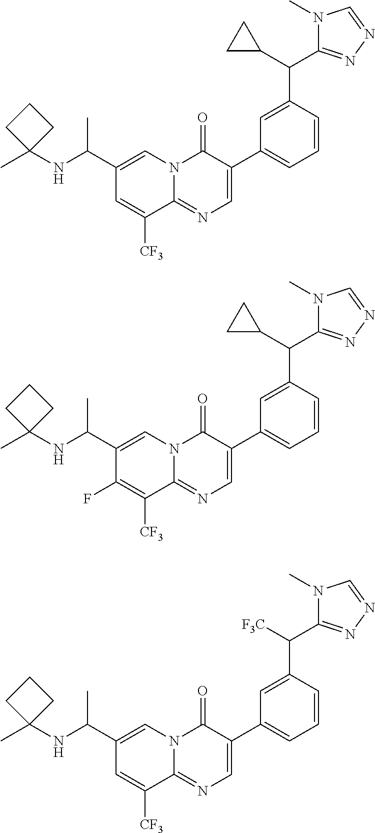 [SMILES: CC(NC1(C)CCC1)c1cc(C(F)(F)F)c2ncc(-c3cccc(C(c4nncn4C)C4CC4)c3)c(=O)n2c1 ; CC(NC1(C)CCC1)c1cn2c(=O)c(-c3cccc(C(c4nncn4C)C4CC4)c3)cnc2c(C(F)(F)F)c1F ; CC(NC1(C)CCC1)c1cc(C(F)(F)F)c2ncc(-c3cccc(C(c4nncn4C)C(F)(F)F)c3)c(=O)n2c1]

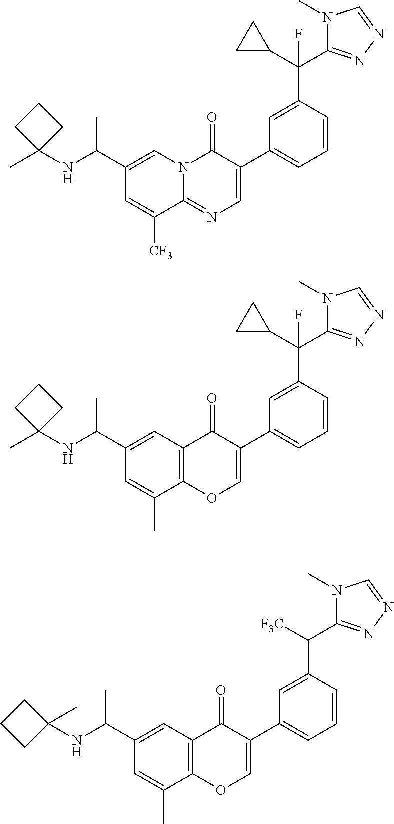 [SMILES: CC(NC1(C)CCC1)c1cc(C(F)(F)F)c2ncc(-c3cccc(C(F)(c4nncn4C)C4CC4)c3)c(=O)n2c1 ; Cc1cc(C(C)NC2(C)CCC2)cc2c(=O)c(-c3cccc(C(F)(c4nncn4C)C4CC4)c3)coc12 ; Cc1cc(C(C)NC2(C)CCC2)cc2c(=O)c(-c3cccc(C(c4nncn4C)C(F)(F)F)c3)coc12]

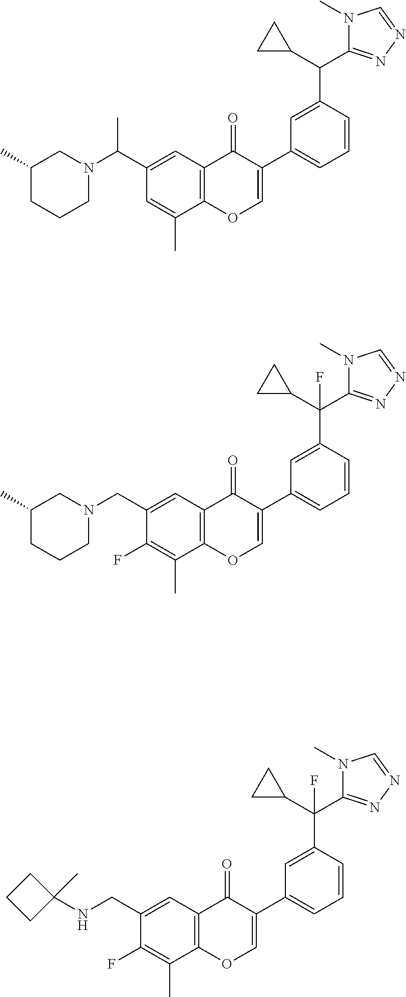 [SMILES: Cc1cc(C(C)N2CCC[C@H](C)C2)cc2c(=O)c(-c3cccc(C(c4nncn4C)C4CC4)c3)coc12 ; Cc1c(F)c(CN2CCC[C@H](C)C2)cc2c(=O)c(-c3cccc(C(F)(c4nncn4C)C4CC4)c3)coc12 ; Cc1c(F)c(CNC2(C)CCC2)cc2c(=O)c(-c3cccc(C(F)(c4nncn4C)C4CC4)c3)coc12]

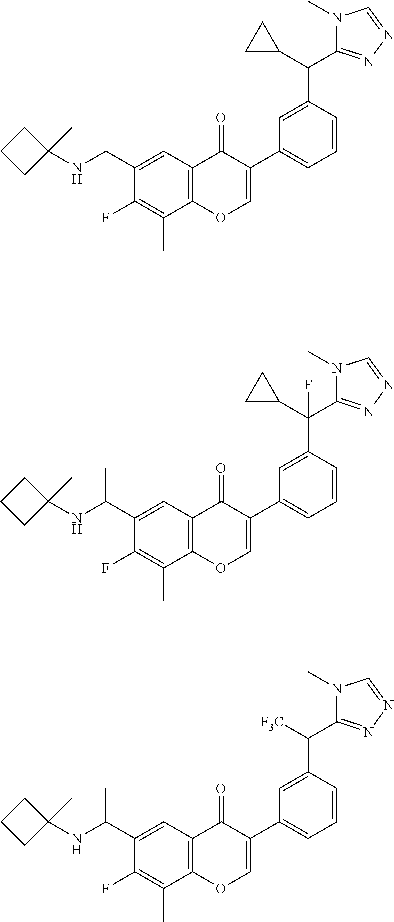 [SMILES: Cc1c(F)c(CNC2(C)CCC2)cc2c(=O)c(-c3cccc(C(c4nncn4C)C4CC4)c3)coc12 ; Cc1c(F)c(C(C)NC2(C)CCC2)cc2c(=O)c(-c3cccc(C(F)(c4nncn4C)C4CC4)c3)coc12 ; Cc1c(F)c(C(C)NC2(C)CCC2)cc2c(=O)c(-c3cccc(C(c4nncn4C)C(F)(F)F)c3)coc12]

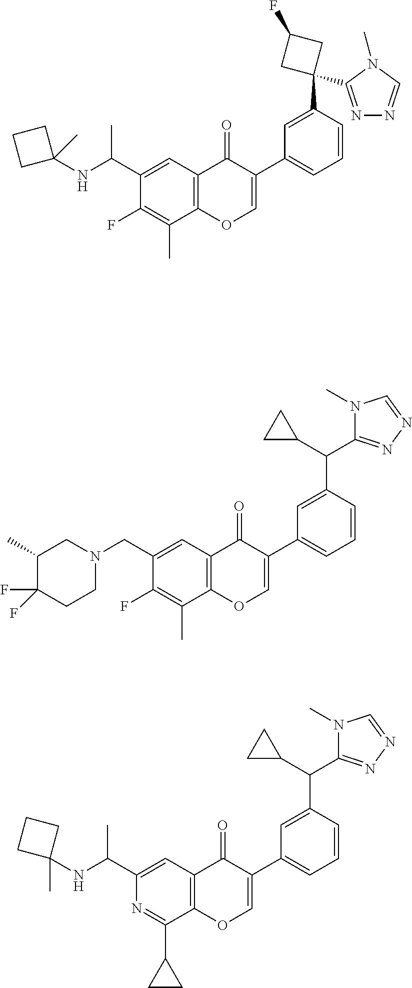 [SMILES: Cc1c(F)c(C(C)NC2(C)CCC2)cc2c(=O)c(-c3cccc([C@]4(c5nncn5C)C[C@H](F)C4)c3)coc12 ; Cc1c(F)c(CN2CCC(F)(F)[C@H](C)C2)cc2c(=O)c(-c3cccc(C(c4nncn4C)C4CC4)c3)coc12 ; CC(NC1(C)CCC1)c1cc2c(=O)c(-c3cccc(C(c4nncn4C)C4CC4)c3)coc2c(C2CC2)n1]

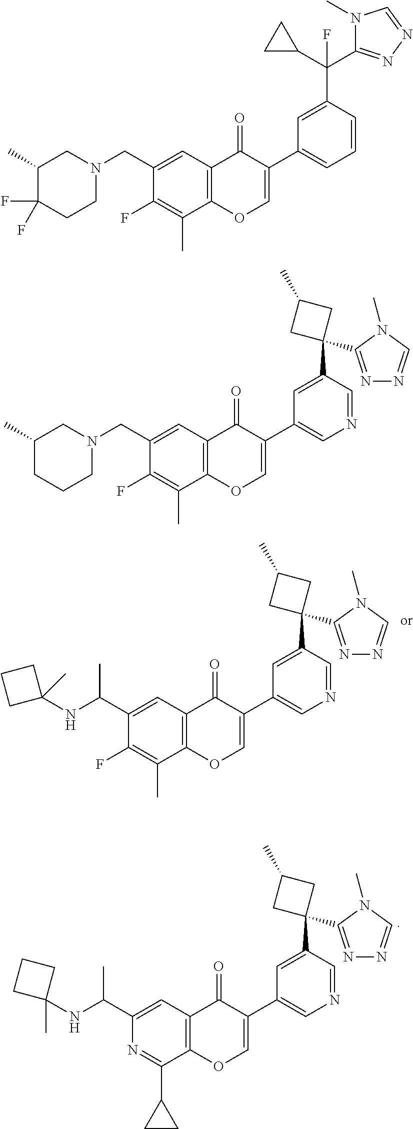 [SMILES: Cc1c(F)c(CN2CCC(F)(F)[C@H](C)C2)cc2c(=O)c(-c3cccc(C(F)(c4nncn4C)C4CC4)c3)coc12 ; Cc1c(F)c(CN2CCC[C@H](C)C2)cc2c(=O)c(-c3cncc([C@]4(c5nncn5C)C[C@@H](C)C4)c3)coc12 ; Cc1c(F)c(C(C)NC2(C)CCC2)cc2c(=O)c(-c3cncc([C@]4(c5nncn5C)C[C@@H](C)C4)c3)coc12 ; CC(NC1(C)CCC1)c1cc2c(=O)c(-c3cncc([C@]4(c5nncn5C)C[C@@H](C)C4)c3)coc2c(C2CC2)n1]

In another aspect, the present application provides a pharmaceutical composition, comprising the compound of formula (I), formula (II), formula (III), formula (IV), formula (V), or formula (VI), or the stereoisomer or pharmaceutically acceptable salt thereof according to the present application and a pharmaceutically acceptable adjuvant.

In another aspect, the present application provides a method for treating diseases or conditions mediated by Cbl-b in mammals, comprising, to mammals in need of the treatment, preferably humans, administering a therapeutically effective amount of the compound of formula (I), formula (II), formula (III), formula (IV), formula (V), or formula (VI), or the stereoisomer or pharmaceutically acceptable salt or pharmaceutical composition thereof.

In another aspect, the present application provides a method for treating or preventing tumors or autoimmune diseases in mammals, comprising, to mammals in need of the treatment or prevention, preferably humans, administering a therapeutically effective amount of the compound of formula (I), formula (II), formula (III), formula (IV), formula (V), or formula (VI), or the stereoisomer or pharmaceutically acceptable salt or pharmaceutical composition thereof.

In another aspect, the present application provides the use of the compound of formula (I), formula (II), formula (III), formula (IV), formula (V), or formula (VI), or the stereoisomer or pharmaceutically acceptable salt or pharmaceutical composition thereof in the manufacture of drugs for the prevention or treatment of diseases or conditions mediated by Cbl-b.

In another aspect, the present application provides the use of the compound of formula (I), formula (II), formula (III), formula (IV), formula (V), or formula (VI), or the stereoisomer or pharmaceutically acceptable salt or pharmaceutical composition thereof in the manufacture of drugs for the prevention or treatment of tumors or autoimmune diseases.

In another aspect, the present application provides the use of the compound of formula (I), formula (II), formula (III), formula (IV), formula (V), or formula (VI), or the stereoisomer or pharmaceutically acceptable salt or pharmaceutical composition thereof in the prevention or treatment of diseases or conditions mediated by Cbl-b.

In another aspect, the present application provides the use of the compound of formula (I), formula (II), formula (III), formula (IV), formula (V), or formula (VI), or the stereoisomer or pharmaceutically acceptable salt or pharmaceutical composition thereof in the prevention or treatment of tumors or autoimmune diseases.

In another aspect, the present application provides the compound of formula (I), formula (II), formula (III), formula (IV), formula (V), or formula (VI), or the stereoisomer or pharmaceutically acceptable salt or pharmaceutical composition thereof for use in the prevention or treatment of diseases or conditions mediated by Cbl-b in mammals, preferably humans.

In another aspect, the present application provides the compound of formula (I), formula (II), formula (III), formula (IV), formula (V), or formula (VI), or the stereoisomer or pharmaceutically acceptable salt or pharmaceutical composition thereof for use in the prevention or treatment of tumors or autoimmune diseases in mammals, preferably humans.

In some embodiments, the disease or condition mediated by Cbl-b is selected from a tumor or an autoimmune disease.

### Definition and Description of Terminology

Unless otherwise stated, the terms used in the present application have the following meanings; the definitions of groups and terms described in the present application, including their definitions as examples, exemplary definitions, preferred definitions, definitions listed in tables, definitions of specific compounds in the examples, etc., may be arbitrarily combined or incorporated with one another. A specific term should not be considered uncertain or unclear unless specifically defined, but should be understood in its ordinary meaning in the art. When a trade name appears herein, it is intended to refer to the corresponding commodity or an active ingredient thereof.

“ [symbol] ” herein indicates a linking site.

The bond “ [symbol] ” depicted by solid and dashed lines herein indicates a single or double bond.

The diagrammatic presentation of the racemate or enantiomerically pure compound herein is from Maehr, J. Chem. Ed. 1985, 62:114-120. Unless otherwise stated, the wedged solid bond and wedged dashed bond ( [symbol] and [symbol] ) are used to represent the absolute configuration of a stereocenter, and the straight solid bond and straight dashed bond ( [symbol] and [symbol] ) are used to represent the relative configuration of a stereocenter (such as cis or trans configuration of alicyclic compounds).

The term “tautomer” refers to a functional group isomer resulting from the rapid movement of an atom in two positions in a molecule. The compounds of the present application may exhibit tautomerism. Tautomeric compounds can exist as two or more interconvertible forms. Tautomers generally exist in equilibrium form, so that the attempts to separate a single tautomer usually result in the formation of a mixture, whose chemical and physical properties are consistent with a mixture of the compounds. The position of equilibrium depends on the chemical properties within the molecule. For example, in many aliphatic aldehydes and ketones such as acetaldehyde, the ketone form is dominant; and in phenols, the enol form is dominant. The present application encompasses all tautomeric forms of the compounds.

The term “stereoisomer” refers to an isomer created as a result of different spatial arrangement of atoms in molecules, including cis and trans isomers, enantiomers and diastereomers.

The compounds of the present application may have asymmetric atoms, such as carbon atoms, sulfur atoms, nitrogen atoms, phosphorus atoms or asymmetric double bonds, and therefore the compounds of the present application may exist in specific geometric or stereoisomeric forms. Specific geometric or stereoisomeric forms may be cis and trans isomers, E- and Z-geometric isomers, (−)- and (+)-enantiomers, (R)- and (S)-enantiomers, diastereomers, (D)-isomers, (L)-isomers, and racemic mixtures or other mixtures thereof, such as enantiomerically or diastereomerically enriched mixtures, and all of the above isomers and mixtures thereof fall within the scope of the definition of the compounds of the present application. Additional asymmetric carbon atoms, asymmetric sulfur atoms, asymmetric nitrogen atoms, or asymmetric phosphorus atoms may be present in substituents such as an alkyl group, and these isomers and mixtures thereof involved in all substituents are also included in the scope of the definition of the compounds of the present application. The compounds containing asymmetric atoms of the present application can be separated in optically active-pure or racemic forms, and the optically active-pure forms can be resolved from the racemic mixture or synthesized by utilizing chiral raw materials or chiral reagents.

The term “substituted” means that any one or more hydrogen atoms on the designated atom are substituted with a substituent, provided that the valence state of the designated atom is normal, and the substituted compound is stable. When the substituent is oxo (i.e., ═O), it means that two hydrogen atoms are substituted, which would not occur on aromatic groups.

The term “optional” or “optionally” means that the subsequently described event or circumstance may or may not occur, and the description includes the occurrence and the non-occurrence of the event or circumstance. For example, the expression “ethyl is “optionally” substituted with halogen” means that ethyl may be unsubstituted (CH₂CH₃), mono-substituted (CH₂CH₂F, CH₂CH₂Cl, etc.), poly-substituted (CHFCH₂F, CH₂CHF₂, CHFCH₂Cl, CH₂CHCl₂, etc.), or completely substituted (CF₂CF₃, CF₂CCl₃, CCl₂CCl₃, etc.). With respect to any group containing one or more substituents, it will be understood by those skilled in the art that any substitution or substitution patterns that are sterically impractical and/or synthetically non-feasible are not intended to be introduced into such group.

Where any variable (such as R^{1a}, R^{2a}) appears more than once in the constitution or structure of a compound, its definition in each case is independent. For example, if a group is substituted with two R^{1a}, then each R^{1a} has an independent option.

When the number of a linking group is 0, such as —(CR¹³R¹⁴)₀—, it means that the linking group is a bond.

When one of the variables is selected from a chemical bond or absent, it means that the two groups to which it is attached are directly connected. For example, when L represents a bond in

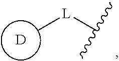

it means that the structure is actually

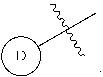

When the linking direction of the linking group referred to herein is not indicated, the linking direction is arbitrary. For example, when L in the structural unit

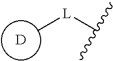

is selected from “—NR⁷CH₂—”, L can be either connected with the ring D from left to right to form a “ring D-NR⁷CH₂—”, or can be connected with the ring D from right to left to form “ring D-CH₂NR⁷—”.

Cₘ-Cₙ herein means that it has an integer number of carbon atoms, wherein the integer number is within the range of m-n. For example, “C₁-C₆” means that the group may have 1, 2, 3, 4, 5, or 6 carbon atoms.

The term “alkyl” refers to a hydrocarbon group of general formula CₙH₂ₙ₊₁, which may be linear or branched. The term “C₁-C₆ alkyl” is to be understood as denoting a linear or branched, saturated monovalent hydrocarbon group having 1, 2, 3, 4, 5 or 6 carbon atoms. Specific examples of the alkyl include, but are not limited to, methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, 1,2-dimethylbutyl, etc. The term “C₁-C₃ alkyl” is to be understood as denoting a linear or branched, saturated monovalent hydrocarbon group having 1, 2 or 3 carbon atoms. The “C₁-C₆ alkyl” may comprise “C₁-C₃ alkyl”.

The term “alkoxy” refers to a monovalent group produced by the loss of a hydrogen atom on a hydroxyl group of a linear or branched alcohol, and is to be understood as “alkyloxy” or “alkyl-O—”. The term “C₁-C₆ alkoxy” is to be understood as “C₁-C₆ alkyloxy” or “C₁-C₆ alkyl-O—”. The term “C₁-C₃ alkoxy” is to be understood as “C₁-C₃ alkyloxy” or “C₁-C₃ alkyl-O—”. The “C₁-C₆ alkoxy” may further comprise “C₁-C₃ alkoxy”.

The term “alkenyl” refers to a linear or branched, monovalent unsaturated aliphatic hydrocarbon group consisting of carbon atoms and hydrogen atoms and having at least one double bond. The term “C₂-C₄ alkenyl” is to be understood as denoting a linear or branched, unsaturated monovalent hydrocarbon group, comprising one or more double bonds and having 2, 3 or 4 carbon atoms. The “C₂-C₄ alkenyl” is preferably C₂ or C₃ alkenyl. It is to be understood that where the alkenyl group contains more than one double bond, the double bonds may be isolated from, or conjugated with, each other. Specific examples of the alkenyl include, but are not limited to, vinyl, allyl, (E)-2-methylvinyl, (Z)-2-methylvinyl, (E)-but-2-enyl, (Z)-but-2-enyl, (E)-but-1-enyl, (Z)-but-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, etc.

The term “alkynyl” refers to a linear or branched, monovalent unsaturated aliphatic hydrocarbon group consisting of carbon atoms and hydrogen atoms and having at least one triple bond. The term “C₂-C₄ alkynyl” is to be understood as denoting a linear or branched, unsaturated monovalent hydrocarbon group, comprising one or more triple bonds and having 2, 3, or 4 carbon atoms. The examples of “C₂-C₄ alkynyl” include, but are not limited to, ethynyl (—C≡CH), propynyl (—C≡CCH₃, —CH₂C≡CH), but-1-ynyl, but-2-ynyl or but-3-ynyl. The “C₂-C₄ alkynyl” may comprise “C₂-C₃ alkynyl”. The examples of “C₂-C₃ alkynyl” include ethynyl (—C≡CH), prop-1-ynyl (—C≡CCH₃), prop-2-ynyl (propargyl).

The term “cycloalkyl” refers to a carbocyclic group that is fully saturated and exists as a monocyclic ring, fused ring, bridged ring or spirocyclic ring and other forms. Unless otherwise indicated, the carbocyclic group is typically a 3- to 10-membered ring. The term “C₃-C₁₀ cycloalkyl” is to be understood as denoting a saturated monovalent monocyclic ring, fused ring, spirocyclic ring or bridged ring, having 3, 4, 5, 6, 7, 8, 9 or 10 carbon atoms. Specific examples of the cycloalkyl include, but are not limited to, cyclopropyl, cyclobutyl, cyclopentyl, cyclohexyl, cycloheptyl, cyclooctyl, cyclononyl, cyclodecyl, norbornyl (bicyclo[2.2.1]heptyl), bicyclo[2.2.2]octyl, adamantyl, spiro[4.5]decanyl, etc. The term “C₃-C₁₀ cycloalkyl” may comprise “C₃-C₈ cycloalkyl”. The “C₃-C₈ cycloalkyl” may comprise “C₃-C₆ cycloalkyl”. The “C₃-C₆ cycloalkyl” may comprise “C₃-C₄ cycloalkyl”. The term “C₃-C₆ cycloalkyl” is to be understood as denoting a saturated monovalent monocyclic or bicyclic hydrocarbon ring, having 3, 4, 5 or 6 carbon atoms. Specific examples include, but are not limited to, cyclopropyl, cyclobutyl, cyclopentyl, cyclohexyl, etc.

The term “cycloalkyloxy” is to be understood as “cycloalkyl-O—”.

The term “cycloalkenyl” refers to a non-aromatic monocyclic or polycyclic hydrocarbon group containing at least one carbon-carbon double bond. “C₃-C₆ cycloalkenyl” refers to a non-aromatic cyclic hydrocarbon group having 3, 4, 5 or 6 carbon atoms as ring atoms and containing at least one carbon-carbon double bond. Specific examples of C₃-C₆ cycloalkenyl include, but are not limited to, cyclopropenyl, cyclobutenyl, cyclopentenyl, cyclopentadienyl, cyclohexenyl, cyclohexadienyl, etc.

The term “heterocyclyl” refers to a fully saturated or partially saturated (not a heteroaromatic group which is aromatic as a whole) monovalent monocyclic, fused ring, spirocyclic or bridged ring group, the ring atoms therein containing 1, 2, 3, 4 or 5 heteroatoms or heteroatomic groups (namely, atomic groups containing heteroatoms), wherein the “heteroatoms or heteroatomic groups” include but are not limited to nitrogen atoms (N), oxygen atoms (O), sulfur atoms (S), phosphorous atoms (P), boron atoms (B), —S(═O)₂—, —S(═O)—, and optionally substituted —NH—, —S(═O)(═NH)—, —C(═O)NH—, —C(═NH)—, —S(═O)₂NH—, S(═O)NH— or —NHC(═O)NH—, etc. The term “4- to 10-membered heterocyclyl” refers to a heterocyclyl group having 4, 5, 6, 7, 8, 9 or 10 ring atoms, the ring atoms therein containing 1, 2, 3, 4 or 5 heteroatoms or heteroatomic groups independently selected from the above-mentioned heteroatoms or heteroatomic groups. The term “4- to 10-membered nitrogen-containing heterocyclyl” refers to a 4-, 5-, 6-, 7-, 8-, 9- or 10-membered heterocyclyl, the ring atoms therein containing at least one N atom. The “4- to 10-membered nitrogen-containing heterocyclyl” includes “6- to 10-membered nitrogen-containing heterocyclyl”. The term “4- to 7-membered monocyclic nitrogen-containing heterocyclyl” refers to a 4-, 5-, 6-, or 7-membered heterocyclyl in a monocyclic ring form, the ring atoms therein containing at least one N atom. The “4- to 10-membered heterocyclyl” includes “6- to 10-membered heterocyclyl”. The “6- to 10-membered heterocyclyl” further includes “6- to 7-membered heterocyclyl”. Specific examples of 4-membered heterocyclyl include, but are not limited to, azetidinyl, thietanyl or oxetanyl; specific examples of 5-membered heterocyclyl include, but are not limited to, tetrahydrofuryl, dioxolyl, pyrrolidyl, imidazolidinyl, pyrazolidinyl, pyrrolinyl, 4,5-dihydroxazolyl or 2,5-dihydro-1H-pyrrolyl; specific examples of 6-membered heterocyclyl include, but are not limited to, tetrahydropyranyl, piperidyl, morpholinyl, dithianyl, thiomorpholinyl, piperazinyl, trithianyl, tetrahydropyridyl or 4H-[1,3,4]thiadiazinyl; specific examples of 7-membered heterocyclyl include, but are not limited to, diazepanyl; specific examples of 8-membered heterocyclyl include, but are not limited to, 6,7-dihydro-5H-pyrrolo[2,1-c][1,2,4]triazolyl. The heterocyclyl may also be a bicyclic group. Specific examples of 5,5-membered bicyclic group include, but are not limited to, hexahydrocyclopenta[c]pyrrol-2(1H)-yl. Specific examples of 5,6-membered bicyclic group include, but are not limited to, hexahydropyrrolo[1,2-a]pyrazin-2(1H)-yl, 5,6,7,8-tetrahydro-[1,2,4]triazolo[4,3-a]pyrazinyl or 5,6,7,8-tetrahydroimidazo[1,5-a]pyrazinyl. Optionally, the heterocyclyl may be a benzo-fused ring group of the above-mentioned 4-7 membered heterocyclyl, specific examples including but not limited to dihydroisoquinolinyl, etc. The “4- to 10-membered heterocyclyl” may comprise “5- to 10-membered heterocyclyl”, “4- to 7-membered heterocyclyl”, “5- to 7-membered heterocyclyl”, “5- to 6-membered heterocyclyl”, “6- to 8-membered heterocyclyl”, “4- to 10-membered heterocycloalkyl”, “5- to 10-membered heterocycloalkyl”, “4- to 7-membered heterocycloalkyl”, “5- to 6-membered heterocycloalkyl”, “6- to 8-membered heterocycloalkyl” and other ranges, and the “4- to 7-membered heterocyclyl” may further comprise “4- to 6-membered heterocyclyl”, “5- to 7-membered heterocyclyl”, “5- to 6-membered heterocyclyl”, “4- to 7-membered heterocycloalkyl”, “4- to 6-membered heterocycloalkyl”, “5- to 6-membered heterocycloalkyl” and other ranges. Although some bicyclic heterocyclyl groups in the present application partially contain one benzene ring or one heteroaromatic ring, the heterocyclyl groups are still non-aromatic as a whole.

The term “heterocyclyloxy” is to be understood as “heterocyclyl-O—”.

The term “heterocycloalkyl” refers to a monovalent cyclic group that is fully saturated and exists as a monocyclic ring, fused ring, bridged ring or spirocyclic ring and other forms, the ring atoms therein containing 1, 2, 3, 4 or 5 heteroatoms or heteroatomic groups (namely, atomic groups containing heteroatoms). The “heteroatoms or heteroatomic groups” include, but are not limited to, nitrogen atoms (N), oxygen atoms (O), sulfur atoms (S), phosphorus atoms (P), boron atoms (B), —S(═O)₂—, —S(═O)—, and optionally substituted —NH—, —S(═O)(═NH)—, —C(═O)NH—, —C(═NH)—, —S(═O)₂NH—, S(═O)NH— or —NHC(═O)NH—, etc. The term “4- to 10-membered heterocycloalkyl” refers to a heterocycloalkyl group having 4, 5, 6, 7, 8, 9 or 10 ring atoms, the ring atoms therein containing 1, 2, 3, 4 or 5 heteroatoms or heteroatomic groups independently selected from the above-mentioned heteroatoms or heteroatomic groups. The “4- to 10-membered heterocycloalkyl” includes “4- to 7-membered heterocycloalkyl”, wherein specific examples of 4-membered heterocycloalkyl include, but are not limited to azetidinyl, oxetanyl, or thietanyl; specific examples of 5-membered heterocycloalkyl include, but are not limited to, tetrahydrofuryl, tetrahydrothienyl, pyrrolidinyl, isoxazolidinyl, oxazolidinyl, isothiazolidinyl, thiazolidinyl, imidazolidinyl, or tetrahydropyrazolyl; specific examples of 6-membered heterocycloalkyl include, but are not limited to, piperidyl, tetrahydropyranyl, tetrahydrothiopyranyl, morpholinyl, piperazinyl, 1,4-thioxanyl, 1,4-dioxanyl, thiomorpholinyl, 1,3-dithianyl, or 1,4-dithianyl; specific examples of 7-membered heterocycloalkyl include, but are not limited to, azepanyl, oxepanyl, or thiepanyl.

The term “aryl” refers to an all-carbon monocyclic or fused polycyclic aromatic ring group having a conjugated π-electron system. The aryl may have 6-14 carbon atoms or 6-10 carbon atoms. The term “C₆-C₁₀ aryl” is to be understood as denoting a monovalent aromatic monocyclic or bicyclic groups having 6 to 10 carbon atoms. especially a ring having 6 carbon atoms (“C₆ aryl”), such as phenyl; or a ring having 10 carbon atoms (“C₁₀ aryl”), such as naphthyl.

The term “aryloxy” is to be understood as “aryl-O—”.

The term “heteroaryl” refers to an aromatic monocyclic or fused polycyclic ring system, containing at least one ring atom selected from N, O, and S, the remaining ring atoms being aromatic ring groups of C. The term “5- to 10-membered heteroaryl” is to be understood as including a monovalent monocyclic or bicyclic aromatic ring system having 5, 6, 7, 8, 9 or 10 ring atoms, especially 5 or 6 or 9 or 10 ring atoms, and containing 1, 2, 3, 4 or 5 heteroatoms, preferably 1, 2 or 3 heteroatoms independently selected from N, O and S. Especially, the heteroaryl is selected from thienyl, furyl, pyrrolyl, oxazolyl, thiazolyl, imidazolyl, pyrazolyl, isoxazolyl, isothiazolyl, oxadiazolyl, triazolyl, thiadiazolyl, etc., and the benzo derivatives thereof, such as benzofuryl, benzothienyl, benzothiazolyl, benzoxazolyl, benzisoxazolyl, benzoimidazolyl, benzotriazolyl, indazolyl, indolyl or isoindolyl; or pyridyl, pyridazinyl, pyrimidyl, pyrazinyl, triazinyl, etc., and the benzo derivatives thereof, such as quinolinyl, quinazolinyl or isoquinolinyl; or azocinyl, indolizinyl, purinyl, etc., and the benzo derivatives thereof; or cinnolinyl, phthalazinyl, quinazolinyl, quinoxalinyl, naphthyridinyl, pteridinyl, carbazolyl, acridinyl, phenazinyl, phenothiazinyl, phenoxazinyl, etc. The term “5- to 10-membered nitrogen-containing heteroaryl” refers to a 5-, 6-, 7-, 8-, 9- or 10-membered heteroaryl, the ring atoms therein containing at least one N atom. The “5- to 10-membered nitrogen-containing heteroaryl” comprises “5- to 6-membered nitrogen-containing heteroaryl”. The term “5- to 6-membered heteroaryl” refers to an aromatic ring system having 5 or 6 ring atoms, and containing 1, 2 or 3 heteroatoms, preferably 1 or 2 heteroatoms independently selected from N, O and S. The term “6-membered heteroaryl” refers to an aromatic ring system having 6 ring atoms, and containing 1, 2 or 3 heteroatoms, preferably 1 or 2 N atoms as heteroatoms. The term “5- to 10-membered heteroaryl” comprises “5- to 6-membered heteroaryl”.

The term “heteroaryloxy” is to be understood as “heteroaryl-O—”.

The term “halo” or “halogen” refers to fluorine (F), chlorine (Cl), bromine (Br) or iodine (I).

The term “hydroxyl” refers to —OH group.

The term “cyano” refers to —CN group.

The term “mercapto” refers to —SH group.

The term “amino” refers to —NH₂ group.

The term “nitro” refers to —NO₂ group.

The term “therapeutically effective amount” means an amount of the compound of the present application that (i) treats or prevents a particular disease, condition or disorder, (ii) attenuates, ameliorates or eliminates one or more symptoms of a particular disease, condition or disorder, or (iii) prevents or delays the onset of one or more symptoms of a particular disease, condition or disorder described herein. The amount of the compound of the present application which constitutes a “therapeutically effective amount” will vary depending on the compound, the disease states and the severity thereof, the manner of administration, and the age of the mammal to be treated, but can be determined routinely by those skilled in the art according to their own knowledge and the present disclosure.

The term “pharmaceutically acceptable” refers to compounds, materials, compositions and/or dosage forms, which are, within the scope of sound medical judgment, suitable for use in contact with human and animal tissues, without excessive toxicity, irritation, allergic reactions or other problems or complications, which is commensurate with a reasonable benefit/risk ratio.

The term “pharmaceutically acceptable salt” refers to salts of pharmaceutically acceptable acids or bases, including salts formed between compounds and inorganic or organic acids, and salts formed between compounds and inorganic or organic bases.

The term “pharmaceutical composition” refers to a mixture of one or more of the compounds or the stereoisomers or salts thereof according to the present application and a pharmaceutically acceptable adjuvant. The pharmaceutical composition is intended to facilitate administering the compound of the present application to an organism.

The term “pharmaceutically acceptable adjuvant” refers to adjuvants which have no significant irritating effect on the organism and do not impair the bioactivity and properties of the active compound. Suitable adjuvants are well known to those skilled in the art, and are such as a carbohydrate, a wax, a water-soluble and/or water-swellable polymer, a hydrophilic or hydrophobic material, gelatin, an oil, a solvent, water, and the like.

The word “comprise” and its variants such as “comprises” or “comprising” are to be understood as an open, non-exclusive meaning, i.e., “including but not limited to”.

The present application also includes isotopically-labeled compounds of the present application which are identical to those recited herein, but have one or more atoms replaced by an atom having an atomic mass or mass number different from the atomic mass or mass number usually found in nature. Examples of isotopes that can be incorporated into the compounds of the present application include isotopes of hydrogen, carbon, nitrogen, oxygen, phosphorus, sulfur, fluorine, iodine and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively.

Certain isotopically-labeled compounds of the present application (e.g., those labeled with ³H and ¹⁴C) are useful in tissue distribution assays of compounds and/or substrates. Tritiated (i.e., ³H) and carbon-14 (i.e., ¹⁴C) isotopes are particularly preferred for their ease of preparation and detectability. Positron emitting isotopes such as ¹⁵O, ¹³N, ¹¹C, and ¹⁸F are useful for positron emission tomography (PET) studies to examine substrate occupancy. The isotopically-labeled compounds of the present application can generally be prepared according to following procedures analogous to those disclosed in the Schemes and/or in the Examples herein below by substituting a non-isotopically-labeled reagent with an isotopically-labeled reagent.

The pharmaceutical composition of the present application may be prepared by combining the compound of the present application with an appropriate pharmaceutically acceptable adjuvant. For example, the pharmaceutical composition of the present application may be formulated into solid, semi-solid, liquid or gaseous preparations, such as tablets, pills, capsules, powders, granules, ointments, emulsions, suspensions, suppositories, injections, inhalants, gels, microspheres and aerosols.

Typical administration routes of the compound, or the stereoisomer or pharmaceutically acceptable salt thereof, or the pharmaceutical composition thereof of the present application include, but are not limited to oral administration, rectal administration, topical administration, administration by inhalation, parenteral administration, sublingual administration, intravaginal administration, intranasal administration, intraocular administration, intraperitoneal administration, intramuscular administration, subcutaneous administration, and intravenous administration.

The pharmaceutical composition of the present application can be manufactured by using well-known methods in the art, such as conventional mixing method, dissolution method, granulation method, emulsification method, and freeze-drying method.

In some embodiments, the pharmaceutical composition is in oral form. For oral administration, the pharmaceutical composition may be formulated by mixing the active compound with a pharmaceutically acceptable adjuvant well-known in the art. Such adjuvants enable the compounds of the present application to be formulated into tablets, pills, lozenges, dragees, capsules, liquids, gels, syrups, suspensions, etc., for oral administration to patients.

A solid oral composition can be prepared by a conventional mixing, filling or tableting method. For example, it can be obtained by mixing the active compound with a solid adjuvant, optionally grinding the resulting mixture, adding other suitable adjuvants, if necessary, and then processing the mixture into granules to obtain cores of tablets or dragees. Suitable adjuvants include, but are not limited to, binders, diluents, disintegrants, lubricants, glidants, flavoring agents, etc.

The pharmaceutical composition can also be suitable for parenteral administration, such as sterile solutions, suspensions or lyophilized products in a suitable unit dosage form.

The daily administration dose of the compound of general formula I in all the administration manners described herein is from 0.01 mg/kg body weight to 200 mg/kg body weight, preferably from 0.05 mg/kg body weight to 50 mg/kg body weight, and more preferably from 0.1 mg/kg body weight to 30 mg/kg body weight, in the form of a single dose or divided doses.

The compounds of the present application can be prepared by various synthetic methods well known to those skilled in the art, including the specific examples listed below, the embodiments formed by the combination with other chemical synthesis methods, and equivalent alternative embodiments well known to those skilled in the art, wherein the preferred embodiments include, but are not limited to, the examples of the present application.

The chemical reactions described in the specific examples of the present application are completed in a suitable solvent, wherein the solvent must be suitable for the chemical changes of the present application and the reagents and materials required thereby. In order to obtain the compounds of the present application, sometimes those skilled in the art need to modify or select synthesis steps or reaction schemes based on the existing examples.

In some embodiments, some of the compounds of general formula (III) of the present application may be prepared by a person skilled in the field of organic synthesis via the following synthetic route 1:

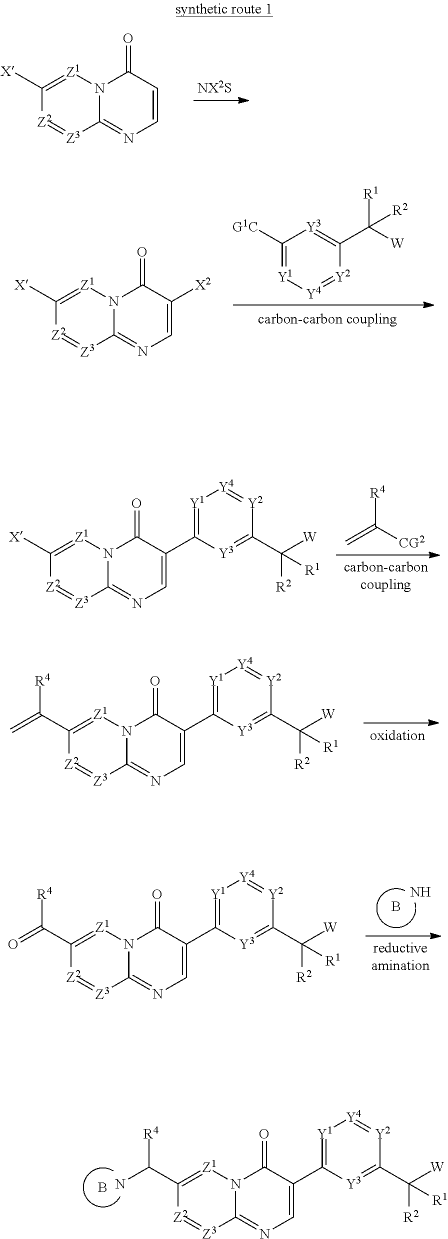

wherein: X′ is halogen or OTf; X² is halogen; CG¹ and CG² are independently selected from B(OR)₂, BF₃K, SnR′₄, ZnX²,

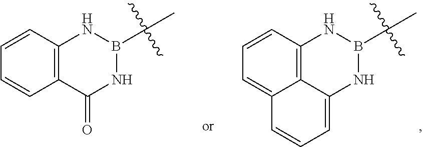

and the R is selected from H or C₁-C₆ alkyl, R′ is selected from C₁-C₆ alkyl, and X² is halogen; Z¹, Z², Z³, Y¹, Y², Y³, Y⁴, W, R¹, R², R⁴ and ring B are as defined in general formula (III).

In some embodiments, some of the compounds of general formula (III) of the present application may be prepared by a person skilled in the field of organic synthesis via the following synthetic route 2:

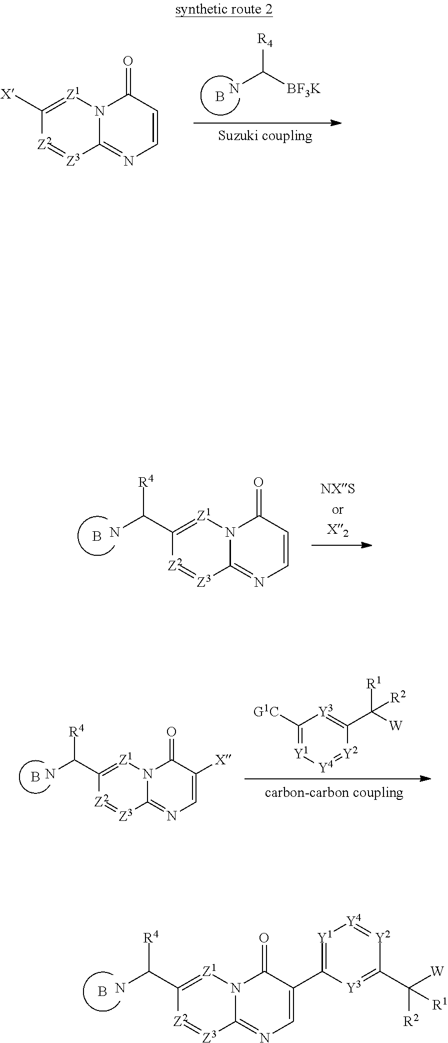

wherein: X′ is halogen or OTf; X″ is halogen; CG¹ is independently selected from B(OR)₂, BF₃K, SnR′₄, ZnX″,

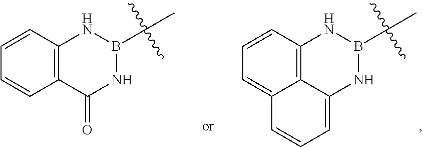

and the R is selected from H or C₁-C₆ alkyl, R′ is selected from C₁-C₆ alkyl, and X″ is halogen; Z¹, Z², Z³, Y¹, Y², Y³, Y⁴, W, R¹, R², R⁴ and ring B are as defined in general formula (III).

In some embodiments, some of the compounds of general formula (VI) of the present application may be prepared by a person skilled in the field of organic synthesis via the following synthetic route 3:

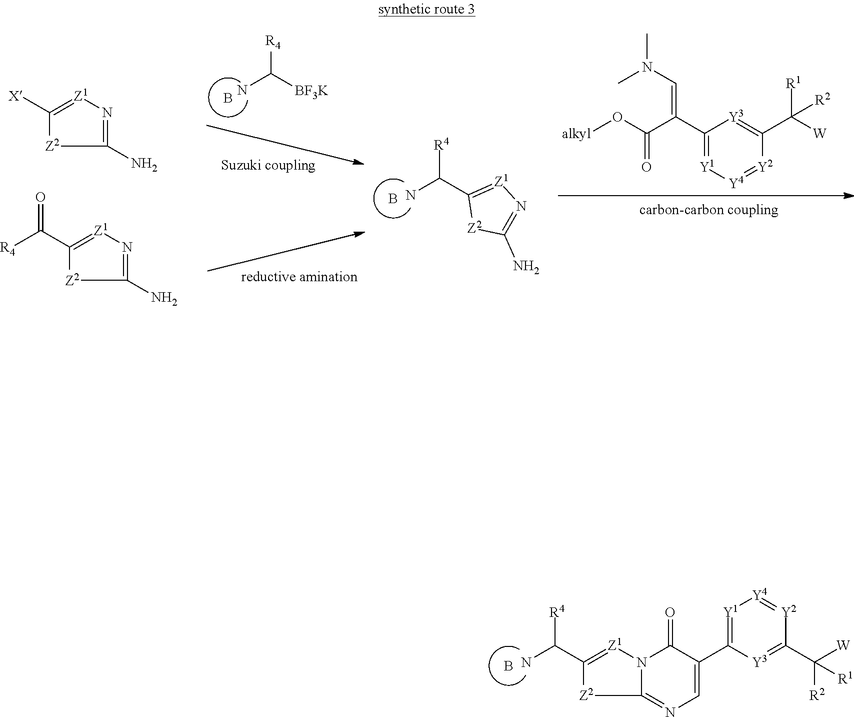

wherein: X′ is halogen or OTf; Z¹, Z², Y¹, Y², Y³, Y⁴, W, R¹, R², R⁴ and ring B are as defined in general formula (VI).

In some embodiments, some of the compounds of general formula (V) of the present application may be prepared by a person skilled in the field of organic synthesis via the following synthetic route 4:

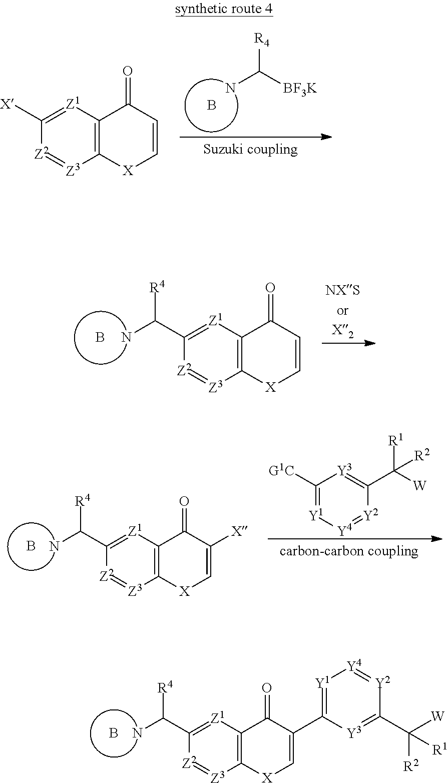

wherein: X′ is halogen or OTf; X″ is halogen; CG¹ is independently selected from B(OR)₂, BF₃K, SnR′₄, ZnX″,

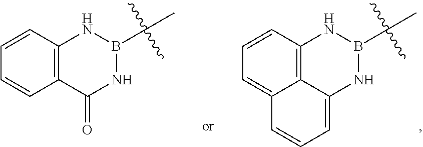

the R is selected from H or C₁-C₆ alkyl, and R′ is selected from C₁-C₆ alkyl; Z¹, Z², Z³, X, Y¹, Y², Y³, Y⁴, W, R¹, R², R⁴ and ring B are as defined in general formula (V).

### EXAMPLES

The present invention will be described in detail with the following examples, but not imply any adverse limitation to the present application. The present invention has been described in detail herein, and the specific examples thereof are also disclosed. Various changes and improvements to the specific examples of the present application would be obvious to those skilled in the art without departing from the spirit and scope of the present application. All reagents used in the present application are commercially available and can be used without further purification.

Unless otherwise specified, the ratios indicated for mixed solvents are volume mixing ratios. Unless otherwise specified, % refers to wt %.

Compounds are named by hand or ChemDraw® software, and commercially available compounds are named by the supplier catalog names.

The structures of the compounds are determined by nuclear magnetic resonance (NMR) and/or mass spectrometry (MS). The NMR shifts are calculated in 10⁻⁶ (ppm). The solvents for NMR assay are deuterated dimethyl sulfoxide, deuterated chloroform, deuterated methanol, etc., and the internal standard is tetramethylsilane (TMS). The “IC₅₀” refers to the half inhibitory concentration, the concentration at which half of the maximal inhibitory effect is achieved.

The eluent hereinafter may be formed from two or more solvents to form a mixed eluent, and the ratio of which is the volume ratio of the solvents. For example, the “0 to 10% methanol/dichloromethane” means that the volume ratio of methanol to dichloromethane in the mixed eluent is 0:100 to 10:90 during gradient elution.

Explanation of terms or abbreviations: B₂Pin₂: pinacol diborate; Pd(dppf)Cl₂: [1,1′-bis(diphenylphosphine) ferrocene]palladium dichloride; NIS: N-iodosuccinimide; DCM: dichloromethane; Pd(PPh₃)₄: tetrakistriphenylphosphine palladium; THF: tetrahydrofuran; XphosPdG₂: chloro(2-dicyclohexylphosphino-2′,4′,6′-triisopropyl-1,1′-biphenyl)[2-(2′-amino-1,1′-biphenyl)]palladium(II); Xphos: 2-dicyclohexylphosphino-2′,4′,6′-triisopropylbiphenyl; SFC: supercritical fluid chromatography; (Boc)₂O: di-tert-butyl dicarbonate; DAMP: N,N-dimethylpyridin-4-amine; TFA: trifluoroacetic acid; Pd(dba)₂: palladium bis-dibenzylideneacetone; P(tBu)₃: tri-tert-butylphosphine; AcOH: acetic acid; Bredereck's regent: 1-tert-butoxy-N,N,N′,N′-tetramethylmethanediamine; dioxane: 1,4-dioxane; t-BuOK: potassium tert-butoxide; LDA: lithium diisopropylamide; HATU: 2-(7-azobenzotriazole)-N,N,N′,N′-tetramethyluronium hexafluorophosphate; DIPEA: N,N-diisopropylethylamine; DMF-DMA: N,N-dimethylformamide dimethyl acetal; T₃P: propyl phosphoric anhydride; TEA: triethylamine; DMSO: dimethyl sulfoxide; DAST: diethylamine sulfur trifluoride; PPh₃: triphenylphosphine; Ziram: zinc dimethyldithiocarbamate; DEAD: diethyl azodicarboxylate; (Rh(OAc)₂)₂: rhodium acetate dimer; D-M's reagent: Dess-Martin periodinane; XtalFluor-E: (diethylamino)difluorosulfonium tetrafluoroborate; Pd(OAc)₂: palladium acetate; P(Cy)₃: tricyclohexylphosphine; DIBAL-H: diisobutylaluminum hydride; DME: 1,2-dimethoxyethane; CAN: cerium ammonium nitrate; IPA: isopropyl alcohol; DEA: diethanolamine; DMF: N,N-dimethylformamide; DBU: 1,8-diazabicyclo[5.4.0]undec-7-ene; MsCl: methanesulfonyl chloride; NBS: N-bromosuccinimide; DCE: dichloroethane; DIEA: N,N-diisopropylethylamine; pyrrolidine: tetrahydropyrrole; pyridine: pyridine; DHP: 3,4-dihydropyran; PPTS: pyridine-p-toluenesulfonate

### Example 1: Preparation of 3-[3-[3-methyl-1-(4-methyl-4H-1,2,4-triazol-3-yl)cyclobutyl]phenyl]-7-[[(3S)-3-methyl-1-piperidyl]methyl]pyrido[1,2-a]pyrimidin-4-one (compound 1)

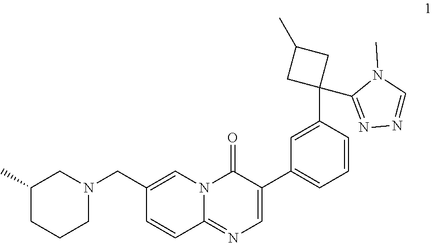 [SMILES: CC1CC(c2cccc(-c3cnc4ccc(CN5CCC[C@H](C)C5)cn4c3=O)c2)(c2nncn2C)C1]

### Synthetic Route:

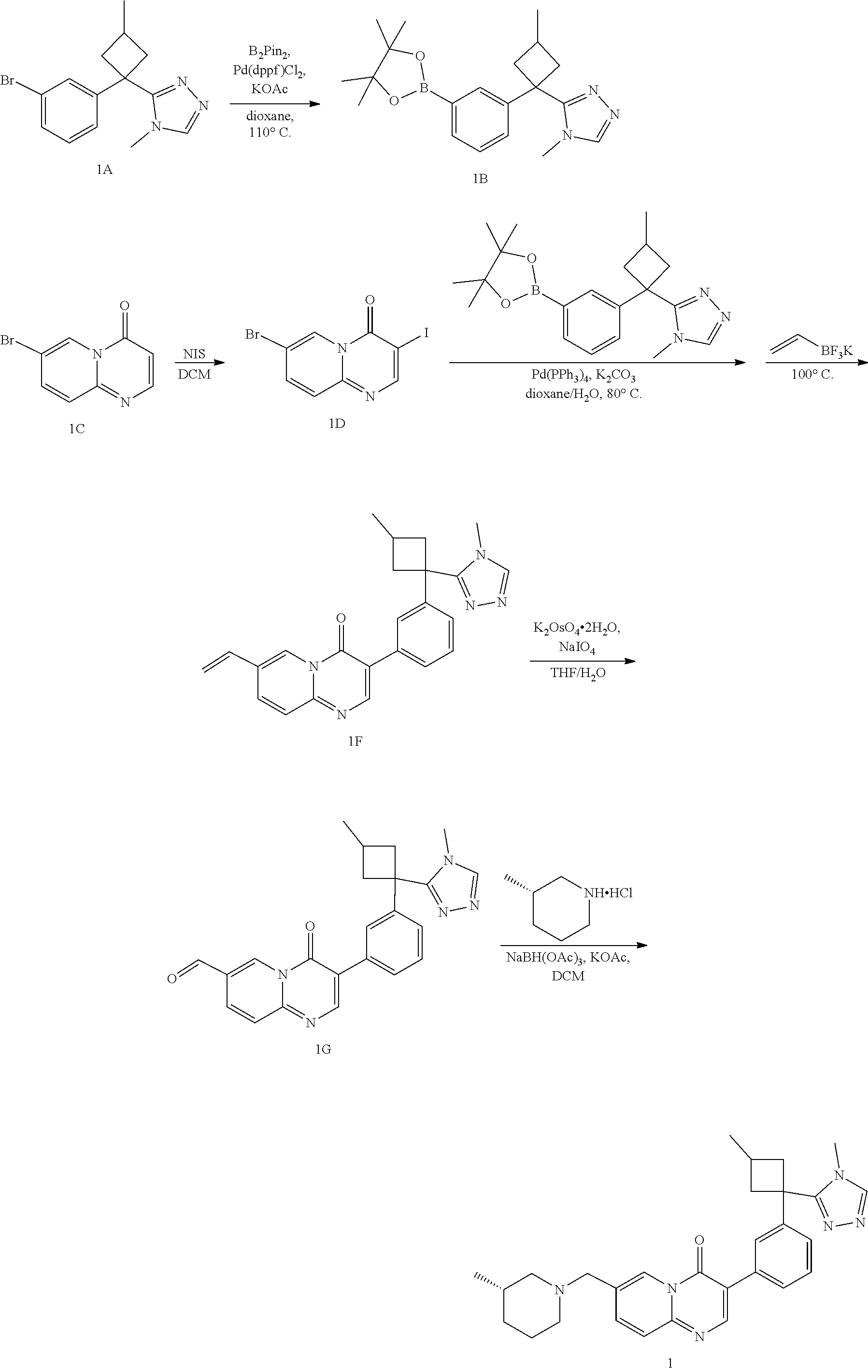

### Step 1: Preparation of 4-methyl-3-(3-methyl-1-(3-(4,4,5,5-tetramethyl-1,3,2-dioxaboran-2-yl)phenyl)cyclobutyl)-4H-1,2,4-triazole (1B)

3-(1-(3-Bromophenyl)-3-methylcyclobutyl)-4-methyl-4H-1,2,4-triazole (400 mg, 1.31 mmol) and B₂Pin₂ (498 mg, 1.96 mmol) were dissolved in 1,4-dioxane (40 mL), potassium acetate (385 mg, 3.92 mmol) and Pd(dppf)Cl₂ (96 mg, 0.13 mmol) were then added, and nitrogen replacement was performed for three times. The mixture was heated to 110° C. under nitrogen protection and stirred for 12 h. The reaction solution was cooled to room temperature, filtered, the filter cake was washed with ethyl acetate, and the filtrate was evaporated to dryness to obtain the title compound 1B as a crude, which was directly used in the next step.

MS m/z (ESI): 354.1 [M+H].

### Step 2: Preparation of 7-bromo-3-iodo-4H-pyrido[1,2-a]pyrimidin-4-one (1D)

7-Bromo-4H-pyrido[1,2-a]pyrimidin-4-one (250 mg, 1.11 mmol) was dissolved in dichloromethane (20 mL), and N-iodosuccinimide (375 mg, 1.67 mmol) was added. The resulting solution was stirred at room temperature overnight, and then saturated aqueous sodium sulfite solution was added to remove excess N-iodosuccinimide. The mixture was extracted with ethyl acetate, and the organic phase was dried over anhydrous magnesium sulfate, filtered, and the solution was evaporated to dryness to obtain the title compound 1D as a crude, which was directly used in the next step.

MS m/z (ESI): 351.1/353.1 [M+H].

### Step 3: Preparation of 3-(3-(3-methyl-1-(4-methyl-4H-1,2,4-triazol-3-yl)cyclobutyl)phenyl)-7-ethenyl-4H-pyrido[1,2-a]pyrimidin-4-one (1F)

To a mixture of 1B (200 mg, 0.57 mmol), 1D (302 mg, 0.86 mmol), potassium carbonate (472 mg, 3.42 mmol) and Pd(PPh₃)₄ (132 mg, 0.11 mmol) were added 1,4-dioxane (15 mL) and water (5 mL), and nitrogen replacement was performed for three times. The mixture was then heated to 80° C. under nitrogen protection and stirred for 3 hours. The reaction solution was cooled to room temperature, ethylene potassium trifluoroborate (131 mg, 0.86 mmol) was added, and the mixture was heated to 100° C., and stirred for 3 hours. The reaction solution was cooled to room temperature, the solvent was removed by rotary evaporation, and the residue was purified by reverse phase column chromatography (chromatographic column: SepaFlash® silica gel column; mobile phase: acetonitrile/water=60:40), to obtain the title compound 1F (180 mg).

MS m/z (ESI): 398.1 [M+H].

### Step 4: Preparation of 3-[3-[3-methyl-1-(4-methyl-4H-1,2,4-triazol-3-yl)cyclobutyl]phenyl]-4-oxo-pyrido[1,2-a]pyrimidine-7-carbaldehyde (1G)

1F (180 mg, 0.45 mmol) was dissolved in tetrahydrofuran (5 mL), then potassium osmate dihydrate and 0.5 mol/L aqueous sodium periodate solution (5 mL) were added, and the mixture was stirred at room temperature for 1 hour. After the reaction was completed, the solid is filtered off, the filter cake was washed with ethyl acetate, the aqueous layer was separated from the filtrate, the organic phase was washed with saturated aqueous sodium sulfite solution, dried over anhydrous magnesium sulfate, filtered, and evaporated to dryness to obtain the title compound 1G (160 mg) as a crude, which was directly used in the next step.

MS m/z (ESI): 400.2 [M+H].

### Step 5: Preparation of 3-[3-[3-methyl-1-(4-methyl-4H-1,2,4-triazol-3-yl)cyclobutyl]phenyl]-7-[[(3S)-3-methyl-1-piperidyl]methyl]pyrido[1,2-a]pyrimidin-4-one (compound 1)

To a solution of 1G (90 mg, 0.23 mmol) in dichloromethane (10 mL) were sequentially added (S)-3-methylpiperidine hydrochloride (46 mg, 0.34 mmol), potassium acetate (44 mg, 0.45 mmol) and sodium acetate borohydride (57 mg, 0.27 mmol). The resulting mixture was stirred overnight under nitrogen protection. The reaction solution was quenched with methanol, the solvent was evaporated to dryness, and the residue was subjected to normal phase column chromatography (methanol:dichloromethane=1:8), and then purified by reverse phase column chromatography (chromatographic column: SepaFlash® silica gel column; mobile phase: acetonitrile/water=60:40), to obtain the title compound 1 (9.1 mg).

MS m/z (ESI): 483.4 [M+H].

¹H NMR (400 MHz, DMSO-d₆) δ 8.99 (s, 1H), 8.60-8.59 (m, 1H), 8.37-8.29 (m, 1H), 7.94 (dd, J=9.1, 1.7 Hz, 1H), 7.89 (brs, 1H), 7.74 (d, J=9.0 Hz, 1H), 7.68-7.62 (m, 1H), 7.47-7.40 (m, 1H), 7.28 (d, J=7.9 Hz, 1H), 3.63-3.53 (m, 2H), 3.28-3.22 (m, 3H), 2.94-2.82 (m, 2H), 2.76-2.67 (m, 2H), 2.60-2.54 (m, 2H), 2.36-2.23 (m, 1H), 1.94 (t, J=10.8 Hz, 1H), 1.70-1.56 (m, 4H), 1.51-1.41 (m, 1H), 1.11-1.08 (m, 3H), 0.91-0.77 (m, 4H).

### Example 2: Preparation of 3-[3-[3-methyl-1-(4-methyl-4H-1,2,4-triazol-3-yl)cyclobutyl]phenyl]-7-[[(3S)-3-methyl-1-piperidyl]methyl]-9-(trifluoromethyl)pyrido[1,2-a]pyrimidin-4-one (compound 2), 3-(3-((1S,3R)-3-methyl-1-(4-methyl-4H-1,2,4-triazol-3-yl)cyclobutyl)phenyl)-7-(((S)-3-methylpiperidin-1-yl)methyl)-9-(trifluoromethyl)-4H-pyrido[1,2-a]pyrimidin-4-one and 3-(3-((1R,3S)-3-methyl-1-(4-methyl-4H-1,2,4-triazol-3-yl)cyclobutyl) phenyl)-7-(((S)-3-methylpiperidin-1-yl)methyl)-9-(trifluoromethyl)-4H-pyrido[1,2-a]pyrimidin-4-one

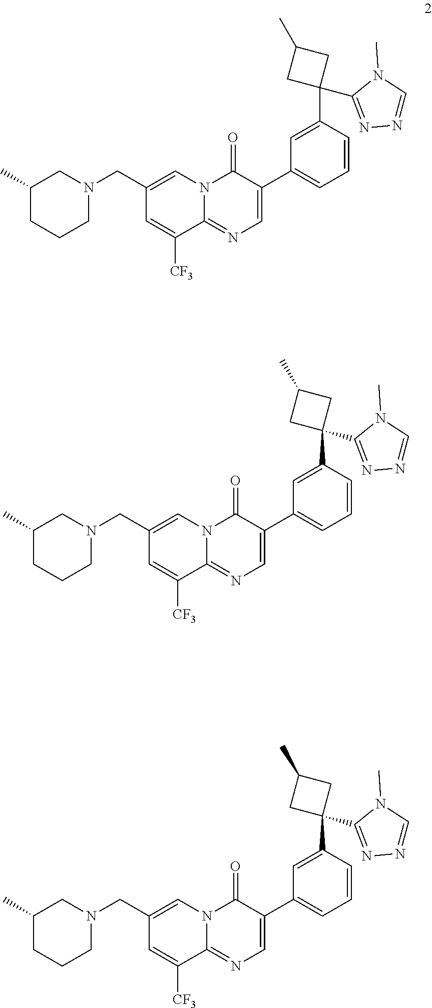 [SMILES: CC1CC(c2cccc(-c3cnc4c(C(F)(F)F)cc(CN5CCC[C@H](C)C5)cn4c3=O)c2)(c2nncn2C)C1 ; C[C@H]1CCCN(Cc2cc(C(F)(F)F)c3ncc(-c4cccc([C@]5(c6nncn6C)C[C@@H](C)C5)c4)c(=O)n3c2)C1 ; C[C@H]1CCCN(Cc2cc(C(F)(F)F)c3ncc(-c4cccc([C@]5(c6nncn6C)C[C@H](C)C5)c4)c(=O)n3c2)C1]

### Synthetic Route:

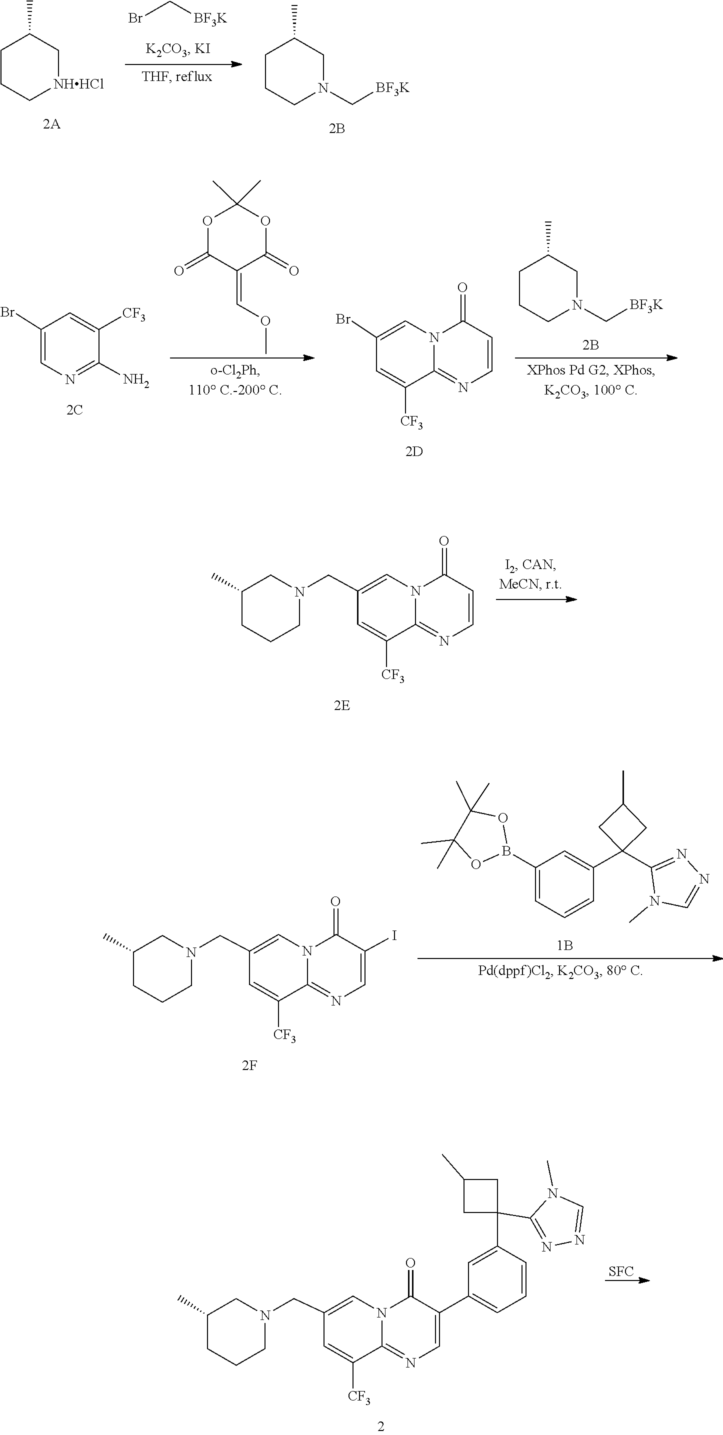

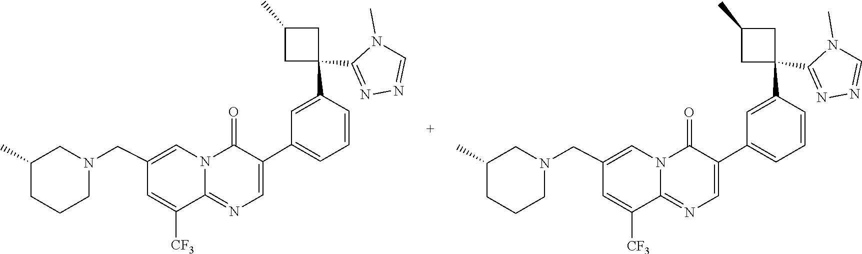 [SMILES: C[C@H]1CCCN(Cc2cc(C(F)(F)F)c3ncc(-c4cccc([C@]5(c6nncn6C)C[C@@H](C)C5)c4)c(=O)n3c2)C1 ; C[C@H]1CCCN(Cc2cc(C(F)(F)F)c3ncc(-c4cccc([C@]5(c6nncn6C)C[C@H](C)C5)c4)c(=O)n3c2)C1]

### Step 1: Preparation of potassium (S)-trifluoro(3-methylpiperidin-1-yl)methyl boronate (2B)

Anhydrous tetrahydrofuran (10 mL) was added into (S)-3-methylpiperidine hydrochloride (2A, 540 mg, 3.98 mmol), potassium (bromomethyl)trifluoroborate (960 mg, 4.78 mmol), potassium carbonate (605 mg, 4.38 mmol) and potassium iodide (67 mg, 0.40 mmol), and nitrogen replacement was performed for three times. The mixture was heated to reflux under nitrogen protection and stirred for 12 h. After the reaction was completed, the mixture was cooled to room temperature, 100 mL of acetone was added, the mixture was filtered, the filter cake was washed with acetone, and the filtrate was evaporated to dryness to obtain the title compound 2B (420 mg) as a crude, which was directly used in the next step.

MS m/z (ESI): 162.1 [M−KF+H]⁺.

### Step 2: Preparation of 7-bromo-9-(trifluoromethyl)-4H-pyrido[1,2-a]pyrimidin-4-one (2D)

To a mixture of 5-bromo-3-(trifluoromethyl)pyridin-2-amine (2C, 8.00 g, 33.2 mmol) and 5-(methoxymethylene)-2,2-dimethyl-1,3-dioxane-4,6-dione (7.42 g, 39.8 mmol) was added 1,2-dichlorobenzene (100 mL), the mixture was heated to 110° C., stirred for 4 hours, then the temperature was heated to 200° C., and stirring was continued for 3 hours. After the reaction was completed, the mixture was cooled to room temperature and separated and purified by normal phase column chromatography (petroleum ether:ethyl acetate=3:1) to obtain the title compound 2D (4.9 g).

MS m/z (ESI): 293.1/295.1 [M+H]⁺.

### Step 3: Preparation of 7-[[(3S)-3-methyl-1-piperidyl]methyl]-9-(trifluoromethyl)pyrido[1,2-a]pyrimidin-4-one (2E)

To a mixture of 2D (410 mg, 1.40 mmol), 2B (460 mg, 2.10 mmol), XphosPdG₂ (110 mg, 0.14 mmol), 2-dicyclohexylphosphine-2′,4′,6′-triisopropylbiphenyl (110 mg, 0.14 mmol) and potassium carbonate (133 mg, 4.20 mmol) was added 1,4-dioxane (20 mL) and water (5 mL), nitrogen replacement was performed for three times, and then the mixture was heated to 100° C. under nitrogen protection and stirred for 4 hours. After the reaction was completed, the mixture was cooled to room temperature and concentrated under reduced pressure. The residue was separated and purified by reverse phase column chromatography (chromatographic column: SepaFlash® silica gel column; mobile phase: acetonitrile/water=60:40), to obtain the title compound 2E (210 mg).

MS m/z (ESI): 326.1 [M+H]⁺.

### Step 4: Preparation of 3-iodo-7-[[(3S)-3-methyl-1-piperidyl]methyl]-9-(trifluoromethyl)pyrido[1,2-a]pyrimidin-4-one (2F)

2E (210 mg, 0.66 mmol) was dissolved in acetonitrile (10 ml), iodine (197 mg, 0.77 mmol) and ceric ammonium nitrate (342 mg, 0.65 mmol) were added, and the mixture was stirred at room temperature for 12 hours. After the reaction was completed, excess iodine was quenched with saturated aqueous sodium sulfite solution, the mixture was extracted with dichloromethane, and the organic phase was dried over anhydrous magnesium sulfate, filtered, and evaporated to dryness to obtain the title compound 2F (240 mg) as a crude, which was directly used in the next step.

MS m/z (ESI): 452.0 [M+H]⁺.

### Step 5: Preparation of 3-[3-[3-methyl-1-(4-methyl-4H-1,2,4-triazol-3-yl)cyclobutyl]phenyl]-7-[[(3S)-3-methyl-1-piperidyl]methyl]-9-(trifluoromethyl)pyrido[1,2-a]pyrimidin-4-one (compound 2), 3-(3-((1S,3R)-3-methyl-1-(4-methyl-4H-1,2,4-triazol-3-yl)cyclobutyl)phenyl)-7-(((S)-3-methylpiperidin-1-yl)methyl)-9-(trifluoromethyl)-4H-pyrido[1,2-a]pyrimidin-4-one and 3-(3-((1R,3S)-3-methyl-1-(4-methyl-4H-1,2,4-triazol-3-yl)cyclobutyl) phenyl)-7-(((S)-3-methyl piperidin-1-yl)methyl)-9-(trifluoromethyl)-4H-pyrido[1,2-a]pyrimidin-4-one

To a solution of 2F (240 mg, 0.53 mmol), 1B (188 mg, 0.53 mmol) and potassium carbonate (221 mg, 1.60 mmol) in dioxane/water (25 mL, 4:1) was added Pd(dppf)Cl₂ (39 mg, 0.53 mmol), and the resulting mixture was heated to 80° C. under nitrogen protection and stirred for 4 hours. The reaction solution was cooled to room temperature, and the solvent was removed by rotary evaporation. The residue was separated and purified by normal phase column chromatography (methanol:dichloromethane=1:10), followed by reverse phase column chromatography (chromatographic column: SepaFlash® silica gel column; mobile phase: acetonitrile/water=75:25), to obtain the title compound 2 (130 mg).

MS m/z (ESI): 551.0 [M+H]⁺.

Compound 2 was separated by SFC (instrument: CAS-SH-ANA-SFC-G (Agilent 1260 with DAD detector); column: ChiralPak AS-3, column length 150 mm, inner diameter 4.6 mm, and particle size 3 μm; mobile phase A: CO₂, mobile phase B: IPA (with 0.05% DEA); gradient: mobile phase B: from 5% to 40% over 4.5 minutes, then eluted with 5% mobile phase B for 1.5 minutes; flow rate: 2.5 mL/min; column temperature: 40° C.; automatic back pressure regulator (ABPR): 100 bar), to obtain the isomer title compound 2-P1 (t_{R}=4.52 min, 70 mg) and the title compound 2-P2 (t_{R}=4.69 min, 15 mg).

### 2-P1:

MS m/z (ESI): 551.2 [M+H]⁺;

¹H NMR (400 MHz, CDCl₃) δ 9.17 (s, 1H), 8.58 (s, 1H), 8.25 (br. s., 1H), 7.97 (s, 1H), 7.88 (s, 1H), 7.63 (d, J=8.0 Hz, 1H), 7.45 (t, J=8.0 Hz, 1H), 7.32 (d, J=8.0 Hz, 1H), 3.56 (br. s., 2H), 3.25 (s, 3H), 2.99-2.84 (m, 2H), 2.84-2.62 (m, 5H), 2.10-1.94 (m, 1H), 1.78-1.53 (m, 6H), 1.19-1.10 (m, 3H), 1.02-0.91 (m, 1H), 0.90-0.82 (m, 3H).

### 2-P2:

MS m/z (ESI): 551.2 [M+H]⁺;

¹H NMR (400 MHz, CDCl₃) δ 9.16 (s, 1H), 8.59-8.55 (m, 1H), 8.25 (s, 1H), 8.03 (s, 1H), 7.71 (s, 1H), 7.62-7.57 (m, 1H), 7.45-7.39 (m, 1H), 7.23-7.16 (m, 1H), 3.56 (s, 2H), 3.31 (s, 3H), 3.26-3.16 (m, 2H), 2.83-2.69 (m, 2H), 2.65-2.54 (m, 1H), 2.41-2.31 (m, 2H), 2.06-1.97 (m, 1H), 1.71-1.54 (m, 5H), 1.39-1.23 (m, 2H), 1.18-1.11 (m, 3H), 0.89-0.85 (m, 3H).

### Example 3: Preparation of (S)-8-methyl-3-(3-(3-methyl-1-(4-methyl-4H-1,2,4-triazol-3-yl)cyclobutyl)phenyl)-6-((3-methylpiperidin-1-yl)methyl)-4H-chromen-4-one (compound 58)

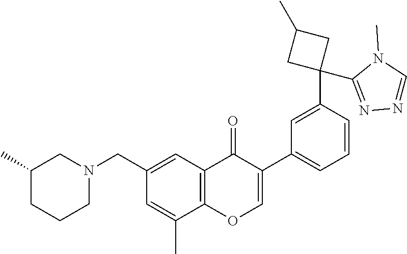 [SMILES: Cc1cc(CN2CCC[C@H](C)C2)cc2c(=O)c(-c3cccc(C4(c5nncn5C)CC(C)C4)c3)coc12]

### Synthetic Route:

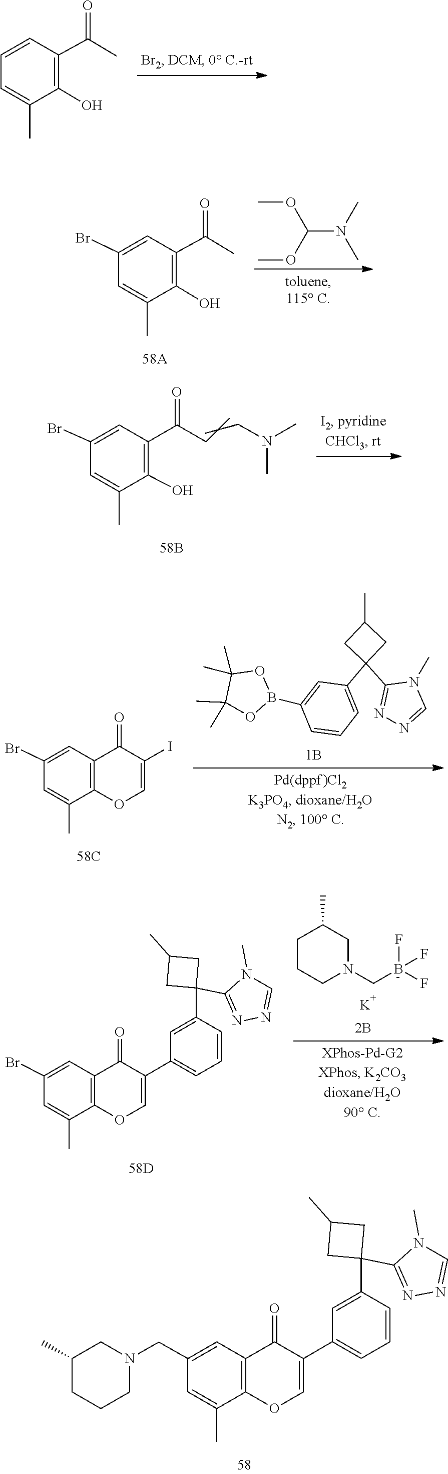

### Step 1: Preparation of 1-(5-bromo-2-hydroxyl-3-methylphenyl)ethanone (58A)

1-(2-Hydroxyl-3-methylphenyl)ethanone (2 g, 13.32 mmol) was dissolved in anhydrous dichloromethane (20 mL). In an ice bath at 0° C., liquid bromine (3.62 g, 22.64 mmol, 1.24 mL) dissolved in anhydrous dichloromethane (20 mL) was slowly added dropwise to the solution. The reaction was continued with stirring at 0° C. in an ice bath for 1 h. After the reaction was completed, once upon the temperature of the reaction solution was heated to room temperature, the excess liquid bromine was quenched with a saturated aqueous sodium metabisulfite solution, and the reaction solution was extracted with dichloromethane. The organic phase was dried over anhydrous sodium sulfate, filtered, and distilled under reduced pressure and spin-dried to obtain a crude product. The crude product was purified by normal phase column chromatography (petroleum ether:ethyl acetate=100:1) to obtain the title compound 58A (3 g).

MS m/z(ESI): 228.8/230.8 [M+H]⁺.

### Step 2: Preparation of 1-(5-bromo-2-hydroxyl-3-methylphenyl)-3-(dimethylamino)prop-2-en-1-one (58B)

At room temperature, N,N-dimethylformamide dimethyl acetal (4.16 g, 34.92 mmol) was added to a sealed tube filled with a solution of 58A (3 g, 13.3 mmol) in anhydrous toluene (60 mL). The reaction solution was then heated to 115° C. and stirred for 16 hours. After the reaction was completed, the reaction solution was cooled to room temperature, water (60 mL) was added, and the mixture was extracted with ethyl acetate (50 mL×3). The organic phase was dried over anhydrous sodium sulfate, filtered, and distilled under reduced pressure to obtain a crude product; the crude product was purified by normal phase column chromatography (petroleum ether:ethyl acetate=73:27), to obtain the title product 58B (640 mg).

MS m/z (ESI): 284.9/285.7 [M+H]⁺.

### Step 3: Preparation of 6-bromo-3 iodo-8-methyl-4H-chromen-4-one (58C)

At room temperature, pyridine (353.54 mg, 4.47 mmol, 360.05 μL) and iodine (1.13 g, 4.47 mmol) were added to a solution of 58B (635 mg, 2.23 mmol) in chloroform (10 mL). The reaction mixture was reacted at room temperature for 16 hours. After the reaction was completed, the excess iodine was quenched with a saturated aqueous sodium sulfite solution, and the mixture was extracted with dichloromethane. The organic phase was dried over anhydrous sodium sulfate, filtered, and distilled under reduced pressure to obtain the title compound 58C (732 mg) as a crude, which was directly used in the next step.

MS m/z (ESI): 364.7/366.7 [M+H]⁺.

### Step 4: Preparation of 6-bromo-8-methyl-3-(3-(3-methyl-1-(4-methyl-4H-1,2,4-triazol-3-yl)cyclobutyl)phenyl)-4H-chromen-4-one (58D)

58C (300 mg, 822.01 μmol), 1B (232.31 mg, 657.60 μmol) and potassium phosphate (261.73 mg, 1.23 mmol) were added to a mixture of 1,4-dioxane (10 mL) and water (1 mL), and nitrogen replacement was performed; under nitrogen atmosphere, Pd(dppf)Cl₂ (6.01 mg, 8.22 mol) was added to the reaction solution. After the addition was completed, the reaction was heated to 100° C. and stirred under a nitrogen atmosphere for 16 hours. After the reaction was completed, the reaction solution was cooled to room temperature, water (15 mL) was added, and the mixture was extracted with ethyl acetate. The organic phase obtained by extraction was dried over anhydrous sodium sulfate, filtered, and distilled under reduced pressure to obtain a crude product, which was purified by normal phase column chromatography (petroleum ether:ethyl acetate=0:100) to obtain the title product 58D (240 mg).

MS m/z(ESI): 464.0/466.0 [M+H]⁺.

### Step 5: Preparation of (S)-8-methyl-3-(3-(3-methyl-1-(4-methyl-4H-1,2,4-triazol-3-yl)cyclobutyl)phenyl)-6-((3-methylpiperidin-1-yl)methyl)-4H-chromen-4-one (compound 58)

58D (120 mg, 258.42 μmol), 2B (79.27 mg, 361.79 μmol) and potassium carbonate (107.15 mg, 775.27 μmol) were added to a mixture of water (1 mL) and 1,4-dioxane (10 mL), and nitrogen replacement was performed. Under nitrogen atmosphere, XphosPdG₂ (20.33 mg, 25.84 mol) and Xphos (24.64 mg, 51.68 μmol) were added to the solution, and the reaction was heated to 90° C. and stirred for 16 hours. After the reaction was completed, the reaction was cooled to room temperature, water (10 mL) was added, and the mixture was extracted with ethyl acetate (10 mL×3). The organic phase was dried over anhydrous sodium sulfate, filtered, and distilled under reduced pressure to obtain a crude product, which was purified by high performance liquid chromatography (chromatographic column: Welch Xtimate C18; column length 150 mm, inner diameter 30 mm, and particle size 5 μm; mobile phase A: water (with 0.225% formic acid); mobile phase B: acetonitrile; gradient: mobile phase B: from 24% to 54% over 7 minutes, then eluted with 100% mobile phase B for 3 minutes; flow rate: 25 mL/min), to obtain the title compound 58 (43.25 mg, 34%).

MS m/z (ESI): 497.2 [M+H]⁺.

¹H NMR (400 MHz, CDCl₃) δ 8.61-8.05 (m, 1H), 8.04-7.94 (m, 2H), 7.75 (br. s., 1H), 7.62 (s, 1H), 7.46-7.35 (m, 3H), 3.81-3.66 (m, 2H), 3.34-3.24 (m, 3H), 3.22-3.13 (m, 1H), 3.05-2.97 (m, 1H), 2.96-2.85 (m, 3H), 2.73-2.64 (m, 2H), 2.52 (s, 3H), 2.39-2.27 (m, 1H), 2.14-2.00 (m, 1H), 1.91-1.62 (m, 5H), 1.19-1.10 (m, 3H), 0.89-0.82 (m, 3H).

### Example 4: Preparation of (S)-6-(3-(3-methyl-1-(4-methyl-4H-1,2,4-triazol-3-yl)cyclobutyl)phenyl)-2-((3-methylpiperidin-1-yl)methyl)-5H-thiazolo[3,2-a]pyrimidin-5-one (compound 59)

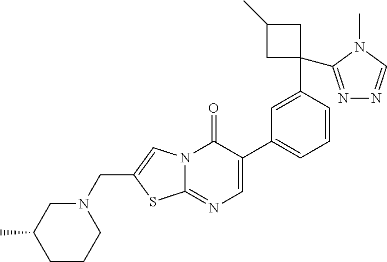 [SMILES: CC1CC(c2cccc(-c3cnc4sc(CN5CCC[C@H](C)C5)cn4c3=O)c2)(c2nncn2C)C1]

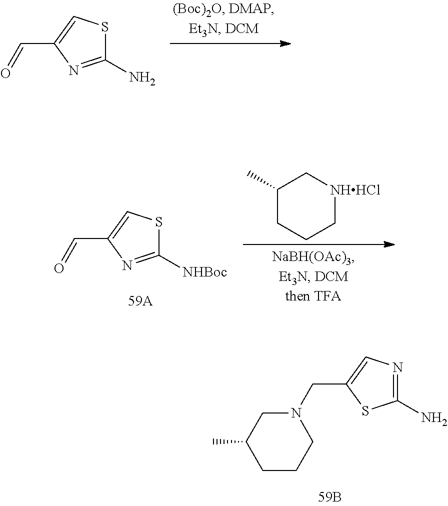

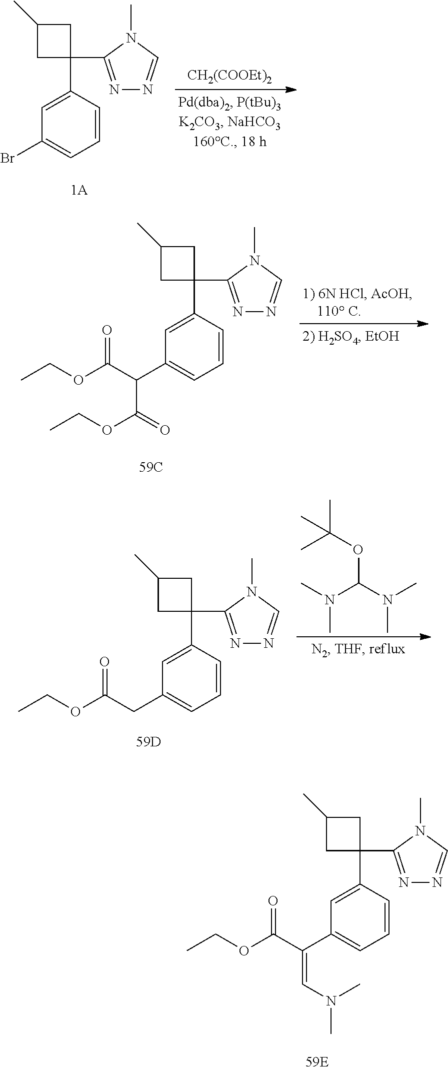

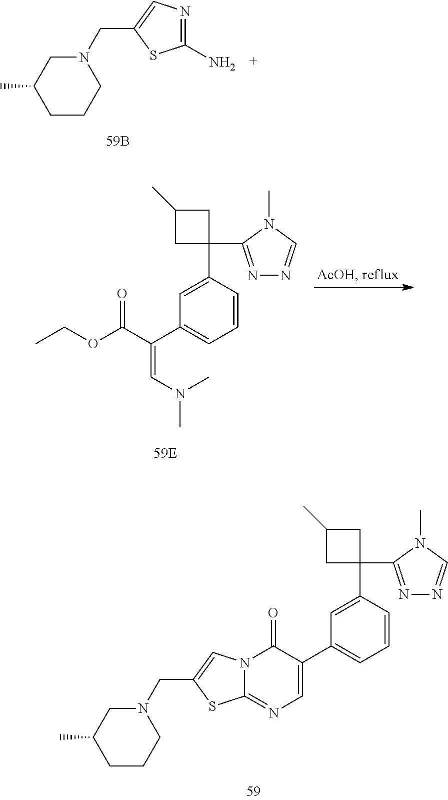 [SMILES: C[C@H]1CCCN(Cc2cnc(N)s2)C1 ; CCOC(=O)/C(=C/N(C)C)c1cccc(C2(c3nncn3C)CC(C)C2)c1 ; CC1CC(c2cccc(-c3cnc4sc(CN5CCC[C@H](C)C5)cn4c3=O)c2)(c2nncn2C)C1]

### Step 1: Preparation of tert-butyl (4-formylthiazol-2-yl)carbamate (59A)

2-Aminothiazole-4-carbaldehyde (400 mg, 3.12 mmol), triethylamine (948 mg, 9.36 mmol) and (Boc)₂O (817 mg, 3.75 mmol) were dissolved in anhydrous dichloromethane (20 mL), then DMAP (76 mg, 0.62 mmol) was added, and the reaction solution was stirred at room temperature for 2 hours. After the reaction was completed, the crude product was obtained by distillation under reduced pressure and spin-drying. The crude product was purified by normal phase column chromatography (petroleum ether:ethyl acetate=1:1) to obtain the title compound 59A (620 mg).

MS m/z(ESI): 229.1 [M+H]⁺.

### Step 2: Preparation of (S)-5-((3-methylpiperidin-1-yl)methyl)thiazol-2-amine (59B)

59A (240 mg, 1.05 mmol), triethylamine (319 mg, 3.15 mmol) and (S)-3-methylpiperidine hydrochloride (214 mg, 1.58 mmol) were dissolved in anhydrous dichloromethane (20 mL), sodium borohydride acetate (334 mg, 1.58 mmol) was then added, and the reaction solution was stirred at room temperature overnight. After the reaction was completed, the mixture was quenched with saturated aqueous ammonium chloride solution, and extracted with dichloromethane, and the organic phase was distilled under reduced pressure and spin-dried to obtain the crude product. Trifluoroacetic acid (5 mL) was added to the crude product and the mixture was stirred for 1 hour. The trifluoroacetic acid was removed by concentration under reduced pressure. The residue was neutralized with saturated sodium bicarbonate solution and extracted with dichloromethane. The organic phase was spin-dried under reduced pressure to obtain a crude, which was purified by reverse phase column chromatography (chromatographic column: SepaFlash® silica gel column; mobile phase: acetonitrile/water=75:25), to obtain the title compound 59B (150 mg).

MS m/z(ESI): 212.1 [M+H]⁺.

### Step 3: Preparation of diethyl 2-(3-(3-methyl-1-(4-methyl-4H-1,2,4-triazol-3-yl)cyclobutyl)phenyl)malonate (59C)

To a mixture of 1A (1 g, 3.27 mmol), diethyl malonate (10.5 mg, 65.3 mmol), potassium carbonate (1.4 g, 9.8 mmol) and sodium bicarbonate (0.82 g, 9.8 mmol) were added bis(dibenzylideneacetonepalladium) (188 mg, 0.33 mmol) and tri-tert-butylphosphine (132 mg, 0.65 mmol) and nitrogen replacement was performed for three times. The reaction was carried out in a sealed tube at 160° C. under nitrogen protection for 18 hours. After the reaction was completed, the mixture was cooled to room temperature and filtered. The filter cake was washed twice with ethyl acetate and the filtrate was evaporated to dryness under reduced pressure. The residue was purified by normal phase column chromatography (dichloromethane:methanol=15:1), to obtain the title compound 59C (1.2 g).

MS m/z(ESI): 386.1 [M+H]⁺.

### Step 4: Preparation of ethyl 2-(3-(3-methyl-1-(4-methyl-4H-1,2,4-triazol-3-yl)cyclobutyl)phenyl)acetate (59D)

10 mL of 6N concentrated hydrochloric acid and 10 mL of glacial acetic acid were added to 59C (1.2 g, 3.11 mmol) and the resulting solution was heated to 110° C. and stirred for 2 hours. After the reaction was completed, the mixture was cooled to room temperature and evaporated to dryness under reduced pressure. The residue was dissolved in ethanol (20 mL), and then 0.2 mL of concentrated sulfuric acid was added. The reaction solution was refluxed for 2 hours, then cooled to room temperature and evaporated to dryness under reduced pressure. The residue was neutralized with saturated sodium bicarbonate solution and extracted with dichloromethane. The organic phase was evaporated to dryness under reduced pressure. The residue was purified by normal phase column chromatography (dichloromethane:methanol=15:1) to obtain the title compound 59D (680 mg).

MS m/z(ESI): 314.2 [M+H]⁺.

### Step 5: Preparation of ethyl 3-(dimethylamino)-2-(3-(3-methyl-1-(4-methyl-4H-1,2,4-triazol-3-yl)cyclobutyl)phenyl)acrylate (59E)

59D (640 mg, 2.04 mmol) and 1-tert-butoxy-N,N,N′,N′-tetramethylmethanediamine (1.1 g, 6.13 mmol) were dissolved in 30 mL of anhydrous tetrahydrofuran, and the resulting solution was refluxed under nitrogen protection for 12 hours. After the reaction was completed, the temperature was lowered to room temperature, low-boiling substances were evaporated under reduced pressure, and the residue was dried under vacuum to obtain the title compound 59E (780 mg) as a crude, which was directly used in the next step.

MS m/z(ESI): 369.1 [M+H]⁺.

### Step 6: Preparation of (S)-6-(3-(3-methyl-1-(4-methyl-4H-1,2,4-triazol-3-yl)cyclobutyl)phenyl)-2-((3-methylpiperidin-1-yl)methyl)-5H-thiazolo[3,2-a]pyrimidin-5-one (compound 59)

59E (104 mg, 0.28 mmol) and 59B (50 mg, 0.24 mmol) were dissolved in 5 mL of glacial acetic acid, and the reaction solution was refluxed for 12 hours. After the reaction was completed, the mixture was cooled to room temperature and concentrated under reduced pressure. The crude was purified by reverse phase column chromatography (chromatographic column: SepaFlash® silica gel column; mobile phase: acetonitrile/water=75:25), to obtain the title compound 59 (6 mg).

MS m/z(ESI): 489.5 [M+H]⁺.

¹H NMR (400 MHz, CDCl₃) δ 8.31-8.21 (m, 2H), 8.14 (s, 1H), 7.73 (s, 1H), 7.61-7.49 (m, 1H), 7.47-7.36 (m, 1H), 7.32-7.24 (m, 1H), 4.24-3.98 (m, 1H), 3.68 (s, 3H), 3.24-3.16 (m, 4H), 2.93-2.73 (m, 4H), 2.05-1.94 (m, 1H), 1.81-1.53 (m, 5H), 1.52-1.38 (m, 1H), 1.08 (s, 3H), 0.86-0.81 (m, 3H)

### Example 5: Preparation of 9-methyl-3-(3-(3-methyl-1-(4-methyl-4H-1,2,4-triazol-3-yl)cyclobutyl)phenyl)-7-(piperidin-3-yl)-4H-pyrido[1,2-a]pyrimidin-4-one (compound 60)

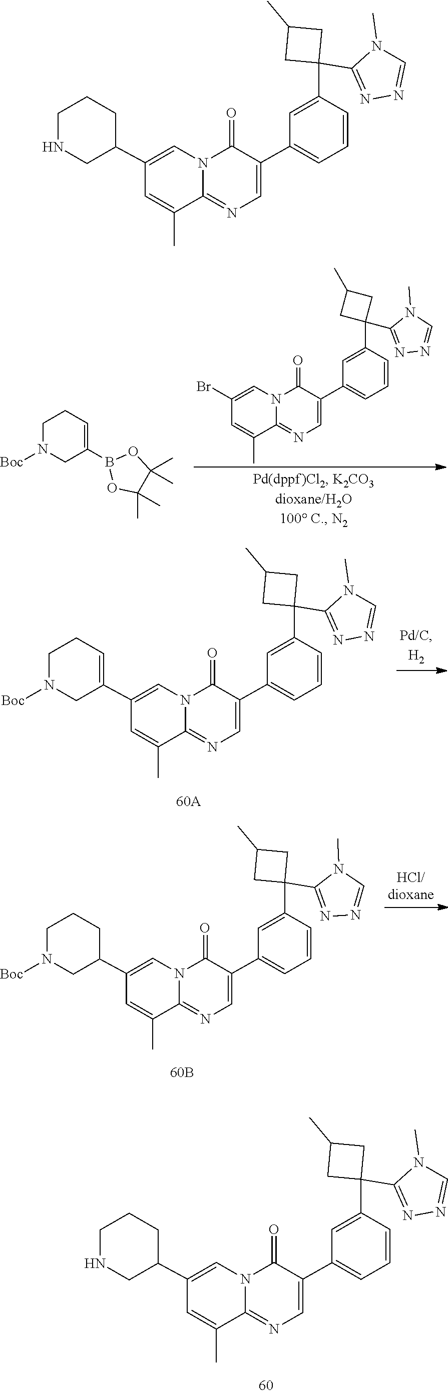 [SMILES: Cc1cc(C2CCCNC2)cn2c(=O)c(-c3cccc(C4(c5nncn5C)CC(C)C4)c3)cnc12 ; CC(C)(C)OC(=O)N1CCC=C(B2OC(C)(C)C(C)(C)O2)C1 ; Cc1cc(Br)cn2c(=O)c(-c3cccc(C4(c5nncn5C)CC(C)C4)c3)cnc12 ; Cc1cc(C2=CCCN(C(=O)OC(C)(C)C)C2)cn2c(=O)c(-c3cccc(C4(c5nncn5C)CC(C)C4)c3)cnc12 ; Cc1cc(C2CCCN(C(=O)OC(C)(C)C)C2)cn2c(=O)c(-c3cccc(C4(c5nncn5C)CC(C)C4)c3)cnc12 ; Cc1cc(C2CCCNC2)cn2c(=O)c(-c3cccc(C4(c5nncn5C)CC(C)C4)c3)cnc12]

### Step 1: Preparation of tert-butyl 5-(9-methyl-3-(3-(3-methyl-1-(4-methyl-4H-1,2,4-triazol-3-yl)cyclobutyl)phenyl)-4-oxo-4H-pyrido[1,2-a]pyrimidin-7-yl)-3,6-1,2,3,6-tetrahydropyridine-1-carboxylate (60A)

7-Bromo-9-methyl-3-(3-(3-methyl-1-(4-methyl-4H-1,2,4-triazol-3-yl)cyclobutyl)phenyl)-4H-pyrido[1,2-a]pyrimidin-4-one was synthesized in a manner similar to step 4 of Example 3, except that 58C (300 mg, 822.01 μmol) was replaced with 7-bromo-3-iodo-4H-pyrido[1,2-a]pyrimidin-4-one (288 mg, 822.01 μmol).

Tert-butyl 5-(4,4,5,5-tetramethyl-1,3,2-dioxaborolan-2-yl)-3,6-dihydropyridine-1(2H)-carboxylate (43.95 mg, 142.13 μmol), 7-bromo-9-methyl-3-(3-(3-methyl-1-(4-methyl-4H-1,2,4-triazol-3-yl)cyclobutyl)phenyl)-4H-pyrido[1,2-a]pyrimidin-4-one (60 mg, 129.21 μmol) and potassium carbonate (53.57 mg, 387.63 μmol) were added to a mixture of dioxane (3 mL) and water (0.6 mL), and nitrogen replacement was performed. Pd(dppf)Cl₂ (9.48 mg, 12.92 μmol) was then added into the reaction solution, and nitrogen replacement was performed. The reaction was heated to 100° C. and stirred under nitrogen protection for 16 hours. After the reaction was completed, the reaction solution was cooled to room temperature, water (15 mL) was added, and the mixture was extracted with ethyl acetate. The organic phase was dried over anhydrous sodium sulfate, filtered, and distilled under reduced pressure to obtain a crude product, which was purified by normal phase column chromatography (petroleum ether:ethyl acetate=1:1) to obtain the title product 60A (90 mg).

MS m/z (ESI): 567.2 [M+H]⁺.

### Step 2: Preparation of tert-butyl 3-(9-methyl-3-(3-(3-methyl-1-(4-methyl-4H-1,2,4-triazol-3-yl)cyclobutyl)phenyl)-4-oxo-4H-pyrido[1,2-a]pyrimidin-7-yl]piperidine-1-carboxylate (60B)

Ethyl acetate (5 mL) and wet palladium carbon (19.29 mg, 15.88 μmol, 10% purity) were added to 60A (90 mg, 158.82 μmol), hydrogen replacement was performed three times, and the mixture was stirred at 25° C. for 12 hours. After the reaction was completed, the mixture was filtered, and concentrated, to obtain the title product 60B (40 mg) as a crude, which was directly used in the next step.

MS m/z (ESI): 569.2 [M+H]⁺.

### Step 3: Preparation of 9-methyl-3-(3-(3-methyl-1-(4-methyl-4H-1,2,4-triazol-3-yl)cyclobutyl)phenyl)-7-(piperidin-3-yl)-4H-pyrido[1,2-a]pyrimidin-4-one (compound 60)

Hydrochloric acid/dioxane (1 mL, 4 mol/L) was added to 60B (35 mg, 61.54 μmol) and stirred at room temperature for 3 hours. After the reaction was completed, the solvent was removed by rotary evaporation to obtain a crude product, which was separated by preparative high performance liquid chromatography (column: Phenomenex Gemini C18, column length 75 mm, inner diameter 40 mm, particle size 3 μm; mobile phase A: water (0.225% FA), mobile phase B: acetonitrile; flow rate: 25 mL/min, gradient: mobile phase B: from 13% to 43% over 7 minutes, then eluted with 100% mobile phase B for 2 minutes; flow rate: 25 mL/min), to obtain the title compound 60 (4.2 mg).

MS m/z (ESI): 469.1 [M+H]⁺;

¹H NMR (400 MHz, DMSO-d₆) δ 8.99-8.85 (m, 1H), 8.51 (d, J=4.8 Hz, 1H), 8.20-7.98 (m, 1H), 7.88-7.66 (m, 1H), 7.60-7.52 (m, 2H), 7.48-7.40 (m, 1H), 7.39-7.35 (m, 0.5H), 7.25-7.20 (m, 0.5H), 3.73-3.38 (m, 2H), 3.35-3.26 (m, 3H), 3.24-3.12 (m, 2H), 2.96-2.90 (m, 2H), 2.72-2.67 (m, 3H), 2.57 (s, 3H), 2.43-1.93 (m, 4H), 1.86-1.71 (m, 1H), 1.19-1.09 (m, 3H).

### Example 6: Preparation of 3-(3-(1-cyclobutyl-1-(4-methyl-4H-1,2,4-triazol-3-yl)ethyl)phenyl)-7-(((S)-3-methylpiperidin-1-yl)methyl)-9-(trifluoromethyl)-4H-pyrido[1,2-a]pyrimidin-4-one (compound 61)

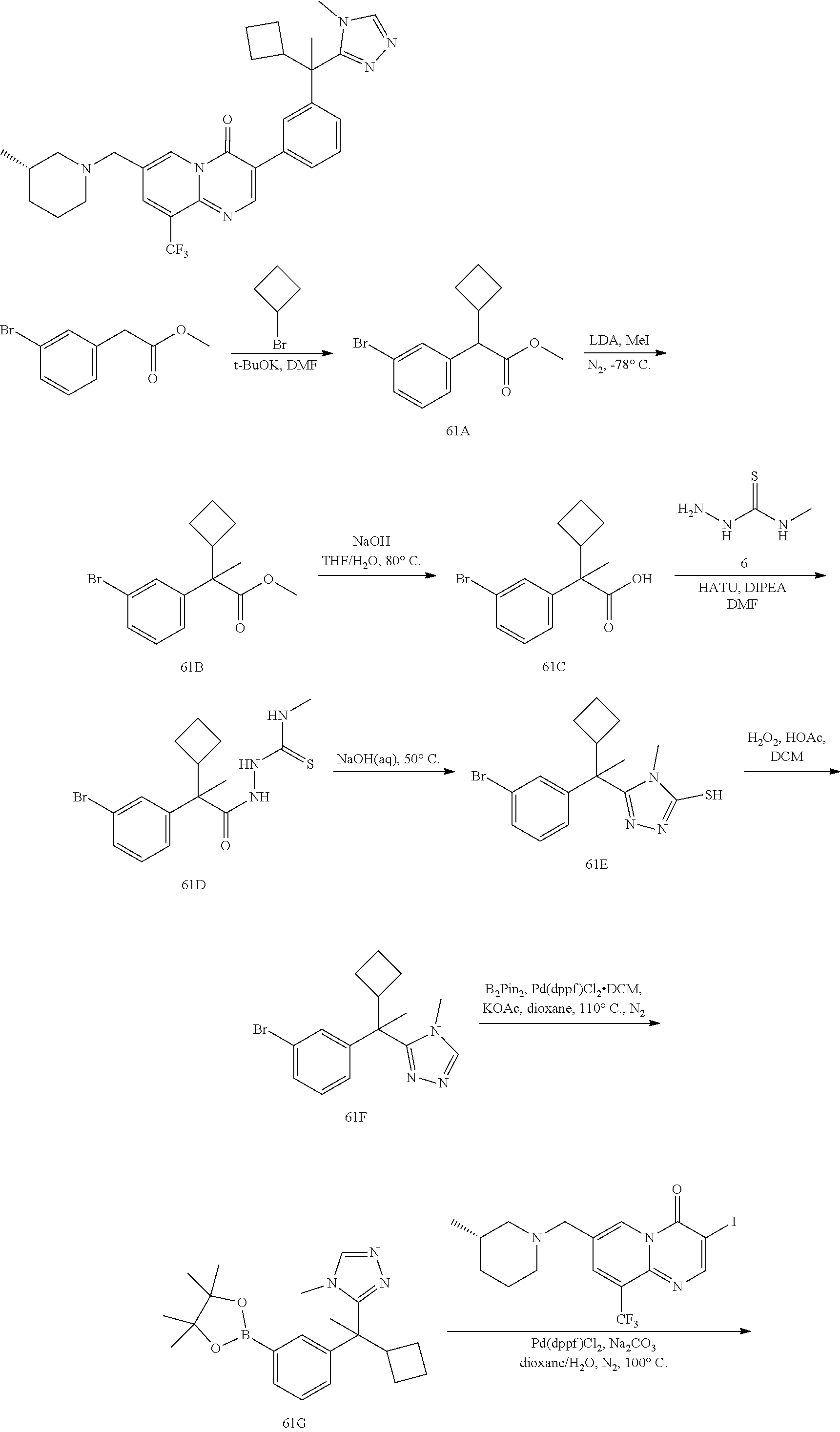

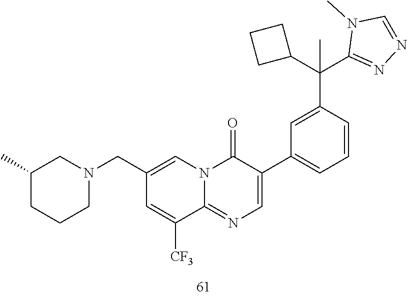 [SMILES: C[C@H]1CCCN(Cc2cc(C(F)(F)F)c3ncc(-c4cccc(C(C)(c5nncn5C)C5CCC5)c4)c(=O)n3c2)C1]

### Step 1: Preparation of methyl 2-(3-bromophenyl)-2-cyclobutyl acetate (61A)

To a solution of methyl 2-(3-bromophenyl)acetate (5 g, 21.83 mmol) in anhydrous DMF (50 mL) was added potassium tert-butoxide (3.18 g, 28.38 mmol) at 0° C. (ice-water bath), and the mixture was stirred at 0° C. (ice-water bath) for 0.5 h. To the reaction solution was added cyclobutyl bromide (3.54 g, 26.19 mmol) at 0° C. (ice-water bath). The reaction solution was returned to room temperature and stirred at room temperature for 12 hours. After the reaction was completed, the mixture was quenched with saturated aqueous ammonium chloride solution, and extracted with ethyl acetate, the organic phase was dried over anhydrous sodium sulfate, filtered, and concentrated under reduced pressure to obtain a crude as a colorless oil, which was purified by normal phase column chromatography (petroleum ether:ethyl acetate=10:1), to obtain the title compound 61A (4.5 g).

MS m/z (ESI): 283.16/284.9 [M+H]⁺.

### Step 2: Preparation of methyl 2-(3-bromophenyl)-2-cyclobutyl propionate (61B)

Methyl 2-(3-bromophenyl)-2-cyclobutylacetate (4.5 g, 15.89 mmol) was dissolved in anhydrous tetrahydrofuran (10 mL), and nitrogen replacement was performed three times. The mixture was cooled at −78° C. (dry ice/ethanol), and lithium diisopropylamide (13 mL, 26 mmol, 2M) was added dropwise to the mixture. The mixture was stirred at −78° C. (dry ice/ethanol) under nitrogen protection for 1 hour, and iodomethane (22.80 g, 160.63 mmol, 10 mL) was then added dropwise to the mixture. The reaction solution was returned to room temperature and stirred under nitrogen protection for 3 hours. After the reaction was completed, the mixture was quenched with saturated aqueous ammonium chloride solution, and extracted with ethyl acetate, the organic phase was dried over anhydrous sodium sulfate, filtered, and concentrated under reduced pressure to obtain a crude, which was separated and purified by normal phase column chromatography (petroleum ether:ethyl acetate=10:1), to obtain the title compound 61B (4.1 g).

MS m/z (ESI): 297.19/296.9 [M+H]⁺.

### Step 3: Preparation of 2-(3-bromophenyl)-2-cyclobutyl propanoic acid (61C)

To a solution of 61B (2 g, 6.73 mmol) in a mixture of water (3 mL) and tetrahydrofuran (20 mL) was added sodium hydroxide (1.35 g, 33.65 mmol), and the resulting mixture was heated to 80° C. and stirred for 12 hours. The reaction solution was cooled to room temperature, 2M aqueous hydrochloric acid solution was used to adjust the pH to 5, the mixture was extracted with ethyl acetate, and the organic phase was dried over anhydrous sodium sulfate, filtered, and concentrated under reduced pressure to obtain the title compound 61C (1.8 g) as a crude, which was directly used in the next step.

MS m/z (ESI): 283.16/283.8 [M+H]⁺.

### Step 4: Preparation of 2-(2-(3-bromophenyl)-2-cyclobutyl propanoyl)-N-methylthiosemicarbazide (61D)

HATU (1.35 g, 33.65 mmol) was added to a mixture of 61C (1.8 g, 6.06 mmol), 4-methyl-3-thioaminourea (1.8 g, 6.06 mmol), DIPEA (764.33 mg, 7.27 mmol), and anhydrous DMF (15 mL), and the mixture was stirred at room temperature for 12 hours. After the reaction was completed, the reaction solution was quenched with water, and extracted with ethyl acetate, the organic phase was dried over anhydrous sodium sulfate, filtered, and concentrated under reduced pressure to obtain a crude, which was separated and purified by normal phase column chromatography (petroleum ether:ethyl acetate=1:1), to obtain the title compound 61D (2 g).

MS m/z (ESI): 370.31/371.9 [M+H]⁺.

### Step 5: Preparation of 5-(1-(3-bromophenyl)-1-cyclobutylethyl)-4-methyl-4H-1,2,4-triazole-3-thiol (61E)

61D (1.5 g, 4.05 mmol) was dissolved in aqueous sodium hydroxide solution (1.5 M, 30 mL), and the mixture was heated to 50° C., and stirred for 12 hours. After the reaction was completed, the reaction solution was cooled to room temperature, 1M aqueous hydrochloric acid solution was used to adjust the pH to 3, the mixture was extracted with ethyl acetate, and the organic phase was dried over anhydrous sodium sulfate, filtered, and concentrated under reduced pressure to obtain the title compound 61E (1.3 g) as a crude, which was directly used in the next step.

MS m/z (ESI): 352.29/351.9 [M+H]⁺.

### Step 6: Preparation of 3-(1-(3-bromophenyl)-1-cyclobutylethyl)-4-methyl-4H-1,2,4-triazole (61F)

At 0° C. (ice-water bath), to a mixture of 61E (1.3 g, 3.69 mmol), acetic acid (6 mL, 3.69 mmol) and dichloromethane (20 mL) was slowly added dropwise a hydrogen peroxide solution (2.060 g, 18.17 mmol, purity: 30%). The reaction solution was returned to room temperature and stirred at room temperature for 12 hours. After the reaction was completed, the mixture was quenched with saturated aqueous sodium thiosulfate solution, and extracted with dichloromethane, the organic phase was dried over anhydrous sodium sulfate, filtered, and concentrated under reduced pressure to obtain a crude, which was separated and purified by normal phase column chromatography (petroleum ether:ethyl acetate=0:1), to obtain the title compound 61F (900 mg).

MS m/z (ESI): 320.23/321.9 [M+H]⁺.

### Step 7: Preparation of 3-(1-cyclobutyl-1-(3-(4,4,5,5-tetramethyl-1,3,2-dioxaborolan-2-yl)phenyl)ethyl)-4-methyl-4H-1,2,4-triazole (61G)

Pd(dppf)Cl₂-DCM (39 mg, 0.53 mmol) was added to a mixture of 61F (900 mg, 2.81 mmol), B₂Pin₂ (1.07 g, 4.22 mmol), potassium acetate (551 mg, 5.61 mmol) and anhydrous dioxane (30 mL), and the resulting mixture was heated to 110° C. under nitrogen protection, and stirred for 12 hours. After the reaction was completed, the mixture was cooled to room temperature, filtered, and concentrated under reduced pressure. The residue was purified by normal phase column chromatography (petroleum ether:ethyl acetate=0:1) to obtain the title compound 61G (540 mg).

MS m/z (ESI): 367.29/368.5 [M+H]⁺.

### Step 8: Preparation of 3-(3-(1-cyclobutyl-1-(4-methyl-4H-1,2,4-triazol-3-yl)ethyl)phenyl)-7-(((S)-3-methylpiperidin-1-yl)methyl)-9-(trifluoromethyl)-4H-pyrido[1,2-a]pyrimidin-4-one (compound 61)

Pd(dppf)Cl₂ (40 mg, 54.67 μmol) was added to a mixture of 61G (200 mg, 544.53 μmol), 2F (246.00 mg, 545.18 μmol), sodium carbonate (170.00 mg, 1.60 mmol), water (0.4 mL) and anhydrous dioxane (2 mL), and the resulting mixture was heated to 100° C. under nitrogen protection and stirred for 12 hours. After the reaction was completed, the mixture was cooled to room temperature, filtered, and concentrated under reduced pressure to obtain a crude product, which was separated by preparative high performance liquid chromatography (chromatographic column: Welch Xtimate C18, column length 150 mm, inner diameter 30 mm, and particle size 5 m; mobile phase A: water (0.225% FA), mobile phase B: acetonitrile; gradient: mobile phase B: from 28% to 58% over 7 minutes, then eluted with 100% mobile phase B for 3.2 minutes; flow rate: 25 mL/min), to obtain the title compound 61 (116 mg).

MS m/z (ESI):564.64/565.1 [M+H]⁺;

¹H NMR (400 MHz, CDCl₃) δ 9.19 (s, 1H), 8.55 (s, 1H), 8.38 (s, 1H), 8.06 (s, 1H), 7.62 (d, J=7.6 Hz, 1H), 7.52 (s, 1H), 7.47-7.41 (m, 1H), 7.10 (d, J=8.0 Hz, 1H), 3.80-3.68 (m, 2H), 3.51-3.40 (m, 1H), 3.13 (s, 3H), 3.06-2.81 (m, 4H), 2.27-2.11 (m, 1H), 2.08-1.78 (m, 8H), 1.76-1.58 (m, 4H), 1.04-0.93 (m, 1H), 0.89 (d, J=6.4 Hz, 3H).

### Example 7: Preparation of 3-(3-(cyclopropyl(4-methyl-4H-1,2,4-triazol-3-yl)methyl)phenyl)-7-(((S)-3-methylpiperidin-1-yl)methyl)-9-trifluoromethyl-4H-pyrido[1,2-a]pyrimidin-4-one (compound 63)

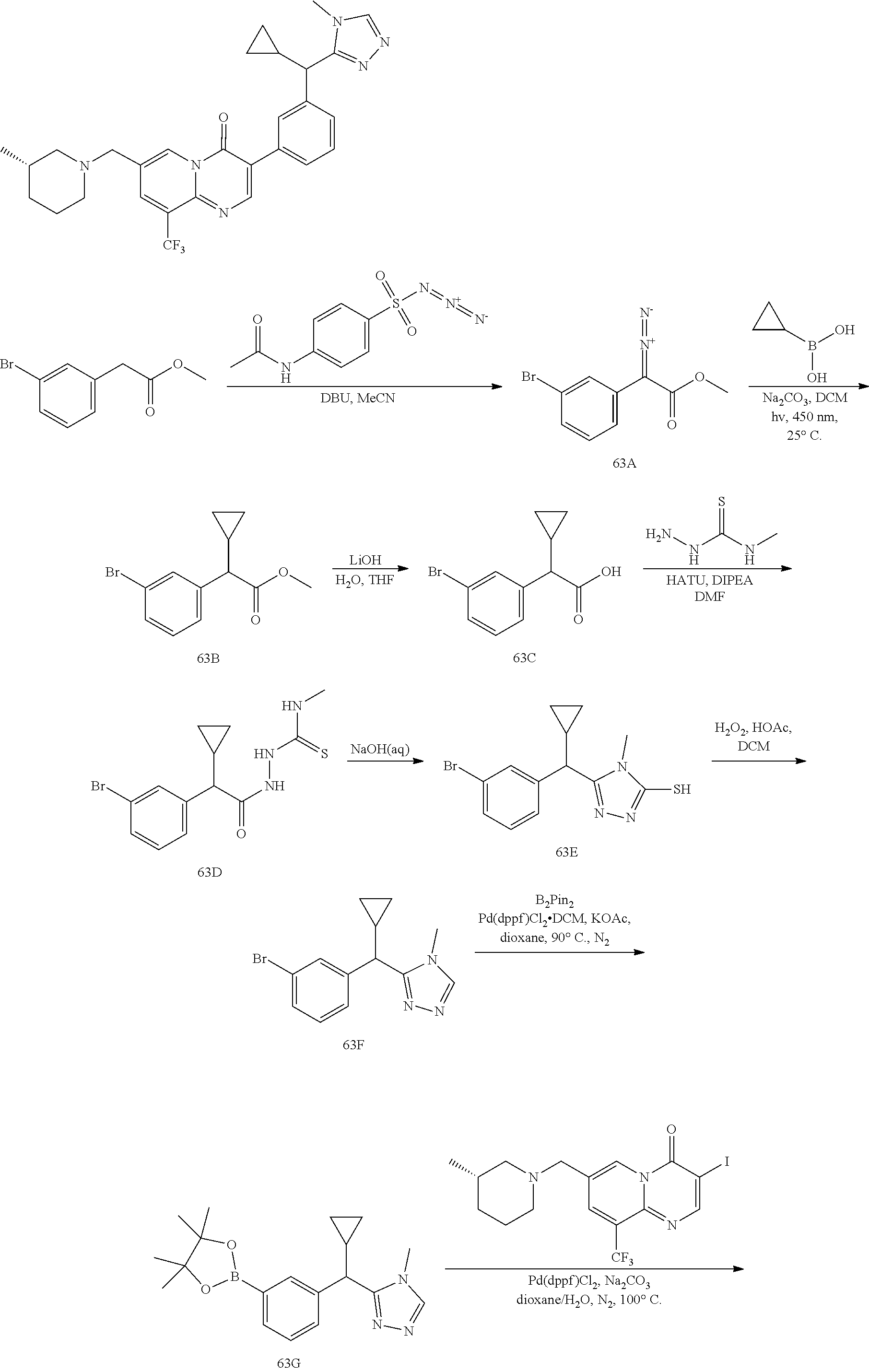

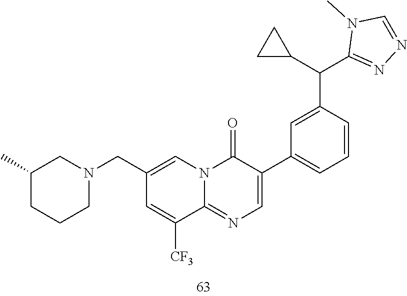 [SMILES: C[C@H]1CCCN(Cc2cc(C(F)(F)F)c3ncc(-c4cccc(C(c5nncn5C)C5CC5)c4)c(=O)n3c2)C1]

### Step 1: Preparation of methyl 2-(3-bromophenyl)-2-diazoacetate (63A)

Methyl 2-(3-bromophenyl)acetate (5 g, 21.83 mmol) and 1,8-diazabicyclo[5.4.0]undec-7-ene (9.97 g, 65.48 mmol, 9.77 mL) were added to acetonitrile (49.98 mL), the temperature was cooled down to 0° C., and 4-acetamidobenzenesulfonyl azide (6.29 g, 26.19 mmol) was added dropwise under nitrogen protection. The reaction solution was heated to 25° C. and stirred under nitrogen protection for 3 hours. After the reaction was completed, saturated aqueous ammonium chloride solution (20 mL) was added, and the mixture was extracted with ethyl acetate three times. The organic phase obtained by extraction was dried over anhydrous sodium sulfate, filtered, and distilled under reduced pressure to obtain a crude product, which was purified by normal phase column chromatography (petroleum ether:ethyl acetate=10:1) to obtain the title compound 63A (5.5 g, yield: 94%) as a solid.

MS m/z: 254.8, 256.8 [M+H]⁺.

¹H NMR (400 MHz, DMSO-d₆) δ 7.77 (s, 1H), 7.45 (d, J=7.0 Hz, 1H), 7.42-7.34 (m, 2H), 3.81 (s, 3H).

### Step 2: Preparation of methyl 2-(3-bromophenyl)-2-cyclopropyl acetate (63B)

To dichloromethane (20 mL) were added cyclopropylboronic acid (1.35 g, 15.68 mmol), 63A (2 g, 7.84 mmol), and sodium carbonate (831.5 mg, 7.84 mmol), nitrogen replacement was performed three times, and the mixture was stirred at 25° C. in a photoreactor (450 nm) for 12 hours. After the reaction was completed, the mixture was distilled under reduced pressure to obtain a crude product, which was purified by normal phase column chromatography (petroleum ether:ethyl acetate=10:1) to obtain the title compound 63B (1.2 g, yield: 28%) as a solid.

MS m/z (ESI): 269.01 [M+H]⁺& 271.01 [M+H]⁺.

### Step 3: Preparation of 2-(3-bromophenyl)-2-cyclopropyl acetic acid (63C)

63B (480 mg, 1.78 mmol) was added to a mixture of water (5 mL) and tetrahydrofuran (5 mL), lithium hydroxide (299.34 mg, 7.13 mmol) was then added, and the mixture was stirred at room temperature for 3 hours. After the reaction was completed, tetrahydrofuran was removed by rotary evaporation, and the mixture was extracted with ethyl acetate three times. The aqueous phase was adjusted with 1M hydrochloric acid to pH 3-4, and then extracted with ethyl acetate, and the organic phase was concentrated to dryness under reduced pressure to obtain the title compound 63C (215 mg) as a solid.

MS m/z (ESI): 255.01 [M+H]⁺ & 257.01 [M+H]⁺.

### Step 4: Preparation of 1-[(2-(3-bromophenyl)-2-cyclopropylacetyl)amino]-3-methyl thiourea (63D)

To DMF (2 mL) was added DIPEA (519.28 mg, 4.02 mmol, 699.83 μL), 63C (205 mg, 803.58 μmol), 4-methylthiosemicarbazide (211.27 mg, 2.01 mmol) and HATU (460.09 mg, 1.21 mmol). The reaction solution was stirred at room temperature for 6 hours. After the reaction was completed, the mixture was diluted with water, and then extracted with ethyl acetate three times. The organic phase was dried over anhydrous sodium sulfate, and filtered, and the filtrate was concentrated to dryness under reduced pressure to obtain a crude product, which was purified by normal phase column chromatography (petroleum ether:ethyl acetate=0:1) to obtain the title product 63D (0.11 g, yield: 8%) as a solid.

MS m/z (ESI): 342.02 [M+H]⁺& 344.02 [M+H]⁺.

### Step 5: Preparation of 5-[(3-bromophenyl)cyclopropylmethyl]-4-methyl-4H-1,2,4-triazol-3-thiol (63E)

63D (110 mg, 321.40 μmol) was added to aqueous sodium hydroxide solution (1 M, 3 mL). The reaction solution was stirred at room temperature for 12 hours. After the reaction was completed, the pH was adjusted to 3-4 with 1M hydrochloric acid. The mixture was extracted with ethyl acetate, the organic phase was washed with saturated brine, dried over anhydrous sodium sulfate, and filtered, and the filtrate was concentrated to dryness under reduced pressure, to obtain the title product 63E (77 mg) as a solid.

### Step 6: Preparation of 3-[(3-bromophenyl)cyclopropylmethyl]-4-methyl-4H-1,2,4-triazole (63F)

63E (77 mg, 237.48 μmol) was added to dichloromethane (2 mL) and acetic acid (285.22 mg, 4.75 mmol) and the mixture was cooled to 0° C. Hydrogen peroxide (40.39 mg, 391.84 μmol, purity 33%) was then added dropwise, and the mixture was returned to room temperature and stirred for 12 hours. After the reaction was completed, saturated aqueous sodium bisulfite and saturated aqueous sodium bicarbonate solutions were added to adjust the pH to about 8. The mixture was extracted with dichloromethane, the organic phase was washed with saturated aqueous sodium bisulfite solution, dried over anhydrous sodium sulfate, and filtered, and the filtrate was concentrated to dryness under reduced pressure, to obtain the title product 63F (62 mg, yield: 90%) as a solid.

MS m/z (ESI): 292.04 [M+H]⁺& 294.04 [M+H]⁺.

### Step 7: Preparation of 3-[cyclopropyl(3-(4,4,5,5-tetramethyl-1,3,2-dioxaborolan-2-yl)phenyl)methyl]-4-methyl-4H-1,2,4 triazole (63G)

63F (57 mg, 195.09 μmol), B₂Pin₂ (148.62 mg, 585.27 μmol) and potassium acetate (45.04 mg, 585.27 μmol) were added to dioxane (2 mL), nitrogen replacement was performed, and Pd(dppf)Cl₂-DCM (15.93 mg, 19.51 μmol) was added. The reaction was stirred at 90° C. for two hours. After the reaction was completed, the reaction solution was diluted with water, and then extracted with ethyl acetate three times. The organic phases were combined, dried over anhydrous sodium sulfate, and filtered, and the filtrate was concentrated to dryness under reduced pressure to obtain a crude product, which was purified by normal phase column chromatography (petroleum ether:ethyl acetate=10:1) to obtain the title product 63G (66 mg, yield: 100%) as a solid.

### Step 8: Preparation of 3-(3-(cyclopropyl(4-methyl-4H-1,2,4-triazol-3-yl)methyl)phenyl)-7-(((S)-3-methylpiperidin-1-yl)methyl)-9-trifluoromethyl-4H-pyrido[1,2-a]pyrimidin-4-one (compound 63)

63G (60 mg, 176.87 μmol), 2F (66.51 mg, 147.39 μmol) and sodium carbonate (46.86 mg, 442.17 μmol) were added to 1,4-dioxane (1 mL) and water (0.2 mL), nitrogen replacement was performed three times, and Pd(dppf)Cl₂ (10.78 mg, 14.74 μmol) was added. The reaction was stirred at 100° C. for three hours. After the reaction was completed, the reaction solution was cooled to room temperature, and filtered, the solvent was removed by rotary evaporation to obtain a crude product, which was separated by preparative high performance liquid chromatography (chromatographic column: Phenomenex C18, column length 80 mm, inner diameter 40 mm, and particle size 3 μm; mobile phase A: water (0.1% aqueous ammonia), mobile phase B: acetonitrile; gradient: mobile phase B: from 51% to 81% over 8 minutes, then eluted with 100% mobile phase B for 4 minutes; flow rate: 30 mL/min), to obtain the title compound 63 (3.49 mg, yield: 6%).

MS m/z (ESI):537.3 [M+H]⁺;

¹H NMR (400 MHz, DMSO-d₆) δ 9.12 (s, 1H), 8.62 (s, 1H), 8.37-8.28 (m, 2H), 7.79-7.74 (m, 1H), 7.68 (m, 1H), 7.42 (m, 1H), 7.29 (m, 1H), 3.67-3.54 (m, 3H), 3.41-3.39 (m, 3H), 2.79 (s, 2H), 1.97 (s, 1H), 1.72-1.40 (m, 6H), 0.90-0.77 (m, 4H), 0.67-0.50 (m, 2H), 0.41-0.30 (m, 2H).

### Example 8: Preparation of 3-(3-(cyclopropyl(4-methyl-4H-1,2,4-triazol-3-yl)methyl)phenyl)-8-methyl-6-(((S)-3-methylpiperidin-1-yl)methyl)-4H-chromen-4-one (compound 64)

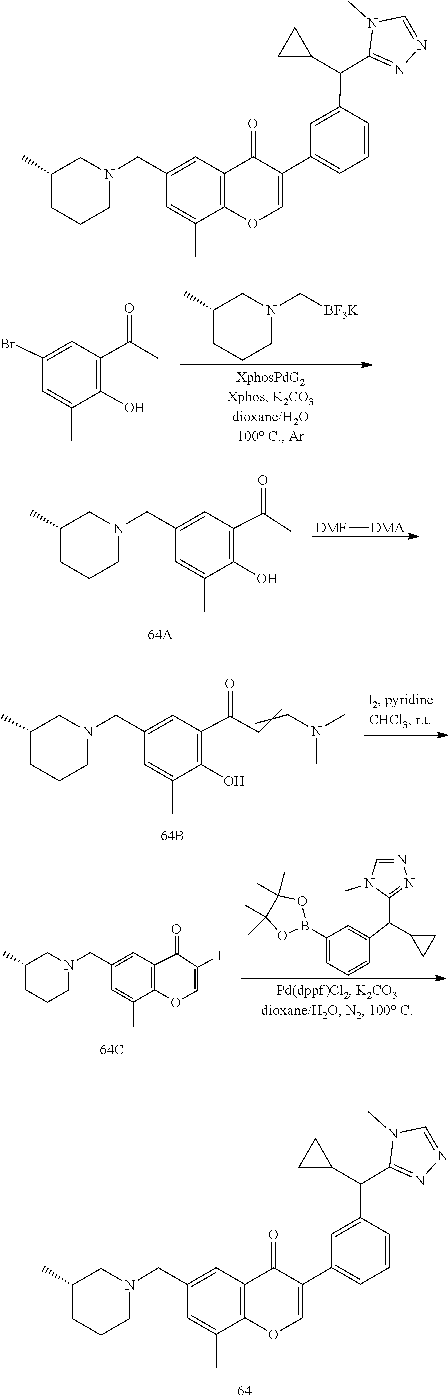

### Step 1: Preparation of (S)-2-hydroxyl-3-methyl-5-(3-methylpiperidin-1-yl)methylacetophenone (64A)

5-Bromo-2-hydroxy-3-methylacetophenone (1 g, 4.37 mmol), 2B (1.43 g, 6.55 mmol), XphosPd G₂ (343.04 mg, 436.55 μmol), potassium carbonate (1.21 g, 8.73 mmol) and Xphos (416.22 mg, 873.09 μmol) were weighed into a reaction tube, which was evacuated and backfilled with argon, water (4 mL) and dioxane (12 mL) were added with a syringe, the mixture was heated to 100° C. and reacted for 1 hour, and cooled to room temperature, and the reaction solution was directly purified by reverse phase column chromatography (chromatographic column: SepaFlash® silica gel column; mobile phase: acetonitrile/water=75:25), and then lyophilized, to obtain the title compound 64A (330 mg, yield: 29%).

MS m/z (ESI):262.17 [M+H]⁺;

### Step 2: Preparation of (S)-3-dimethylamino-1-(2-hydroxyl-3-methyl-5-((3-methylpiperidin-1-yl)methyl)phenyl)prop-2-en-1-one (64B)

64A (330 mg, 1.26 mmol) was dissolved in DMF-DMA (6 mL), and the mixture was heated to 90° C., and reacted for 8 hours. The mixture was cooled to room temperature, concentrated and azeotroped with chloroform three times to obtain 64B (399 mg) as a crude, which was directly used in the next step.

MS m/z (ESI):317.22 [M+H]⁺;

### Step 3: Preparation of (S)-3-iodo-8-methyl-6-((3-methylpiperidin-1-yl)methyl)-4H-chromen-4-one (64C)

64B (399 mg, 1.26 mmol) was dissolved in chloroform (4 mL), and iodine (640.06 mg, 2.52 mmol) and pyridine (199.48 mg, 2.52 mmol, 203.15 μL) were sequentially added under ice-water bath, and the mixture was reacted at room temperature for 1 hour, quenched with saturated aqueous sodium bisulfite solution (1 mL), filtered, and the filtrate was concentrated to obtain a crude, which was purified by reverse phase column chromatography (chromatographic column: SepaFlash® silica gel column; mobile phase: acetonitrile/water=7:3) and then lyophilized, to obtain the title compound 64C (152 mg, yield: 30%).

### Step 4: Preparation of 3-(3-(cyclopropyl(4-methyl-4H-1,2,4-triazol-3-yl)methyl)phenyl)-8-methyl-6-(((S)-3-methylpiperidin-1-yl)methyl)-4H-chromen-4-one (compound 64)

In a dry reaction tube, 63G (32.02 mg, 94.40 μmol), 64C (25 mg, 62.93 μmol), Pd(dppf)Cl₂ (4.57 mg, 6.29 μmol) and potassium carbonate (8.70 mg, 62.93 μmol) were added, the tube was evacuated and backfilled with nitrogen, water (0.5 mL) and 1,4-dioxane (2 mL) were added via a syringe, and the mixture was heated to 100° C. for 1 hour, cooled to room temperature, and purified by reverse phase column chromatography (chromatographic column: SepaFlash® silica gel column; mobile phase: acetonitrile/water=8:2) and then lyophilized, to obtain the title compound 64 (10.5 mg, yield: 35%).

MS m/z (ESI):483.27 [M+H]⁺;

¹H NMR (400 MHz, DMSO-d₆) δ 8.57 (s, 1H), 8.35 (s, 1H), 7.87 (s, 1H), 7.61 (s, 1H), 7.52 (s, 1H), 7.47-7.38 (m, 2H), 7.31 (d, J=7.4 Hz, 1H), 3.61-3.51 (m, 3H), 3.42 (s, 3H), 2.72 (s, 2H), 2.45 (s, 3H), 2.10-1.75 (m, 2H), 1.64-1.48 (m, 5H), 0.85-0.76 (m, 4H), 0.66-0.50 (m, 2H), 0.40-0.30 (m, 2H).

### Example 9: Preparation of (S)-8-methyl-3-(5-(3-methyl-1-(4-methyl-4H-1,2,4-triazol-3-yl)cyclobutyl)pyridin-3-yl)-6-((3-methylpiperidin-1-yl)methyl)-4H-chromen-4-one (compound 65)

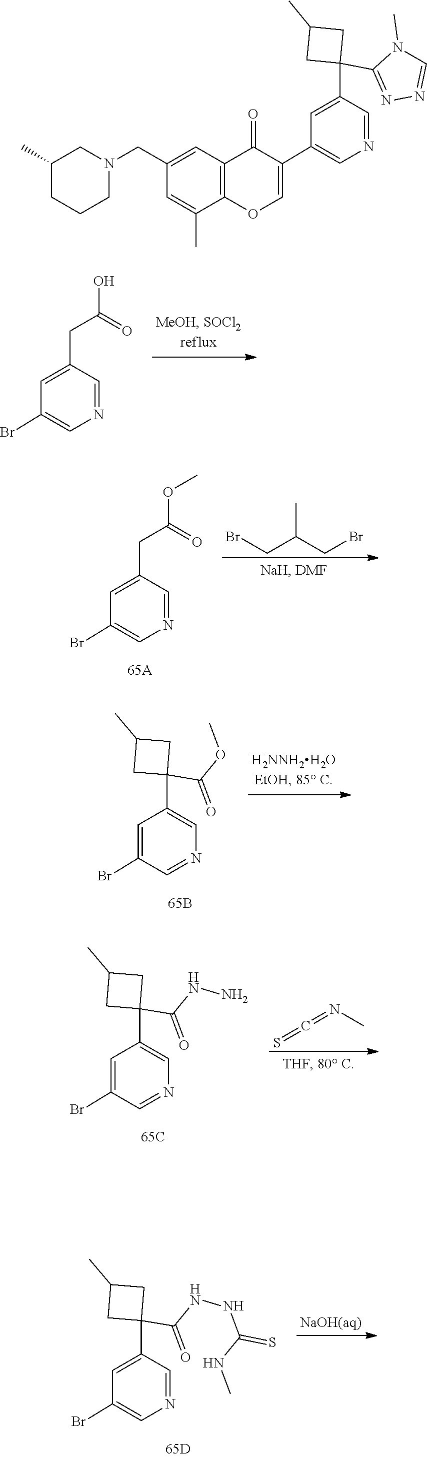

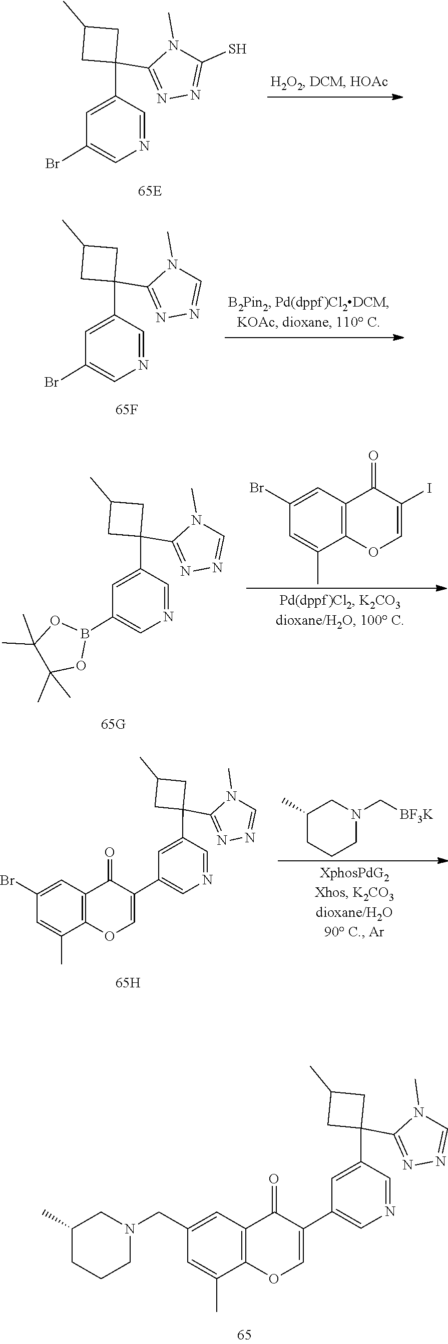

### Step 1: Preparation of methyl 2-(5-bromopyridin-3-yl)acetate (65A)

2-(5-Bromopyridin-3-yl)acetic acid (2.4 g, 11.11 mmol) was dissolved in methanol (5 mL), thionyl chloride (524.16 mg, 4.41 mmol) was slowly added dropwise, and the mixture was heated to 80° C. and refluxed, and stirred for 12 hours. The reaction solution was cooled to room temperature, ethyl acetate (20 mL) and water (10 mL) were added to the reaction solution, the organic phase was washed with saturated aqueous sodium bicarbonate solution (20 mL×3) and saturated brine, the organic phase was dried over anhydrous sodium sulfate, filtered, and concentrated to dryness under reduced pressure to obtain the title compound 65A (2.27 g, yield: 86%) as a crude, which was directly used in the next step.

MS m/z (ESI): 230.06/231.9 [M+H]⁺.

¹H NMR (400 MHz, CDCl₃) δ 8.67-8.55 (m, 1H), 8.48-8.38 (m, 1H), 7.86-7.78 (m, 1H), 3.74 (s, 3H), 3.63 (s, 2H)

### Step 2: Preparation of methyl 1-(5-bromopyridin-3-yl)-3-methylcyclobutane-1-carboxylate (65B)

At 0° C. (ice-water bath), sodium hydride (790 mg, 19.75 mmol, purity: 60%) was added to 65A (2.27 g, 9.87 mmol) and anhydrous DMF (15 mL), and the mixture was stirred at 0° C. (ice-water bath) for 0.5 h. 1,3-Dibromo-2-methylpropane (3, 2.56 g, 11.84 mmol) was added to the mixture at 0° C. (ice-water bath), and the reaction solution was stirred at 0° C. (ice-water bath) for 1.5 hours. The reaction was quenched with saturated aqueous ammonium chloride solution, and extracted with ethyl acetate, the organic phase was dried over anhydrous sodium sulfate, filtered, and concentrated to dryness under reduced pressure to obtain a target compound as a crude, which was purified by normal phase column chromatography (petroleum ether:ethyl acetate=10:1), to obtain the title compound 65B (1.3 g, yield: 46%).

MS m/z (ESI): 284.15/285.9 [M+H]⁺.

¹H NMR (400 MHz, CDCl₃) δ 8.61-8.56 (m, 2H), 7.88-7.83 (m, 1H), 3.66 (s, 3H), 2.72-2.61 (m, 2H), 2.54-2.44 (m, 2H), 2.12-1.99 (m, 1H), 1.12 (d, J=4.0 Hz, 3H)

### Step 3: Preparation of 1-(5-bromopyridin-3-yl)-3-methylcyclobutane-1-carbohydrazide (65C)

Hydrazine hydrate (12.340 g, 209.53 mmol, purity: 85%) was added to 65B (1.3 g, 4.58 mmol) and ethanol (15 mL), and the mixture was heated to 85° C. and stirred for 12 hours. The reaction solution was cooled to room temperature, the mixture was directly evaporated to dryness, to obtain the title compound 65C (1.3 g) as a crude, which was directly used in the next step.

MS m/z (ESI): 284.15/285.7 [M+H]⁺.

### Step 4: Preparation of 2-(1-(5-bromopyridin-3-yl)-3-methylcyclobutane-1-carbonyl)-N-methylhydrazine-1-thioamide (65D)

Methyl isothiocyanate (1.00 g, 13.73 mmol) was added to 65C (1.3 g, 4.58 mmol) and anhydrous tetrahydrofuran (15 mL), and the mixture was heated to 80° C. and stirred for 3 hours. The reaction solution was cooled to room temperature, and ethyl acetate (20 mL) was added. The mixture was stirred in an ice-water bath for 1 hour, and filtered, the filter cake was washed with ethyl acetate (20 mL×2), and then the filter cake was collected and dried, to obtain the title compound 65D (1.5 g) as a crude, which was directly used in the next step.

MS m/z (ESI): 357.27/359.0 [M+H]⁺.

### Step 5: Preparation of 5-(1-(3-bromopyridin-3-yl)-3-methylcyclobutyl)-4-methyl-4H-1,2,4-triazol-3-thiol (65E)

65D (1.5 g, 4.20 mmol) was dissolved in aqueous sodium hydroxide solution (1.0 M, 25 mL), nitrogen replacement was performed three times, and the mixture was stirred at room temperature for 12 hours. The reaction solution was adjusted to pH 3 with 1M aqueous hydrochloric acid solution, the mixture was extracted with ethyl acetate (30 mL×3), and the organic phase was dried over anhydrous sodium sulfate, filtered, and concentrated to dryness under reduced pressure to obtain the title compound 65E (1.3 g) as a crude, which was directly used in the next step.

MS m/z (ESI): 339.25/341.0 [M+H]⁺.

### Step 6: Preparation of 3-bromo-5-(3-methyl-1-(4-methyl-4H-1,2,4-triazol-3-yl)cyclobutyl)pyridine (65F)

At 0° C. (ice-water bath), hydrogen peroxide (9.2 g, 81.15 mmol, purity: 30%) and glacial acetic acid (177.53 mg, 2.96 mmol, 3 mL) were slowly added dropwise to a mixture of 65E (1.3 g, 3.83 mmol) and dichloromethane (15 mL). The reaction solution was returned to room temperature and stirred at room temperature for 12 hours. The mixture was quenched with saturated sodium thiosulfate aqueous solution and extracted with dichloromethane (20 mL×3). The organic phase was dried over anhydrous sodium sulfate, filtered, and spin-dried to obtain the target compound as a crude, which was separated by preparative high performance liquid chromatography (chromatographic column: Welch Xtimate C18, column length 150 mm, inner diameter 30 mm, and particle size 5 μm; mobile phase A: water (0.1% aqueous ammonia+0.1% ammonium bicarbonate), mobile phase B: acetonitrile; gradient: mobile phase B: from 20% to 50% over 8 minutes, then eluted with 100% mobile phase B for 2.5 minutes; flow rate: 30 mL/min), to obtain the title compound 65F (521 mg, yield: 45%).

MS m/z (ESI): 307.2/308.8 [M+H]⁺.

¹H NMR (400 MHz, CDCl₃) δ 8.62-8.56 (m, 2H), 8.01 (s, 1H), 7.79-7.75 (m, 1H), 3.24 (s, 3H), 2.91-2.80 (m, 2H), 2.75-2.66 (m, 3H), 1.18-1.14 (m, 3H).

### Step 7: Preparation of 3-(3-methyl-1-(4-methyl-4H-1,2,4-triazol-3-yl)cyclobutyl)-5-(4,4,5,5-tetramethyl-1,3,2-dioxaborolan-2-yl)pyridine (65G)

Referring to the synthesis method in step 7 of Example 6, the title compound 65G (20 mg, yield: 58%) was prepared using the same method, except that 61F was replaced with 65F (30 mg, 97.66 μmol).

MS m/z (ESI): 355.0 [M+H]⁺.

### Step 8: Preparation of 6-bromo-8-methyl-3-(5-(3-methyl-1-(4-methyl-4H-1,2,4-triazol-3-yl)cyclobutyl)pyridin-3-yl)-4H-chromen-4-one (65H)

The preparation method was the same as compound 58D. Starting with 65G (20 mg, 56.46 μmol), the title compound 65H (15 mg, yield: 57%) was finally obtained.

MS m/z (ESI): 465.1[M+H]⁺.

### Step 9: Preparation of (S)-8-methyl-3-(5-(3-methyl-1-(4-methyl-4H-1,2,4-triazol-3-yl)cyclobutyl)pyridin-3-yl)-6-((3-methylpiperidin-1-yl)methyl)-4H-chromen-4-one (compound 65)

The preparation method is the same as compound 58. 65H (15 mg, 32.23 μmol) and 2B (10.59 mg, 48.35 μmol) were reacted at 90° C. to finally obtain the title compound 65 (6.8 mg, yield: 42%).

MS m/z (ESI):498.28 [M+H]⁺;

¹H NMR (400 MHz, DMSO-d₆) δ 8.78 (d, J=5.7 Hz, 1H), 8.70-8.65 (m, 1H), 8.55 (d, J=2.4 Hz, 1H), 8.35 (s, 1H), 8.05 (t, J=2.2 Hz, 1H), 7.89 (d, J=2.5 Hz, 1H), 7.66 (s, 1H), 3.54 (s, 2H), 3.28 (s, 3H), 2.96 (d, J=3.6 Hz, 2H), 2.78-2.70 (m, 2H), 2.61 (d, J=6.6 Hz, 3H), 2.49 (s, 3H) 1.90 (t, J=11.3 Hz, 1H), 1.69-1.49 (m, 5H), 1.12 (d, J=5.1 Hz, 3H), 0.83 (d, J=5.3 Hz, 4H).

### Example 10: Preparation of (S)-8-methyl-3-(3-(3-methyl-1-(4-methyl-4H-1,2,4-triazol-3-yl)cyclobutyl)-5-trifluoromethylphenyl)-6-((3-methylpiperidin-1-yl)methyl)-4H-chromen-4-one (compound 66)

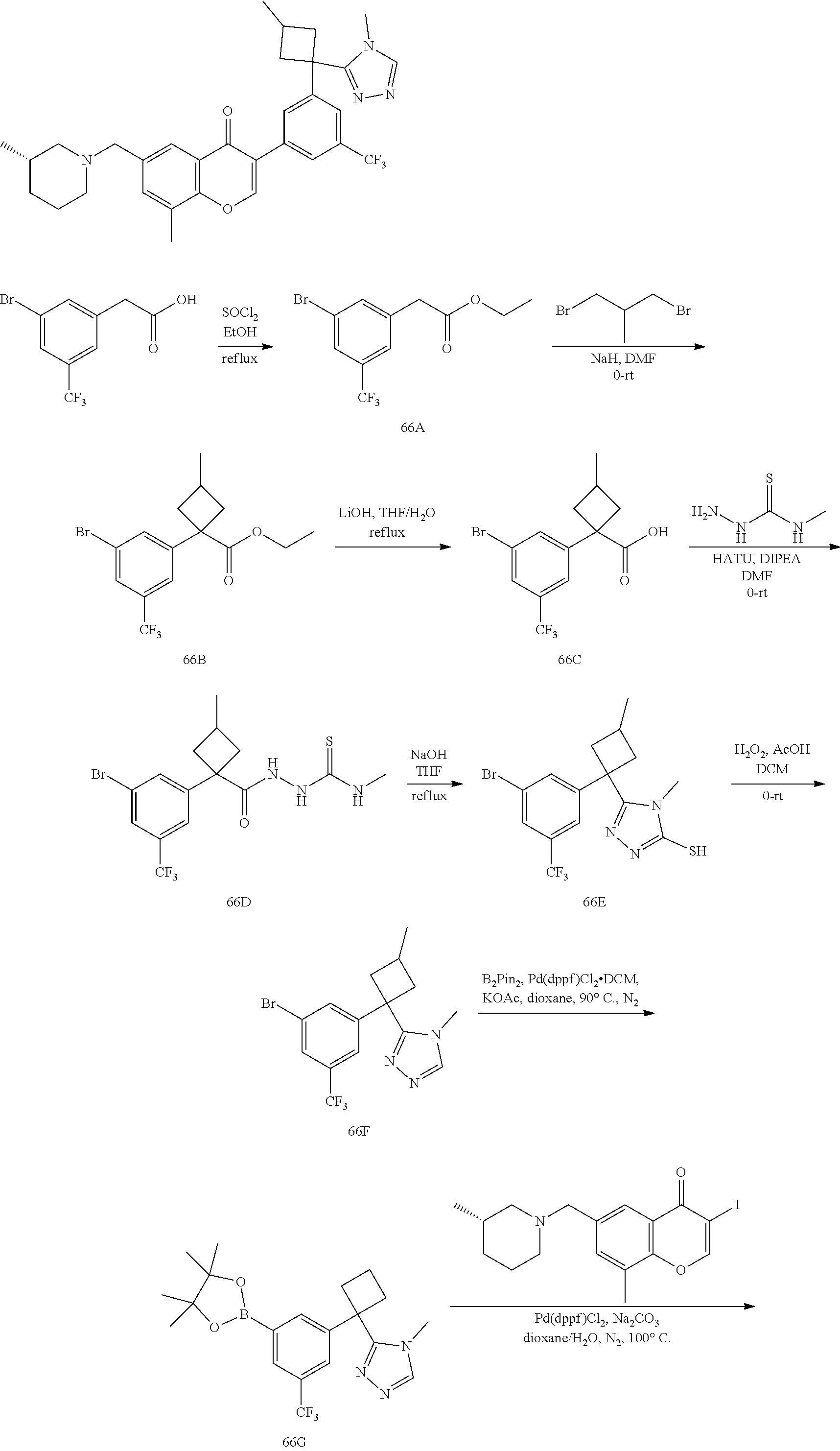

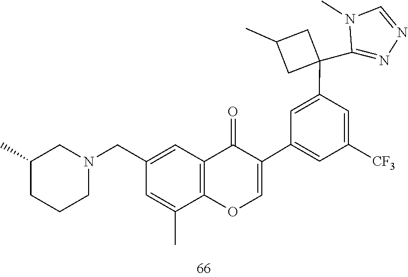 [SMILES: Cc1cc(CN2CCC[C@H](C)C2)cc2c(=O)c(-c3cc(C(F)(F)F)cc(C4(c5nncn5C)CC(C)C4)c3)coc12]

### Step 1 to step 3: Preparation of 1-(3-bromo-5-(trifluoromethyl)phenyl)-3-methylcyclobutylcarboxylic acid (66C)

The preparation method of step 1 to step 2 is the same as compound 65B. The preparation method of step 3 is the same as 63C. Starting with 2-(3-bromo-5-(trifluoromethyl)phenyl)acetic acid (1 g, 3.53 mmol), the title compound 66C (170 mg, yield: 14%) was finally obtained through three steps of reaction.

MS m/z (ESI): 335.0/337.0[M−H]⁻;

### Step 4 to step 7: Preparation of 4-methyl-3-(3-methyl-1-(3-(4,4,5,5-tetramethyl-1,3,2-dioxaborolan-2-yl)-5-(trifluoromethyl)phenyl)cyclobutyl)-4H-1,2,4-triazole (66G)

The preparation method is the same as compound 63G. Starting with 66C (270 mg, 0.80 mmol), the title compound 66G (33.77 mg, yield: 10%) was finally obtained through four steps of reaction.

MS m/z (ESI): 422.2[M+H]⁺;

### Step 8: Preparation of (S)-8-methyl-3-(3-(3-methyl-1-(4-methyl-4H-1,2,4-triazol-3-yl)cyclobutyl)-5-trifluoromethylphenyl)-6-((3-methylpiperidin-1-yl)methyl)-4H-chromen-4-one (compound 66)

The preparation method is the same as compound 63. 64C (18 mg, 45.31 μmol) and 66G (19.09 mg, 45.31 μmol) were reacted to finally obtain the title compound 66 (11 mg, yield: 43%).

MS m/z (ESI):565.25 [M+H]⁺;

¹H NMR (400 MHz, DMSO-d₆) δ 8.80 (s, 1H), 8.34 (s, 1H), 8.0-7.75 (m, 2H), 7.75-7.5 (m, 2H), 7.5-7.36 (s, 1H), 3.54 (s, 2H), 3.28 (s, 3H), 2.96 (s, 2H), 2.8-2.7 (m, 4H), 2.6 (s, 3H), 2.4 (m, 1H), 1.90 (s, 1H), 1.75-1.5 (m, 5H), 1.26 (s, 3H), 0.83 (m, 4H).

### Example 11: Preparation of (S)-8-methyl-3-(3-(2-(4-methyl-4H-1,2,4-triazol-3-yl)propan-2-yl)phenyl)-6-((3-methylpiperidin-1-yl)methyl)-4H-chromen-4-one (compound 67)

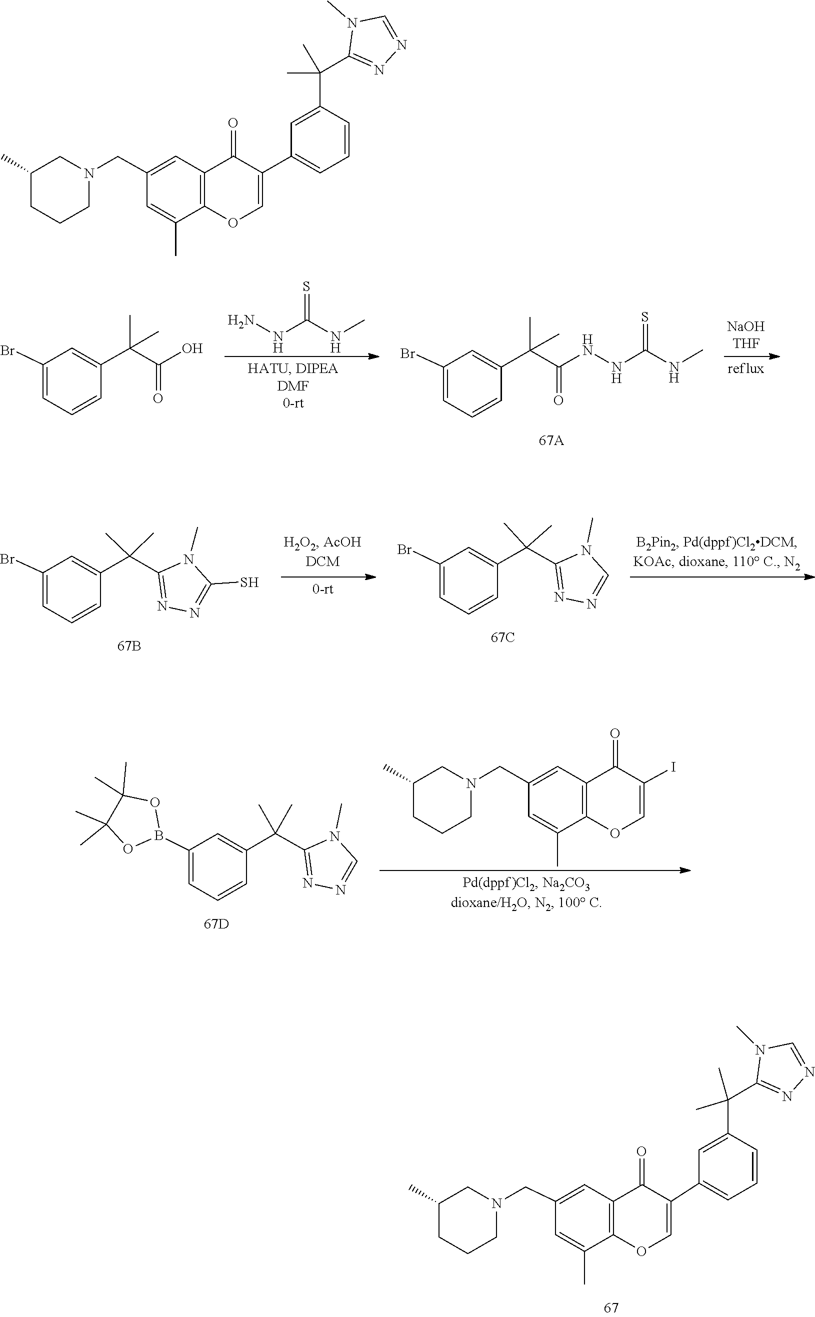

### Step 1 to step 4: Preparation of 4-methyl-3-(2-(3-(4,4,5,5-tetramethyl-1,3,2-dioxaborolan-2-yl)phenyl)propan-2-yl)-4H-1,2,4-triazole (67D)

Referring to the synthesis method in step 4 to step 7 of Example 10, the title compound 67D (116 mg, yield: 35%) was prepared using the same method, except that 66C was replaced with 2-(3-bromophenyl)-2-methylpropanoic acid (250 mg, 1.03 mmol).

MS m/z (ESI): 328.2[M+H]⁺;

### Step 5: Preparation of (S)-8-methyl-3-(3-(2-(4-methyl-4H-1,2,4-triazol-3-yl)propan-2-yl)phenyl)-6-((3-methylpiperidin-1-yl)methyl)-4H-chromen-4-one (67)

The preparation method is the same as compound 63. 64C (15 mg, 37.76 μmol) and 67D (12.36 mg, 37.76 μmol) were reacted to finally obtain the title compound 67 (6 mg, yield: 34%).

MS m/z (ESI):471.27 [M+H]⁺;

¹H NMR (400 MHz, DMSO-d₆) δ 8.63 (s, 1H), 8.38 (s, 1H), 7.87 (s, 1H), 7.63 (s, 1H), 7.54-7.36 (m, 3H), 7.17 (dt, J=7.6, 1.7 Hz, 1H), 3.53 (s, 2H), 3.15 (s, 3H), 2.71 (d, J=13.6 Hz, 2H), 2.50 (s, 3H), 1.91 (d, J=12.6 Hz, 1H), 1.77 (s, 6H), 1.72-1.42 (m, 5H), 0.83 (d, J=5.3 Hz, 4H).

### Example 12: Preparation of (1S,3R)-3-(4-methyl-4H-1,2,4-triazol-3-yl)-3-(3-(8-methyl-6-(((S)-3-methylpiperidin-1-yl)methyl)-4-oxo-4H-chromen-3-yl)phenyl)cyclobutylacetonitrile (compound 68)

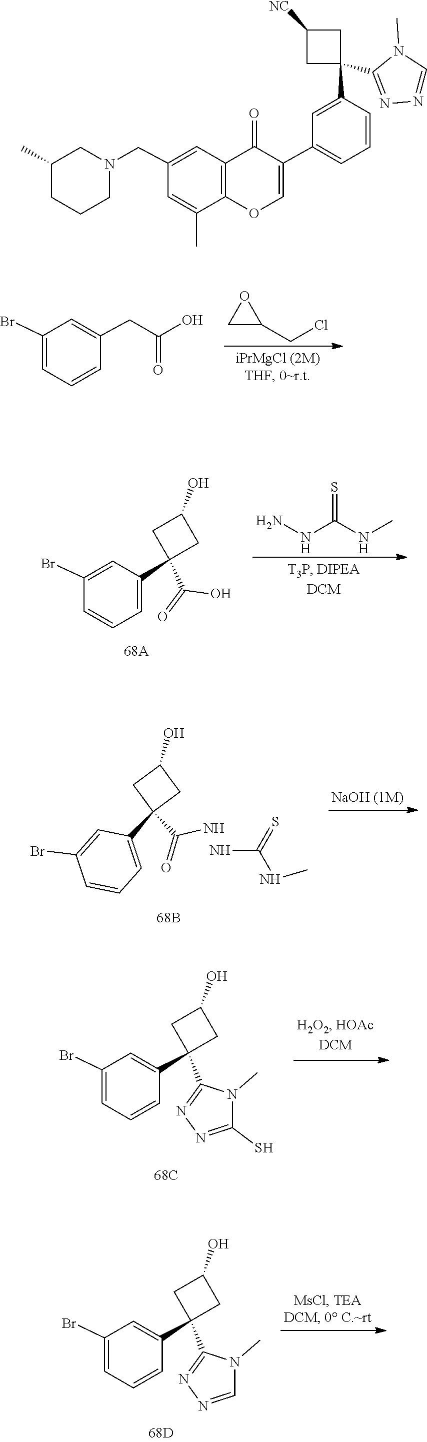

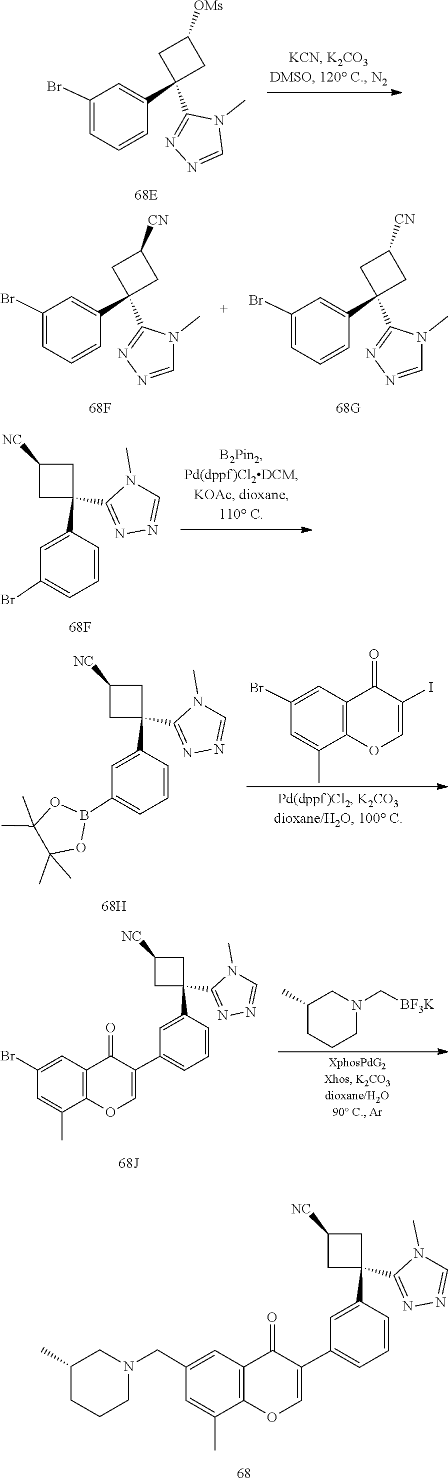

### Step 1: Preparation of cis-1-(3-bromophenyl)-3-hydroxylcyclobutane carboxylic acid (68A)

2-(3-Bromophenyl)acetic acid (20 g, 93.00 mmol) was dissolved in tetrahydrofuran (200 mL), isopropylmagnesium chloride (2 M, 103.93 mL) was slowly added at 0° C. (ice-water bath), and the reaction was returned to room temperature and stirred for 1 hour. Epichlorohydrin (16.01 g, 172.99 mmol, 13.53 mL) was added and stirred at room temperature for 3 hours. Isopropylmagnesium chloride (2 M, 103.93 mL) was then added dropwise to the above-mentioned reaction solution and the mixture was stirred at room temperature overnight. After the reaction was completed, the pH was adjusted to 1-2 with 2M HCl solution, and then the solution was extracted with dichloromethane (500 mL×3). The organic phase was combined, dried over anhydrous sodium sulfate, and filtered, and the solvent was removed by rotary evaporation to obtain a crude product, which was purified by normal phase column chromatography (0-50% ethyl acetate/petroleum ether), to obtain the title compound 68A (10.1 g, yield: 40%).

¹H NMR (400 MHz, DMSO-d₆) δ 12.43 (br. s., 1H), 7.53-7.49 (m, 1H), 7.48-7.42 (m, 1H), 7.40-7.26 (m, 2H), 5.31-5.10 (m, 1H), 3.92-3.80 (m, 1H), 2.78-2.69 (m, 2H), 2.54-2.51 (m, 2H).

### Step 2: Preparation of cis-2-(1-(3-bromophenyl)-3-hydroxylcyclobutanecarbonyl)-N-methylhydrazine thioformamide (68B)

68A (10.1 g, 37.25 mmol) was dissolved in dichloromethane (100 mL), and DIPEA (14.44 g, 111.76 mmol), T₃P (17.78 g, 55.88 mmol) and 1-amino-3-methylthiourea (4.70 g, 44.71 mmol) were sequentially added, and the reaction solution was stirred at room temperature overnight. The mixture was quenched with water (100 mL) to precipitate a white precipitate, which was filtered and the filter cake was collected to obtain the title compound 68B (10.32 g, yield: 77%) as a white solid.

MS m/z (ESI): 358.3/359.7 [M+H]⁺.

### Step 3: Preparation of cis-3-(3-bromophenyl)-3-(5-mercapto-4-methyl-4H-1,2,4-triazol-3-yl)cyclobutanol (68C)

68B (10.32 g, 28.81 mmol) was dissolved in sodium hydroxide solution (1 M, 173.87 mL), nitrogen replacement was performed three times, and the mixture was stirred at room temperature overnight. The reaction solution was adjusted to pH 3 with 2 M hydrochloric acid, a white precipitate was precipitated and filtered, and the filter cake was collected to obtain a white solid. The filtrate was extracted with ethyl acetate (100 mL×3), the organic phase was dried over anhydrous sodium sulfate, filtered, and the solvent was removed by rotary evaporation to obtain the title compound 68C (8.66 g in total, yield: 88%) as a white solid.

MS m/z (ESI): 340.2/341.8 [M+H]⁺.

### Step 4: Preparation of cis-3-(3-bromophenyl)-3-(4-methyl-4H-1,2,4-triazol-3-yl)cyclobutanol (68D)

68C (8.66 g, 25.45 mmol) was dissolved in dichloromethane (100 mL), glacial acetic acid (38.21 g, 636.32 mmol) was added, and 30% hydrogen peroxide (37.71 g, 332.59 mmol) was slowly added dropwise at 0° C. (ice-water bath). After the addition was completed, the reaction solution was returned to room temperature and reacted for 2 hours. The reaction solution was quenched with saturated aqueous sodium sulfite solution, extracted with dichloromethane (100 mL×3), and a white precipitate was precipitated and filtered. The filter cake was dissolved in methanol (200 mL), stirred at 60° C. for half an hour, filtered, and the filtrate was concentrated to dryness under reduced pressure to obtain the title compound 68D (2.39 g, yield: 30%) as a white solid.

MS m/z (ESI): 308.2/309.7 [M+H]⁺.

### Step 5: Preparation of cis-3-(3-bromophenyl)-3-(4-methyl-4H-1,2,4-triazol-3-yl)cyclobutylmethanesulfonate (68E)

68D (2.39 g, 7.76 mmol) was dissolved in dichloromethane (30 mL), triethylamine (9.42 g, 93.06 mmol) was added, the mixture was cooled to 0° C. (ice-water bath), methanesulfonyl chloride (11.33 g, 98.91 mmol) was added, and the reaction was allowed to react at room temperature for 2 hours. The reaction solution was quenched with water (50 mL), extracted with dichloromethane (50 mL×2), the organic phase was dried over anhydrous sodium sulfate, and filtered, and the solvent was removed by rotary evaporation to obtain a crude product, which was separated by normal phase column chromatography (0-11% methanol/dichloromethane), to obtain the title compound 68E (2.18 g, yield: 73%).

MS m/z (ESI): 386.3/387.8 [M+H]⁺.

### Step 6: Preparation of trans-3-(3-bromophenyl)-3-(4-methyl-4H-1,2,4-triazol-3-yl)cyclobutylacetonitrile (68F) and cis-3-(3-bromophenyl)-3-(4-methyl-4H-1,2,4-triazol-3-yl)cyclobutylacetonitrile (68G)

68E (2.11 g, 5.46 mmol) was dissolved in dimethyl sulfoxide (50 mL), potassium carbonate (1.51 g, 10.93 mmol) and potassium cyanide (1.74 g, 26.72 mmol) were sequentially added, and the reaction solution was stirred at 120° C. under nitrogen protection for 32 hours. After cooled to room temperature, the reaction solution was poured into ethyl acetate (80 mL), washed with water (80 mL×2), and the organic phase was dried over anhydrous sodium sulfate, filtered, and the solvent was removed by rotary evaporation to obtain a crude product, which was separated by normal phase column chromatography (0-20% methanol/dichloromethane) to obtain a yellow oil (616 mg). The yellow oil was further purified by reverse phase column chromatography (chromatographic column: SepaFlash® silica gel column; eluent: 15% to 30% (v/v) acetonitrile/water), to obtain a refined product, which was lyophilized in a dry ice/ethanol bath (−78° C.) to obtain the title compound. 68F (218 mg, yield: 13%) and the title compound 68G (124 mg, yield: 7%).

### Compound 68F:

MS m/z (ESI): 317.2/318.8 [M+H]⁺;

¹H NMR (400 MHz, DMSO-d₆) δ 8.43 (s, 1H), 7.53-7.48 (m, 1H), 7.44-7.40 (m, 1H), 7.37-7.32 (m, 1H), 7.23-7.19 (m, 1H), 3.63-3.36 (m, 1H), 3.33-3.30 (m, 2H), 3.17 (s, 3H), 3.06-2.96 (m, 2H).

### Compound 68G:

MS m/z (ESI): 317.2/318.8 [M+H]⁺;

¹H NMR (400 MHz, DMSO-d₆) δ 8.38 (s, 1H), 7.54-7.47 (m, 2H), 7.37-7.32 (m, 1H), 7.29-7.24 (m, 1H), 3.62-3.52 (m, 1H), 3.29-3.22 (m, 2H), 3.19-3.16 (m, 3H), 3.15-3.07 (m, 2H).

### Step 7 to step 8: Preparation of trans-3-(3-(6-bromo-8-methyl-4-oxo-4H-chromen-3-yl)phenyl)-3-(4-methyl-4H-1,2,4-triazol-3-yl)cyclobutyl acetonitrile (68J)

Referring to the synthesis method in step 7 to step 8 of Example 9, the title compound 68J (15 mg, yield: 57%) was prepared using the same method, except that 65F was replaced with 68F (17.6 mg, 55.52 μmol).

MS m/z (ESI): 475.1[M+H]⁺;

### Step 9: Preparation of trans-3-(4-methyl-4H-1,2,4-triazol-3-yl)-3-(3-(8-methyl-6-(((S)-3-methylpiperidin-1-yl)methyl)-4-oxo-4H-chromen-3-yl)phenyl)cyclobutylacetonitrile (compound 68)

Referring to the synthesis method in step 9 of Example 9, except that, 68J (15 mg, 31.65 mol), instead of 65H, was reacted with 2B (10.37 mg, 47.33 μmol) to finally obtain the title compound 68 (5.1 mg, yield: 32%).

MS m/z (ESI):508.26 [M+H]⁺;

¹H NMR (400 MHz, DMSO-d₆) δ 8.67 (s, 1H), 8.45 (s, 1H), 7.89 (d, J=2.1 Hz, 1H), 7.69-7.62 (m, 1H), 7.55-7.37 (m, 3H), 7.25-7.17 (m, 1H), 3.53 (s, 2H), 3.46-3.37 (m, 4H), 3.28 (s, 3H), 3.09-3.05 (m, 1H), 2.78-2.70 (m, 2H), 2.51 (s, 3H), 1.89 (d, J=11.6 Hz, 1H), 1.67-1.47 (m, 5H), 0.83 (d, J=5.3 Hz, 4H).

### Example 13: Preparation of 3-(3-((1R,3S)-3-fluoro-1-(4-methyl-4H-1,2,4-triazol-3-yl)cyclobutyl)phenyl)-8-methyl-6-(((S)-3-methylpiperidin-1-yl)methyl)-4H-chromen-4-one (compound 69)

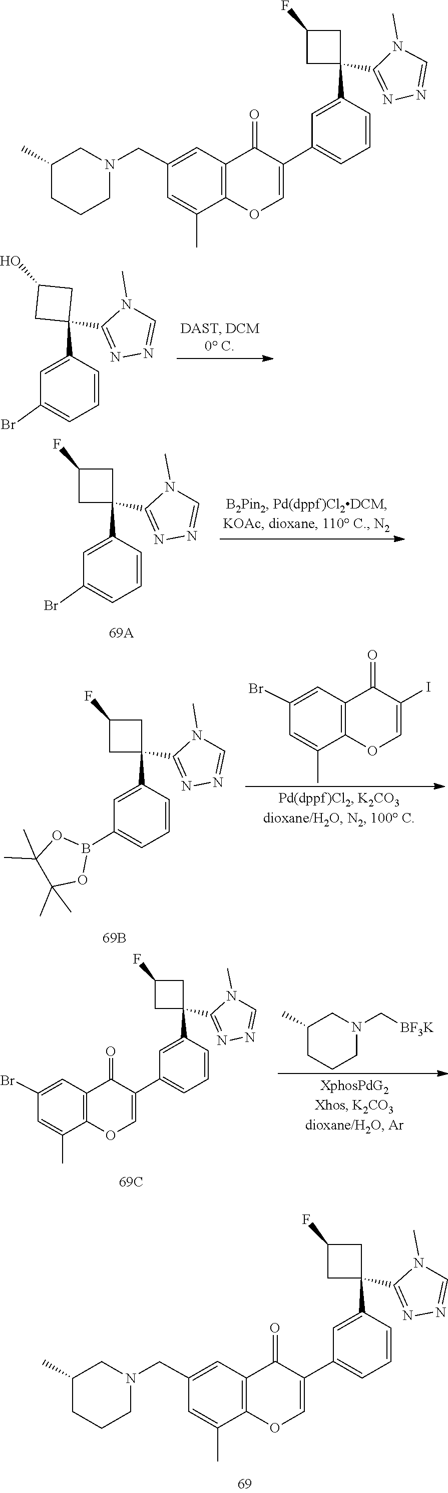

### Step 1: Preparation of 3-(trans-1-(3-bromophenyl)-3-fluorocyclobutyl)-4-methyl-4H-1,2,4-triazole (69A)

Cis-3-(3-bromophenyl)-3-(4-methyl-4H-1,2,4-triazol-3-yl)cyclobutanol (100 mg, 324.49 mol) was dissolved in dichloromethane (5 mL), the mixture was cooled to 0° C., and diethylaminosulfur trifluoride (78.46 mg, 486.74 μmol) was added dropwise under argon atmosphere. The mixture was stirred for 1 hour and quenched with saturated aqueous NaHCO₃ solution (1 mL), concentrated, and purified by reverse phase column chromatography (chromatographic column: SepaFlash® silica gel column; mobile phase: acetonitrile/water=7:3), to obtain the title compound 69A (51 mg, yield: 51%).

MS m/z (ESI): 310.03/312.03 [M+H]⁺.

### Step 2 to step 3: Preparation of 6-bromo-3-(3-(trans-3-fluoro-1-(4-methyl-4H-1,2,4-triazol-3-yl)cyclobutyl)phenyl)-8-methyl-4H-chromen-4-one (69C)

Referring to the synthesis method in step 7 to step 8 of Example 9, the title compound 69C (15 mg, yield: 20%) was prepared using the same method, except that 65F was replaced with 69A (50 mg, 160.26 μmol).

MS m/z (ESI): 468.1[M+H]⁺.

### Step 4: Preparation of 3-(3-(trans-3-fluoro-1-(4-methyl-4H-1,2,4-triazol-3-yl)cyclobutyl)phenyl)-8-methyl-6-(((S)-3-methylpiperidin-1-yl)methyl)-4H-chromen-4-one (compound 69)

Referring to the synthesis method in step 9 of Example 9, except that, 69C (15 mg, 32.03 mol), instead of 65H, was reacted with 2B (10.53 mg, 48.04 μmol) to finally obtain the title compound 69 (2.2 mg, yield: 14%).

MS m/z (ESI):501.26 [M+H]⁺.

¹H NMR (400 MHz, DMSO-d₆) δ 8.66 (s, 1H), 8.43 (s, 1H), 7.88 (d, J=2.1 Hz, 1H), 7.64 (d, J=2.1 Hz, 1H), 7.53-7.47 (m, 3H), 7.20 (dt, J=7.4, 1.8 Hz, 1H), 5.20-5.01 (m, 1H), 3.51 (d, J=14.7 Hz, 5H), 2.91-2.81 (m, 2H), 2.74 (t, J=10.7 Hz, 2H), 2.50 (s, 3H), 2.05-1.87 (m, 2H), 1.69-1.49 (m, 6H), 0.83 (d, J=5.3 Hz, 4H).

### Example 14: Preparation of 8-methyl-3-(3-(2-(4-methyl-4H-1,2,4-triazol-3-yl)oxetan-2-yl)phenyl)-6-(((S)-3-methylpiperidin-1-yl)methyl)-4H-chromen-4-one (compound 70)

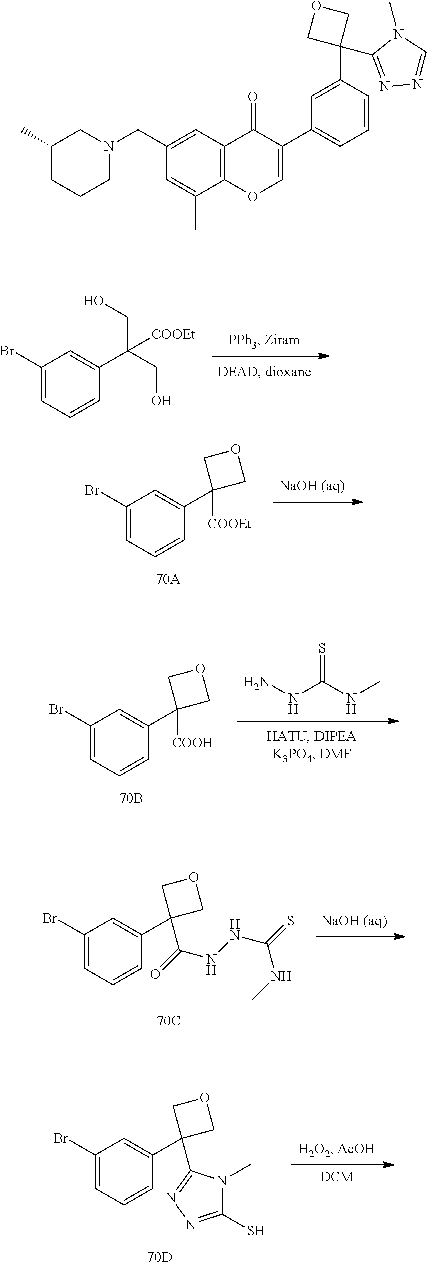

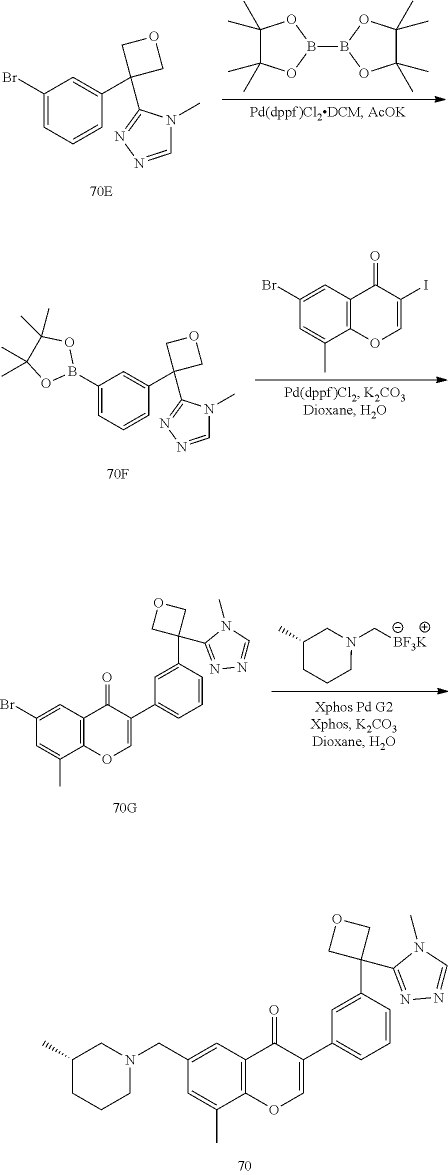

### Step 1: Preparation of ethyl 3-(3-bromophenyl)oxetane-3-carboxylate (70A)

Ethyl 2-(3-bromophenyl)-3-hydroxy-2-(hydroxymethyl)propionate (3.0 g, 10.38 mmol) and triphenylphosphine (5.44 g, 20.75 mmol) were dissolved in anhydrous dioxane (30.0 mL). Zinc dimethyldithiocarbamate (2.71 g, 15.56 mmol) and DEAD (3.61 g, 20.75 mmol) were sequentially added dropwise to the reaction solution in an ice bath at 0° C. and under argon-blowing conditions. The reaction solution was slowly heated to room temperature and stirred for 16 hour. Upon the raw materials disappeared as monitored by TLC, the reaction was terminated, filtered and distilled to dryness under reduced pressure to obtain the title compound 70A (2.81 g, yield: 99.9%) as a crude, which was directly used in the next step without further purification.

### Step 2: Preparation of 3-(3-bromophenyl)oxetane-3-carboxylic acid (70B)

At room temperature, 70A (2.81 g, 10.38 mmol) was dissolved in acetonitrile (30.0 mL) and aqueous sodium hydroxide solution (30.0 mL, 3.0 M) was added, and the reaction solution was reacted at 65° C. for 1.0 hour. After the reaction was completed, the reaction solution was cooled down to 0° C., saturated aqueous citric acid solution was added to adjust the pH to acidity, and the mixture was extracted three times with dichloromethane. The organic phase was dried over anhydrous sodium sulfate, filtered, and distilled under reduced pressure to obtain a crude product, which was purified by reverse phase column chromatography (chromatographic column: SepaFlash® silica gel column; mobile phase: acetonitrile/water=20:80), to obtain the title compound 70B (750.0 mg, yield: 29.6%).

MS m/z (ESI): 255.0/257.0 [M−H]⁻.

### Step 3: Preparation of 2-(3-(3-bromophenyl)oxetane-3-carbonyl)-N-methylhydrazine-1-thioamide (70C)

70B (750.0 mg, 2.92 mmol), 4-methylthiosemicarbazide (368.16 mg, 3.50 mmol), HATU, potassium phosphate (1.32 g, 3.5 mmol) and DIPEA (1.3 g, 8.75 mmol, 1.52 mL) were added to DMF (8.0 mL). The reaction was allowed to proceed at room temperature for 1.0 hour. After the reaction was completed, water (60.0 mL) was added, and the mixture was extracted with dichloromethane (6.0 mL×3). The organic phase obtained by extraction was dried over anhydrous sodium sulfate, filtered, and distilled under reduced pressure to obtain the title product 70C (900.0 mg, yield: 90%), which was directly used in the next step without further purification.

MS m/z(ESI): 344.0/346.0 [M+H]⁺.

### Step 4: Preparation of 5-(3-(3-bromophenyl)oxetan-3-yl)-4-methyl-4H-1,2,4-triazole-3-thiol (70D)

70C (900.0 mg, 2.61 mmol) was added to aqueous sodium hydroxide solution (10.0 mL, 3.0 M), and the reaction solution was reacted at 80° C. for 1.0 hour. After the reaction was completed, the reaction solution was cooled down to 0° C., saturated aqueous citric acid solution was added to adjust the pH to acidity, and the mixture was extracted three times with dichloromethane. The organic phase obtained by extraction was dried over anhydrous sodium sulfate, filtered, and distilled under reduced pressure to obtain the title product 70D (750.0 mg, yield: 87.9%), which was directly used in the next step without further purification.

MS m/z(ESI): 326.0/328.0 [M+H]⁺.

### Step 5: Preparation of 3-(3-(3-bromophenyl)oxetan-3-yl)-4-methyl-4H-1,2,4-triazole (70E)

At room temperature, 70D (750.0 mg, 2.31 mmol) was dissolved in dichloromethane (6.0 mL), and glacial acetic acid (0.5 mL) and hydrogen peroxide (9.0 mL) were sequentially and slowly added dropwise to the reaction solution. The reaction solution was reacted at room temperature for 1.0 hour. After the reaction was completed, water (60.0 mL) was added, and the mixture was extracted with dichloromethane (6.0 mL×3). The organic phase obtained by extraction was dried over anhydrous sodium sulfate, filtered, and distilled under reduced pressure to obtain the title product 70E (600.0 mg, yield: 88.7%), which was directly used in the next step without further purification.

MS m/z(ESI): 294.0/296.0 [M+H]⁺.

### Step 6: Preparation of 4-methyl-3-(3-(3-(4,4,5,5-tetramethyl-1,3,2-dioxaborolan-2-yl)phenyl)oxetan-3-yl)-4H-1,2,4-triazole (70F)

Pd(dppf)Cl₂-DCM (17.2 mg, 0.068 mmol) was added to a mixture of 70E (100 mg, 0.34 mmol), B₂Pin₂ (129.53 mg, 0.5 mmol), potassium acetate (100 mg, 1.02 mmol) and anhydrous dioxane (3.0 mL), and the resulting mixture was heated to 100° C. under nitrogen protection, and stirred for 12 hours. After the reaction was completed, the mixture was cooled to room temperature, filtered, and concentrated under reduced pressure. The residue was purified by normal phase column chromatography (petroleum ether:ethyl acetate=0:1) to obtain the title compound 70F (80.0 mg, yield: 69%).

MS m/z (ESI): 342.0 [M+H]⁺.

### Step 7: Preparation of 6-bromo-8-methyl-3-(3-(3-(4-methyl-4H-1,2,4-triazol-3-yl)oxetan-3-yl)phenyl)-4H-chromen-4-one (70G)

70F (20.0 mg, 58.61 μmol), 58C (21.39 mg, 58.61 μmol) and potassium carbonate (16.1 mg, 117.23 μmol) were added to a mixture of water (0.2 mL) and 1,4-dioxane (1.0 mL), and nitrogen replacement was performed. Under nitrogen atmosphere, Pd(dppf)Cl₂ (8.51 mg, 11.72 mol) was added, and the resulting mixture was heated to 100° C. under nitrogen protection and stirred for 1.0 h. After the reaction was completed, the mixture was cooled to room temperature, filtered, and concentrated under reduced pressure to obtain a crude product, which was purified by reverse phase column chromatography (chromatographic column: SepaFlash® silica gel column; mobile phase: acetonitrile/water=60:40), to obtain the title compound 70G (20.0 mg, yield: 75.5%).

MS m/z (ESI): 452.0/454.0 [M+H]⁺.

### Step 8: Preparation of 8-methyl-3-(3-(2-(4-methyl-4H-1,2,4-triazol-3-yl)oxetan-2-yl)phenyl)-6-(((S)-3-methylpiperidin-1-yl)methyl)-4H-chromen-4-one (compound 70)

70G (20 mg, 44.22 μmol), 2B (29.0 mg, 132.66 μmol) and potassium carbonate (18.3 mg, 132.66 μmol) were added to a mixture of water (0.2 mL) and 1,4-dioxane (1.0 mL), and nitrogen replacement was performed. Under nitrogen atmosphere, XphosPd G₂ (6.96 mg, 8.84 mol) and Xphos (8.43 mg, 17.69 μmol) were added to the solution, and the reaction solution was heated to 100° C. and stirred for 1.0 hour. After the reaction was completed, the reaction solution was cooled to room temperature, water (10 mL) was added, and the mixture was extracted with ethyl acetate (10 mL×3). The organic phase was dried over anhydrous sodium sulfate, filtered, and distilled under reduced pressure to obtain a crude product, which was purified by high performance liquid chromatography (chromatographic column: Welch Xtimate C18; column length 150 mm, inner diameter 30 mm, and particle size 5 μm; mobile phase A: water (with 0.225% aqueous ammonia); mobile phase B: acetonitrile; gradient: mobile phase B: from 5% to 95% over 18 minutes; flow rate: 25 mL/min), to obtain the title compound 70 (4.1 mg, 19%).

MS m/z (ESI): 485.42 [M+H]⁺.

¹H NMR (400 MHz, DMSO-d₆) δ 8.63 (s, 1H), 8.48 (s, 1H), 7.85 (s, 1H), 7.61 (s, 1H), 7.53 (s, 3H), 7.28 (s, 1H), 5.39 (s, 2H), 5.10 (s, 2H), 3.50 (s, 2H), 3.29 (s, 3H), 2.70 (s, 2H), 1.57 (s, 4H), 1.23 (s, 3H), 0.79 (s, 3H).

### Example 15: Preparation of 8-methyl-3-(3-(2-(4-methyl-4H-1,2,4-triazol-3-yl)oxetan-2-yl)phenyl)-6-(((S)-3-methylpiperidin-1-yl)methyl)-4H-chromen-4-one (compound 71)

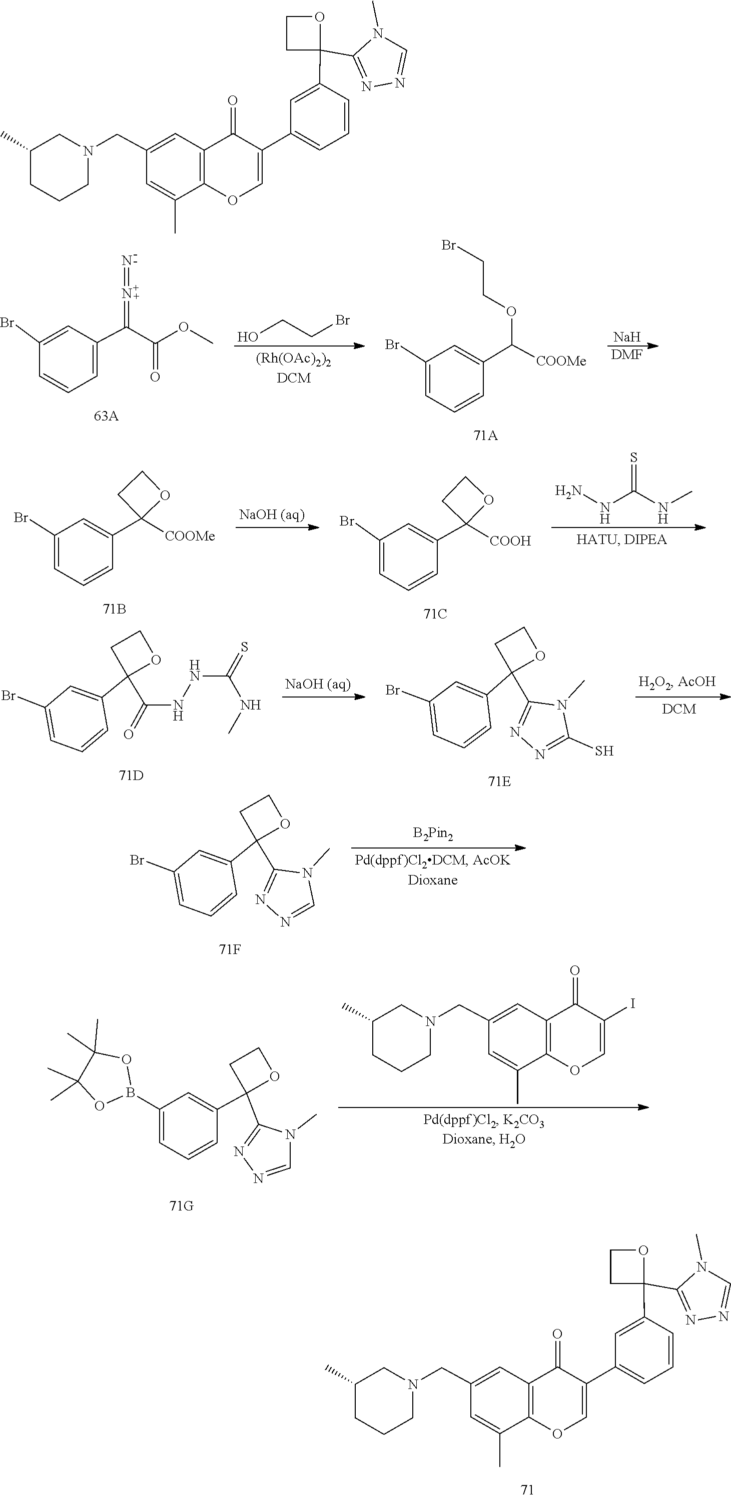

### Step 1: Preparation of methyl 2-(2-bromoethoxy)-2-(3-bromophenyl) acetate (71A)

63A (1.0 g, 3.92 mmol) was dissolved in anhydrous dichloromethane (5.0 mL). In an ice bath at 0° C., the solution was added dropwise to a mixture of bromoethanol (499 mg, 3.92 mmol) and dimeric rhodium acetate (173 mg, 0.392 mmol) dissolved in dichloromethane (5.0 mL). The reaction solution was slowly heated to room temperature and stirred for 1 hour. After the reaction was completed as monitored by TLC, the solution was filtered and the filtrate was distilled under reduced pressure to obtain the crude product, which was purified by reverse phase column chromatography (chromatographic column: SepaFlash® silica gel column; mobile phase: acetonitrile/water=55:45), to obtain the title compound 71A (1.10 g, yield: 79.7%).

### Step 2: Preparation of methyl 2-(3-bromophenyl)oxetane-2-carboxylate (71B)

NaH (102.0 mg, 2.84 mmol, 60% dispersed in mineral oil) was added to a sealed tube, and argon replacement was performed four times for protection. DMF (1.0 mL) was added in an ice bath, and then 71A (1.0 g, 2.84 mmol) was dissolved in DMF (9.0 mL) and slowly added dropwise to the sealed tube. After the addition was completed, the reaction solution was slowly heated to room temperature and stirred for 2.0 hours. After the reaction was completed, the reaction solution was added dropwise to ice water (100.0 mL), and the mixture was extracted with ethyl acetate (10.0 mL×3). The organic phase was dried over anhydrous sodium sulfate, filtered, and distilled under reduced pressure to obtain the title product 71B (695.0 mg, yield: 90%), which was directly used in the next step without further purification.

### Step 3: Preparation of 2-(3-bromophenyl)oxetane-2-carboxylic acid (71C)

At room temperature, 71B (695.0 mg, 2.56 mmol) was dissolved in acetonitrile (10.0 mL) and aqueous sodium hydroxide solution (10.0 mL, 3.0 M) was added, and the mixture was reacted at 65° C. for 1.0 hour. After the reaction was completed, the reaction solution was cooled down to 0° C., saturated aqueous citric acid solution was added to adjust the pH to acidity, and the mixture was extracted three times with dichloromethane. The organic phase was dried over anhydrous sodium sulfate, filtered, and distilled under reduced pressure to obtain the title compound 71C (594.0 mg, yield: 90%) as a crude, which was directly used in the next step without further purification.

MS m/z (ESI): 255.0/257.0 [M−H]⁻.

### Step 4: Preparation of 2-(2-(3-bromophenyl)oxetane-2-carbonyl)-N-methylhydrazine-1-thioamide (71D)

71C (594.0 mg, 2.31 mmol), 4-methylthiosemicarbazide (292.0 mg, 2.77 mmol), HATU (1.05 g, 2.77 mmol), potassium phosphate (1.05 g, 2.77 mmol) and DIPEA (896.0 mg, 6.93 mmol, 1.21 mL) were added to DMF (6.0 mL). The reaction was carried out at room temperature for 1.0 hour. After the reaction was completed, water (60.0 mL) was added, and the mixture was extracted with dichloromethane (6.0 mL×3). The organic phase obtained by extraction was dried over anhydrous sodium sulfate, filtered, and distilled under reduced pressure to obtain the title product 71D (720.0 mg, yield: 90%), which was directly used in the next step without further purification.

MS m/z(ESI): 344.0/346.0 [M+H]⁺.

### Step 5: Preparation of 5-(2-(3-bromophenyl)oxetan-2-yl)-4-methyl-4H-1,2,4-triazole-3-thiol (71E)

71D (720.0 mg, 2.09 mmol) was added to aqueous sodium hydroxide solution (10.0 mL, 3.0 M), and the reaction solution was reacted at 80° C. for 1.0 hour. After the reaction was completed, the reaction solution was cooled down to 0° C., saturated aqueous citric acid solution was added to adjust the pH to acidity, and the mixture was extracted three times with dichloromethane. The organic phase obtained by extraction was dried over anhydrous sodium sulfate, filtered, and distilled under reduced pressure to obtain the title product 71E (614.0 mg, yield: 90%), which was directly used in the next step without further purification.

MS m/z(ESI): 326.0/328.0 [M+H]⁺.

### Step 6: Preparation of 3-(2-(3-bromophenyl)oxetan-2-yl)-4-methyl-4H-1,2,4-triazole (71F)

At room temperature, 71E (614.0 mg, 1.88 mmol) was dissolved in dichloromethane (6.0 mL), and glacial acetic acid (0.5 mL) and hydrogen peroxide (3.0 mL) were sequentially and slowly added dropwise to the reaction solution. The reaction solution was reacted at room temperature for 1.0 hour. After the reaction was completed, water (60.0 mL) was added, and the mixture was extracted with dichloromethane (6.0 mL×3). The organic phase obtained by extraction was dried over anhydrous sodium sulfate, filtered, and distilled under reduced pressure to obtain the title product 71F (500.0 mg, yield: 90%), which was directly used in the next step without further purification.

MS m/z(ESI): 294.0/296.0 [M+H]⁺.

### Step 7: Preparation of 4-methyl-3-(2-(3-(4,4,5,5-tetramethyl-1,3,2-dioxaborolan-2-yl)phenyl)oxetan-2-yl)-4H-1,2,4-triazole (71G)

Pd(dppf)Cl₂-DCM (17.2 mg, 0.068 mmol) was added to a mixture of 71F (100 mg, 0.34 mmol), B₂Pin₂ (129.53 mg, 0.5 mmol), potassium acetate (100.0 mg, 1.02 mmol) and anhydrous dioxane (3.0 mL), and the resulting mixture was heated to 100° C. under nitrogen protection, and stirred for 12 hours. After the reaction was completed, the reaction mixture was cooled to room temperature, filtered, and concentrated under reduced pressure. The residue was purified by normal phase column chromatography (petroleum ether:ethyl acetate=0:1) to obtain the title compound 71G (80.0 mg, yield: 68%).

MS m/z (ESI): 342.0 [M+H]⁺.

### Step 8: Preparation of 8-methyl-3-(3-(2-(4-methyl-4H-1,2,4-triazol-3-yl)oxetan-2-yl)phenyl)-6-(((S)-3-methylpiperidin-1-yl)methyl)-4H-chromen-4-one (compound 71)

71G (25.7 mg, 75.52 μmol), 64C (15.0 mg, 37.76 μmol) and potassium carbonate (15.63 mg, 113.28 μmol) were added to a mixture of water (0.3 mL) and 1,4-dioxane (1.5 mL), and nitrogen replacement was performed. Under nitrogen atmosphere, Pd(dppf)Cl₂ (5.48 mg, 7.55 mol) was added, and the resulting mixture was heated to 100° C. under nitrogen protection and stirred for 1.0 h. After the reaction was completed, the mixture was cooled to room temperature, filtered, and concentrated under reduced pressure to obtain a crude product, which was separated by preparative high performance liquid chromatography (chromatographic column: Welch Xtimate C18, column length 150 mm, inner diameter 30 mm, and particle size 5 μm; mobile phase A: water (0.225% aqueous ammonia), mobile phase B: acetonitrile; gradient: mobile phase B: from 5% to 95% over 24 minutes), to obtain the title compound 71 (6.86 mg, yield: 37%).

MS m/z (ESI): 485.2 [M+H]⁺.

¹H NMR (400 MHz, DMSO-d₆) δ 8.62 (s, 1H), 8.46 (s, 1H), 7.85 (s, 1H), 7.59 (d, J=8.1 Hz, 2H), 7.51 (q, J=8.3, 7.6 Hz, 2H), 7.29 (d, J=6.7 Hz, 1H), 4.68 (dt, J=22.3, 7.5 Hz, 2H), 3.88 (s, 1H), 3.51 (s, 2H), 3.29 (s, 3H), 2.93-2.86 (m, 1H), 2.70 (s, 2H), 2.46 (s, 3H), 1.92-1.81 (m, 1H), 1.66-1.54 (m, 4H), 1.45 (d, J=11.8 Hz, 1H), 0.84 (s, 1H), 0.79 (d, J=4.6 Hz, 3H).

### Example 16: Preparation of 8-((dimethylamino)methyl)-9-methyl-3-(3-(3-methyl-1-(4-methyl-4H-1,2,4-triazol-3-yl)cyclobutyl)phenyl)-4H-pyrido[1,2-a]pyrimidin-4-one (compound 72)

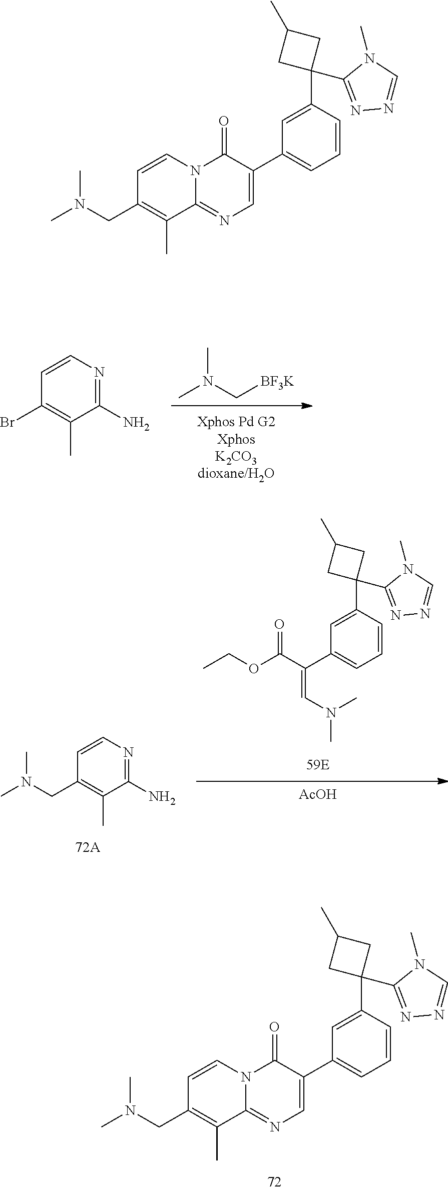

### Step 1: Preparation of 4-((dimethylamino)methyl)-3-methylpyridin-2-amine (72A)

4-Bromo-3-methylpyridin-2-amine (1 g, 5.35 mmol), potassium ((dimethylamino)methyl)trifluoroborate (1.06 g, 6.42 mmol), XPhos Pd G2 (421 mg, 535 μmol), potassium carbonate (2.21 g, 16.05 mmol) and Xphos (511 mg, 1.07 mmol) were added to a reaction tube, which was evacuated and backfilled with argon, water (8 mL) and dioxane (30 mL) were added with a syringe, the mixture was heated to 100° C. and reacted for 12 hours, and cooled to room temperature, and the reaction solution was directly purified by reverse phase column chromatography (chromatographic column: SepaFlash® silica gel column; mobile phase: acetonitrile/water=60:40), and then lyophilized, to obtain the title compound 72A (410 mg, yield: 46%).

MS m/z (ESI):166.1 [M+H]⁺;

### Step 2: Preparation of 8-((dimethylamino)methyl)-9-methyl-3-(3-(3-methyl-1-(4-methyl-4H-1,2,4-triazol-3-yl)cyclobutyl)phenyl)-4H-pyrido[1,2-a]pyrimidin-4-one (compound 72)

59E (89.2 mg, 242.08 μmol) and 72A (40.0 mg, 242.08 μmol) were added to glacial acetic acid (3 mL), and the reaction solution was heated to 110° C. and stirred for 2 hours. After the reaction was completed, the reaction solution was distilled to obtain a crude product, which was purified by high performance liquid chromatography (chromatographic column: Welch Xtimate C18; column length 150 mm, inner diameter 30 mm, and particle size 5 μm; mobile phase A: water (with 0.225% aqueous ammonia); mobile phase B: acetonitrile; gradient: mobile phase B: from 5% to 95% over 24 minutes, flow rate: 24 mL/min), to obtain the title compound 72 (20.0 mg, 18.6%).

MS m/z (ESI): 443.2 [M+H]⁺.

¹H NMR (400 MHz, DMSO-d6) δ 9.00 (s, 1H), 8.64 (s, 1H), 8.31 (d, J=30.4 Hz, 1H), 7.87-7.63 (m, 2H), 7.44 (d, J=7.6 Hz, 2H), 7.12 (s, 1H), 3.59 (s, 1H), 3.27 (s, 3H), 3.22 (s, 2H), 3.12 (s, 1H), 2.87 (s, 1H), 2.57 (s, 3H), 2.27 (s, 6H), 1.17 (d, J=49.7 Hz, 1H), 1.09 (s, 3H).

### Example 17: Preparation of (S)-3-(3-(3,3-difluoro-1-(4-methyl-4H-1,2,4-triazol-3-yl)cyclobutyl)phenyl)-8-methyl-6-((3-methylpiperidin-1-yl)methyl)-4H-chromen-4-one (compound 73)

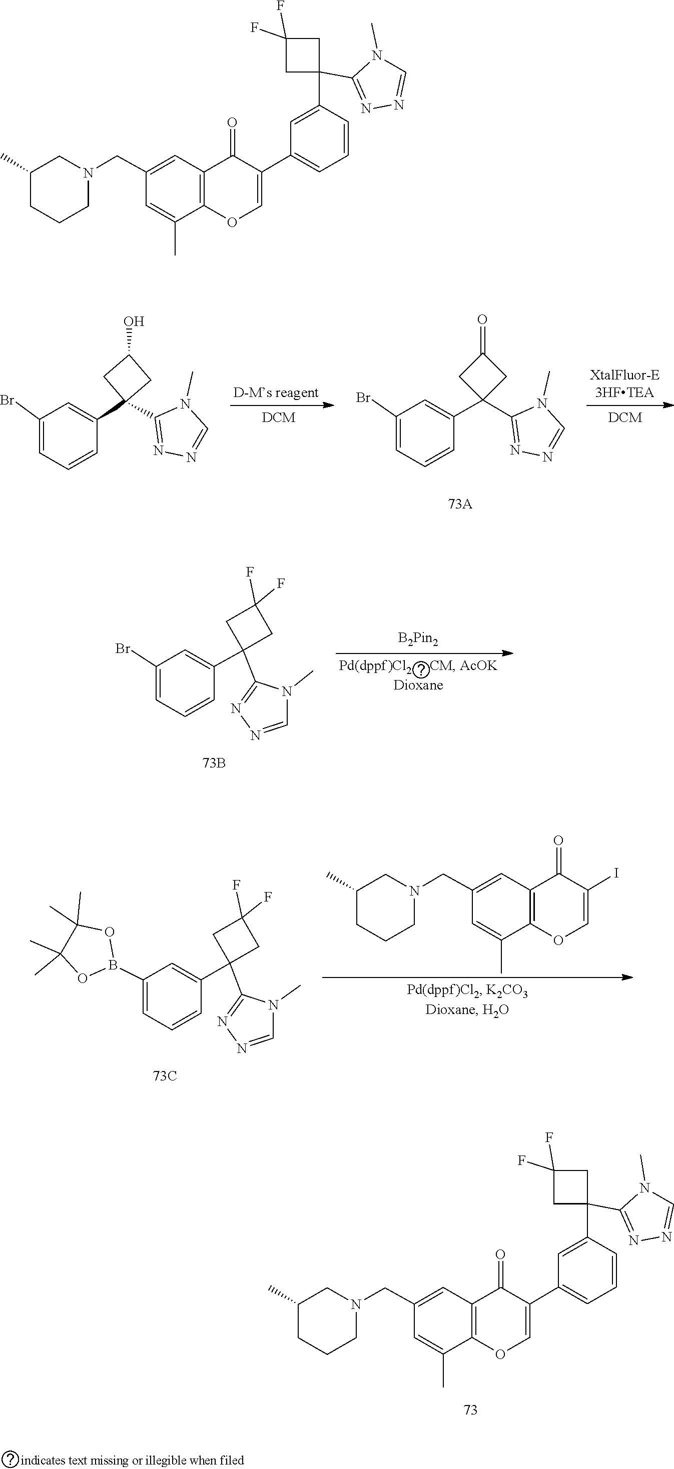

### Step 1: Preparation of 3-(3-bromophenyl)-3-(4-methyl-4H-1,2,4-triazol-3-yl)cyclobutan-1-one (73A)

(1S,3S)-3-(3-Bromophenyl)-3-(4-methyl-4H-1,2,4-triazol-3-yl)cyclobutanol (500.0 mg, 1.62 mmol) was dissolved in anhydrous dichloromethane (20 mL). In an ice bath at 0° C., Dess-Martin periodinane (1.38 g, 3.24 mmol) was added in batches to the solution. After the addition, the reaction solution was slowly heated to room temperature and stirred for 2.0 hours. After the reaction was completed, the reaction residue was filtered out and the filtrate was rinsed with dichloromethane. The organic phase was dried over anhydrous sodium sulfate, filtered, and distilled to dryness under reduced pressure to obtain a crude product, which was purified by reverse phase column chromatography (chromatographic column: SepaFlash® silica gel column; mobile phase: acetonitrile/water=45:55), to obtain the title compound 73A (400.0 mg, yield: 80%).

MS m/z(ESI): 306/308 [M+H]⁺.

### Step 2: Preparation of 3-(1-(3-bromophenyl)-3,3-difluorocyclobutyl)-4-methyl-4H-1,2,4-triazole (73B)

In an ice bath, triethylamine hydrogen fluoride (663.0 mg, 3.92 mmol) was added to dichloromethane (5.0 mL), and argon replacement was performed four times for protection. Under argon-blowing conditions, to the solution were sequentially added XtalFluor-E (897.5 mg, 3.92 mmol) and 73A (400.0 mg, 1.31 mmol). The reaction solution was then slowly heated to room temperature and stirred for 2.0 hours. After the reaction was completed, the reaction solution was cooled to 0° C., saturated aqueous ammonium chloride solution (60 mL) was added to quench the reaction, and the mixture was extracted with dichloromethane (50 mL×3). The organic phase was dried over anhydrous sodium sulfate, filtered, and distilled to dryness under reduced pressure to obtain a crude product, which was purified by reverse phase column chromatography (chromatographic column: SepaFlash® silica gel column; mobile phase: acetonitrile/water=60:40), to obtain the title compound 73B (20.0 mg, yield: 9.3%).

MS m/z (ESI): 328.0/330.0 [M+H]⁺.

### Step 3: Preparation of 3-(3,3-difluoro-1-(3-(4,4,5,5-tetramethyl-1,3,2-dioxaborolan-2-yl)phenyl)cyclobutyl)-4-methyl-4H-1,2,4-triazole (73C)

To a mixture of 73B (10.0 mg, 30.47 μmol), B₂Pin₂ (9.92 mg, 36.57 μmol), potassium acetate (8.96 mg, 91.42 μmol) and anhydrous dioxane (1.0 mL) was added Pd(dppf)Cl₂-DCM (4.42 mg, 6.09 μmol), and the resulting mixture was heated to 100° C. under nitrogen protection and stirred for 12 hours. After the reaction was completed, the mixture was cooled to room temperature, filtered, and concentrated under reduced pressure. The residue was purified by normal phase column chromatography (petroleum ether:ethyl acetate=0:1) to obtain the title compound 73C (10.0 mg, yield: 87%).

MS m/z (ESI): 376.1 [M+H]⁺.

### Step 4: Preparation of (S)-3-(3-(3,3-difluoro-1-(4-methyl-4H-1,2,4-triazol-3-yl)cyclobutyl)phenyl)-8-methyl-6-((3-methylpiperidin-1-yl)methyl)-4H-chromen-4-one (compound 73)

73C (10.0 mg, 26.65 μmol), 64C (10.59 mg, 26.65 μmol) and potassium carbonate (7.36 mg, 53.3 μmol) were added to a mixture of water (0.2 mL) and 1,4-dioxane (1.0 mL), and nitrogen replacement was performed. Under nitrogen atmosphere, Pd(dppf)Cl₂ (3.87 mg, 5.33 mol) was added, and the resulting mixture was heated to 100° C. under nitrogen protection and stirred for 1.0 h. After the reaction was completed, the mixture was cooled to room temperature, filtered, and concentrated under reduced pressure to obtain a crude product, which was separated by preparative high performance liquid chromatography (chromatographic column: Welch Xtimate C18, column length 150 mm, inner diameter 30 mm, and particle size 5 μm; mobile phase A: water (with 0.225% aqueous ammonia), mobile phase B: acetonitrile; gradient: mobile phase B: from 5% to 95% over 18 minutes), to obtain the title compound 73 (2.21 mg, yield: 16%).

MS m/z (ESI): 519.46 [M+H]⁺.

¹H NMR (400 MHz, DMSO-d₆) δ 8.62 (s, 1H), 8.40 (s, 1H), 7.86 (s, 1H), 7.61 (s, 1H), 7.57 (s, 1H), 7.53-7.43 (m, 2H), 7.30 (d, J=7.5 Hz, 1H), 3.72 (s, 2H), 3.50 (s, 2H), 3.45 (d, J=13.0 Hz, 1H), 3.31 (s, 3H), 2.71 (s, 2H), 1.87 (s, 1H), 1.57 (s, 3H), 1.23 (s, 2H), 0.79 (s, 3H).

### Example 18: Preparation of (S)-8-cyclopropyl-3-(3-(3-methyl-1-(4-methyl-4H-1,2,4-triazol-3-yl)cyclobutyl)phenyl)-6-((3-methylpiperidin-1-yl)methyl)-4H-chromen-4-one (compound 74)

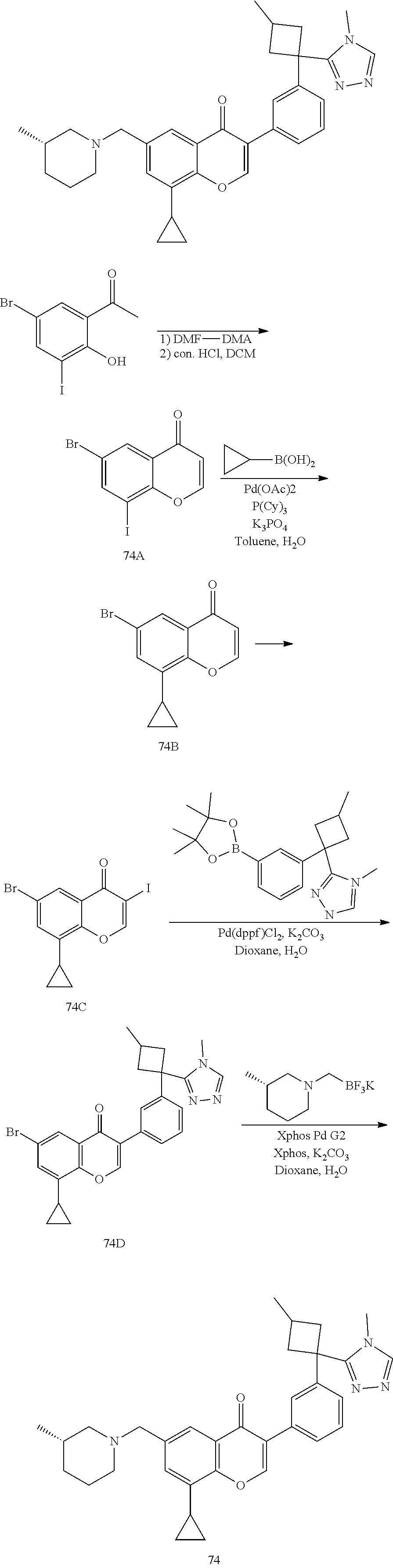

### Step 1: Preparation of 6-bromo-8-iodo-4H-chromen-4-one (74A)

1-(5-bromo-2-hydroxyl-3-iodophenyl)ethyl-1-one (341 mg, 1.00 mmol) was dissolved in DMF-DMA (6 mL), and the mixture was heated to 90° C., and reacted for 2 hours. The mixture was cooled to room temperature, concentrated and azeotroped with chloroform three times to obtain a brown gum. The gum was dissolved in dichloromethane (20 mL), 4 mL of concentrated hydrochloric acid was added, and the mixture was stirred at room temperature for 3 hours. The mixture was diluted with water, and liquid separation was performed. The aqueous phase was extracted with dichloromethane three times, and the organic phase was washed with saturated aqueous sodium bicarbonate solution, dried over MgSO₄, filtered, and concentrated to dryness under reduced pressure to obtain the title compound 74A (310 mg, yield: 89%) as a crude.

MS m/z(ESI): 351.0/353.0 [M+H]⁺.

### Step 2: Preparation of 6-bromo-8-cyclopropyl-4H-chromen-4-one (74B)

74A (300.0 mg, 854.86 μmol), cyclopropyl boronic acid (73.43 mg, 854.86 μmol) and potassium phosphate (544.36 mg, 2.56 mmol) were added to a mixture of water (1.6 mL) and toluene (8.0 mL), and nitrogen replacement was performed. Under nitrogen atmosphere, palladium acetate (38.38 mg, 170.97 μmol) and tricyclohexyl phosphine (95.89 mg, 341.94 μmol) were added to the solution, and the reaction system was heated to 90° C. and stirred for 16 hours. The crude product was purified by reverse phase column chromatography (chromatographic column: SepaFlash® silica gel column; mobile phase: acetonitrile/water=55:45), to obtain the title compound 74B (141.0 mg, yield: 62.2%).

MS m/z(ESI): 265.0/267.0 [M+H]⁺.

### Step 3: Preparation of 6-bromo-8-cyclopropyl-3-iodo-4H-chromen-4-one (74C)

At room temperature, 74B (141.0 mg, 531.87 μmol), cerium ammonium nitrate (282.0 mg, 531.87 μmol) and iodine (162.0 mg, 638.24 μmol) were sequentially added to acetonitrile (3.0 mL). The reaction solution was then heated to 80° C. and stirred for 16 hours. After the reaction was completed, the reaction solution was cooled to room temperature, water (60 mL) was added, and the mixture was extracted with ethyl acetate (50 mL×3). The organic phase was dried over anhydrous sodium sulfate, filtered, and distilled under reduced pressure to obtain a crude product, which was purified by normal phase column chromatography (petroleum ether:ethyl acetate=90:10), to obtain the title product 74C (50.0 mg, yield: 24%).

MS m/z (ESI): 391.0/393.0 [M+H]⁺.

### Step 4: Preparation of 6-bromo-8-cyclopropyl-3-(3-(3-methyl-1-(4-methyl-4H-1,2,4-triazol-3-yl)cyclobutyl)phenyl)-4H-chromen-4 one (74D)

74C (50.0 mg, 127.88 μmol), 1B (45.1 mg, 127.88 μmol) and potassium carbonate (35.29 mg, 255.76 μmol) were added to a mixture of water (0.2 mL) and 1,4-dioxane (1.0 mL), and nitrogen replacement was performed. Under nitrogen atmosphere, Pd(dppf)Cl₂ (18.56 mg, 25.58 mol) was added, and the resulting mixture was heated to 85° C. under nitrogen protection and stirred for 1.0 h. The crude product was purified by reverse phase column chromatography (chromatographic column: SepaFlash® silica gel column; mobile phase: acetonitrile/water=50:50), to obtain the title compound 74D (20.0 mg, yield: 32.0%).

MS m/z (ESI): 490/492 [M+H]⁺.

### Step 5: Preparation of (S)-8-cyclopropyl-3-(3-(3-methyl-1-(4-methyl-4H-1,2,4-triazol-3-yl)cyclobutyl)phenyl)-6-((3-methylpiperidin-1-yl)methyl)-4H-chromen-4-one (compound 74)

74D (20 mg, 40.78 μmol), 2B (26.81 mg, 122.35 μmol) and potassium carbonate (16.88 mg, 122.35 μmol) were added to a mixture of water (0.2 mL) and 1,4-dioxane (1.0 mL), and nitrogen replacement was performed. Under nitrogen atmosphere, XphosPd G₂ (6.42 mg, 8.16 mol) and Xphos (7.78 mg, 16.31 μmol) were added to the solution, and the reaction solution was heated to 100° C. and stirred for 16 hours. After the reaction was completed, the reaction solution was cooled to room temperature, water (10 mL) was added, and the mixture was extracted with ethyl acetate (10 mL×3). The organic phase was dried over anhydrous sodium sulfate, filtered, and distilled under reduced pressure to obtain a crude product, which was separated by preparative high performance liquid chromatography (chromatographic column: Welch Xtimate C18, column length 150 mm, inner diameter 30 mm, and particle size 5 μm; mobile phase A: water (with 0.225% aqueous ammonia), mobile phase B: acetonitrile; gradient: mobile phase B: from 5% to 95% over 24 minutes, flow rate: 24 mL/min), to obtain the title compound 74 (5.5 mg, yield: 25.8%).

MS m/z (ESI):523.54 [M+H]⁺;

¹H NMR (400 MHz, DMSO-d6) δ 8.63 (s, 1H), 8.32 (d, J=30.9 Hz, 1H), 7.85 (s, 1H), 7.64 (s, 1H), 7.44 (d, J=12.4 Hz, 2H), 7.30 (s, 2H), 3.51 (s, 1H), 3.28 (s, 1H), 3.22 (s, 3H), 2.87 (s, 1H), 2.67 (s, 1H), 2.54 (s, 2H), 2.35 (s, 1H), 1.58 (s, 4H), 1.07 (s, 5H), 0.81 (s, 8H).

### Example 19: Preparation of (S)-8-cyclopropyl-3-(5-(3-methyl-1-(4-methyl-4H-1,2,4-triazol-3-yl)cyclobutyl)pyridin-3-yl)-6-((3-methylpiperidin-1-yl)methyl)-4H-chromen-4-one (compound 75)

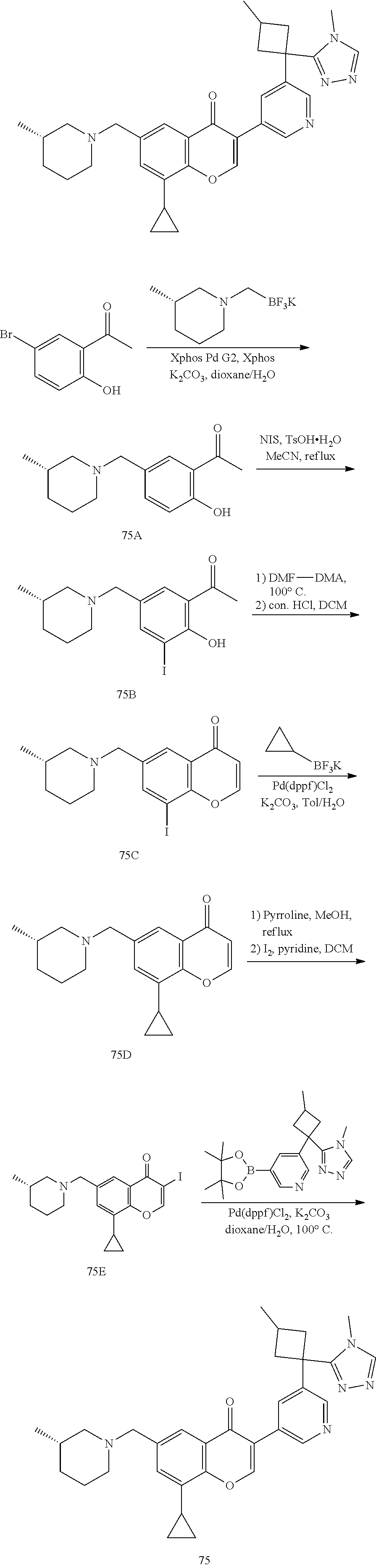

### Step 1: Preparation of (S)-1-(2-hydroxyl-5-((3-methylpiperidin-1-yl)methyl)phenyl)ethan-1-one (75A)

1-(5-Bromo-2-hydroxyphenyl)ethan-1-one (1.2 g, 5.58 mmol), 2B (1.3 g, 6.14 mmol) and potassium carbonate (2.3 g, 16.74 mmol) were added to a mixture of water (15 mL) and 1,4-dioxane (60 mL), and nitrogen replacement was performed. Under nitrogen atmosphere, XphosPd G2 (438.5 mg, 558.03 mol) and Xphos (532.0 mg, 1.12 mmol) were added to the solution, and the reaction was heated to 100° C. and stirred for 16 hours. After the reaction was completed, the reaction was cooled to room temperature, water (50 mL) was added, and the mixture was extracted with ethyl acetate (50 mL×3). The organic phase was dried over anhydrous sodium sulfate, filtered, and distilled under reduced pressure to obtain a crude product, which was separated by normal phase column chromatography (dichloromethane/[methanol (0.2M NH₃)]=10:1) to obtain the title compound 75A (950 mg, yield: 69%).

MS m/z (ESI):248.3 [M+H]⁺;

### Step 2: Preparation of (S)-1-(2-hydroxyl-3-iodo-5-((3-methylpiperidin-1-yl)methyl)phenyl)ethan-1-one (75B)

75A (0.95 g, 3.84 mmol) was dissolved in acetonitrile (50 mL), then p-toluenesulfonic acid monohydrate (1.1 g, 5.76 mmol) and NIS (1.3 g, 5.76 mmol) were added, and the mixture was refluxed overnight under nitrogen protection. The reaction solution was cooled to room temperature, quenched with saturated aqueous sodium sulfite solution, extracted with dichloromethane, and the solvent was evaporated under reduced pressure. The residue was purified by reverse phase column chromatography (chromatographic column: SepaFlash® silica gel column; mobile phase: acetonitrile/water=60:40), to obtain the title compound 75B (750.0 mg, yield: 52%).

MS m/z (ESI):374.3 [M+H]⁺;

### Step 3: Preparation of (S)-8-iodo-6-((3-methylpiperidin-1-yl)methyl)-4H-chromen-4-one (75C)

A mixture of 75B (0.75 g, 2.01 mmol) and DMF-DMA was heated to 100° C., stirred for 1 hour, cooled to room temperature, and low-boiling substances were removed by distillation under reduced pressure. The residue was dissolved in dichloromethane, and concentrated hydrochloric acid was added. The mixture was stirred for 2 hours, neutralized to neutrality with 4N NaOH (a.q.) under an ice-water bath, and then saturated aqueous sodium bicarbonate solution was added to adjust to alkalinity, and the mixture was extracted with dichloromethane three times, and concentrated to dryness under reduced pressure. The residue was purified by reverse phase column chromatography (chromatographic column: SepaFlash® silica gel column; mobile phase: acetonitrile/water=60:40), to obtain the title compound 75C (700.0 mg, yield: 91%).

MS m/z (ESI):384.2 [M+H]⁺;

### Step 4: Preparation of (S)-8-cyclopropyl-6-((3-methylpiperidin-1-yl)methyl)-4H-chromen-4-one (75D)

To a mixture of 75C (700 mg, 1.83 mmol), potassium cyclopropyl trifluoroborate (541 mg, 3.65 mmol) and potassium carbonate (757 mg, 5.48 mmol) were added water (5 mL) and toluene (20 mL), and nitrogen replacement was performed. Under nitrogen atmosphere, Pd(dppf)Cl₂ (134 mg, 183 μmol) was added, and the resulting mixture was heated to 100° C. under nitrogen protection and stirred for 4 hours. After the reaction was completed, the mixture was cooled to room temperature, the aqueous phase was separated, and the solution was concentrated under reduced pressure to obtain a crude product, and the residue was separated by reverse phase column chromatography (chromatographic column: SepaFlash® silica gel column; mobile phase: acetonitrile/water=60:40), to obtain the title compound 75D (440.0 mg, yield: 81%).

MS m/z (ESI):298.0 [M+H]⁺;

### Step 5: Preparation of (S)-8-cyclopropyl-3-iodo-6-((3-methylpiperidin-1-yl)methyl)-4H-chromen-4-one (75E)

75D (150 mg, 504.39 μmol) was dissolved in methanol (10 mL), and then dihydropyrrole (102 mg, 1.43 mmol) was added. The resulting solution was refluxed for 1 hour, cooled, and concentrated to dryness under reduced pressure. The residue was dissolved in dichloromethane (10 mL), pyridine (120 mg, 1.51 mmol) and iodine (384 mg, 1.51 mmol) were added, and the mixture was stirred for 2 hours. After the reaction was completed, the mixture was quenched with saturated aqueous sodium sulfite solution, extracted with dichloromethane three times, dried over anhydrous magnesium sulfate, and concentrated to dryness under reduced pressure to obtain the title compound 75E (200.0 mg, yield: 94%) as a crude.

MS m/z (ESI):424.0 [M+H]⁺;

### Step 6: Preparation of (S)-8-cyclopropyl-3-(5-(3-methyl-1-(4-methyl-4H-1,2,4-triazol-3-yl)cyclobutyl)pyridin-3-yl)-6-((3-methylpiperidin-1-yl)methyl)-4H-chromen-4-one (compound 75)

Referring to the synthesis method in step 4 of Example 17, the title compound 75 (6.8 mg, yield: 42%) was prepared using the same method, except that, 73C in step 4 was replaced with 65G (10.96 mg, 30.95 μmol), and 64C was replaced with 75E (13.10 mg, 30.95 mol).

MS m/z (ESI): 524.3 [M+H]⁺;

¹H NMR (400 MHz, DMSO-d₆) δ 8.78 (s, 1H), 8.67 (s, 1H), 8.52 (s, 1H), 8.32 (s, 1H), 8.03 (s, 1H), 7.84 (s, 1H), 7.32 (s, 1H), 3.56 (s, 2H), 3.50 (s, 3H), 2.93-2.9 (m, 2H), 2.72-2.65 (m, 2H), 2.58-2.56 (m, 2H), 2.39-2.30 (m, 2H), 1.90-1.80 (m, 1H), 1.65-1.40 (m, 5H), 1.10-1.00 (s, 5H), 0.85-0.75 (m, 6H).

### Example 20: 3-(3-(cyclopropylfluoro(4-methyl-4H-1,2,4-triazol-3-yl)methyl)phenyl)-8-methyl-6-((S)-3-methylpiperidin-1-yl)methyl)-4H-chromen-4-one (compound 120)

### Synthetic Route:

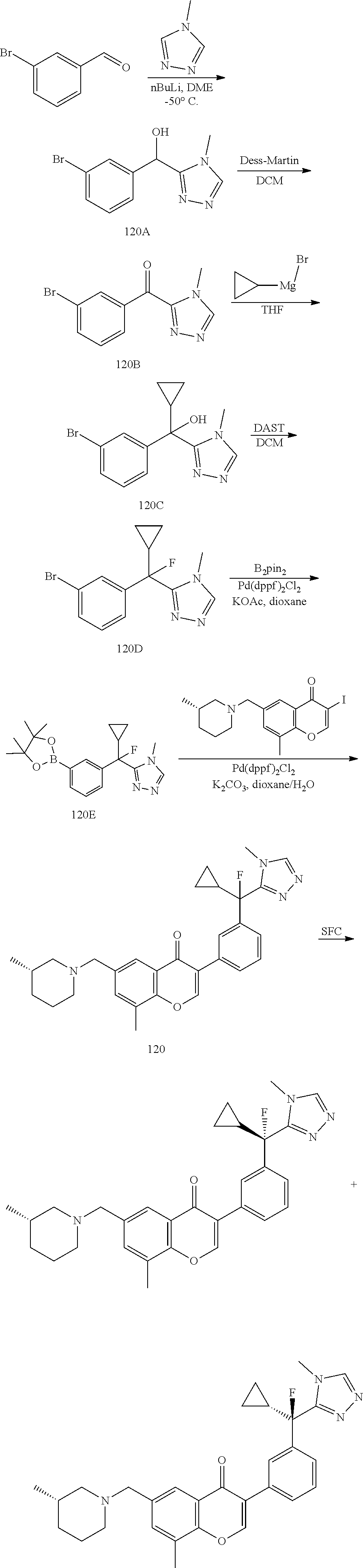

### Step 1: (3-bromophenyl)(4-methyl-4H-1,2,4-triazol-3-yl)methanol (120A)

Under argon protection at −50° C., n-butyllithium (190.56 mg, 2.97 mmol, 0.518 mL) was added to a mixture of 4-methyl-1,2,4-triazole (247.01 mg, 2.97 mmol) in DME solution (20 mL), the mixture was stirred for 1 hour, and m-bromobenzaldehyde (500 mg, 2.7 mmol) was added and reacted for 1 h, and then the solution was slowly heated to 0° C. and stirred for 1 h. After the reaction was completed, the reaction was quenched with saturated aqueous NH₄Cl solution and extracted with ethyl acetate (100 mL). The organic phase was dried over anhydrous sodium sulfate, filtered and concentrated. The crude was purified by reverse phase column chromatography (SepaFlash® silica gel column; mobile phase: acetonitrile/water=60:40), to obtain the title compound 120A (189 mg).

MS m/z (ESI):268/270 [M+H]⁺;

### Step 2: (3-bromophenyl)(4-methyl-4H-1,2,4-triazol-3-yl)methanone (120B)

120A (189 mg, 704.94 μmol) was dissolved in DCM (10 mL), Dess-Martin reagent (1.23 g, 1.41 mmol) was added, and the mixture was stirred at room temperature for 4 hours. The reaction was quenched with saturated aqueous Na₂SO₃ solution, filtered, separated and extracted with ethyl acetate (100 mL). The organic phase was dried over anhydrous sodium sulfate, filtered and concentrated. The crude was purified by reverse phase column chromatography (SepaFlash® silica gel column; mobile phase: acetonitrile/water=80:20), to obtain the title compound 120B (162 mg).

MS m/z (ESI):266/268 [M+H]⁺;

### Step 3: (3-bromophenyl)(cyclopropyl)(4-methyl-4H-1,2,4-triazol-3-yl)methanol (120C)

120B (162 mg, 608.81 μmol) was dissolved in THF (2 mL), and cyclopropylmagnesium bromide (132.67 mg, 913.21 μmol, 0.456 mL) was added to the mixture at 0° C. under argon atmosphere, and the mixture was heated to room temperature and stirred for 1 hour. The reaction was quenched with saturated aqueous NH₄Cl solution, concentrated and purified by silica gel column chromatography to obtain the title compound 120C (102 mg, 330.98 μmol, yield: 54.37%).

MS m/z (ESI):308/310 [M+H]⁺;

### Step 4: 3-((3-bromophenyl)(cyclopropyl)fluoromethyl)-4-methyl-4H-1,2,4-triazole (120D)

120C (102 mg, 330.98 μmol) was dissolved in DCM (2 mL), cooled in an ice bath, and DAST (106.70 mg, 661.97 μmol) was added via a syringe, and then the mixture was stirred for 1 hour. The reaction was quenched with saturated aqueous NaHCO₃ solution and purified by reverse phase column chromatography (SepaFlash® silica gel column; mobile phase acetonitrile/water=80:20), to obtain the title compound 120D (51 mg).

MS m/z (ESI):310/312 [M+H]⁺;

### Step 5: 3-(cyclopropylfluoro(3-(4,4,5,5-tetramethyl-1,3,2-dioxaborolan-2-yl)phenyl)methyl)-4-methyl-4H-1,2,4-triazole (120E)

120D (38 mg, 122.52 μmol), Pd(dppf)Cl₂ (8.89 mg, 12.25 μmol), potassium acetate (12.02 mg, 122.52 μmol) and B₂Pin₂ (46.67 mg, 183.8 μmol) were mixed, evacuated and backfilled with argon, and then 1,4-dioxane (2 mL) was added, and the mixture was heated to 100° C. and reacted for 5 hours. The mixture was cooled to room temperature and filtered to obtain 120E as a crude, which was directly used in the next step without purification.

MS m/z (ESI):358.2 [M+H]⁺;

### Step 6: 3-(3-(cyclopropylfluoro(4-methyl-4H-1,2,4-triazol-3-yl)methyl) phenyl)-8-methyl-6-((S)-3-methylpiperidin-1-yl)methyl)-4H-chromen-4-one (compound 120)

To a reaction tube were added 64C (35 mg, 88.11 μmol), 120E (44.06 mg, 123.35 μmol), Pd(dppf)Cl₂ (6.39 mg, 8.81 μmol) and K₂CO₃ (24.35 mg, 176.21 μmol), the tube was evacuated and backfilled with argon, water (0.5 mL) and dioxane (2 mL) were added via a syringe, and the mixture was heated to 100° C. and reacted for 1 hour. After the reaction was completed, the mixture was cooled to room temperature and purified by reverse phase column chromatography (SepaFlash® silica gel column; mobile phase: acetonitrile/water=85:15), to obtain the title product 120 (30 mg).

MS m/z (ESI):501.3 [M+H]⁺;

¹H NMR (400 MHz, DMSO-d₆) δ 8.65 (s, 1H), 8.55 (s, 1H), 7.86 (s, 1H), 7.70-7.50 (m, 3H), 7.42 (s, 1H), 7.19 (d, J=7.7 Hz, 1H), 3.51 (s, 2H), 3.31 (s, 3H), 2.80-2.66 (m, 2H), 2.48 (s, 3H), 2.20-2.00 (m, 1H), 1.95-1.80 (m, 1H), 1.69-1.42 (m, 5H), 0.90-0.75 (m, 4H), 0.75-0.60 (m, 2H), 0.60-0.48 (m, 1H), 0.48-0.40 (s, 1H).

### Step 7: 3-(3-((S)-cyclopropylfluoro(4-methyl-4H-1,2,4-triazol-3-yl)methyl) phenyl)-8-methyl-6-((S)-3-methylpiperidin-1-yl)methyl)-4H-chromen-4-one and 3-(3-((R)-cyclopropylfluoro(4-methyl-4H-1,2,4-triazol-3-yl)methyl)phenyl)-8-methyl-6-(((S)-3-methylpiperidin-1-yl)methyl)-4H-chromen-4-one

Compound 120 (27 mg) was purified and separated by supercritical fluid chromatography (chromatographic column: OD-H; column length 150 mm, inner diameter 4.6 mm; mobile phase A: IPA (with 0.05% DEA), mobile phase B: supercritical carbon dioxide; gradient: mobile phase B: from 60% to 60%; Flow rate: 2.5 mL/min;), to obtain the title compound 120-P1 (12 mg) and the title compound 120-P2 (8 mg).
120-P1 retention time: 1.673 min

MS m/z (ESI):501.3 [M+H]⁺;
120-P2 retention time: 2.097 min

MS m/z (ESI):501.3 [M+H]⁺;

### Example 21: 7-fluoro-8-methyl-3-(3-(cis-3-methyl-1-(4-methyl-4H-1,2,4-triazol-3-yl)cyclobutyl)phenyl)-6-(((s)-3-methylpiperidin-1-yl)methyl)-4H-chromen-4-one (compound 121)

### Synthetic Route:

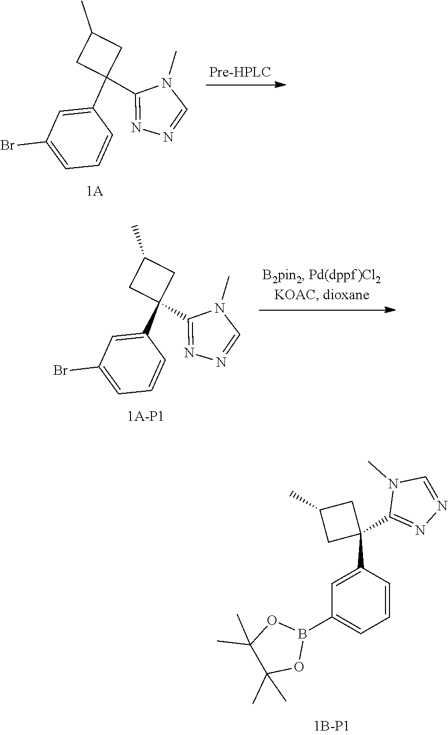 [SMILES: CC1CC(c2cccc(Br)c2)(c2nncn2C)C1 ; Cn1cnnc1[C@]1(c2cccc(Br)c2)C[C@H](C)C1 ; Cn1cnnc1[C@]1(c2cccc(B3OC(C)(C)C(C)(C)O3)c2)C[C@H](C)C1]

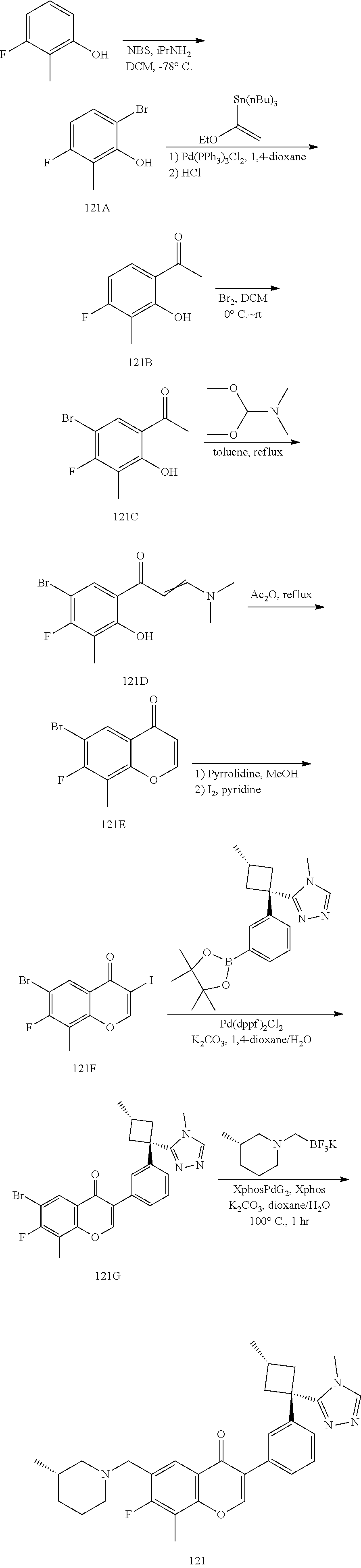

### Step 1: Preparation of cis-3-(1-(3-bromophenyl)-3-methylcyclobutyl)-4-methyl-4H-1,2,4-triazole (1A-P1)

1A (2 g, 6.54 mmol) was separated and purified by preparative chromatography (chromatographic column: Welch Xtimate C18, column length 150 mm, inner diameter 30 mm, and particle size 5 μm; mobile phase A: water (with 0.225% NH₃), mobile phase B: acetonitrile; gradient: mobile phase B: from 5% to 95% over 18 minutes), to obtain the title compound 1A-P1 (610 mg, yield: 31%).

MS m/z (ESI):; 305.7, 307.7 [M+H]⁺

¹H NMR (400 MHz, CDCl3) δ 7.97 (s, 1H), 7.54-7.51 (m, 1H), 7.41-7.37 (m, 1H), 7.25-7.18 (m, 2H), 3.18 (s, 3H), 2.85-2.75 (m, 2H), 2.70-2.58 (m, 3H), 1.13 (d, J=4.0 Hz, 3H).

### Step 2: 4-methyl-3-(cis-3-methyl-1-(3-(4,4,5,5-tetramethyl-1,3,2-dioxaborolan-2-yl)phenyl)cyclobutyl)-4H-1,2,4-triazole (1B-P1)

A mixture of 1A-P1 (100 mg, 326.58 μmol), Pd(dppf)Cl₂ (23.70 mg, 32.66 μmol) and potassium acetate (32.05 mg, 326.58 μmol) was evacuated and backfilled with argon, and then dioxane (2 mL) was added, and the mixture was heated to 100° C. and reacted for 5 hours. After the reaction was completed, the mixture was cooled to room temperature and filtered to obtain the title compound 1B-P1 as a crude, which was directly used in the next step. MS m/z (ESI):354.2 [M+H]⁺;

### Step 1: Preparation of 6-bromo-3-fluoro-2-methylphenol (121A)

Isopropylamine (1.76 g, 29.73 mmol) was slowly added to the starting materials 3-fluoro-2-methylphenol (5 g, 29.73 mmol) and dichloromethane (80 mL). The mixture was cooled in a −78° C. (dry ice/ethanol) bath. NBS (5.29 g, 29.73 mmol) was slowly added to the mixture in portions. The mixture was stirred in a −78° C. (dry ice/ethanol) bath for 0.5 hours. The reaction mixture was returned to room temperature. Aqueous hydrochloric acid solution (2 M, 100 mL) was added and stirred for 10 minutes. The organic phase was separated and spin-dried to obtain a white solid. Petroleum ether (100 mL) was added to the solid and stirred for 10 minutes. The mixture was filtered and the filtrate was spin-dried to obtain the title compound 121A (6.9 g) as a crude, which was directly used in the next step without purification.

¹H NMR (400 MHz, CDCl₃) δ 7.27-7.22 (m, 1H), 6.60-6.55 (m, 1H), 5.65 (s, 1H), 2.23-2.21 (m, 3H).

### Step 2: Preparation of 1-(4-fluoro-2-hydroxyl-3-methylphenyl)ethan-1-one (121B)

Under nitrogen flow, Pd(PPh₃)₂Cl₂ (826.77 mg, 1.18 mmol) was added to 121A (6.9 g), tributyl (1-ethoxyethylene)tin (15.340 g, 42.48 mmol, 14.35 mL) and 1,4-dioxane (100 mL), nitrogen replacement was performed three times, and the mixture was heated to 100° C. and stirred for 12 hours. After the reaction was completed, the reaction mixture was returned to room temperature, and aqueous hydrochloric acid solution (2 M, 13 mL) was added and stirred for 1 hour. The reaction solution was fully quenched with saturated aqueous potassium fluoride solution, and extracted with ethyl acetate (100 mL×3), the organic phase was dried over anhydrous sodium sulfate, filtered, concentrated, and purified by normal phase column chromatography (eluted with pure PE), to obtain the title compound 121B (3.5 g).

MS m/z (ESI): 168.16/168.7 [M+H]⁺.

¹H NMR (400 MHz, CDCl₃) δ 12.92-12.89 (m, 1H), 7.65-7.55 (m, 1H), 6.64-6.57 (m, 1H), 2.61 (s, 3H), 2.18-2.15 (m, 3H)

### Step 3: Preparation of 1-(5-bromo-4-fluoro-2-hydroxyl-3-methylphenyl) ethan-1-one (121C)

At 0° C. (ice water bath), a mixture of liquid bromine (4.37 g, 27.32 mmol, 1.50 mL) and anhydrous DCM (10 mL) was slowly added dropwise to a solution of 121B (3 g, 17.84 mmol) in anhydrous DCM (30 mL). After the dropwise addition was completed, the reaction solution was stirred at 0° C. (ice-water bath) for 1.5 hours. After the reaction was completed, the reaction solution was fully quenched with an excess of aqueous sodium metabisulfite solution at 0° C. (ice-water bath), and extracted with dichloromethane (50 mL×3), and the organic phase was dried over anhydrous sodium sulfate, filtered, concentrated under reduced pressure, and purified by normal phase column chromatography (eluted with pure PE) to obtain the title compound 121C (2 g).

¹H NMR (400 MHz, CDCl₃) δ 12.83-12.72 (m, 1H), 7.84-7.75 (m, 1H), 2.62 (s, 3H), 2.22-2.16 (m, 3H)

### Step 4: Preparation of 1-(5-bromo-4-fluoro-2-hydroxyl-3-methylphenyl)-3-(dimethylamino)-prop-2-en-1-one (121D)

N,N-Dimethylformamide dimethyl acetal (3.38 g, 28.33 mmol) was added to 121C (2 g, 8.10 mmol) and toluene (30 mL), and the mixture was heated to 115° C. and stirred for 12 hours. After the reaction was completed, the reaction was returned to room temperature, quenched with water (20 mL), and extracted with ethyl acetate (30 mL×3), and the organic phase was dried over anhydrous sodium sulfate, filtered, concentrated under reduced pressure, and purified by normal phase column chromatography (petroleum ether:ethyl acetate=5:1), to obtain the title compound 121D (1 g).

MS m/z (ESI): 302.14/303.8 [M+H]⁺.

¹H NMR (400 MHz, CDCl₃) δ 7.90 (d, J=12.0 Hz, 1H), 7.71 (d, J=8.0 Hz, 1H), 5.62 (d, J=12.0 Hz, 1H), 3.22 (s, 3H), 3.11-2.93 (m, 3H), 2.27-2.11 (m, 3H)

### Step 5: Preparation of 6-bromo-7-fluoro-8-methyl-4H-chromen-4-one (121E)

121D (1 g, 3.31 mmol) was dissolved in acetic anhydride (20 mL), and the mixture was heated to 140° C. and refluxed with stirring for 12 h. After the reaction was completed, the reaction was returned to room temperature, extracted with water (20 mL) and ethyl acetate (30 mL×3), and the organic phase was dried over anhydrous sodium sulfate, filtered, and spun and concentrated under reduced pressure, and the crude was purified by normal phase column chromatography (petroleum ether:ethyl acetate=5:1), to obtain the title compound 121E (760 mg).

MS m/z (ESI): 257.06/258.9 [M+H]⁺.

¹H NMR (400 MHz, CDCl₃) δ 8.30 (d, J=8.0 Hz, 1H), 7.91 (d, J=8.0 Hz, 1H), 6.37-6.34 (m, 1H), 2.44-2.41 (m, 3H)

### Step 6: Preparation of 6-bromo-7-fluoro-3-iodo-8-methyl-4H-chromen-4-one (121F)

121E (256 mg, 1.0 mmol) and tetrahydropyrrole (142 mg, 2.0 mmol) were added to methanol (2 mL), and the mixture was heated to 60° C. and stirred for 1 hour. After the reaction was completed, the reaction solution was cooled to room temperature and concentrated under reduced pressure to obtain the crude product. The crude, iodine (279.18 mg, 1.1 mmol), and pyridine (237.0 mg, 3.0 mmol) were added to chloroform (2 mL), and the resulting mixture was stirred at room temperature for 1 hour. After the reaction was completed, the mixture was cooled to room temperature and concentrated under reduced pressure to obtain a crude product, which was separated by normal phase flash column chromatography (PE:EA=3:1) to obtain the title compound 121F (271.1 mg, yield: 71.0%).

MS m/z (ESI): 383.1 [M+H]⁺.

### Step 7: 6-bromo-7-fluoro-8-methyl-3-(3-(cis-3-methyl-1-(4-methyl-4H-1,2,4-triazol-3-yl)cyclobutyl)phenyl)-4H-chromen-4-one (121G)

To a reaction tube were added 121F (55 mg, 143.62 μmol), 1B-P1 (50.74 mg, 143.62 mol), Pd(dppf)Cl₂ (10.42 mg, 14.36 μmol) and potassium carbonate (39.70 mg, 287.24 μmol), the tube was evacuated and backfilled with argon, water (0.5 mL) and 1,4-dioxane (2 mL) were added via a syringe, and the mixture was heated to 100° C. and reacted for 1 hour. After the reaction was completed, the mixture was cooled to room temperature and purified by reverse phase column chromatography (SepaFlash® silica gel column; mobile phase: acetonitrile/water=80:20), to obtain the title product 121G (44 mg).

MS m/z (ESI):482.1/484.1 [M+H]⁺;

### Step 8: 7-fluoro-8-methyl-3-(3-(cis-3-methyl-1-(4-methyl-4H-1,2,4-triazol-3-yl)cyclobutyl)phenyl)-6-(((s)-3-methylpiperidin-1-yl)methyl)-4H-chromen-4-one (compound 121)

To a reaction tube were added 121G (10 mg, 20.73 μmol), 2B (6.81 mg, 31.10 μmol), potassium carbonate (5.73 mg, 41.46 μmol), XPhos Pd G2 (1.63 mg, 2.07 μmol) and XPhos (1.97 mg, 4.14 μmol), the tube was evacuated and backfilled with argon, water (0.5 mL) and 1,4-dioxane (2 mL) were added via a syringe, and the mixture was heated to 100° C. and reacted for 1 hour. After the reaction was completed, the mixture was cooled to room temperature and purified by reverse phase column chromatography (SepaFlash® silica gel column; mobile phase: acetonitrile/water=85:15), to obtain the title product 121 (2 mg).

MS m/z (ESI):515.3 [M+H]⁺;

¹H NMR (400 MHz, DMSO-d₆) δ 8.65 (s, 1H), 8.29 (s, 1H), 8.00 (s, 1H), 7.62 (s, 1H), 7.45 (s, 2H), 7.30 (s, 1H), 3.59 (s, 2H), 3.22 (s, 3H), 2.81 (d, J=48.5 Hz, 5H), 2.37 (s, 3H), 1.91 (s, 1H), 1.67-1.37 (m, 6H), 1.23 (s, 1H), 1.15-1.0 (m, 3H), 0.9-0.75 (m, 4H).

### Example 22: 7-fluoro-8-methyl-3-(3-(cis-3-methyl-1-(4-methyl-4H-1,2,4-triazol-3-yl)cyclobutyl)phenyl)-6-(((1-methylcyclobutyl)amino)methyl)-4H-chromen-4-one (compound 122)

### Synthetic Route:

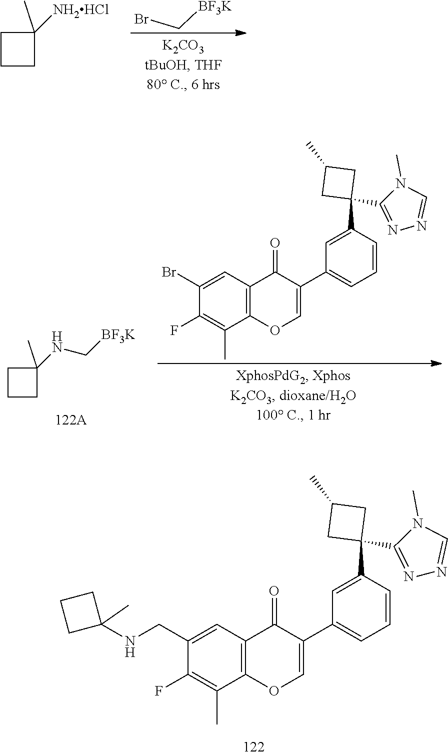

### Step 1: 1-methyl-N-((trifluoro-λ⁴-boranyl)methyl)cyclobutyl-1-amine potassium salt (122A)

1-Methylcyclobutylamine hydrochloride (100 mg, 822.31 μmol, HCl) was dissolved in tert-butanol (2 mL), and THF (0.5 mL), potassium (bromomethyl)trifluoroborate (165.15 mg, 822.31 μmol) and potassium carbonate (340.95 mg, 2.47 mmol) were added to the mixture, and the reaction was heated to 80° C. and stirred for 6 h. After the reaction was completed, the reaction solution was concentrated and the residue was dissolved in acetone (2 mL), stirred for 30 minutes, filtered and concentrated to obtain the title compound 122A (168 mg) as a crude, which was directly used in the next step without purification.

MS m/z (ESI): 148.1 [M−KF+H]⁺;

### Step 2: 7-fluoro-8-methyl-3-(3-(cis-3-methyl-1-(4-methyl-4H-1,2,4-triazol-3-yl)cyclobutyl)phenyl)-6-(((1-methylcyclobutyl)amino)methyl)-4H-chromen-4-one (compound 122)

A mixture of 121G (10 mg, 20.73 μmol), 122A (23.12 mg), XphosPdG2 (2.95 mg, 3.76 mol), Xphos (3.58 mg, 7.52 μmol) and potassium carbonate (10.39 mg, 75.17 μmol) was evacuated and backfilled with argon, water (0.5 mL) and 1,4-dioxane (2 mL) were added to the mixture, and the reaction was heated to 100° C. for 1 hour. After the reaction was completed, the reaction solution was cooled to room temperature, water (10 mL) was added, and the mixture was extracted with ethyl acetate (10 mL×3). The organic phase was dried over anhydrous sodium sulfate, filtered and concentrated. The residue was purified by reverse phase column chromatography (SepaFlash® silica gel column; mobile phase: acetonitrile/water=80:20), to obtain the title compound 122 (5 mg).

MS m/z (ESI):501.3 [M+H]⁺;

¹H NMR (400 MHz, DMSO-d₆) δ 8.64 (s, 1H), 8.31 (s, 1H), 8.14 (d, J=8.2 Hz, 1H), 7.62 (s, 1H), 7.44 (d, J=4.8 Hz, 2H), 7.30 (s, 1H), 3.74 (s, 2H), 3.23 (s, 3H), 3.16 (s, 1H), 2.90-2.84 (m, 2H), 2.37 (s, 3H), 2.00-1.95 (m, 2H), 1.76-1.68 (m, 4H), 1.31-1.23 (m, 6H), 1.08 (d, J=4.7 Hz, 3H).

### Example 23: 8-methyl-3-(3-(cis-3-methyl-1-(4-methyl-4H-1,2,4-triazol-3-yl)cyclobutyl)phenyl)-6-(1-((1-methylcyclobutyl)amino)ethyl)-4H-chromen-4-one (compound 123)

### Synthetic Route:

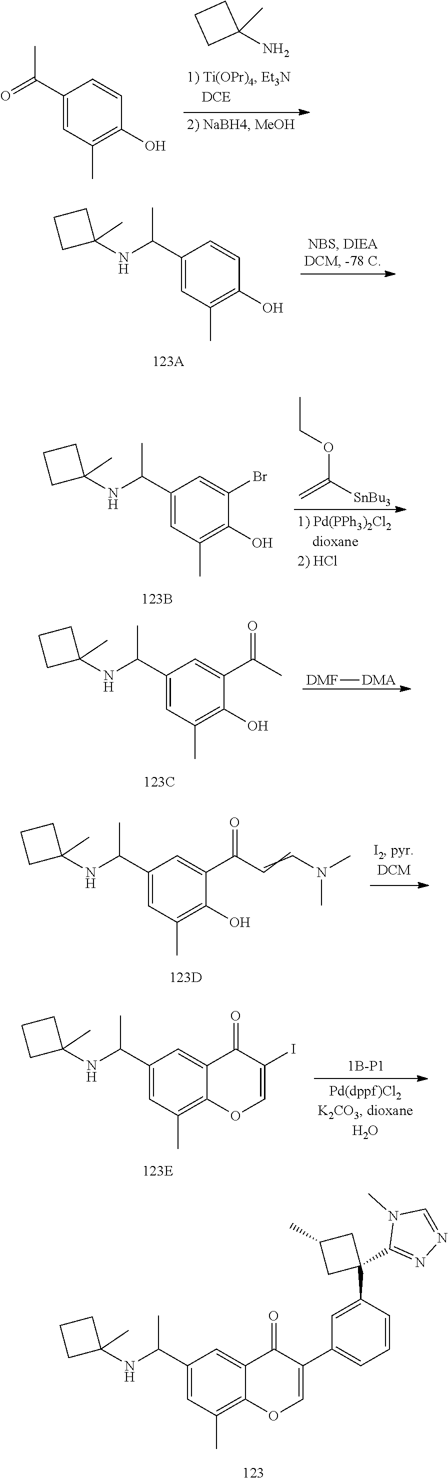

### Step 1: 2-methyl-4-(1-((1-methylcyclobutyl)amino)ethyl)phenol (123A)

A mixture of 1-methylcyclobutylamine (971.74 mg, 7.99 mmol, HCl), 1-(4-hydroxy-3-methylphenyl)ethanone (300 mg, 2.00 mmol), titanium tetraisopropoxide (2.27 g, 7.99 mmol), triethylamine (1.01 g, 9.99 mmol, 1.39 mL) and DCE (10 mL) was heated to 80° C. overnight. After the reaction was completed, the mixture was cooled to room temperature and then cooled in an ice bath. MeOH (2 mL) and NaBH₄ (377.89 mg, 9.99 mmol) were added, and the reaction was stirred for 2 hours. After the reaction was completed, the reaction was quenched with water, filtered through diatomaceous earth, and extracted with ethyl acetate (5 mL×3), and the organic phase was dried over anhydrous sodium sulfate, filtered, and concentrated, and the crude was purified by reverse phase column chromatography (SepaFlash® silica gel column; mobile phase: acetonitrile/water=50:50), to obtain the title compound 123A (270 mg).

MS m/z (ESI):220.2 [M+H]⁺;

### Step 2: 2-bromo-6-methyl-4-(1-((1-methylcyclobutyl)amino)ethyl)phenol (123B)

123A (270 mg, 1.23 mmol) was dissolved in DCM (4.66 mL), the reaction was cooled to −78° C. in a dry ethanol bath, DIEA (249.14 mg, 2.46 mmol, 335.77 μL) and NBS (328.67 mg, 1.85 mmol) were sequentially added, and the reaction was maintained at −78° C. for 2 hours. Aqueous HCl (1 M) was added to quench the reaction. Water (10 mL) and DCM (10 mL×3) were added for extraction. The organic phase was dried over anhydrous sodium sulfate, filtered and concentrated. The residue was purified by reverse phase column chromatography (SepaFlash® silica gel column; mobile phase: acetonitrile/water=50:50), to obtain the title product 123B (170 mg).

MS m/z (ESI):298.1/300.1 [M+H]⁺;

### Step 3: 1-(2-hydroxyl-3-methyl-5-(1-((1-methylcyclobutyl)amino)ethyl) phenyl)ethan-1-one (123C)

The mixture of 123B (50 mg, 167.66 μmol) and Pd(PPh₃)₂Cl₂ (19.37 mg, 16.77 μmol) was evacuated and backfilled with argon, and then tri-n-butyl(1-ethoxyvinyl)tin (121.10 mg, 335.33 μmol) and dioxane (2 mL) were added to the mixture and the reaction was heated to 100° C. overnight. The reaction was cooled to room temperature, and 1M aqueous HCl (1 mL) was added and stirred for 1 hour. The reaction solution was fully quenched with saturated aqueous potassium fluoride solution, and extracted with ethyl acetate (100 mL×3). The organic phase was dried over anhydrous sodium sulfate, filtered and concentrated, and then purified by reverse phase column chromatography (SepaFlash® silica gel column; mobile phase: acetonitrile/water=60:40), to obtain the title product 123C (22 mg).

MS m/z (ESI):262.2 [M+H]⁺;

### Step 4: 3-(Dimethylamino)-1-(2-hydroxyl-3-methyl-5-(1-((1-methylcyclobutyl) amino)ethyl)phenyl)prop-2-en-1-one (123D)

123C (27 mg, 103.31 μmol) was dissolved in DMF-DMA (3 mL), and the mixture was heated 80° C., and reacted for 1 hour. After the reaction was completed, the mixture was cooled to room temperature and concentrated to obtain the title product 123D (32 mg) as a crude, which was directly used in the next step without purification.

MS m/z (ESI):317.2 [M+H]⁺;

### Step 5: 3-iodo-8-methyl-6-(1-((1-methylcyclobutyl)amino)ethyl)-4H-chromen-4-one (123E)

123D (32 mg, 101.13 μmol) was dissolved in DCM (5 mL), I₂ (35.29 mg, 139.05 μmol) and pyridine (11.00 mg, 139.05 μmol, 11.20 μL) were sequentially added, and the mixture was stirred for 1 hour, and then purified by reverse phase column chromatography (SepaFlash® silica gel column; mobile phase: acetonitrile/water=80:20), to obtain the title product 123E (13 mg).

MS m/z (ESI):398.1 [M+H]⁺;

### Step 6: 8-methyl-3-(3-(cis-3-methyl-1-(4-methyl-4H-1,2,4-triazol-3-yl)cyclobutyl)phenyl)-6-(1-((1-methylcyclobutyl)amino)ethyl)-4H-chromen-4-one (compound 123)

To a reaction tube were added 123E (13 mg, 32.73 μmol), 1B-P1 (17.34 mg, 49.09 mol), Pd(dppf)Cl₂ (2.37 mg, 3.27 μmol) and potassium carbonate (9.05 mg, 65.45 μmol), the tube was evacuated and backfilled with argon, water (0.5 mL) and dioxane (2 mL) were added via a syringe, and the mixture was heated to 100° C. and reacted for 1 hour. After the reaction was completed, the mixture was cooled to room temperature, filtered, and concentrated under reduced pressure, and then purified by reverse phase column chromatography (SepaFlash® silica gel column; mobile phase: acetonitrile/water=85:15), to obtain the title product 123 (3.7 mg).

MS m/z (ESI):497.3 [M+H]⁺;

¹H NMR (400 MHz, DMSO-d₆) δ 8.61 (s, 1H), 8.29 (s, 1H), 8.00 (s, 1H), 7.77 (s, 1H), 7.63 (s, 1H), 7.50-7.40 (m, 2H), 7.30 (s, 1H), 4.00 (s, 1H), 3.23 (s, 3H), 2.87 (s, 2H), 2.54 (d, J=6.7 Hz, 3H), 2.48 (s, 3H), 1.85-1.71 (s, 2H), 1.60-1.47 (s, 2H), 1.42-1.21 (m, 6H), 1.20-1.05 (m, 6H).

### Example 24: Preparation of 3-(3-(cyclopropyl(4-methyl-4H-1,2,4-triazol-3-yl)methyl)phenyl)-7-fluoro-8-methyl-6-(((S)-3-methylpiperidin-1-yl)methyl)-4H-chromen-4-one (compound 124)

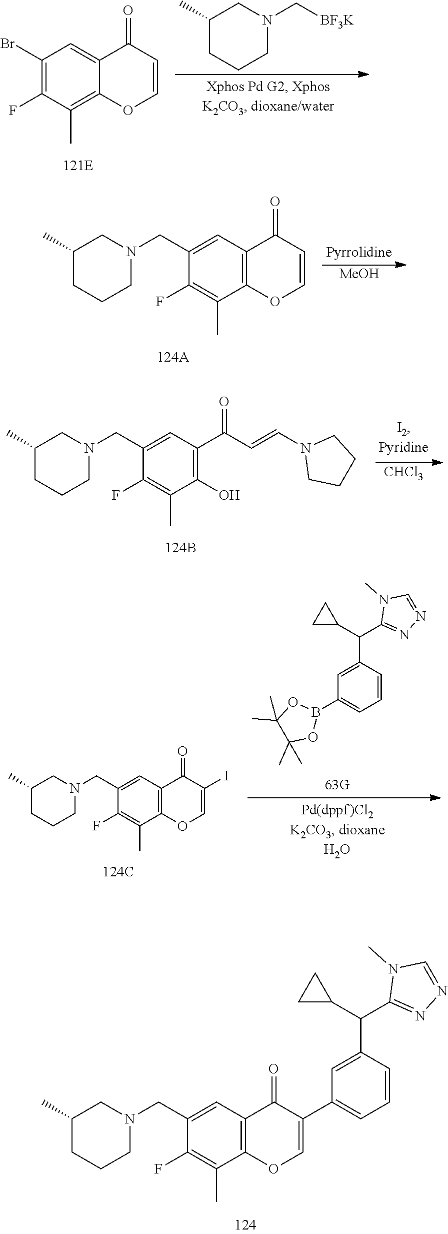

### Step 1: Preparation of (S)-7-fluoro-8-methyl-6-((3-methylpiperidin-1-yl)methyl)-4H-chromen-4-one (124A)

Intermediate 121E (200 mg, 778.04 μmol), 2B (339.40 mg, 1.56 mmol), XPhos Pd G2 (61.22 mg, 77.80 μmol), XPhos (74.18 mg, 155.61 μmol) and potassium carbonate (214.74 mg, 1.56 mmol) were added to a mixture of water (0.4 mL) and 1,4-dioxane (2.0 mL), and nitrogen replacement was performed. The resulting mixture was heated to 100° C. under nitrogen protection and stirred for 12 h. After the reaction was completed, the mixture was cooled to room temperature, filtered, and concentrated under reduced pressure to obtain a crude product, which was separated by preparative high performance liquid chromatography (chromatographic column: Welch Xtimate C18; column length 150 mm, inner diameter 30 mm, and particle size 5 μm; mobile phase A: water (with 0.225% NH₃), mobile phase B: acetonitrile; gradient: mobile phase B: from 5% to 95% over 18 minutes), to obtain the title compound 124A (150 mg).

MS m/z (ESI): 290.2 [M+H]⁺.

### Step 2: Preparation of (S,E)-1-(4-fluoro-2-hydroxyl-3-methyl-5-((3-methylpiperidin-1-yl)methyl)phenyl)-3-(pyrrolidin-1-yl)prop-2-en-1-one (124B)

124A (150 mg, 518.41 μmol), and tetrahydropyrrole (36.87 mg, 518.41 μmol) were added to methanol (2 mL), and the resulting mixture was heated to 60° C. and stirred for 1 hour. After the reaction was completed, the reaction solution was cooled to room temperature and concentrated under reduced pressure to obtain the title compound 124B (180.0 mg) as a crude, which was directly used in the next step without further purification.

MS m/z (ESI): 361.2 [M+H]⁺.

### Step 3: Preparation of (S)-7-fluoro-3-iodo-8-methyl-6-((3-methylpiperidin-1-yl)methyl)-4H-chromen-4-one (124C)

124B (180 mg, 499.36 μmol), iodine (253.48 mg, 998.71 μmol), and pyridine (79.00 mg, 998.71 μmol, 80.45 L) were added to chloroform (2 mL), and the resulting mixture was stirred at room temperature for 1 hour. After the reaction was completed, the mixture was cooled to room temperature, and concentrated under reduced pressure to obtain a crude product, which was separated by preparative high performance liquid chromatography (chromatographic column: Welch Xtimate C18; column length 150 mm, inner diameter 30 mm, and particle size 5 μm; mobile phase A: water (with 0.225% NH₃), mobile phase B: acetonitrile; gradient: mobile phase B: from 5% to 95% over 18 minutes), to obtain the title compound 124C (80 mg).

MS m/z (ESI): 416.1 [M+H]⁺.

### Step 4: Preparation of 3-(3-(cyclopropyl(4-methyl-4H-1,2,4-triazol-3-yl)methyl)phenyl)-7-fluoro-8-methyl-6-(((S)-3-methylpiperidin-1-yl)methyl)-4H-chromen-4-one (compound 124)

124C (60 mg, 144.49 μmol), 63G (49.02 mg, 144.49 μmol), Pd(dppf)Cl₂ (10.57 mg, 14.45 μmol) and potassium carbonate (39.88 mg, 288.99 μmol) were added to a mixture of water (0.4 mL) and 1,4-dioxane (2.0 mL), and nitrogen replacement was performed. The resulting mixture was heated to 100° C. under nitrogen protection and stirred for 2 h. After the reaction was completed, the mixture was cooled to room temperature, filtered, and concentrated under reduced pressure to obtain a crude product, which was separated by preparative high performance liquid chromatography (chromatographic column: Welch Xtimate C18; column length 150 mm, inner diameter 30 mm, and particle size 5 μm; mobile phase A: water (with 0.225% NH₃), mobile phase B: acetonitrile; gradient: mobile phase B: from 5% to 95% over 18 minutes), to obtain the title compound 124 (15 mg).

MS m/z (ESI): 501.3 [M+H]⁺.

1H NMR (400 MHz, DMSO-d₆) δ 8.59 (s, 1H), 8.35 (s, 1H), 8.00 (d, J=9.1 Hz, 1H), 7.51 (s, 1H), 7.48-7.36 (m, 2H), 7.31 (d, J=7.7 Hz, 1H), 3.59 (s, 2H), 3.55 (d, J=8.9 Hz, 1H), 3.41 (s, 3H), 2.37 (s, 3H), 2.34-2.31 (m, 2H), 1.98-1.87 (m, 2H), 1.72-1.55 (m, 5H), 0.88-0.84 (m, 1H), 0.81 (d, J=5.7 Hz, 3H), 0.65-0.50 (m, 4H).

### Example 25: Preparation of 8-methyl-3-(3-(cis-3-methyl-1-(4-methyl-4H-1,2,4-triazol-3-yl)cyclobutyl)phenyl)-6-((1-methylcyclobutyl)amino)methyl)-4H-pyrano[2,3-c]pyridin-4-one (compound 125)

### Synthetic Route:

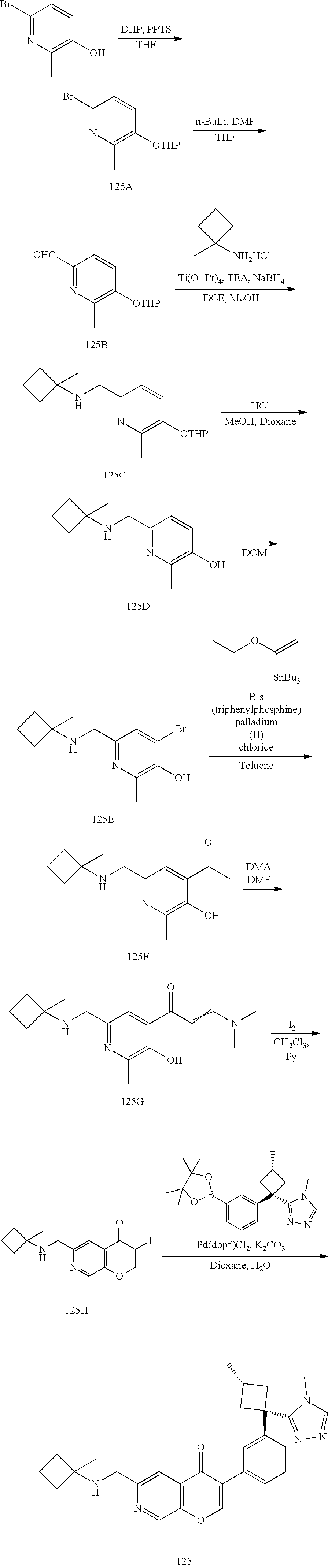

### Step 1: Preparation of 6-bromo-2-methyl-3-((tetrahydro-2H-pyran-2-yl)oxy)pyridine (125A)

The starting materials 6-bromo-2-methylpyridin-3-ol (100.0 mg, 0.53 mmol), and DHP (134.21 mg, 1.60 mmol) were added to tetrahydrofuran (2.0 mL), and then PPTS (13.37 mg, 53.19 μmol) was added, and the mixture was heated to 70° C. and stirred for 16 hours. The crude product was purified by reverse phase column chromatography (chromatographic column: SepaFlash® silica gel column; mobile phase: acetonitrile/water=80:20), to obtain the title compound 125A (82.0 mg).

MS m/z (ESI): 186.96/188.96 [M+H-THP]⁺.

### Step 2: Preparation of 6-methyl-5-((tetrahydro-2H-pyran-2-yl)oxy) pyridinealdehyde (125B)

125A (82.0 mg, 0.3 mmol) was added to tetrahydrofuran (3.0 mL), nitrogen replacement was performed, and the reaction solution was cooled down to −78° C., and then n-butyl lithium (0.12 mL, 0.3 mmol) was added, the mixture was stirred for 0.5 h, and then DMF (22.02 mg, 0.3 mmol) was added, and stirred at −78° C. for 1 h. After the reaction was completed, the reaction was quenched with saturated aqueous ammonium chloride solution, and extracted with ethyl acetate (5 mL×3). The organic phase was dried over anhydrous sodium sulfate, filtered, and distilled under reduced pressure to obtain a crude product, which was purified by normal phase column chromatography (petroleum ether:ethyl acetate=90:10), to obtain the title compound 125B (53.0 mg).

MS m/z (ESI): 272.0 [M+H]⁺.

### Step 3: Preparation of 1-methyl-N-((6-methyl-5-((tetrahydro-2H-pyran-2-yl)oxy)pyridin-2-ylmethyl)cyclobutyl-1-amine (compound 125C)

125B (53.0 mg, 0.24 mmol), 1-methylcyclobutylamine hydrochloride (87.39 mg, 0.72 mmol) and tetraisopropyl titanate (272.33 mg, 0.96 mmol) were added to DCE (2.0 mL), followed by triethylamine (121.2 mg, 1.2 mmol), and the resulting mixture was heated to 80° C. and stirred for 16 hours. After the reaction of the raw materials was completed as monitored by TLC, methanol (2.0 mL) was added to the reaction solution, followed by sodium borohydride (45.31 mg, 1.2 mmol), and the mixture was stirred at room temperature for 3 h. After the reaction was completed, the reaction was quenched with water, and extracted with ethyl acetate (5 mL×3). The organic phase was dried over anhydrous sodium sulfate, filtered, and distilled under reduced pressure to obtain a crude product, which was purified by reverse phase column chromatography (chromatographic column: SepaFlash® silica gel column; mobile phase: acetonitrile/water=65:35), to obtain the title compound 125C (48.0 mg).

MS m/z (ESI): 207.2 [M+H-THP]⁺

### Step 4: Preparation of 2-methyl-6-(((1-methylcyclobutyl)amino) methyl)pyridin-3-ol (compound 125D)

At room temperature, 125C (48.0 mg, mol) was added to methanol (1.0 mL), and upon a clear solution was obtained, a solution of hydrogen chloride in dioxane (4.0 M, 2.0 mL) was added. The reaction solution was then heated to 45° C. and stirred for 5 hours. After the reaction was completed, the reaction solution was directly distilled under reduced pressure to obtain the title compound 125D (34.0 mg) as a crude, which was used without further purification.

MS m/z (ESI): 207.14 [M+H]⁺.

### Step 5: Preparation of 4-bromo-2-methyl-6-(((1-methyl cyclobutyl)amino) methyl)pyridin-3-ol (compound 125E)

125D (34.0 mg, 165.0 μmol) and diisopropylamine (33.4 mg, 330.0 μmol) were added to dichloromethane (1.0 mL), and nitrogen replacement was performed and the reaction solution was cooled down to −72° C. Then, NBS (32.3 mg, 181.5 μmol) was added and the mixture was stirred at −72° C. for 2 hours. After the reaction was completed, the reaction was quenched with water, and extracted with dichloromethane (5 mL×3). The organic phase was dried over anhydrous sodium sulfate, filtered, and distilled under reduced pressure to obtain a crude product, which was purified by reverse phase column chromatography (chromatographic column: SepaFlash® silica gel column; mobile phase: acetonitrile/water=50:50), to obtain the title compound 125E (37.0 mg).

MS m/z (ESI): 285.05/287.05 [M+H]⁺.

### Step 6: Preparation of 1-(3-hydroxyl-2-methyl-6-(((1-methylcyclobutyl) amino)methyl)pyridin-4-yl)ethan-1-one (125F)

125E (37.0 mg, 0.132 mmol) and tributyl(1-ethoxyethylene)tin (95.5 mg, 0.264 mmol) were added to toluene (1.0 mL), and nitrogen replacement was performed. Under nitrogen atmosphere, bistriphenylphosphine palladium dichloride (9.25 mg, 13.2 μmol) was added, and the resulting mixture was heated to 100° C. under nitrogen protection and stirred for 16 hours. The crude product was purified by reverse phase column chromatography (chromatographic column: SepaFlash® silica gel column; mobile phase: acetonitrile/water=70:30), and the concentrated intermediate was stirred in a solution of hydrogen chloride in dioxane (4.0 M, mL) at room temperature for 1 hour. The reaction solution was concentrated to obtain the title compound 125F (29.0 mg).

MS m/z (ESI): 249.15 [M+H]⁺.

### Step 7: Preparation of 3-(dimethylamino)-1-(3-hydroxyl-2-methyl-6-((1-methylcyclobutyl)amino)methyl)pyridin-4-yl)prop-2-en-1-one (125G)

125F (29.0 mg, 117.0 μmol) was added to N,N-dimethylformamide dimethyl acetal (1.0 mL), and the reaction solution was heated to 100° C. and stirred for 1 hour. After the reaction was completed, the reaction solution was directly distilled under reduced pressure to obtain the title compound 125G (35.0 mg) as a crude, which was directly used in the next step without further purification.

MS m/z (ESI): 304.19 [M+H]⁺.

### Step 8: Preparation of 3-iodo-8-methyl-6-(((1-methylcyclobutyl)amino) methyl)-4H-pyrano[2,3-c]pyridin-4-one (125H)

125G (35.0 mg, 0.115 mmol) and iodine (58.7 mg, 0.23 mmol) were added to chloroform (2.0 mL), followed by pyridine (0.3 mL), and the mixture was stirred at room temperature for 1 hour. After the reaction was completed, the reaction solution was quenched with saturated aqueous sodium bisulfite solution (1 mL), filtered, and the filtrate was concentrated to obtain a crude, which was purified by reverse phase column chromatography (chromatographic column: SepaFlash® silica gel column; mobile phase: acetonitrile/water=80:20), to obtain the title compound 125H (30.0 mg).

MS m/z (ESI): 385.03 [M+H]⁺.

### Step 9: Preparation of 8-methyl-3-(3-(cis-3-methyl-1-(4-methyl-4H-1,2,4-triazol-3-yl)cyclobutyl)phenyl)-6-((1-methylcyclobutyl)amino)methyl)-4H-pyrano[2,3-c]pyridin-4-one (compound 125)

125H (30.0 mg, 77.9 μmol), 1B-P1 (33.0 mg, 93.5 μmol) and potassium carbonate (21.5 mg, 156.0 μmol) were added to a mixture of water (0.4 mL) and 1,4-dioxane (2.0 mL), and nitrogen replacement was performed. Under nitrogen atmosphere, Pd(dppf)Cl₂ (6.0 mg, 8.0 μmol) was added, and the resulting mixture was heated to 100° C. under nitrogen protection and stirred for 1.0 h. After the reaction was completed, the reaction solution was cooled to room temperature, filtered, and concentrated. The crude product was purified by reverse phase column chromatography (chromatographic column: SepaFlash® silica gel column; mobile phase: acetonitrile/water=70:30), to obtain the title compound 125 (7.1 mg).

MS m/z (ESI): 484.26 [M+H]⁺.

### Example 26: Preparation of 3-(3-(cis-3-methyl-1-(4-methyl-4H-1,2,4-triazol-3-yl)cyclobutyl)phenyl)-7-(1-((1-methylcyclobutyl)amino)ethyl)-9-(trifluoromethyl)-4H-pyrido[1,2-a]pyrimidin-4-one (compound 126)

### Synthetic Route:

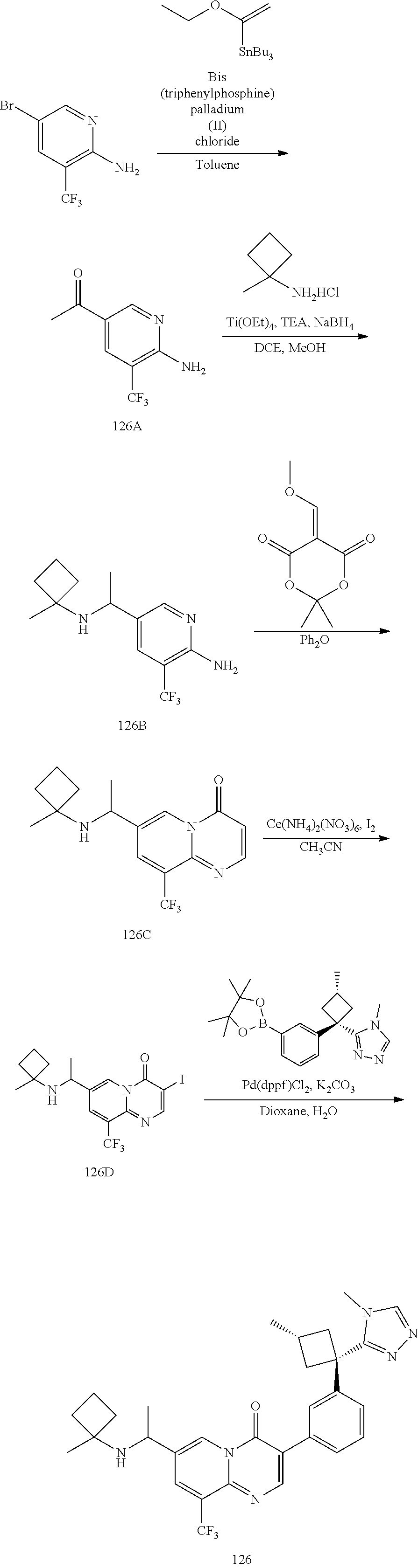

### Step 1: Preparation of 1-(6-amino-5-(trifluoromethyl)pyridin-3-yl)ethan-1-one (126A)

5-Bromo-3-(trifluoromethyl)pyridin-2-amine (300.0 mg, 1.24 mmol), and tributyl(1-ethoxyethylene)tin (674.32 mg, 1.87 mmol) were added to toluene (10.0 mL), and nitrogen replacement was performed. Under nitrogen atmosphere, bistriphenylphosphine palladium dichloride (87.37 mg, 124.48 μmol) was added, and the resulting mixture was heated to 100° C. under nitrogen protection and stirred for 16.0 hours. After the reaction was completed, the reaction was quenched with water, and extracted with ethyl acetate (5 mL×3). The organic phase was dried over anhydrous sodium sulfate, filtered, and concentrated. The crude product was purified by reverse phase column chromatography (chromatographic column: SepaFlash® silica gel column; mobile phase: acetonitrile/water=70:30), and the concentrated intermediate was stirred with a solution of hydrogen chloride in dioxane (4.0 M, 5.0 mL) at room temperature for 1.0 hour. The reaction solution was concentrated to obtain the title compound 126A (180.0 mg).

MS m/z (ESI): 205.15 [M+H]⁺.

### Step 2: Preparation of 5-(1-((1-methylcyclobutyl)amino)ethyl)-3-(trifluoromethyl)pyridin-2-amine (126B)

126A (180.0 mg, 0.88 mmol), 1-methylcyclobutylamine hydrochloride (321.67 mg, 2.65 mmol) and tetraethyl titanate (1.0 g, 3.53 mmol) were added to DCE (4.0 mL), followed by triethylamine (446.1 mg, 4.41 mmol), and the resulting mixture was heated to 80° C. and stirred for 16.0 hours. Upon the raw materials disappeared as monitored by TLC, methanol (4.0 mL) was added to the reaction solution, followed by sodium borohydride (116.79 mg, 4.41 mmol), and the mixture was stirred at room temperature for 3.0 h. After the reaction was completed, the reaction was quenched with water, filtered through diatomaceous earth, and extracted with ethyl acetate (5 mL×3), and the organic phase was dried over anhydrous sodium sulfate, filtered, and concentrated, and the crude was purified by reverse phase column chromatography (chromatographic column: SepaFlash® silica gel column; mobile phase: acetonitrile/water=65:35), to obtain the title compound 126B (180.0 mg).

MS m/z (ESI): 274.3 [M+H]⁺.

### Step 3: Preparation of 7-(1-((1-methylcyclobutyl)amino)ethyl)-9-(trifluoromethyl)-4H-pyrido[1,2-a]pyrimidin-4-one (compound 126C)

126B (180.0 mg, 658.63 μmol) and 5-(methoxymethylene)-2,2-dimethyl-1,3-dioxane-4,6-dione (122.61 mg, 658.63 μmol) were added to diphenyl ether (3.0 mL), and the temperature was heated to 120° C. and stirred for 1.0 hour. Upon the raw materials disappeared as monitored by TLC, the reaction solution was heated to 210° C. and stirred for 1.0 hour. Then the reaction was cooled to room temperature and the crude product was purified by reverse phase column chromatography (chromatographic column: SepaFlash® silica gel column; mobile phase: acetonitrile/water=60:40), to obtain the title compound 126C (20.0 mg).

MS m/z (ESI): 326.33 [M+H]⁺.

### Step 4: Preparation of 3-iodo-7-(1-((1-methyl cyclobutyl)amino)ethyl)-9-(trifluoromethyl)-4H-pyrido[1,2-a]pyrimidin-4-one (compound 126D)

At room temperature, 126C (20.0 mg, 61.48 μmol), cerium ammonium nitrate (32.58 mg, 561.48 μmol) and iodine (15.6 mg, 61.48 μmol) were sequentially added to acetonitrile solution (3.0 mL). The reaction solution was then stirred for 6 hours. After the reaction was completed, saturated aqueous sodium sulfite solution (10 mL) was added and the mixture was extracted with dichloromethane (50 mL×3). The organic phase was dried over anhydrous sodium sulfate, filtered, and distilled under reduced pressure to obtain a crude product, which was purified by reverse phase column chromatography (chromatographic column: SepaFlash® silica gel column; mobile phase: acetonitrile/water=50:50), to obtain the title compound 126D (10.0 mg).

MS m/z (ESI): 452.22 [M+H]⁺.

### Step 5: Preparation of 3-(3-(cis-3-methyl-1-(4-methyl-4H-1,2,4-triazol-3-yl)cyclobutyl)phenyl)-7-(1-((1-methylcyclobutyl)amino)ethyl)-9-(trifluoromethyl)-4H-pyrido[1,2-a]pyrimidin-4-one (compound 126)

126D (10.0 mg, 22.16 μmol), 1B-P1 (11.74 mg, 33.24 μmol) and potassium carbonate (6.12 mg, 44.32 μmol) were added to a mixture of water (0.4 mL) and 1,4-dioxane (2.0 mL), and nitrogen replacement was performed. Under nitrogen atmosphere, Pd(dppf)Cl₂ (1.62 mg, 2.22 mol) was added, and the resulting mixture was heated to 100° C. under nitrogen protection and stirred for 1.0 h. After the reaction was completed, the resulting crude product was purified by reverse phase column chromatography (chromatographic column: SepaFlash® silica gel column; mobile phase: acetonitrile/water=50:50), to obtain the title compound 126 (2.4 mg).

MS m/z (ESI): 551.64 [M+H]⁺.

¹H NMR (400 MHz, DMSO-d6) δ 9.29 (s, 1H), 8.68 (s, 1H), 8.61 (s, 1H), 8.31 (s, 1H), 7.93 (s, 1H), 7.69 (d, J=7.7 Hz, 1H), 7.48 (t, J=7.8 Hz, 1H), 7.30 (d, J=7.8 Hz, 1H), 4.18 (d, J=6.2 Hz, 1H), 3.25 (s, 3H), 2.90 (s, 2H), 2.57 (d, J=6.5 Hz, 3H), 1.91 (t, J=9.7 Hz, 1H), 1.77 (t, J=9.4 Hz, 2H), 1.59 (d, J=6.9 Hz, 2H), 1.50 (s, 1H), 1.34 (d, J=6.7 Hz, 3H), 1.15 (s, 3H), 1.11 (d, J=4.7 Hz, 3H).

### Example 27: Preparation of 8-methyl-6-(((S)-3-methylpiperidin-1-yl)methyl)-3-(3-(1,2,2,2-tetrafluoro-1-(4-methyl-4H-1,2,4-triazol-3-yl)ethyl)phenyl)-4H-chromen-4-one (compound 128)

### Synthetic Route:

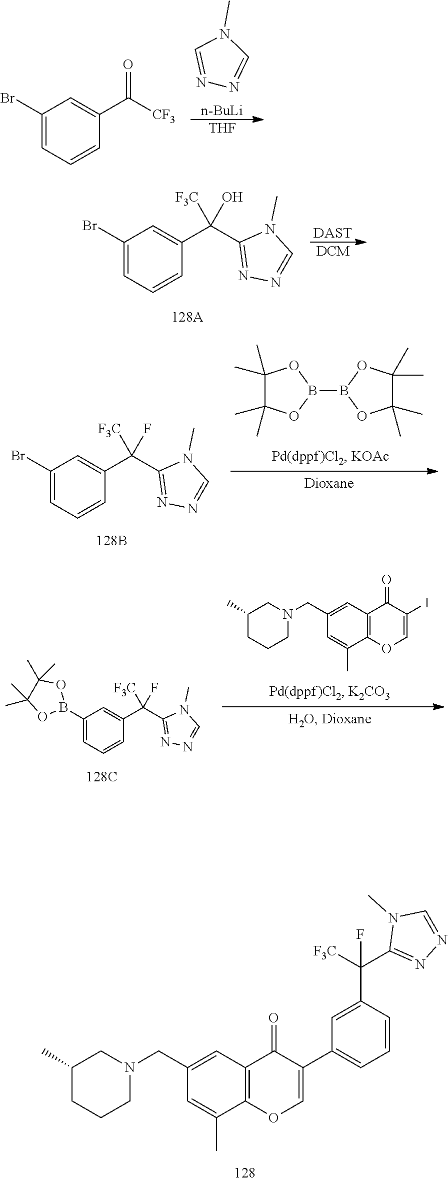

### Step 1: Preparation of 1-(3-bromophenyl)-2,2,2-trifluoro-1-(4-methyl-4H-1,2,4-triazol-3-yl)ethan-1-ol (128A)

4-Methyl-4H-1,2,4-triazole (102.46 mg, 1.23 mmol) was dissolved in tetrahydrofuran (10.0 mL). After the reaction solution was cooled down to −60° C., a solution of n-butyllithium in n-hexane (2.5M, 0.492 mL, 1.23 mmol) was added dropwise to the reaction solution. After the dropwise addition was completed, the reaction solution was kept stirring at −60° C. for 1.0 hour. Then, a solution of 1-(3-bromophenyl)-2,2,2-trifluoroethan-1-one (312.0 mg, 1.23 mmol) dissolved in tetrahydrofuran (1.5 mL) was added dropwise. After the dropwise addition was completed, the reaction solution was transferred to room temperature and reacted for 0.5 hours. After the reaction was completed, the reaction solution was cooled down to 0° C. in an ice bath, and then a saturated aqueous ammonium chloride solution was added and stirred for ten minutes. After stirring, the mixture was extracted three times with ethyl acetate (60.0 mL), the resulting organic phase was dried and concentrated, and the crude was washed twice with petroleum ether (60.0 mL) to obtain the title compound 128A (250.0 mg).

MS m/z (ESI): 336.0/338.0 [M+H]⁺.

### Step 2: Preparation of 3-(1-(3-bromophenyl)-1,2,2,2-tetrafluoroethyl)-4-methyl-4H-1,2,4-triazole (128B)

128A (100.0 mg, 0.3 mmol) was dissolved in dichloromethane (3.0 mL). After the reaction solution was cooled down to 0° C., DAST (0.079 mL, 0.6 mmol) was added dropwise to the reaction solution. After the dropwise addition was completed, the reaction solution was transferred to room temperature and reacted for 0.5 h. After the reaction was completed, the reaction solution was cooled down to 0° C. in an ice bath, and then a saturated aqueous sodium bicarbonate solution was added and stirred for ten minutes. After stirring, the reaction was extracted three times with dichloromethane (30.0 mL). The resulting organic phase was dried over anhydrous sodium sulfate, filtered and concentrated. The crude was purified by normal phase column chromatography (dichloromethane:methanol=95:5), to obtain the title compound 128B (60.0 mg).

MS m/z (ESI): 338.0/340.0 [M+H]⁺.

### Step 3: Preparation of 4-methyl-3-(1,2,2,2-tetrafluoro-1-(3-(4,4,5,5-tetramethyl-1,3,2-dioxaborolan-2-yl)phenyl)ethyl)-4H-1,2,4-triazole (128C)

To a mixture of 128B (60.0 mg, 177.46 μmol), bis(pinacolato)diboron (67.61 mg, 266.19 mol), potassium acetate (52.17 mg, 532.39 μmol) and anhydrous dioxane (2.0 mL) was added Pd(dppf)Cl₂ (12.88 mg, 17.75 μmol), and the resulting mixture was heated to 100° C. under nitrogen protection and stirred for 1 hour. After the reaction was completed, the mixture was cooled to room temperature, filtered, and concentrated under reduced pressure. The residue was purified by normal phase column chromatography (petroleum ether:ethyl acetate=0:1) to obtain the title compound 128C (60.0 mg).

MS m/z (ESI): 386.1 [M+H]⁺.

### Step 4: Preparation of 8-methyl-6-(((S)-3-methylpiperidin-1-yl)methyl)-3-(3-(1,2,2,2-tetrafluoro-1-(4-methyl-4H-1,2,4-triazol-3-yl)ethyl)phenyl)-4H-chromen-4-one (compound 128)

128C (60.0 mg, 155.78 μmol), 64C (41.26 mg, 103.85 μmol) and potassium carbonate (28.66 mg, 207.7 μmol) were added to a mixture of water (0.2 mL) and 1,4-dioxane (1.0 mL), and nitrogen replacement was performed. Under nitrogen atmosphere, Pd(dppf)Cl₂ (7.6 mg, 10.39 mol) was added, and the resulting mixture was heated to 100° C. under nitrogen protection and stirred for 1.0 h. After the reaction was completed, the mixture was cooled to room temperature, filtered, and concentrated under reduced pressure to obtain a crude, which was separated by preparative high performance liquid chromatography (chromatographic column: Welch Xtimate C18; column length 150 mm, inner diameter 30 mm, and particle size 5 μm; mobile phase A: water (with 0.225% NH₃), mobile phase B: acetonitrile; gradient: mobile phase B: from 5% to 95% over 18 minutes), to obtain the title compound 128 (25.0 mg).

MS m/z (ESI): 529.3 [M+H]⁺.

¹H NMR (400 MHz, DMSO-d₆) δ 8.78 (s, 1H), 8.71 (s, 1H), 7.87 (s, 1H), 7.81 (d, J=7.7 Hz, 1H), 7.66 (t, J=8.5 Hz, 3H), 7.41 (d, J=7.8 Hz, 1H), 3.52 (s, 2H), 2.73 (t, J=11.0 Hz, 2H), 2.50 (s, 3H), 1.89 (t, J=10.7 Hz, 1H), 1.63 (dd, J=29.5, 10.5 Hz, 4H), 1.48 (q, J=12.2 Hz, 1H), 0.87 (s, 1H), 0.82 (d, J=5.0 Hz, 3H).

### Example 28: 3-(3-(cyclopropylfluoro(4-methyl-4H-1,2,4-triazol-3-yl)methyl) phenyl)-6-(((R)-4,4-difluoro-3-methylpiperidin-1-yl)methyl)-8-methyl-4H-chromen-4-one (compound 129)

### Synthetic Route:

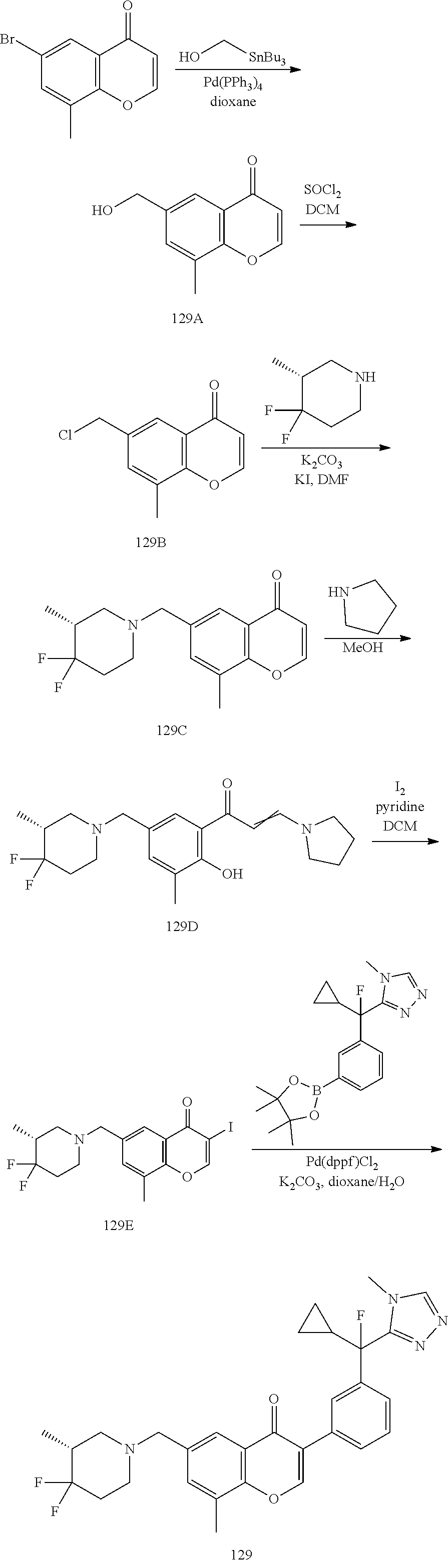

### Step 1: 6-(hydroxymethyl)-8-methyl-4H-chromen-4-one (129A)

With reference to the synthetic steps of 74A in Example 18, 58A was used instead of 1-(5-bromo-2-hydroxy-3-iodophenyl)ethyl-1-one to synthesize 6-bromo-8-methyl-4H-chromen-4-one. MS m/z(ESI): 239.0, 241.0[M+H]⁺.

To the dry reaction tube were added 6-bromo-8-methyl-4H-chromen-4-one (500 mg, 2.09 mmol), Pd(PPh₃)₄ (241.68 mg, 209.15 μmol), (tributyltin)methanol (2.01 g, 6.27 mmol) and dioxane (5 mL), the tube was evacuated and backfilled with argon, and the mixture was heated to 80° C., and reacted for 8 hours. After the reaction was completed, the mixture was cooled to room temperature and purified by reverse phase column chromatography (SepaFlash® silica gel column; mobile phase: acetonitrile/water=60:40), to obtain the title product 129A (180 mg).

MS m/z(ESI): 191 [M+H]⁺;

### Step 2: (6-(chloromethyl)-8-methyl-4H-chromen-4-one (129B)

129A (100 mg, 525.78 μmol) was dissolved in DCM (2 mL), and dithionyl chloride (125.10 mg, 1.05 mmol, 76.38 μL) was added dropwise to the mixture and the reaction was stirred at room temperature for 3 hours. After the reaction was completed, the reaction solution was concentrated to obtain the title compound 129B (109.7 mg) as a crude, which was directly used in the next step without purification.

MS m/z(ESI): 209[M+H]⁺;

### Step 3: (R)-6-((4,4-difluoro-3-methylpiperidin-1-yl)methyl)-8-methyl-4H-chromen-4-one (129C)

A mixture of (3R)-4,4-difluoro-3-methylpiperidine (65 mg, 480.93 μmol) and 129B (100.34 mg, 480.93 μmol) was dissolved in DMF (2 mL), and KI (7.98 mg, 48.09 μmol) and K₂CO₃ (132.93 mg, 961.86 μmol) were added to the mixture and the reaction was stirred at room temperature for 6 hours. After the reaction was completed, the resulting mixture was extracted with water and ethyl acetate, and washed with saturated brine. The organic phase was dried over anhydrous sodium sulfate, filtered and concentrated. The residue was purified by reverse phase column chromatography (SepaFlash® silica gel column; mobile phase: acetonitrile/water=75:25), to obtain the title product 129C (71 mg).

MS m/z(ESI): 308.1[M+H]⁺;

### Step 4: (R)-1-(5-((4,4-difluoro-3-methylpiperidin-1-yl)methyl)-2-hydroxyl-3-methylphenyl)-3-(pyrrolidin-1-yl)prop-2-en-1-one (129D)

129C (71 mg, 231.02 μmol) was dissolved in methanol (2 mL), pyrrolidine (49.29 mg, 693.06 μmol) was added and dissolved, and the mixture was heated to 50° C. and stirred for 1 hour. After the reaction was completed, the reaction solution was cooled to room temperature and concentrated to obtain the title compound 129D (87 mg) as a crude, which was directly used in the next step without purification.

MS m/z(ESI): 379.2[M+H]⁺;

### Step 5: (R)-6-((4,4-difluoro-3-methylpiperidin-1-yl)methyl)-3-iodo-8-methyl-4H-chromen-4-one (129E)

129D (87 mg, 229.88 μmol) was dissolved in DCM (5 mL), I₂ (116.69 mg, 459.76 μmol) and pyridine (36.37 mg, 459.76 μmol, 37.04 μL) were added to the mixture, and the reaction was stirred for 1 hour. After the reaction was completed, the reaction solution was concentrated and purified by reverse phase column chromatography (SepaFlash® silica gel column; mobile phase: acetonitrile/water=80:20), to obtain the title product 129E (60 mg).

MS m/z(ESI): 434[M+H]⁺;

### Step 6: 3-(3-(cyclopropylfluoro(4-methyl-4H-1,2,4-triazol-3-yl)methyl) phenyl)-6-(((R)-4,4-difluoro-3-methylpiperidin-1-yl)methyl)-8-methyl-4H-chromen-4-one (129)

To a reaction tube were added 129E (50 mg, 115.41 μmol), 120E (57.72 mg, 161.58 mol), Pd(dppf)Cl₂ (8.37 mg, 11.54 μmol) and K₂CO₃ (31.90 mg, 230.82 μmol), the tube was evacuated and backfilled with argon, water (0.5 mL) and dioxane (2 mL) were added via a syringe, and the mixture was heated to 100° C. and reacted for 1 hour. The mixture was cooled to room temperature, filtered, and concentrated, and then purified by reverse phase column chromatography (SepaFlash® silica gel column; mobile phase: acetonitrile/water=90:10), to obtain the title product 129 (18 mg).

MS m/z(ESI): 537.3[M+H]⁺;

1H NMR (400 MHz, DMSO-d₆) δ 8.64 (s, 1H), 8.54 (s, 1H), 7.88 (s, 1H), 7.64 (s, 1H), 7.60-7.46 (m, 2H), 7.42 (s, 1H), 7.19 (d, J=7.7 Hz, 1H), 3.61 (s, 2H), 3.32 (s, 3H), 2.81-2.65 (m, 2H), 2.48 (s, 3H), 2.31-2.21 (m, 1H), 2.15-1.95 (m, 4H), 1.30-1.20 (m, 1H), 0.91 (d, J=6.5 Hz, 3H), 0.75-0.6 (m, 2H), 0.58-0.50 (m, 1H), 0.48-0.38 (m, 1H).

With reference to the synthesis method of the above examples and the aforementioned general synthesis route 1, synthesis route 2, synthesis route 3 or synthesis route 4, the following compounds were synthesized, and their structures and mass spectrometry data are as follows:

| | | m/z |
|---|---|---|
| Compound | Structure | [M + H] |
| 3 | 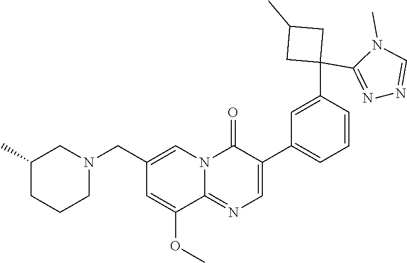 [SMILES: COc1cc(CN2CCC[C@H](C)C2)cn2c(=O)c(-c3cccc(C4(c5nncn5C)CC(C)C4)c3)cnc12] | 513.3 |
| 4 | 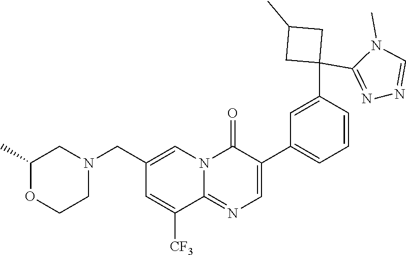 [SMILES: CC1CC(c2cccc(-c3cnc4c(C(F)(F)F)cc(CN5CCO[C@H](C)C5)cn4c3=O)c2)(c2nncn2C)C1] | 553.2 |
| 5 | 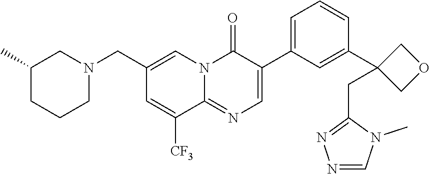 [SMILES: C[C@H]1CCCN(Cc2cc(C(F)(F)F)c3ncc(-c4cccc(C5(Cc6nncn6C)COC5)c4)c(=O)n3c2)C1] | 553.2 |
| 6 | 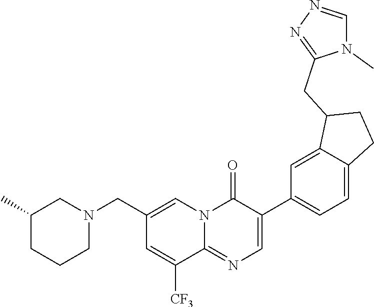 [SMILES: C[C@H]1CCCN(Cc2cc(C(F)(F)F)c3ncc(-c4ccc5c(c4)C(Cc4nncn4C)CC5)c(=O)n3c2)C1] | 537.3 |
| 7 | 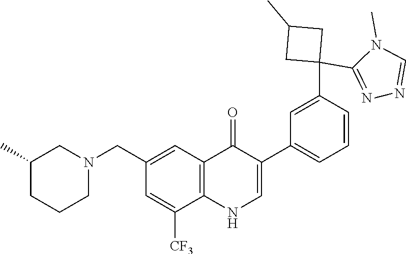 [SMILES: CC1CC(c2cccc(-c3c[nH]c4c(C(F)(F)F)cc(CN5CCC[C@H](C)C5)cc4c3=O)c2)(c2nncn2C)C1] | 482.3 |
| 8 | 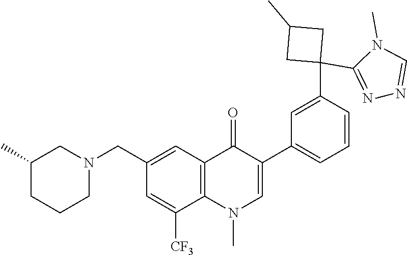 [SMILES: CC1CC(c2cccc(-c3cn(C)c4c(C(F)(F)F)cc(CN5CCC[C@H](C)C5)cc4c3=O)c2)(c2nncn2C)C1] | 496.3 |
| 9 | 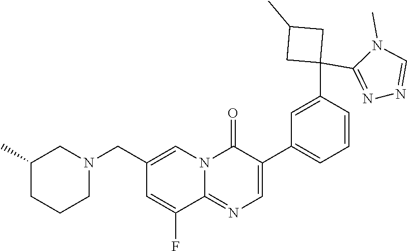 [SMILES: CC1CC(c2cccc(-c3cnc4c(F)cc(CN5CCC[C@H](C)C5)cn4c3=O)c2)(c2nncn2C)C1] | 501.2 |
| 10 | 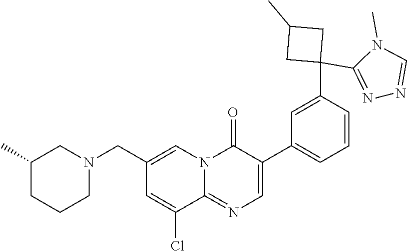 [SMILES: CC1CC(c2cccc(-c3cnc4c(Cl)cc(CN5CCC[C@H](C)C5)cn4c3=O)c2)(c2nncn2C)C1] | 517.2 |
| 11 | 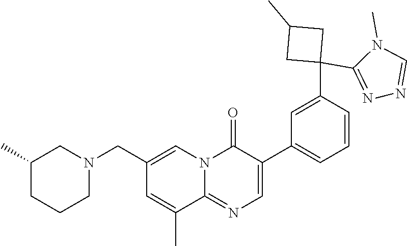 [SMILES: Cc1cc(CN2CCC[C@H](C)C2)cn2c(=O)c(-c3cccc(C4(c5nncn5C)CC(C)C4)c3)cnc12] | 497.2 |
| 12 | 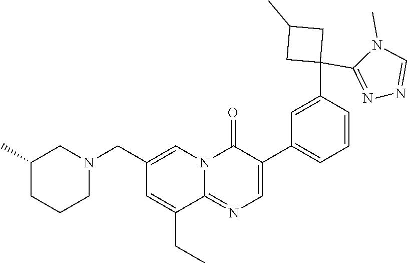 [SMILES: CCc1cc(CN2CCC[C@H](C)C2)cn2c(=O)c(-c3cccc(C4(c5nncn5C)CC(C)C4)c3)cnc12] | 511.3 |
| 13 | 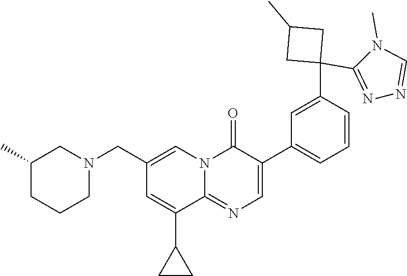 [SMILES: CC1CC(c2cccc(-c3cnc4c(C5CC5)cc(CN5CCC[C@H](C)C5)cn4c3=O)c2)(c2nncn2C)C1] | 523.3 |
| 14 | 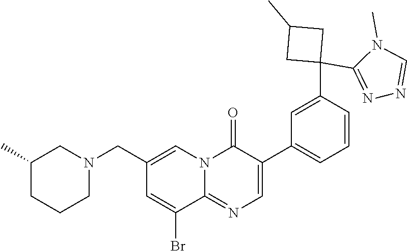 [SMILES: CC1CC(c2cccc(-c3cnc4c(Br)cc(CN5CCC[C@H](C)C5)cn4c3=O)c2)(c2nncn2C)C1] | 561.1/563.1 |
| 15 | 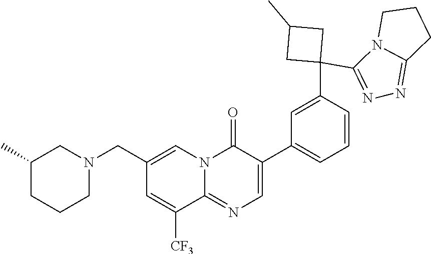 [SMILES: CC1CC(c2cccc(-c3cnc4c(C(F)(F)F)cc(CN5CCC[C@H](C)C5)cn4c3=O)c2)(c2nnc3n2CCC3)C1] | 577.2 |
| 16 | 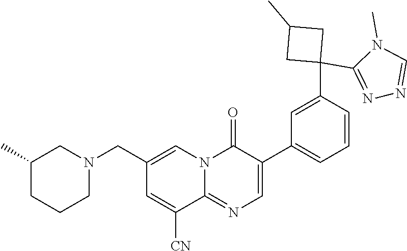 [SMILES: CC1CC(c2cccc(-c3cnc4c(C#N)cc(CN5CCC[C@H](C)C5)cn4c3=O)c2)(c2nncn2C)C1] | 508.3 |
| 17 | 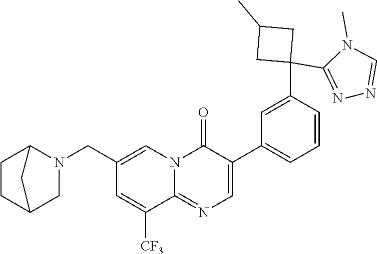 [SMILES: CC1CC(c2cccc(-c3cnc4c(C(F)(F)F)cc(CN5CC6CCC5C6)cn4c3=O)c2)(c2nncn2C)C1] | 549.1 |
| 18 | 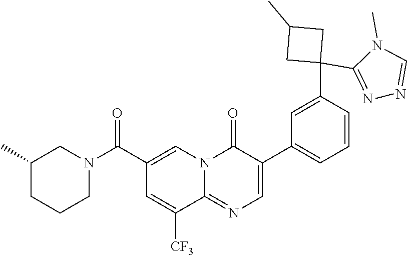 [SMILES: CC1CC(c2cccc(-c3cnc4c(C(F)(F)F)cc(C(=O)N5CCC[C@H](C)C5)cn4c3=O)c2)(c2nncn2C)C1] | 565.1 |
| 19 | 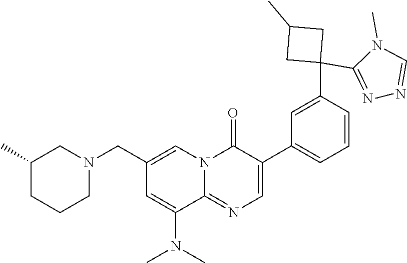 [SMILES: CC1CC(c2cccc(-c3cnc4c(N(C)C)cc(CN5CCC[C@H](C)C5)cn4c3=O)c2)(c2nncn2C)C1] | 526.2 |
| 20 | 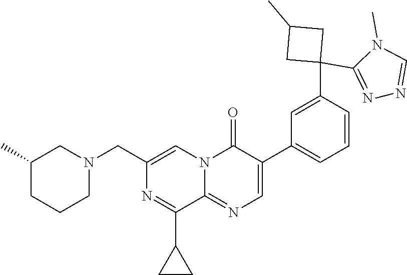 [SMILES: CC1CC(c2cccc(-c3cnc4c(C5CC5)nc(CN5CCC[C@H](C)C5)cn4c3=O)c2)(c2nncn2C)C1] | 524.3 |
| 21 | 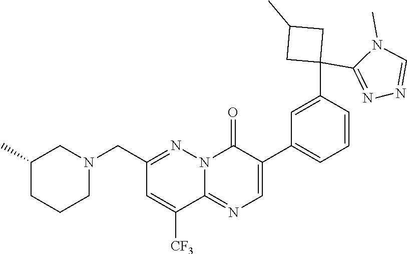 [SMILES: CC1CC(c2cccc(-c3cnc4c(C(F)(F)F)cc(CN5CCC[C@H](C)C5)nn4c3=O)c2)(c2nncn2C)C1] | 552.1 |
| 22 | 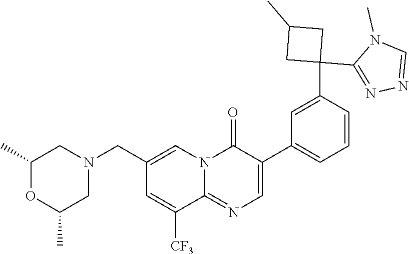 [SMILES: CC1CC(c2cccc(-c3cnc4c(C(F)(F)F)cc(CN5C[C@@H](C)O[C@@H](C)C5)cn4c3=O)c2)(c2nncn2C)C1] | 567.1 |
| 23 | 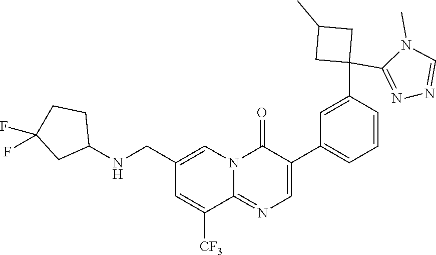 [SMILES: CC1CC(c2cccc(-c3cnc4c(C(F)(F)F)cc(CNC5CCC(F)(F)C5)cn4c3=O)c2)(c2nncn2C)C1] | 573.1 |
| 24 | 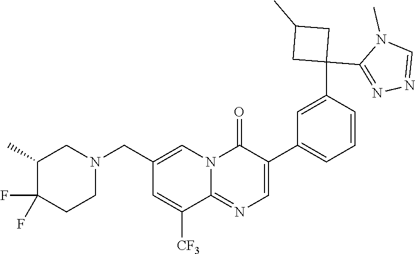 [SMILES: CC1CC(c2cccc(-c3cnc4c(C(F)(F)F)cc(CN5CCC(F)(F)[C@H](C)C5)cn4c3=O)c2)(c2nncn2C)C1] | 587.1 |
| 25 | 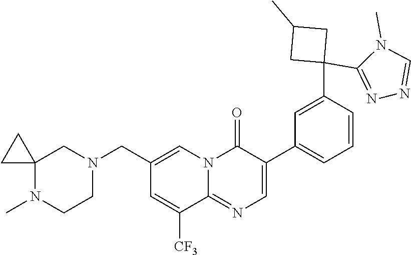 [SMILES: CC1CC(c2cccc(-c3cnc4c(C(F)(F)F)cc(CN5CCN(C)C6(CC6)C5)cn4c3=O)c2)(c2nncn2C)C1] | 578.1 |
| 26 | 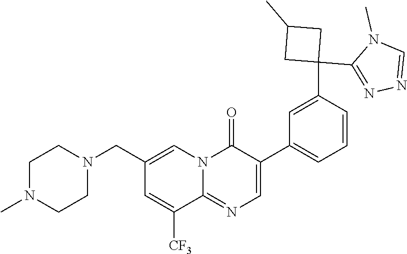 [SMILES: CC1CC(c2cccc(-c3cnc4c(C(F)(F)F)cc(CN5CCN(C)CC5)cn4c3=O)c2)(c2nncn2C)C1] | 552.2 |
| 27 | 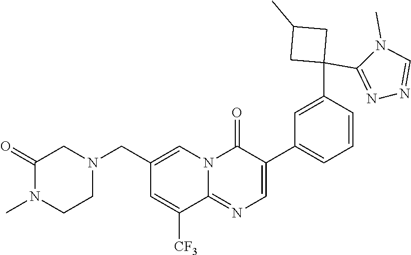 [SMILES: CC1CC(c2cccc(-c3cnc4c(C(F)(F)F)cc(CN5CCN(C)C(=O)C5)cn4c3=O)c2)(c2nncn2C)C1] | 566.3 |
| 28 | 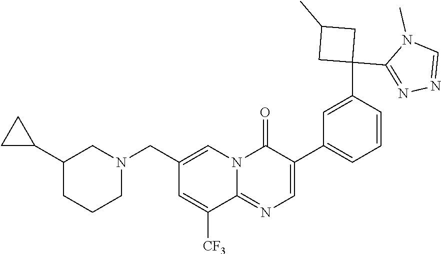 [SMILES: CC1CC(c2cccc(-c3cnc4c(C(F)(F)F)cc(CN5CCCC(C6CC6)C5)cn4c3=O)c2)(c2nncn2C)C1] | 577.1 |
| 29 | 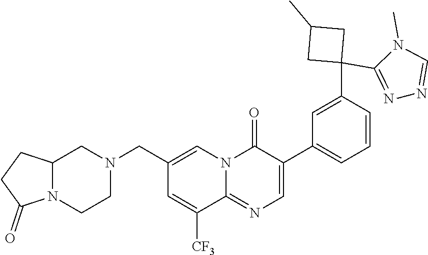 [SMILES: CC1CC(c2cccc(-c3cnc4c(C(F)(F)F)cc(CN5CCN6C(=O)CCC6C5)cn4c3=O)c2)(c2nncn2C)C1] | 592.1 |
| 30 | 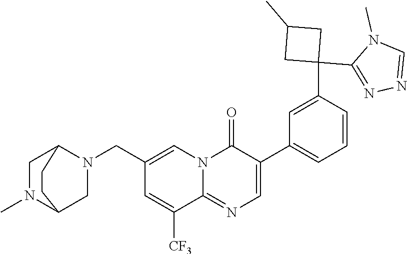 [SMILES: CC1CC(c2cccc(-c3cnc4c(C(F)(F)F)cc(CN5CC6CCC5CN6C)cn4c3=O)c2)(c2nncn2C)C1] | 578.2 |
| 31 | 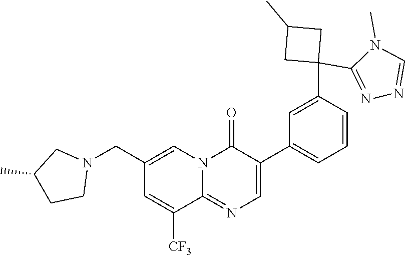 [SMILES: CC1CC(c2cccc(-c3cnc4c(C(F)(F)F)cc(CN5CC[C@H](C)C5)cn4c3=O)c2)(c2nncn2C)C1] | 537.1 |
| 32 | 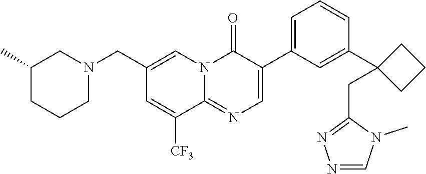 [SMILES: C[C@H]1CCCN(Cc2cc(C(F)(F)F)c3ncc(-c4cccc(C5(Cc6nncn6C)CCC5)c4)c(=O)n3c2)C1] | 551.1 |
| 33 | 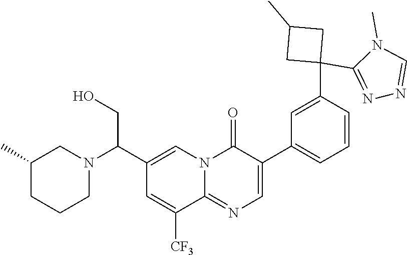 [SMILES: CC1CC(c2cccc(-c3cnc4c(C(F)(F)F)cc(C(CO)N5CCC[C@H](C)C5)cn4c3=O)c2)(c2nncn2C)C1] | 581.1 |
| 34 | 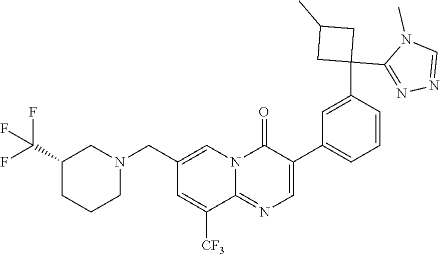 [SMILES: CC1CC(c2cccc(-c3cnc4c(C(F)(F)F)cc(CN5CCC[C@H](C(F)(F)F)C5)cn4c3=O)c2)(c2nncn2C)C1] | 605.3 |
| 35 | 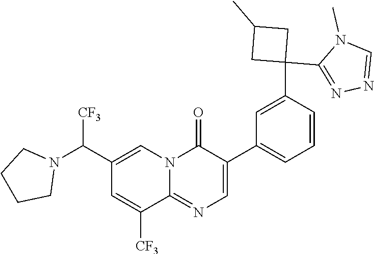 [SMILES: CC1CC(c2cccc(-c3cnc4c(C(F)(F)F)cc(C(N5CCCC5)C(F)(F)F)cn4c3=O)c2)(c2nncn2C)C1] | 591.2 |
| 36 | 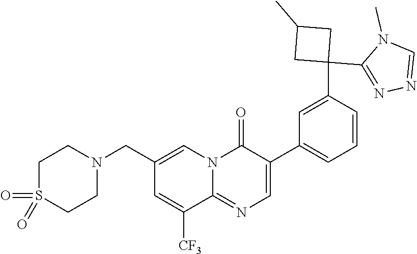 [SMILES: CC1CC(c2cccc(-c3cnc4c(C(F)(F)F)cc(CN5CCS(=O)(=O)CC5)cn4c3=O)c2)(c2nncn2C)C1] | 587.2 |
| 37 | 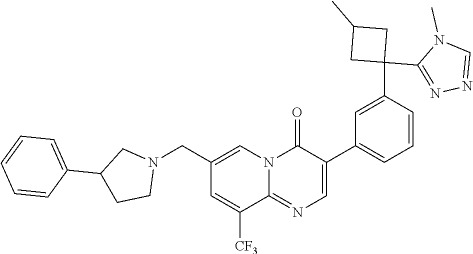 [SMILES: CC1CC(c2cccc(-c3cnc4c(C(F)(F)F)cc(CN5CCC(c6ccccc6)C5)cn4c3=O)c2)(c2nncn2C)C1] | 599.1 |
| 38 | 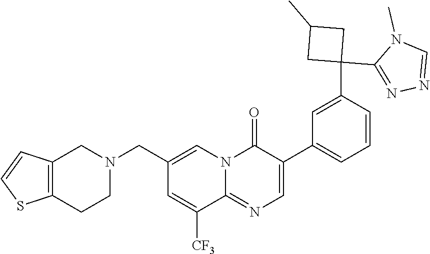 [SMILES: CC1CC(c2cccc(-c3cnc4c(C(F)(F)F)cc(CN5CCc6sccc6C5)cn4c3=O)c2)(c2nncn2C)C1] | 591.2 |
| 39 | 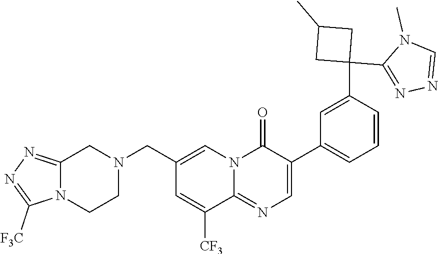 [SMILES: CC1CC(c2cccc(-c3cnc4c(C(F)(F)F)cc(CN5CCn6c(nnc6C(F)(F)F)C5)cn4c3=O)c2)(c2nncn2C)C1] | 644.2 |
| 40 | 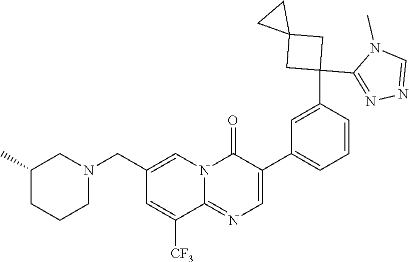 [SMILES: C[C@H]1CCCN(Cc2cc(C(F)(F)F)c3ncc(-c4cccc(C5(c6nncn6C)CC6(CC6)C5)c4)c(=O)n3c2)C1] | 563.2 |
| 41 | 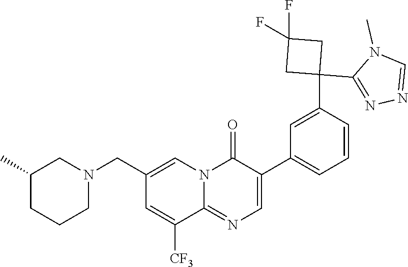 [SMILES: C[C@H]1CCCN(Cc2cc(C(F)(F)F)c3ncc(-c4cccc(C5(c6nncn6C)CC(F)(F)C5)c4)c(=O)n3c2)C1] | 573.2 |
| 42 | 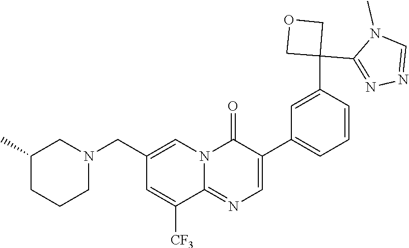 [SMILES: C[C@H]1CCCN(Cc2cc(C(F)(F)F)c3ncc(-c4cccc(C5(c6nncn6C)COC5)c4)c(=O)n3c2)C1] | 539.2 |
| 43 | 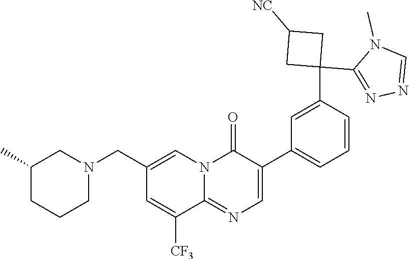 [SMILES: C[C@H]1CCCN(Cc2cc(C(F)(F)F)c3ncc(-c4cccc(C5(c6nncn6C)CC(C#N)C5)c4)c(=O)n3c2)C1] | 562.1 |
| 44 | 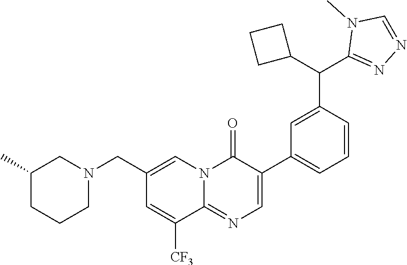 [SMILES: C[C@H]1CCCN(Cc2cc(C(F)(F)F)c3ncc(-c4cccc(C(c5nncn5C)C5CCC5)c4)c(=O)n3c2)C1] | 551.1 |
| 45 | 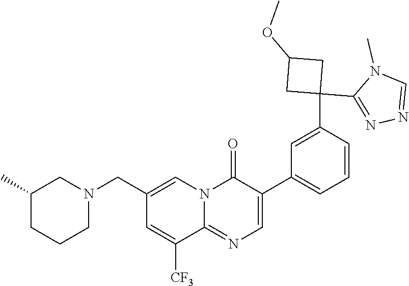 [SMILES: COC1CC(c2cccc(-c3cnc4c(C(F)(F)F)cc(CN5CCC[C@H](C)C5)cn4c3=O)c2)(c2nncn2C)C1] | 567.1 |
| 46 | 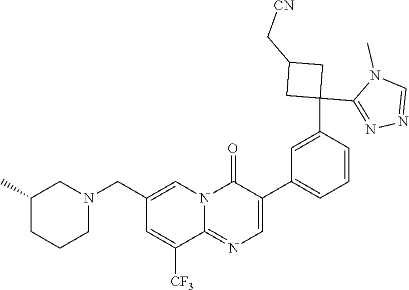 [SMILES: C[C@H]1CCCN(Cc2cc(C(F)(F)F)c3ncc(-c4cccc(C5(c6nncn6C)CC(CC#N)C5)c4)c(=O)n3c2)C1] | 576.1 |
| 47 | 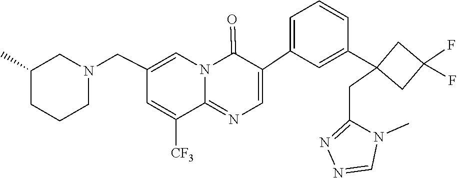 [SMILES: C[C@H]1CCCN(Cc2cc(C(F)(F)F)c3ncc(-c4cccc(C5(Cc6nncn6C)CC(F)(F)C5)c4)c(=O)n3c2)C1] | 587.4 |
| 48 | 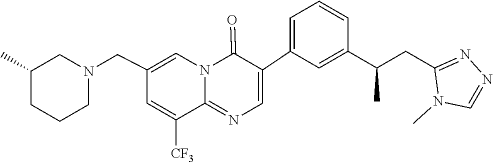 [SMILES: C[C@H]1CCCN(Cc2cc(C(F)(F)F)c3ncc(-c4cccc([C@H](C)Cc5nncn5C)c4)c(=O)n3c2)C1] | 525.1 |
| 49 | 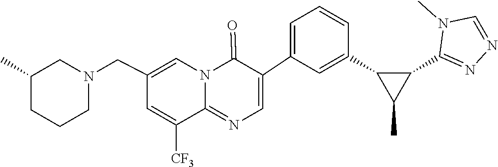 [SMILES: C[C@H]1CCCN(Cc2cc(C(F)(F)F)c3ncc(-c4cccc([C@H]5[C@H](C)[C@H]5c5nncn5C)c4)c(=O)n3c2)C1] | 537.2 |
| 50 | 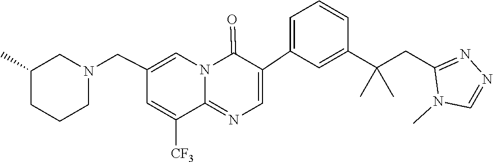 [SMILES: C[C@H]1CCCN(Cc2cc(C(F)(F)F)c3ncc(-c4cccc(C(C)(C)Cc5nncn5C)c4)c(=O)n3c2)C1] | 539.3 |
| 51 | 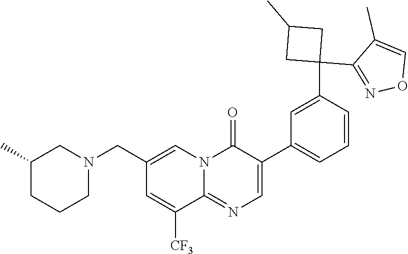 [SMILES: Cc1conc1C1(c2cccc(-c3cnc4c(C(F)(F)F)cc(CN5CCC[C@H](C)C5)cn4c3=O)c2)CC(C)C1] | 551.1 |
| 52 | 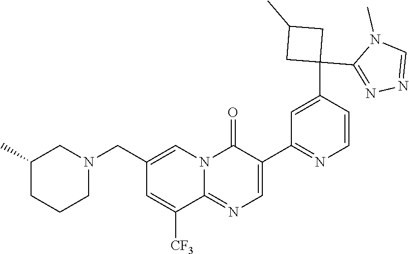 [SMILES: CC1CC(c2ccnc(-c3cnc4c(C(F)(F)F)cc(CN5CCC[C@H](C)C5)cn4c3=O)c2)(c2nncn2C)C1] | 552.1 |
| 53 | 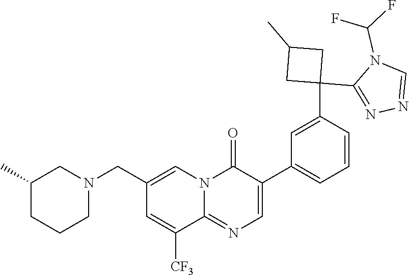 [SMILES: CC1CC(c2cccc(-c3cnc4c(C(F)(F)F)cc(CN5CCC[C@H](C)C5)cn4c3=O)c2)(c2nncn2C(F)F)C1] | 587.3 |
| 54 | 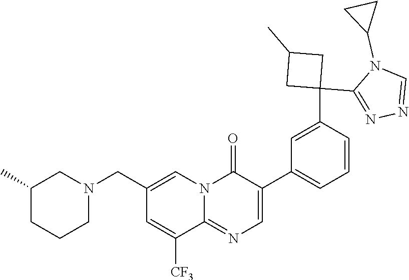 [SMILES: CC1CC(c2cccc(-c3cnc4c(C(F)(F)F)cc(CN5CCC[C@H](C)C5)cn4c3=O)c2)(c2nncn2C2CC2)C1] | 577.1 |
| 55 | 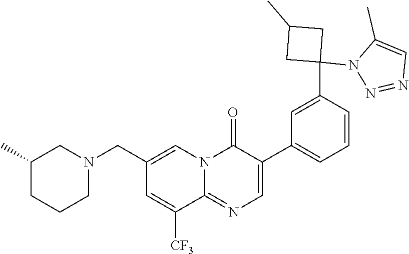 [SMILES: Cc1cnnn1C1(c2cccc(-c3cnc4c(C(F)(F)F)cc(CN5CCC[C@H](C)C5)cn4c3=O)c2)CC(C)C1] | 551.3 |
| 56 | 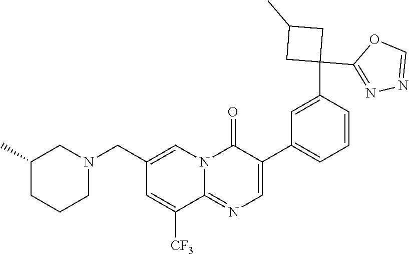 [SMILES: CC1CC(c2cccc(-c3cnc4c(C(F)(F)F)cc(CN5CCC[C@H](C)C5)cn4c3=O)c2)(c2nnco2)C1] | 538.3 |
| 57 | 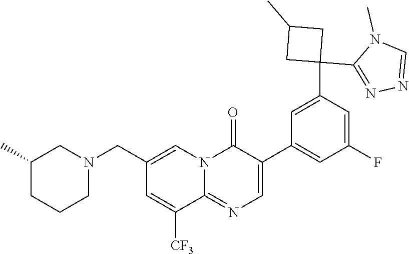 [SMILES: CC1CC(c2cc(F)cc(-c3cnc4c(C(F)(F)F)cc(CN5CCC[C@H](C)C5)cn4c3=O)c2)(c2nncn2C)C1] | 569.2 |
| 62 | 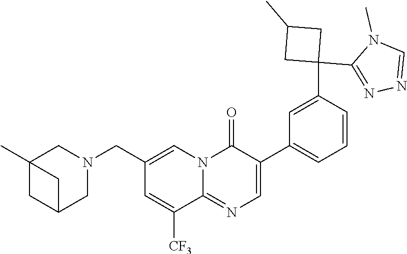 [SMILES: CC1CC(c2cccc(-c3cnc4c(C(F)(F)F)cc(CN5CC6CC(C)(C6)C5)cn4c3=O)c2)(c2nncn2C)C1] | 563.1 |
| 76 | 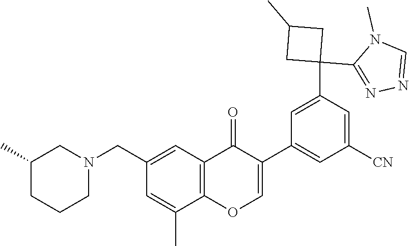 [SMILES: Cc1cc(CN2CCC[C@H](C)C2)cc2c(=O)c(-c3cc(C#N)cc(C4(c5nncn5C)CC(C)C4)c3)coc12] | 522.0 |
| 78 | 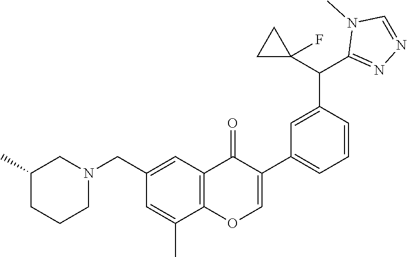 [SMILES: Cc1cc(CN2CCC[C@H](C)C2)cc2c(=O)c(-c3cccc(C(c4nncn4C)C4(F)CC4)c3)coc12] | 501.0 |
| 79 | 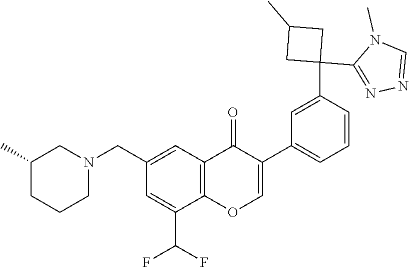 [SMILES: CC1CC(c2cccc(-c3coc4c(C(F)F)cc(CN5CCC[C@H](C)C5)cc4c3=O)c2)(c2nncn2C)C1] | 533.0 |
| 80 | 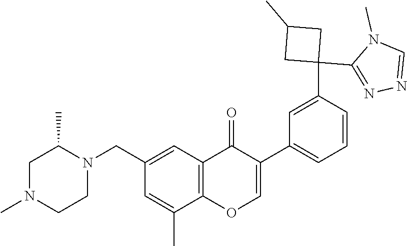 [SMILES: Cc1cc(CN2CCN(C)C[C@@H]2C)cc2c(=O)c(-c3cccc(C4(c5nncn5C)CC(C)C4)c3)coc12] | 512.1 |
| 81 | 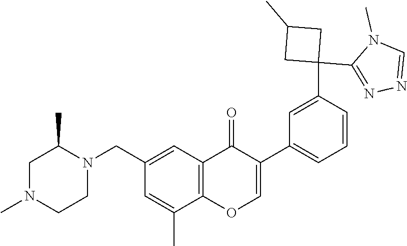 [SMILES: Cc1cc(CN2CCN(C)C[C@H]2C)cc2c(=O)c(-c3cccc(C4(c5nncn5C)CC(C)C4)c3)coc12] | 512.0 |
| 82 | 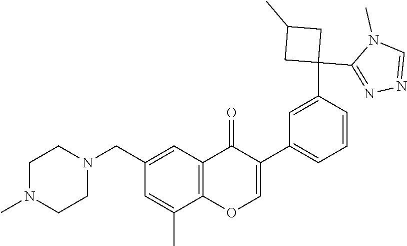 [SMILES: Cc1cc(CN2CCN(C)CC2)cc2c(=O)c(-c3cccc(C4(c5nncn5C)CC(C)C4)c3)coc12] | 498.1 |
| 83 | 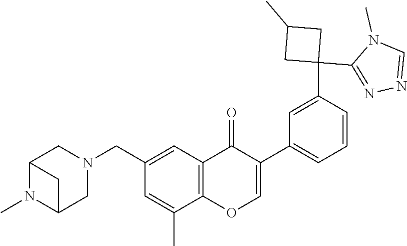 [SMILES: Cc1cc(CN2CC3CC(C2)N3C)cc2c(=O)c(-c3cccc(C4(c5nncn5C)CC(C)C4)c3)coc12] | 510.0 |
| 84 | 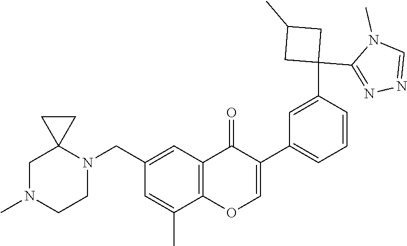 [SMILES: Cc1cc(CN2CCN(C)CC23CC3)cc2c(=O)c(-c3cccc(C4(c5nncn5C)CC(C)C4)c3)coc12] | 524.0 |
| 85 | 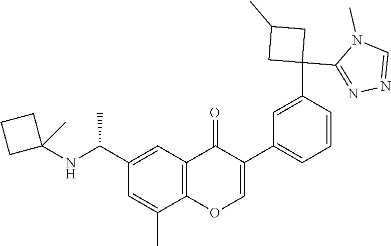 [SMILES: Cc1cc([C@@H](C)NC2(C)CCC2)cc2c(=O)c(-c3cccc(C4(c5nncn5C)CC(C)C4)c3)coc12] | 497.4 |
| 86 | 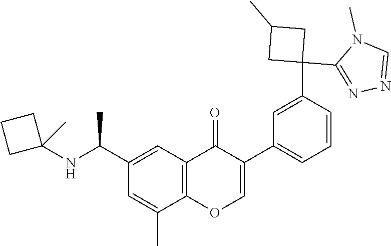 [SMILES: Cc1cc([C@H](C)NC2(C)CCC2)cc2c(=O)c(-c3cccc(C4(c5nncn5C)CC(C)C4)c3)coc12] | 497.1 |
| 87 | 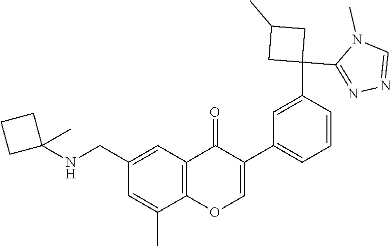 [SMILES: Cc1cc(CNC2(C)CCC2)cc2c(=O)c(-c3cccc(C4(c5nncn5C)CC(C)C4)c3)coc12] | 483.0 |
| 88 | 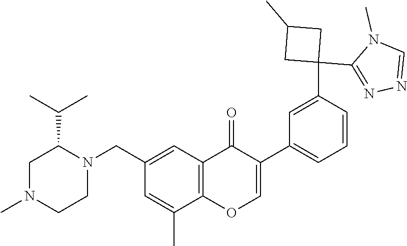 [SMILES: Cc1cc(CN2CCN(C)C[C@@H]2C(C)C)cc2c(=O)c(-c3cccc(C4(c5nncn5C)CC(C)C4)c3)coc12] | 540.1 |
| 89 | 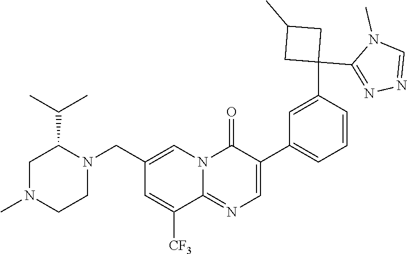 [SMILES: CC1CC(c2cccc(-c3cnc4c(C(F)(F)F)cc(CN5CCN(C)C[C@@H]5C(C)C)cn4c3=O)c2)(c2nncn2C)C1] | 594.3 |
| 90 | 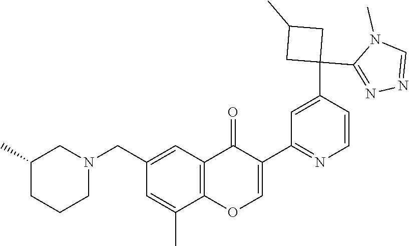 [SMILES: Cc1cc(CN2CCC[C@H](C)C2)cc2c(=O)c(-c3cc(C4(c5nncn5C)CC(C)C4)ccn3)coc12] | 498.0 |
| 91 | 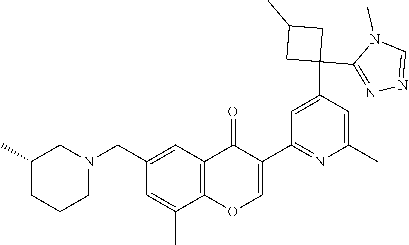 [SMILES: Cc1cc(C2(c3nncn3C)CC(C)C2)cc(-c2coc3c(C)cc(CN4CCC[C@H](C)C4)cc3c2=O)n1] | 512.0 |
| 92 | 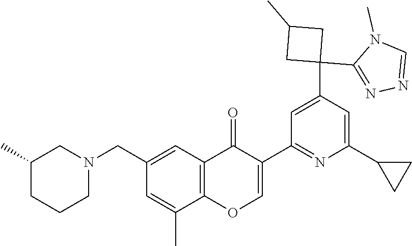 [SMILES: Cc1cc(CN2CCC[C@H](C)C2)cc2c(=O)c(-c3cc(C4(c5nncn5C)CC(C)C4)cc(C4CC4)n3)coc12] | 538.2 |
| 93 | 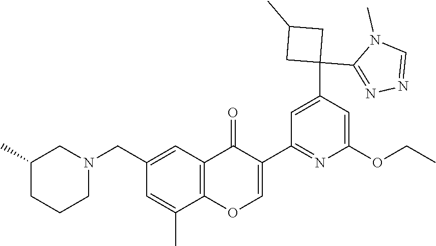 [SMILES: CCOc1cc(C2(c3nncn3C)CC(C)C2)cc(-c2coc3c(C)cc(CN4CCC[C@H](C)C4)cc3c2=O)n1] | 542.1 |
| 94 | 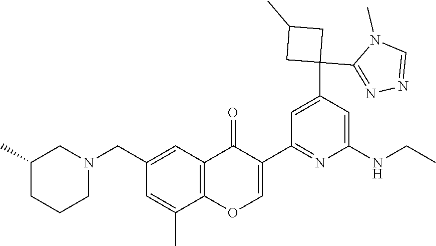 [SMILES: CCNc1cc(C2(c3nncn3C)CC(C)C2)cc(-c2coc3c(C)cc(CN4CCC[C@H](C)C4)cc3c2=O)n1] | 541.1 |
| 95 | 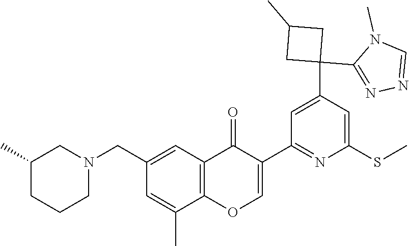 | 544.4 |
| 96 | 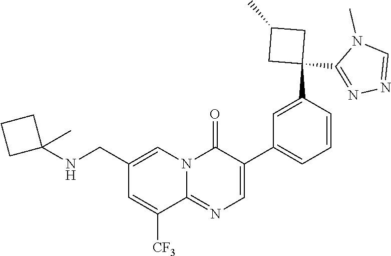 [SMILES: Cn1cnnc1[C@]1(c2cccc(-c3cnc4c(C(F)(F)F)cc(CNC5(C)CCC5)cn4c3=O)c2)C[C@H](C)C1] | 537.0 |
| 97 | 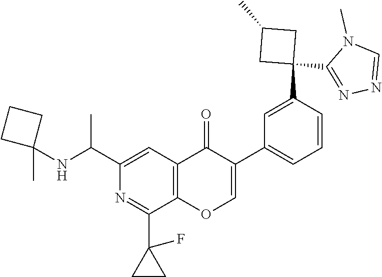 [SMILES: CC(NC1(C)CCC1)c1cc2c(=O)c(-c3cccc([C@]4(c5nncn5C)C[C@@H](C)C4)c3)coc2c(C2(F)CC2)n1] | 542.3 |
| 98 | 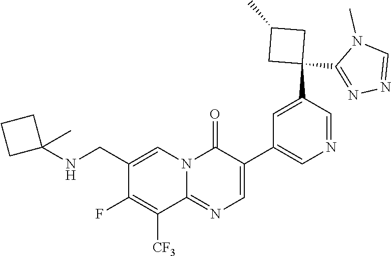 [SMILES: Cn1cnnc1[C@]1(c2cncc(-c3cnc4c(C(F)(F)F)c(F)c(CNC5(C)CCC5)cn4c3=O)c2)C[C@H](C)C1] | 555.2 |
| 99 | 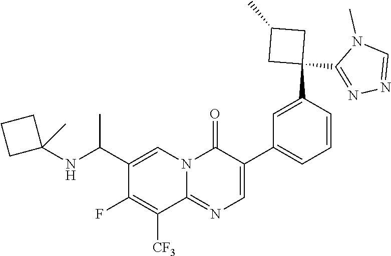 [SMILES: CC(NC1(C)CCC1)c1cn2c(=O)c(-c3cccc([C@]4(c5nncn5C)C[C@@H](C)C4)c3)cnc2c(C(F)(F)F)c1F] | 569.3 |
| 100 | 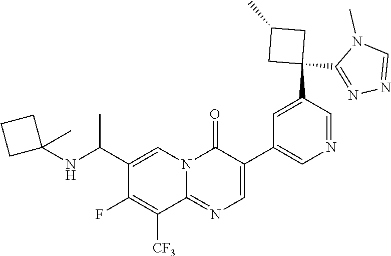 [SMILES: CC(NC1(C)CCC1)c1cn2c(=O)c(-c3cncc([C@]4(c5nncn5C)C[C@@H](C)C4)c3)cnc2c(C(F)(F)F)c1F] | 570.0 |
| 102 | 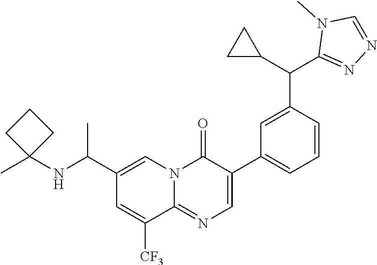 [SMILES: CC(NC1(C)CCC1)c1cc(C(F)(F)F)c2ncc(-c3cccc(C(c4nncn4C)C4CC4)c3)c(=O)n2c1] | 537.0 |
| 103 | 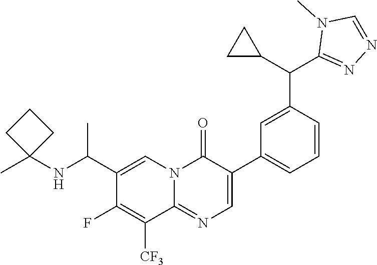 [SMILES: CC(NC1(C)CCC1)c1cn2c(=O)c(-c3cccc(C(c4nncn4C)C4CC4)c3)cnc2c(C(F)(F)F)c1F] | 555.1 |
| 104 | 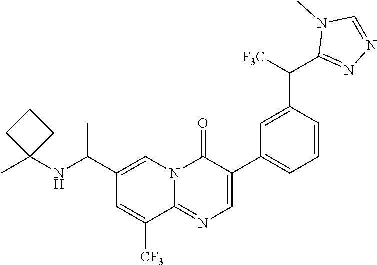 [SMILES: CC(NC1(C)CCC1)c1cc(C(F)(F)F)c2ncc(-c3cccc(C(c4nncn4C)C(F)(F)F)c3)c(=O)n2c1] | 565.3 |
| 105 | 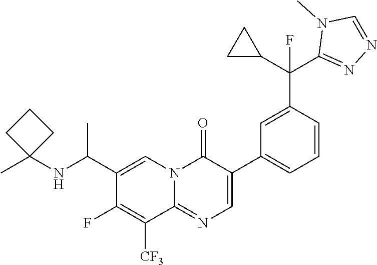 [SMILES: CC(NC1(C)CCC1)c1cn2c(=O)c(-c3cccc(C(F)(c4nncn4C)C4CC4)c3)cnc2c(C(F)(F)F)c1F] | 573.2 |
| 106 | 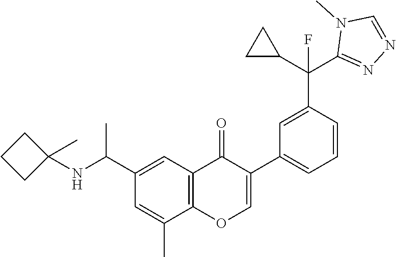 [SMILES: Cc1cc(C(C)NC2(C)CCC2)cc2c(=O)c(-c3cccc(C(F)(c4nncn4C)C4CC4)c3)coc12] | 501.0 |
| 107 | 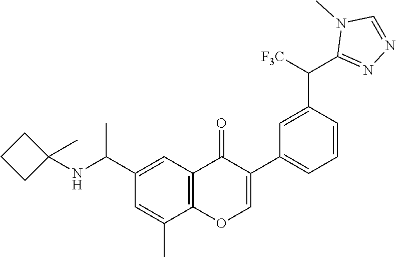 [SMILES: Cc1cc(C(C)NC2(C)CCC2)cc2c(=O)c(-c3cccc(C(c4nncn4C)C(F)(F)F)c3)coc12] | 511.1 |
| 108 | 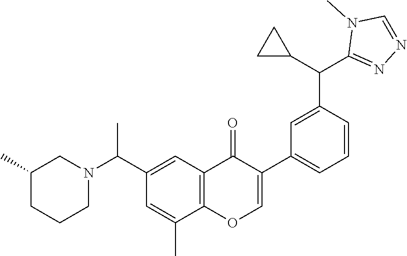 [SMILES: Cc1cc(C(C)N2CCC[C@H](C)C2)cc2c(=O)c(-c3cccc(C(c4nncn4C)C4CC4)c3)coc12] | 497.3 |
| 109 | 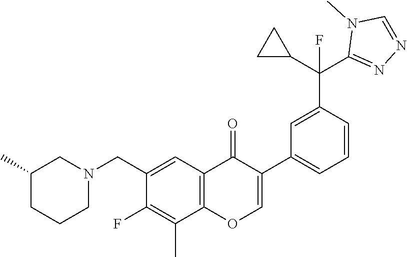 [SMILES: Cc1c(F)c(CN2CCC[C@H](C)C2)cc2c(=O)c(-c3cccc(C(F)(c4nncn4C)C4CC4)c3)coc12] | 518.2 |
| 110 | 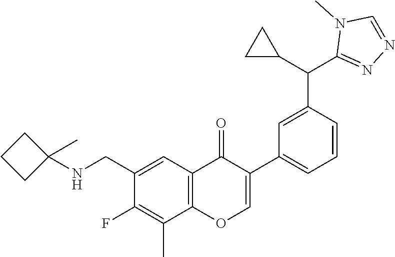 [SMILES: Cc1c(F)c(CNC2(C)CCC2)cc2c(=O)c(-c3cccc(C(c4nncn4C)C4CC4)c3)coc12] | 487.0 |
| 111 | 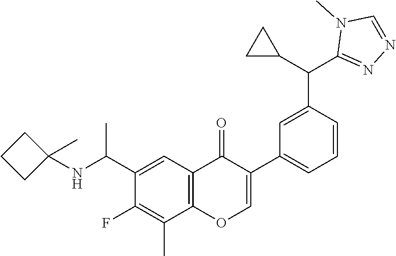 [SMILES: Cc1c(F)c(C(C)NC2(C)CCC2)cc2c(=O)c(-c3cccc(C(c4nncn4C)C4CC4)c3)coc12] | 501.0 |
| 112 | 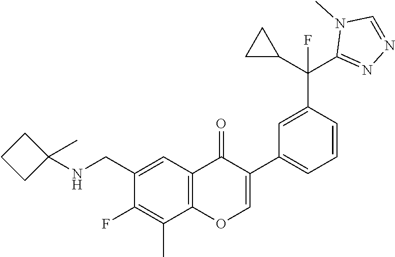 [SMILES: Cc1c(F)c(CNC2(C)CCC2)cc2c(=O)c(-c3cccc(C(F)(c4nncn4C)C4CC4)c3)coc12] | 519.1 |
| 113 | 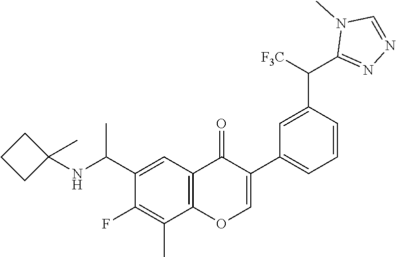 [SMILES: Cc1c(F)c(C(C)NC2(C)CCC2)cc2c(=O)c(-c3cccc(C(c4nncn4C)C(F)(F)F)c3)coc12] | 529.2 |
| 114 | 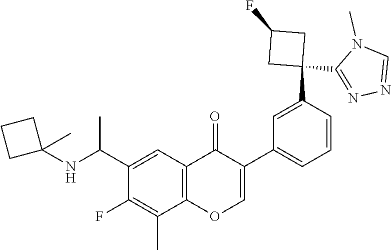 [SMILES: Cc1c(F)c(C(C)NC2(C)CCC2)cc2c(=O)c(-c3cccc([C@]4(c5nncn5C)C[C@H](F)C4)c3)coc12] | 519.2 |
| 115 | 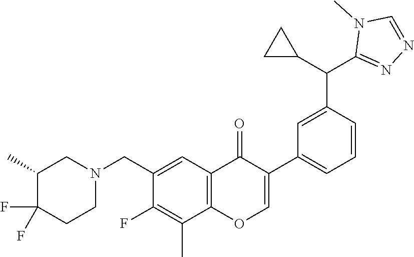 [SMILES: Cc1c(F)c(CN2CCC(F)(F)[C@H](C)C2)cc2c(=O)c(-c3cccc(C(c4nncn4C)C4CC4)c3)coc12] | 537.4 |
| 116 | 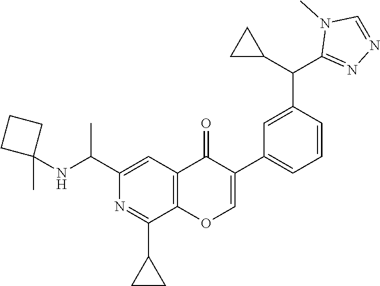 [SMILES: CC(NC1(C)CCC1)c1cc2c(=O)c(-c3cccc(C(c4nncn4C)C4CC4)c3)coc2c(C2CC2)n1] | 510.4 |
| 117 | 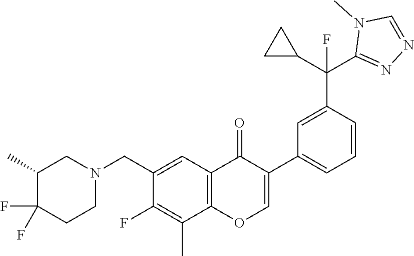 [SMILES: Cc1c(F)c(CN2CCC(F)(F)[C@H](C)C2)cc2c(=O)c(-c3cccc(C(F)(c4nncn4C)C4CC4)c3)coc12] | 555.3 |
| 118 | 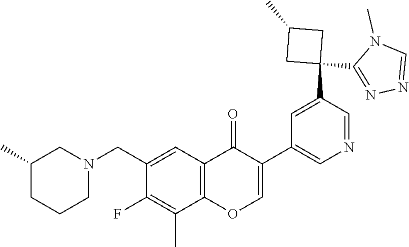 [SMILES: Cc1c(F)c(CN2CCC[C@H](C)C2)cc2c(=O)c(-c3cncc([C@]4(c5nncn5C)C[C@@H](C)C4)c3)coc12] | 516.2 |
| 119 | 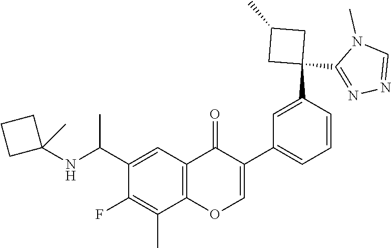 [SMILES: Cc1c(F)c(C(C)NC2(C)CCC2)cc2c(=O)c(-c3cccc([C@]4(c5nncn5C)C[C@@H](C)C4)c3)coc12] | 515.1 |
| 127 | 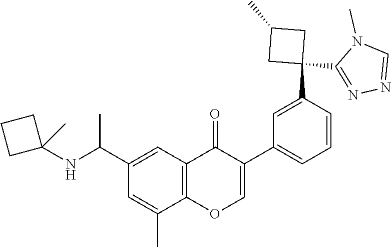 [SMILES: Cc1cc(C(C)NC2(C)CCC2)cc2c(=O)c(-c3cccc([C@]4(c5nncn5C)C[C@@H](C)C4)c3)coc12] | 483.3 |
| 130 | 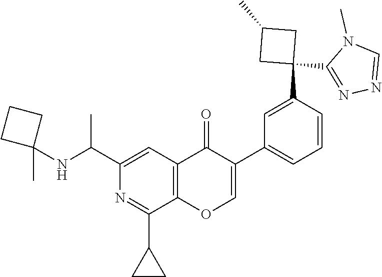 [SMILES: CC(NC1(C)CCC1)c1cc2c(=O)c(-c3cccc([C@]4(c5nncn5C)C[C@@H](C)C4)c3)coc2c(C2CC2)n1] | 524.1 |

### Test Examples of Biological Activity and Related Properties

### Test Example 1: Cbl-b Activity Assay

Experiment purpose: Testing of inhibitory activity of compounds on the interaction between Cbl-b protein and UbcH5B-Ub

Experimental method: Eu-Ubquitin-UbcH5B was prepared by incubating Eu-Ubquitin (Cisbio) with UbcH5B (ENZO), E1 (ENZO) at 37° C. for 4 hours. Eu-Ubquitin-UbcH5B was aliquoted and stored at −80° C. Cbl-b activity assay was performed in 384-well plates (Perkin Elmer). 100 nL of 3-fold serial dilutions of compounds (final concentration 10 μM-0.5 nM, starting concentration 10 μM, 3-fold dilution, 10 points, 10th point being 0.5 nM) and 5 μL of 50 nM Biotin-Cbl-b protein (Sigma) were incubated for 1 hour at room temperature, and the reaction buffer was 50 mM HEPES pH 7.0 (Gibco), 100 mM NaCl (Sigma), 0.01% Triton X-100 (Sigma), 0.01% BSA (Sigma) and 1 mM DTT (Invitrogen). 5 μL of Src mixed solution (40 nM Src (R&D), 2 mM ATP (Sigma), 10 mM MgCl₂ (Sigma)) was added to the reaction plate and incubated at room temperature for 3 hours. 10 μL of detection solution (12.5 nM Strepdividin-XL665 (Cisbio), 500 nM Eu-Ubquitin-UbcH5B, 120 nM EDTA (Invitrogen), 0.004% BSA (Sigma)) was added to the reaction plate, incubated overnight at room temperature, and the HTRF signal (665 nm/615 nm) was read on Envision (Perkin Elmer). IC₅₀ was calculated using IDBS XLfit.

The experimental results are shown in Table 1.

TABLE 1

| Compound number | IC₅₀ (nM) |
|---|---|
| 1 | 76.6 |
| 2 | 40.3 |
| 2-P1 | 27.1 |
| 2-P2 | 30.5 |
| 3 | 295.9 |
| 4 | 267.9 |
| 5 | 449.6 |
| 8 | 366.2 |
| 9 | 130.1 |
| 10 | 42.9 |
| 11 | 31.2 |
| 12 | 63.9 |
| 13 | 31.9 |
| 14 | 29.3 |
| 17 | 167.5 |
| 58 | 9.7 |
| 59 | 43.1 |
| 62 | 23.6 |
| 63 | 41.6 |
| 64 | 38.4 |
| 65 | 17.0 |
| 66 | 88.9 |
| 67 | 96.4 |
| 68 | 17.8 |
| 69 | 15.0 |
| 71 | 203.2 |
| 73 | 64.9 |
| 74 | 19.1 |
| 75 | 7.4 |
| 88 | 32.7 |
| 89 | 41.6 |
| 120 | 17.5 |
| 120-P1 | 212.4 |
| 120-P2 | 7.8 |
| 121 | 10.1 |
| 122 | 12.3 |
| 123 | 5.4 |
| 124 | 19.7 |
| 126 | 11.1 |
| 127 | 13.3 |
| 128 | 28.6 |
| 129 | 17.8 |

### Test Example 2: Jurkat T Activation Assay

Experiment purpose: Testing of effect of compounds on IL-2 release and activation of Jurkat T cells

Experimental method: 96-well cell plates (Corning) were coated with 2 μg/mL Anti-Human CD3 Clone OKT3 (BD) at 37° C. for 4 hours. 3-fold serial dilutions of compounds (final concentration 10 μM-4.6 nM, starting concentration 10 μM, 3-fold dilution, 8 points, 8th point being 4.6 nM) and 220 μL of 1.11×10⁶/mL Jurkat T cells (ATCC) were incubated for 1 hour, 5 μL of 45 μg/mL Anti-Human CD28 Clone CD28.2 (BD) was added, mixed evenly, 100 μL was transferred to the aforementioned CD3-coated cell plate, cultured in a cell incubator at 37° C. for 48 hours, and the supernatant was collected and the IL-2 release was detected using an IL-2 ELISA kit (BD). EC₅₀ was analyzed using Prism.

The experimental results are shown in Table 2.

TABLE 2

| Compound number | EC₅₀ (nM) |
|---|---|
| 2 | 634.2 |
| 2-P1 | 425.9 |
| 2-P2 | 400.8 |
| 58 | 112.1 |
| 62 | 524.9 |
| 65 | 265.0 |
| 68 | 456.4 |
| 69 | 552.3 |
| 120 | 687.9 |
| 121 | 250.5 |
| 122 | 554.6 |
| 123 | 53.6 |
| 124 | 382.6 |
| 126 | 80.5 |
| 127 | 208.6 |
| 128 | 279.3 |

1. A compound of formula (I), or a stereoisomer or pharmaceutically acceptable salt thereof: 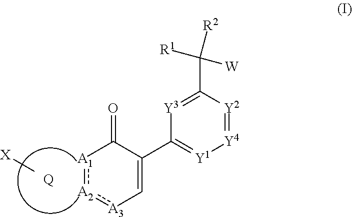
wherein 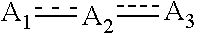
is selected from any of the following: i) C═C-A₃, wherein the A₃ is selected from CR^{11a}R^{11b}, NR¹², O or S; ii) A₁-C=A₃, wherein the A₁ is selected from C or N, and A₃ is selected from CR^{11c} or N; iii) A₁-A₂-A₃, wherein the A₁ and A₂ are independently selected from C(R¹¹)ₙ or N, and A₃ is selected from CR^{11a}R^{11b}, NR¹², O or S;
n is selected from 0 or 1;
R^{11a}, R^{11b}, R^{11c}, R¹¹, and R¹² are each independently selected from H, halogen, OH, CN, NH₂, NH(C₁-C₆ alkyl), N(C₁-C₆ alkyl)₂, C₁-C₆ alkyl, C₁-C₆ alkoxy, C₃-C₆ cycloalkyl or C₃-C₆ cycloalkyloxy, wherein the C₁-C₆ alkyl, C₁-C₆ alkoxy, C₃-C₆ cycloalkyl or C₃-C₆ cycloalkyloxy is optionally substituted with R^{11d};
ring Q is selected from phenyl, 5- to 6-membered heteroaryl or 5- to 7-membered heterocyclyl, wherein the phenyl, 5- to 6-membered heteroaryl or 5- to 7-membered heterocyclyl is optionally substituted with R¹⁰;
R¹⁰ is selected from halogen, ═O, OH, NH₂, NO₂, CN, C₁-C₆ alkyl, C₂-C₄ alkenyl, C₂-C₄ alkynyl, C₁-C₆ alkoxy, C₃-C₆ cycloalkyl, C₃-C₆ cycloalkyloxy, C₃-C₆ cycloalkyl-NH—, 4- to 7-membered heterocyclyl, 4- to 7-membered heterocyclyloxy or 4- to 7-membered heterocyclyl-NH—, wherein the NH₂, C₁-C₆ alkyl, C₂-C₄ alkenyl, C₂-C₄ alkynyl, C₁-C₆ alkoxy, C₃-C₆ cycloalkyl, C₃-C₆ cycloalkyloxy, C₃-C₆ cycloalkyl-NH—, 4- to 7-membered heterocyclyl, 4- to 7-membered heterocyclyloxy or 4- to 7-membered heterocyclyl-NH— is optionally substituted with R^{10a};
Y¹, Y², Y³ and Y⁴ are independently selected from CR^{b} or N;
X is selected from halogen, CN, OH, COOH, CONH₂, C₁-C₆ alkyl, C₁-C₆ alkoxy, 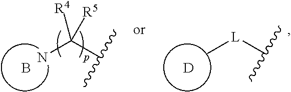
wherein the C₁-C₆ alkyl or C₁-C₆ alkoxy is optionally substituted with R^{e}, ring B is selected from the following groups optionally substituted with R³: 4- to 10-membered nitrogen-containing heterocyclyl or 5- to 10-membered nitrogen-containing heteroaryl, ring D is selected from the following groups optionally substituted with R⁶: C₃-C₁₀ cycloalkyl, 4- to 10-membered heterocyclyl, phenyl or 5- to 10-membered heteroaryl, and ring D is connected to L via a non-N atom, with L being selected from a bond, —NR⁷—, —NR⁷CR⁸R⁹—, —O—, —C(═O)—, —C(═O)NH— or —CR⁸R⁹—;
R^{b} is selected from H, halogen, OH, CN, C₁-C₆ alkyl, C₁-C₆ alkoxy, C₁-C₆ alkylthio, NH₂, NH(C₁-C₆ alkyl), N(C₁-C₆ alkyl)₂, NHC(O)(C₁-C₆ alkyl), NHC(O)—O(C₁-C₆ alkyl), N(C₁-C₆ alkyl)C(O)—O(C₁-C₆ alkyl), NHS(O)₂(C₁-C₆ alkyl), C₃-C₆ cycloalkyl, C₃-C₆ cycloalkyl-O—, C₃-C₆ cycloalkyl-NH—, N(C₃-C₆ cycloalkyl)₂, NHC(O)—C₃-C₆ cycloalkyl, NHS(O)₂—C₃-C₆ cycloalkyl, 4- to 7-membered heterocyclyl, 4- to 7-membered heterocyclyloxy, 4- to 7-membered heterocyclyl-NH—, N(4- to 7-membered heterocyclyl)₂, NHC(O)-4- to 7-membered heterocyclyl, NHS(O)₂-4- to 7-membered heterocyclyl, C₆-C₁₀ aryl, C₆-C₁₀ aryloxy, C₆-C₁₀ aryl-NH—, N(C₆-C₁₀ aryl)₂, NHC(O)—C₆-C₁₀ aryl, NHS(O)₂—C₆-C₁₀ aryl, 5- to 10-membered heteroaryl, 5- to 10-membered heteroaryloxy or 5- to 10-membered heteroaryl-NH—, N(5- to 10-membered heteroaryl)₂, NHC(O)-5- to 10-membered heteroaryl, or NHS(O)₂-5- to 10-membered heteroaryl, wherein the C₁-C₆ alkyl, C₁-C₆ alkoxy, C₁-C₆ alkylthio, C₃-C₆ cycloalkyl, 4- to 7-membered heterocyclyl, C₆-C₁₀ aryl or 5- to 10-membered heteroaryl is optionally substituted with R^{2a};
or two R^{b} together with the C atom to which they are attached form C₃-C₆ cycloalkenyl, phenyl, 4- to 7-membered heterocyclyl or 5- to 6-membered heteroaryl, wherein the C₃-C₆ cycloalkenyl, phenyl, 4- to 7-membered heterocyclyl or 5- to 6-membered heteroaryl is optionally substituted with R^{2a};
R⁴, R⁵, R⁷, R⁸ and R⁹ are each independently selected from H, halogen, OH, C₁-C₆ alkyl or C₁-C₆ alkoxy, wherein the C₁-C₆ alkyl or C₁-C₆ alkoxy is optionally substituted with R^{4a};
or R⁸ and R⁹ together with the atom to which they are attached form C₃-C₆ cycloalkyl or 4- to 7-membered heterocyclyl, wherein the C₃-C₆ cycloalkyl or 4- to 7-membered heterocyclyl is optionally further substituted with R^{8a};
or R⁴ and R⁵ together with the atom to which they are attached form C₃-C₆ cycloalkyl or 4- to 7-membered heterocyclyl, wherein the C₃-C₆ cycloalkyl or 4- to 7-membered heterocyclyl is optionally further substituted with R^{8a}; or when p is selected from 2, two R⁴ together with the atom to which they are attached form C₃-C₆ cycloalkyl or 4- to 7-membered heterocyclyl, wherein the C₃-C₆ cycloalkyl or 4- to 7-membered heterocyclyl is optionally further substituted with R^{8a};
or R⁴ and R⁵ together form ═O;
R³ and R⁶ are independently selected from halogen, CN, ═O, OH, NO₂, C₁-C₆ alkyl, OR^{6a}, SR^{6a}, N(R^{6a})₂, S(O)₂R^{6a}, S(O)₂N(R^{6a})₂, S(O)R^{6a}, S(O)N(R^{6a})₂, C(O)R^{6a}, C(O)OR^{6a}, C(O)N(R^{6a})₂, C(O)N(R^{6a})OR^{6a}, OC(O)R^{6a}, OC(O)N(R^{6a})₂, N(R^{6a})C(O)OR^{6a}, N(R^{6a})C(O)R^{6a}, N(R^{6a})C(O)N(R^{6a})₂, N(R^{6a})C(NR^{6a})N(R^{6a})₂, N(R^{6a})S(O)₂N(R^{6a})₂, N(R^{6a})S(O)₂R^{6a}, C₃-C₁₀ cycloalkyl, 4- to 7-membered heterocyclyl, 6- to 10-membered aryl or 5- to 10-membered heteroaryl, wherein the C₁-C₆ alkyl, C₃-C₁₀ cycloalkyl, 4- to 7-membered heterocyclyl, C₆-C₁₀ aryl or 5- to 10-membered heteroaryl is optionally further substituted with R^{3a};
each R^{6a} is independently selected from H, C₁-C₆ alkyl, phenyl, 4- to 7-membered heterocyclyl or 5- to 6-membered heteroaryl, wherein the C₁-C₆ alkyl, phenyl, 4- to 7-membered heterocyclyl or 5- to 6-membered heteroaryl is optionally further substituted with R^{6b}, or two R^{6a} on one N atom together with the N atom to which they are attached form 4- to 7-membered heterocyclyl or 5- to 6-membered heteroaryl, wherein the 4- to 7-membered heterocyclyl or 5- to 6-membered heteroaryl is optionally further substituted with R^{6b};
R^{3a}, R^{4a}, R^{6b} and R^{e} are independently selected from halogen, OH, CN, ═O, C₁-C₆ alkyl, NH₂, NH(C₁-C₆ alkyl), N(C₁-C₆ alkyl)₂, COOH or C₁-C₆ alkoxy, wherein the C₁-C₆ alkyl or C₁-C₆ alkoxy is optionally further substituted with R^{f};
R^{f} is selected from halogen, OH, ═O, NH₂, NH(C₁-C₆ alkyl) or N(C₁-C₆ alkyl)₂;
R^{11d}, R^{2a} and R^{10a} are independently selected from halogen, OH, CN, ═O, NH₂, NH(C₁-C₆ alkyl), N(C₁-C₆ alkyl)₂, C₁-C₆ alkyl, halo C₁-C₆ alkyl, C₁-C₆ alkoxy or halo C₁-C₆ alkoxy;
R¹ and R² are independently selected from H, halogen, CN, NH₂, NH(C₁-C₆ alkyl), N(C₁-C₆ alkyl)₂, C₁-C₆ alkyl, C₁-C₆ alkoxy, C₃-C₁₀ cycloalkyl or 4- to 10-membered heterocyclyl, wherein the C₁-C₆ alkyl, C₁-C₆ alkoxy, C₃-C₁₀ cycloalkyl or 4- to 10-membered heterocyclyl is optionally substituted with R^{1a},
or R¹ and R² together with the atom to which they are attached form C₃-C₁₀ cycloalkyl or 4- to 10-membered heterocyclyl, wherein the C₃-C₁₀ cycloalkyl or 4- to 10-membered heterocyclyl is optionally substituted with Rib;
or R¹ and R^{b} together with the atom and bond to which they are respectively attached form C₃-C₆ cycloalkenyl or 4- to 7-membered heterocyclyl, wherein the C₃-C₆ cycloalkenyl or 4- to 7-membered heterocyclyl is optionally substituted with Rid;
R^{1a} and R^{1b} are independently selected from halogen, OH, CN, ═O, NH₂, NH(C₁-C₆ alkyl), N(C₁-C₆ alkyl)₂, C₁-C₆ alkyl or C₁-C₆ alkoxy, wherein the C₁-C₆ alkyl or C₁-C₆ alkoxy is optionally further substituted with R^{1c};
R^{1c} is selected from halogen, OH, CN, ═O, NH₂ or COOH;
W is selected from (CR¹³R¹⁴)ₖW₁; the W₁ is selected from 5- to 10-membered heteroaryl or 4- to 10-membered heterocyclyl, wherein the 5- to 10-membered heteroaryl or 4- to 10-membered heterocyclyl is optionally substituted with R¹⁵; R¹³ and R¹⁴ are independently selected from H, halogen, OH, C₁-C₆ alkyl or C₁-C₆ alkoxy; R¹⁵ is selected from halogen, OH, NH₂, NH(C₁-C₆ alkyl), N(C₁-C₆ alkyl)₂, C₁-C₆ alkyl, C₃-C₁₀ cycloalkyl or 4- to 7-membered heterocyclyl, wherein the C₁-C₆ alkyl, C₃-C₁₀ cycloalkyl or 4- to 7-membered heterocyclyl is optionally substituted with R^{15a};
or R¹ and R¹³ together with the atom and bond to which they are respectively attached form C₃-C₆ cycloalkyl or 4- to 7-membered heterocyclyl, wherein the C₃-C₆ cycloalkyl or 4- to 7-membered heterocyclyl is optionally substituted with R^{13a};
R^{8a}, R^{13a} and R^{1d} are independently selected from halogen, OH, CN, C₁-C₆ alkyl or C₁-C₆ alkoxy, wherein the C₁-C₆ alkyl or C₁-C₆ alkoxy is optionally further substituted with halogen; R^{15a} is selected from halogen, ═O, OH, CN or C₁-C₆ alkyl;
k is selected from 0 or 1;
p is selected from 0, 1 or 2.

2. The compound of formula (I), or the stereoisomer or pharmaceutically acceptable salt thereof according to claim 1, wherein 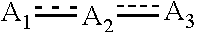
is selected from A₁-C=A₃, wherein the A₁ is selected from C or N, and A₃ is selected from CR^{11c} or N; or the A₁ is selected from N, and A₃ is selected from CR^{1c} or N; or the A₁ is selected from N, and A₃ is selected from N or C—F; or the A₁ is selected from N, and A₃ is selected from N; and/or
wherein 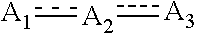
is selected from C═C-A₃, wherein the A₃ is selected from CR^{11a}R^{11b}, NR¹², O or S; or the A₃ is selected from NR¹², O or S; or the A₃ is selected from NH, N—CH₃, O or S; or the A₃ is selected from O.

3. (canceled)

4. The compound of formula (I), or the stereoisomer or pharmaceutically acceptable salt thereof according to claim 1, wherein ring Q is selected from the following groups optionally substituted with R¹⁰: phenyl, pyridyl, pyrimidyl, pyrazolyl, imidazolyl, oxazolyl, isoxazolyl, thiazolyl, isothiazolyl or 5- to 7-membered heterocyclyl, wherein the 5- to 7-membered heterocyclyl comprises 1 or 2 N atoms as heteroatoms; or,
ring Q is selected from the following groups optionally substituted with R¹⁰: phenyl, 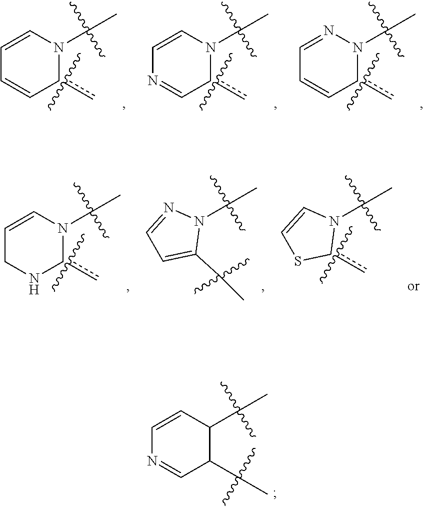
or,
ring Q is selected from the following groups optionally substituted with R¹⁰: phenyl, 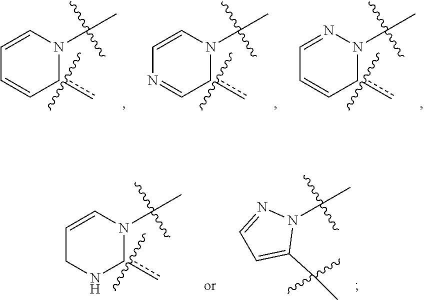
or,
ring Q is selected from 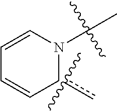
optionally substituted with R¹⁰; or,
ring Q is selected from phenyl optionally substituted with R¹⁰.

5. The compound of formula (I), or the stereoisomer or pharmaceutically acceptable salt thereof according to claim 1, wherein R¹⁰ is selected from halogen, ═O, NH₂, CN, C₁-C₆ alkyl, C₂-C₄ alkynyl, C₁-C₆ alkoxy, C₃-C₆ cycloalkyl, C₃-C₆ cycloalkyl-O— or C₃-C₆ cycloalkyl-NH—, wherein the NH₂, C₁-C₆ alkyl, C₂-C₄ alkynyl, C₁-C₆ alkoxy or C₃-C₆ cycloalkyl is optionally substituted with R^{10a}; or,
R¹⁰ is selected from halogen, ═O, NH₂, C₁-C₆ alkyl, C₂-C₄ alkynyl, C₁-C₆ alkoxy, C₃-C₆ cycloalkyl, C₃-C₆ cycloalkyl-O— or C₃-C₆ cycloalkyl-NH—, wherein the NH₂, C₁-C₆ alkyl, C₂-C₄ alkynyl, C₁-C₆ alkoxy or C₃-C₆ cycloalkyl is optionally substituted with R^{10a}; or,
R¹⁰ is selected from halogen, ═O, NH₂, C₁-C₆ alkyl, C₂-C₄ alkynyl, C₁-C₆ alkoxy, or C₃-C₆ cycloalkyl, wherein the NH₂, C₁-C₆ alkyl, C₂-C₄ alkynyl, C₁-C₆ alkoxy or C₃-C₆ cycloalkyl is optionally substituted with R^{10a}; or,
R¹⁰ is selected from halogen, NH₂, CN, C₁-C₆ alkyl, C₁-C₆ alkoxy or C₃-C₆ cycloalkyl, wherein the NH₂ or C₁-C₆ alkyl is optionally substituted with R^{10a}; or,
R¹⁰ is selected from halogen, C₁-C₆ alkyl optionally substituted with halogen, C₁-C₆ alkoxy or C₃-C₆ cycloalkyl.

6. The compound of formula (I), or the stereoisomer or pharmaceutically acceptable salt thereof according to claim 1, wherein R^{10a} is selected from halogen, OH, NH₂, C₁-C₆ alkyl, halo C₁-C₆ alkyl, C₁-C₆ alkoxy or halo C₁-C₆ alkoxy; or,
R^{10a} is selected from halogen, OH, C₁-C₆ alkyl or halo C₁-C₆ alkyl; or,
R^{10a} is selected from halogen or C₁-C₆ alkyl; or,
R^{10a} is selected from F or CH₃.

7. The compound of formula (I), or the stereoisomer or pharmaceutically acceptable salt thereof according to claim 1, wherein Y¹, Y² and Y⁴ are independently selected from CR^{b} or N, and Y³ is selected from CR^{b}; or,
Y¹ and Y² are independently selected from CR^{b} or N, and Y³ and Y⁴ are independently selected from CR^{b}; or,
Y¹, Y², Y³ and Y⁴ are all CR^{b}; or,
Y¹ and Y² are both N, and Y³ and Y⁴ are independently selected from CR^{b}; or,
Y¹ is N, and Y², Y³ and Y⁴ are independently selected from CR^{b}; or,
Y¹, Y² and Y³ are all CR^{b}, and Y⁴ is N; or,
Y¹, Y² and Y³ are all CH, and Y⁴ is CR^{b}; or,
Y¹ is N, Y² and Y³ are both CH, and Y⁴ is CR^{b}; or,
Y¹, Y² and Y³ are all CH, and Y⁴ is N.

8. The compound of formula (I), or the stereoisomer or pharmaceutically acceptable salt thereof according to claim 1, wherein X is 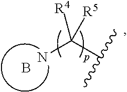
wherein ring B is selected from 5- to 10-membered nitrogen-containing heteroaryl, 4- to 7-membered monocyclic nitrogen-containing heterocyclyl or 6- to 10-membered nitrogen-containing heterocyclyl which is optionally substituted with R³; or,
ring B is selected from the following groups optionally substituted with R³; tetrahydropyrrolyl, piperidyl, piperazinyl, morpholinyl, 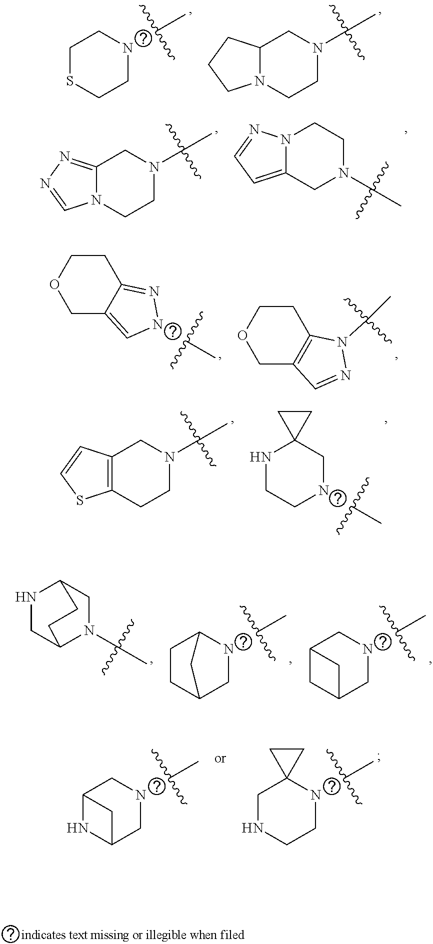
or,
ring B is selected from the following groups optionally substituted with R³: tetrahydropyrrolyl, piperidyl, piperazinyl, morpholinyl, 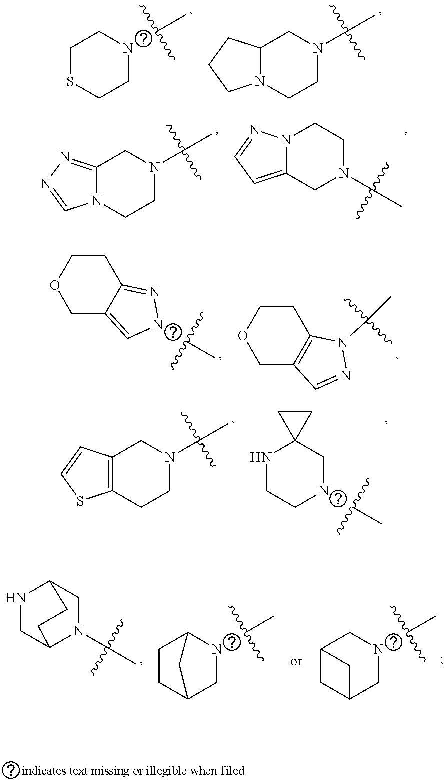
or,
ring B is selected from the following groups optionally substituted with R³: tetrahydropyrrolyl, piperidyl, piperazinyl, morpholinyl, 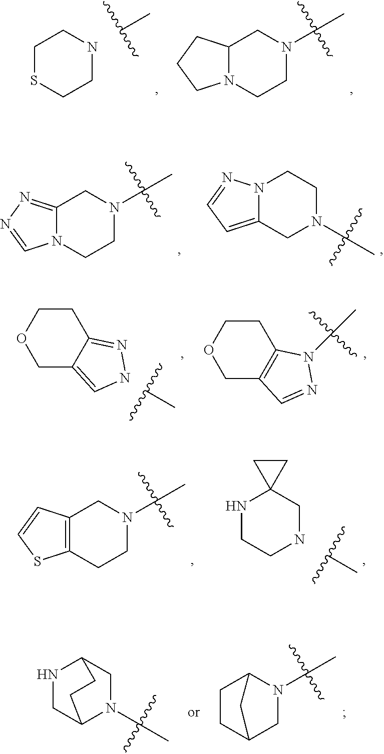
or,
ring B is selected from the following groups optionally substituted with R³: tetrahydropyrrolyl, piperidyl, piperazinyl, morpholinyl,

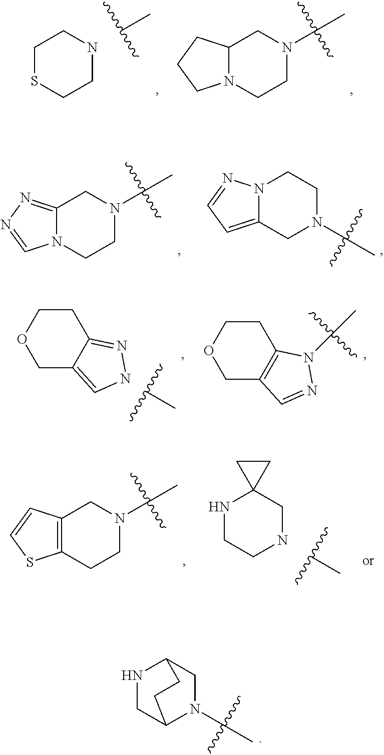

9. The compound of formula (I), or the stereoisomer or pharmaceutically acceptable salt thereof according to claim 1, wherein R³ is selected from halogen, OH, ═O, C₁-C₃ alkyl, C₃-C₆ cycloalkyl or phenyl, wherein the C₁-C₃ alkyl, C₃-C₆ cycloalkyl or phenyl is optionally further substituted with R^{3a}; or, R³ is selected from ═O, OH, F, methyl, isopropyl, CF₃, cyclopropyl or phenyl; or, R³ is selected from ═O, OH, F, methyl, CF₃, cyclopropyl or phenyl; and/or
wherein R⁴ and R⁵ are independently selected from H, halogen, OH or C₁-C₃ alkyl optionally substituted with R^{4a}; or R⁴ and R⁵ together with the atom to which they are attached form C₃-C₆ cycloalkyl optionally substituted with R^{8a}; or R⁴ and R⁵ together form ═O; or,
R⁴ and R⁵ are independently selected from H, methyl, hydroxymethyl or CF₃, or R⁴ and R⁵ together with the atom to which they are attached form cyclopropyl, or R⁴ and R⁵ together form ═O.

10. (canceled)

11. The compound of formula (I), or the stereoisomer or pharmaceutically acceptable salt thereof according to claim 1, wherein 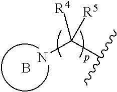
is selected from the following groups: 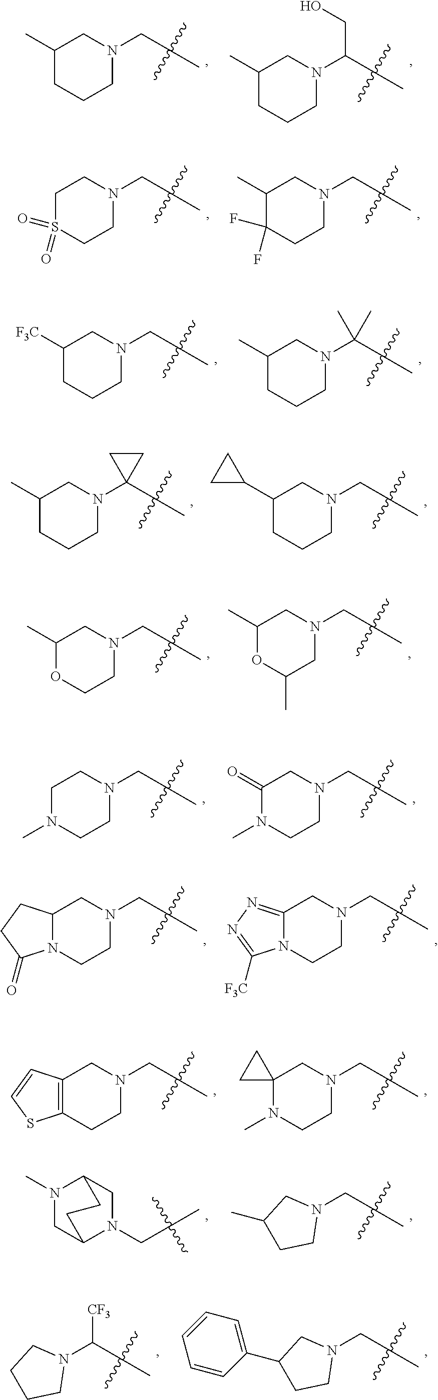 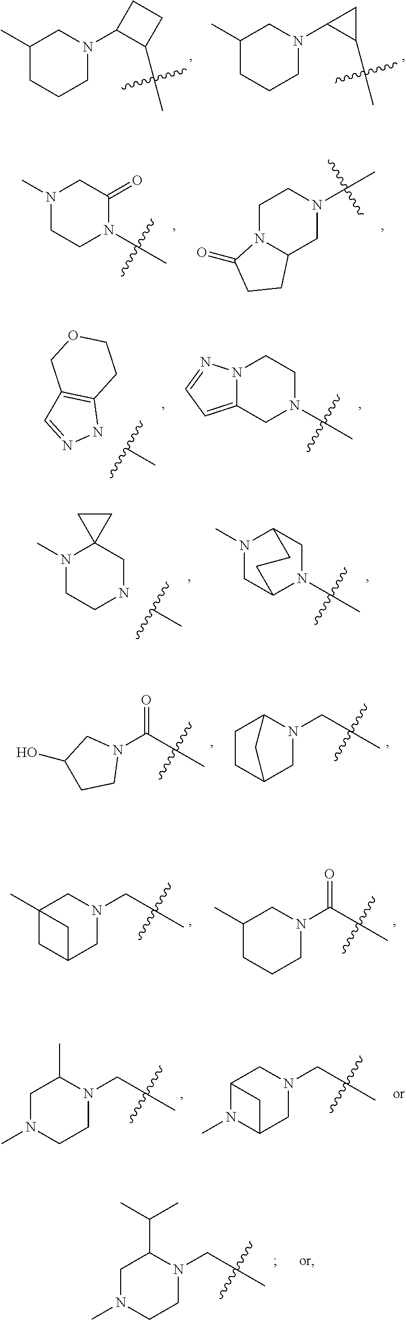
is selected from the following groups: 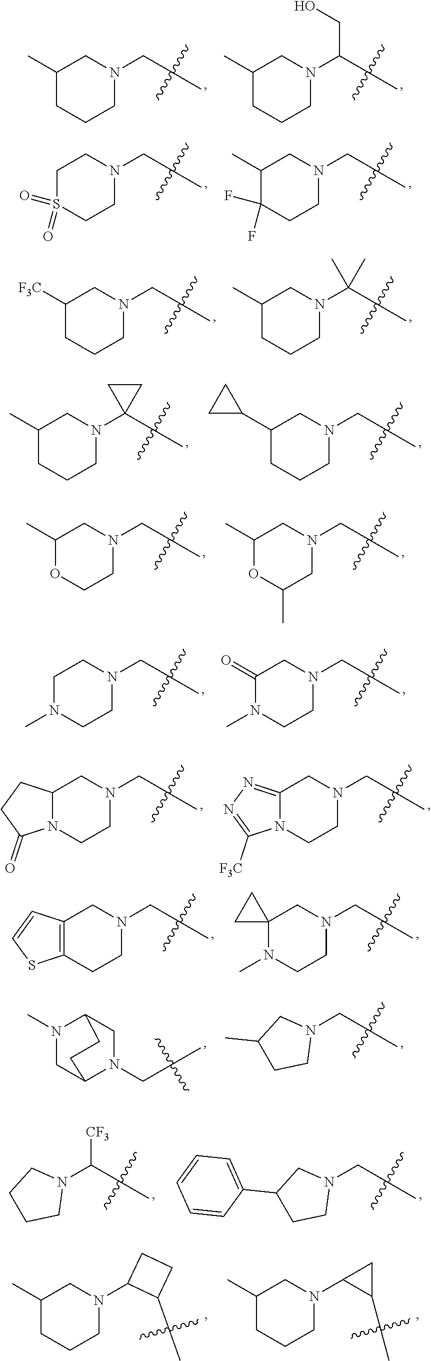 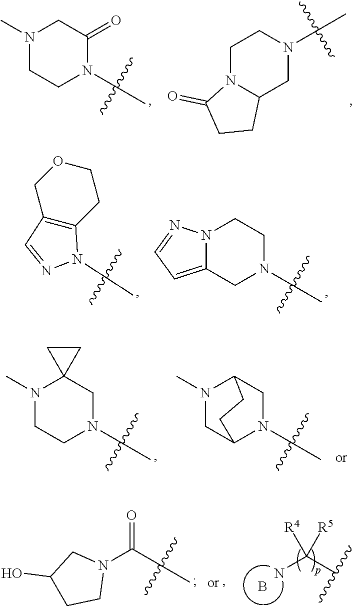
is selected from the following groups:

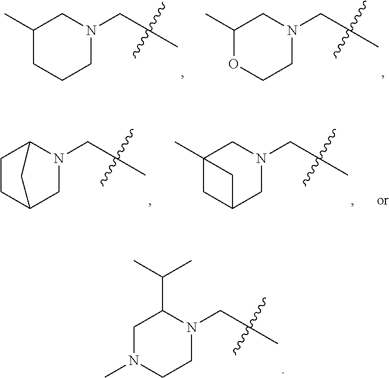

12. The compound of formula (I), or the stereoisomer or pharmaceutically acceptable salt thereof according to claim 1, wherein X is 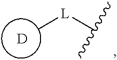
wherein ring D is selected from the following groups optionally substituted with R⁶: C₃-C₆ cycloalkyl, 5- to 6-membered heteroaryl, 4- to 7-membered monocyclic nitrogen-containing heterocyclyl or 6- to 10-membered nitrogen-containing heterocyclyl, and ring D is connected to L via a non-N atom; or,
ring D is selected from the following groups optionally substituted with R⁶: cyclopropyl, cyclobutyl, cyclopentyl, tetrahydropyrrolyl, piperidyl, piperazinyl, 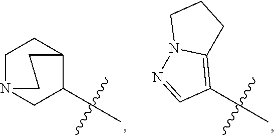 [SMILES: *C1CN2CCC1CC2 ; *c1cnn2c1CCC2]
pyrazolyl, imidazolyl, oxazolyl, isoxazolyl, triazolyl, thiazolyl, isothiazolyl or pyridyl; or,
ring D is selected from the following groups optionally substituted with R⁶: cyclobutyl, cyclopentyl, piperidyl, pyridyl,

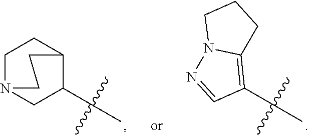 [SMILES: *C1CN2CCC1CC2 ; *c1cnn2c1CCC2]

13. The compound of formula (I), or the stereoisomer or pharmaceutically acceptable salt thereof according to claim 1, wherein R⁶ is selected from halogen, OH, CN, ═O or C₁-C₃ alkyl optionally substituted with R^{3a}; or, R⁶ is selected from halogen, ═O, OH or C₁-C₃ alkyl; or, R⁶ is selected from F or methyl.

14. The compound of formula (I), or the stereoisomer or pharmaceutically acceptable salt thereof according to claim 1, wherein L is selected from a bond, —NR⁷—, —NR⁷CR⁸R⁹—, —O— or —CR⁸R⁹—; or,
L is selected from a bond, —NR⁷—, —NR⁷CH₂—, —O— or —CR⁸R⁹—; or,
L is selected from a bond, —NH—, —NHCH₂—, —NHCH(CH₃)—, —O—, —C(F)₂— or —CH₂—; or,
L is selected from a bond, —NH—, —NHCH₂—, —O—, —C(F)₂— or —CH₂—.

15. The compound of formula (I), or the stereoisomer or pharmaceutically acceptable salt thereof according to claim 1, wherein 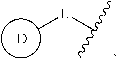
is selected from the following groups: 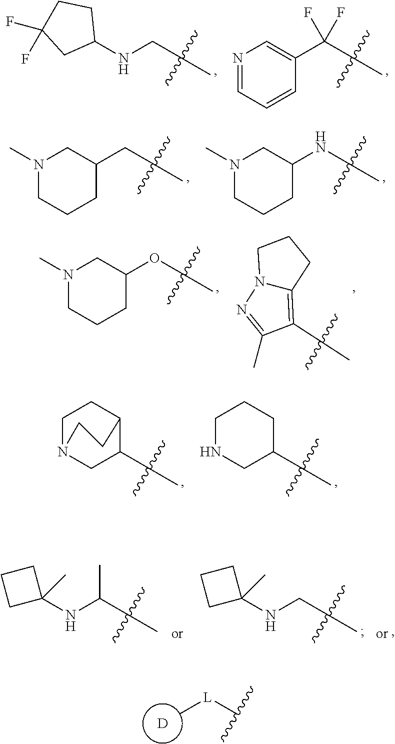
is selected from the following groups: 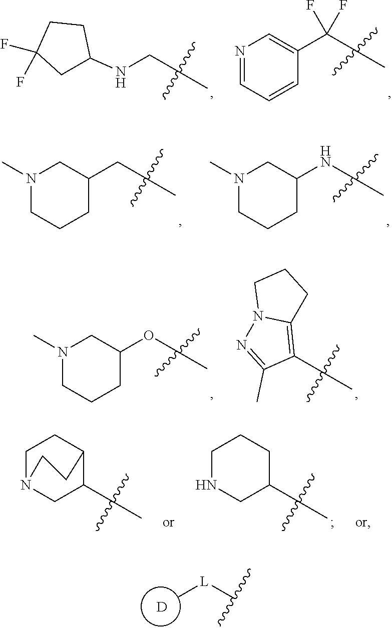
is selected from the following groups: 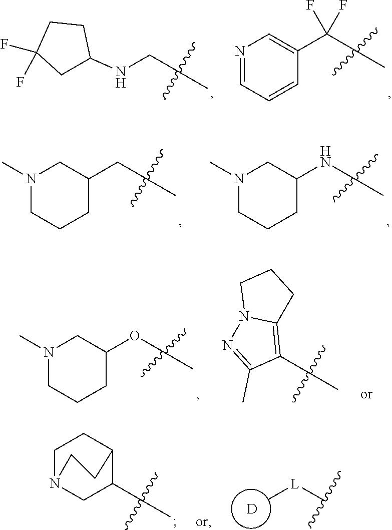
is selected from 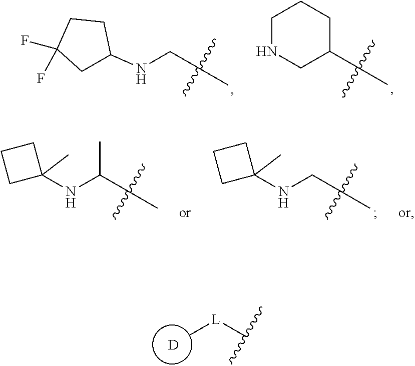
is selected from H or H

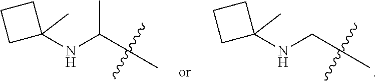

16. The compound of formula (I), or the stereoisomer or pharmaceutically acceptable salt thereof according to claim 1, wherein R¹ and R² together with the atom to which they are attached form C₃-C₆ cycloalkyl or 4- to 7-membered heterocyclyl, and the C₃-C₆ cycloalkyl or 4- to 7-membered heterocyclyl is optionally substituted with R^{1b}; or,
R¹ and R² together with the atom to which they are attached form the following groups optionally substituted with R^{1b}: cyclobutyl, spiro[2,3]hexyl or oxetanyl; or,
R¹ and R² are independently selected from H, halogen, CN, C₁-C₃ alkyl, C₁-C₃ alkoxy or C₃-C₆ cycloalkyl, wherein the C₁-C₃ alkyl, C₁-C₃ alkoxy or C₃-C₆ cycloalkyl is optionally substituted with R^{1a}; or,
R¹ and R² are independently selected from H, C₁-C₃ alkyl or C₃-C₆ cycloalkyl, wherein the C₁-C₃ alkyl or C₃-C₆ cycloalkyl is optionally substituted with R^{1a}; or,
R¹ and R² are independently selected from H or C₁-C₃ alkyl, wherein the C₁-C₃ alkyl is optionally substituted with R^{1a}; or,
R¹ and R² are independently selected from H, F, cyclopropyl, methyl or 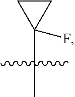 [SMILES: *C1(F)CC1]
or
R¹ and R² together with the atom to which they are attached form the following groups:

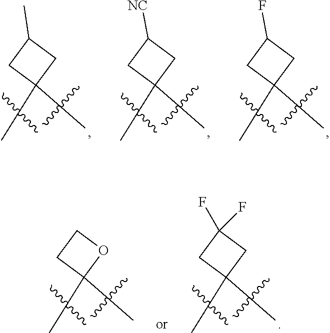

17. The compound of formula (I), or the stereoisomer or pharmaceutically acceptable salt thereof according to claim 1, wherein
W₁ is selected from 5- to 10-membered heteroaryl or 6- to 10-membered heterocyclyl optionally substituted with R¹⁵; or,
W₁ is selected from 5- to 10-membered heteroaryl optionally substituted with R¹⁵; or,
W₁ is selected from 5-membered heteroaryl or 8-membered heterocyclyl optionally substituted with R¹⁵; or,
W₁ is selected from the following groups optionally substituted with R¹⁵: pyrrolyl, thienyl, furyl, pyrazolyl, imidazolyl, thiazolyl, isothiazolyl, thiadiazolyl, triazolyl, oxazolyl, isoxazolyl, oxadiazolyl or 6,7-dihydro-5H-pyrrolo[2,1-c][1,2,4]triazolyl; or,
W₁ is selected from the following groups optionally substituted with R¹⁵: pyrrolyl, thienyl, furyl, pyrazolyl, imidazolyl, thiazolyl, isothiazolyl, thiadiazolyl, triazolyl, oxazolyl, isoxazolyl or oxadiazolyl; or,
W₁ is selected from the following groups optionally substituted with R¹⁵: triazolyl, oxazolyl, isoxazolyl, oxadiazolyl or 6,7-dihydro-5H-pyrrolo[2,1-c][1,2,4]triazolyl; or,
W₁ is selected from the following groups optionally substituted with R¹⁵: triazolyl, oxazolyl, isoxazolyl or oxadiazolyl.

18. The compound of formula (I), or the stereoisomer or pharmaceutically acceptable salt thereof according to claim 1, wherein R¹⁵ is selected from halogen, OH, NH₂, C₁-C₃ alkyl or C₃-C₆ cycloalkyl, wherein the C₁-C₃ alkyl or C₃-C₆ cycloalkyl is optionally substituted with R^{15a}; or,
R¹⁵ is selected from OH, C₁-C₃ alkyl or C₃-C₆ cycloalkyl, wherein the C₁-C₃ alkyl or C₃-C₆ cycloalkyl is optionally substituted with R^{15a}; or,
R¹⁵ is selected from methyl or cyclopropyl, wherein the methyl or cyclopropyl is optionally substituted with R^{15a}; or,
R¹⁵ is selected from methyl, CHF₂ or cyclopropyl.

19. The compound of formula (I), or the stereoisomer or pharmaceutically acceptable salt thereof according to claim 1, wherein the compound of formula (I), or the stereoisomer or pharmaceutically acceptable salt thereof is selected from a compound of formula (II), or a stereoisomer or pharmaceutically acceptable salt thereof: 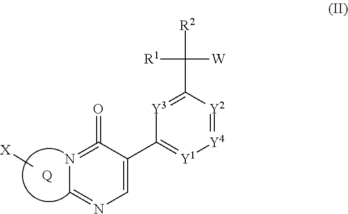
wherein, Y¹, Y², Y³, Y⁴, X, Q, W, R¹ and R² are as defined in claim 1; or
wherein the compound of formula (I), or the stereoisomer or pharmaceutically acceptable salt thereof is selected from a compound of formula (III), or a stereoisomer or pharmaceutically acceptable salt thereof: 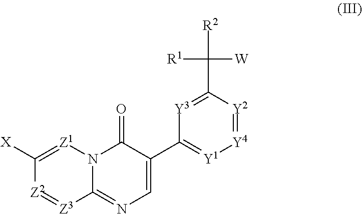
wherein, Z₁, Z₂ and Z₃ are independently selected from CH, CR¹⁰ or N; R¹⁰, Y¹, Y², Y³, Y⁴, X, W, R¹ and R² are as defined in claim 1; or
wherein the compound of formula (I), or the stereoisomer or pharmaceutically acceptable salt thereof is selected from a compound of formula (V), or a stereoisomer or pharmaceutically acceptable salt thereof: 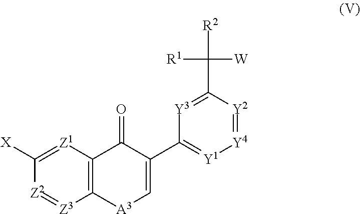
wherein, Z¹, Z² and Z³ are independently selected from CH, CR¹⁰ or N; A³ is selected from CR^{11a}R^{11b}, NR¹², O or S; R¹⁰, R^{11a}, R^{11b}, R¹², Y¹, Y², Y³, Y⁴, X, W, R¹ and R² are as defined in claim 1; or
wherein the compound of formula (I), or the stereoisomer or pharmaceutically acceptable salt thereof is selected from a compound of formula (VI), or a stereoisomer or pharmaceutically acceptable salt thereof: 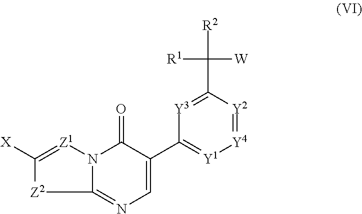
wherein, Z¹ is selected from CH, CR¹⁰ or N; Z² is selected from CR¹⁰, N, O or S; R¹⁰, Y¹, Y², Y³, Y⁴, X, W, R¹ and R² are as defined in claim 1.

20.-22. (canceled)

23. The compound of formula (I), or the stereoisomer or pharmaceutically acceptable salt thereof according to claim 1, wherein the compound is selected from one of the following structures or a pharmaceutically acceptable salt thereof:

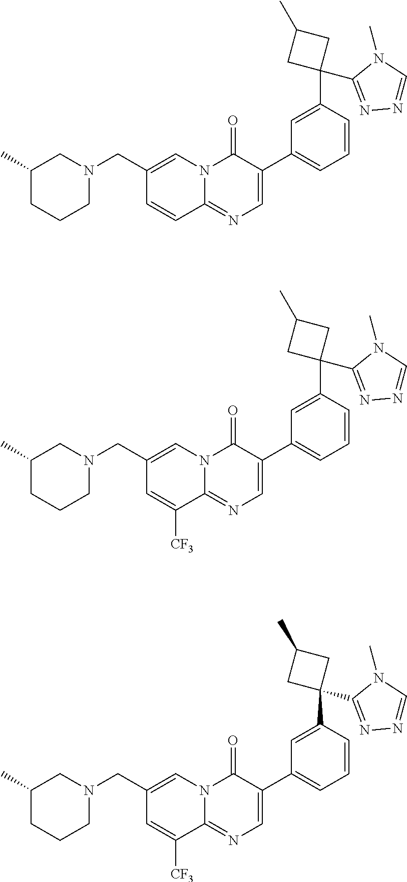 [SMILES: CC1CC(c2cccc(-c3cnc4ccc(CN5CCC[C@H](C)C5)cn4c3=O)c2)(c2nncn2C)C1 ; CC1CC(c2cccc(-c3cnc4c(C(F)(F)F)cc(CN5CCC[C@H](C)C5)cn4c3=O)c2)(c2nncn2C)C1 ; C[C@H]1CCCN(Cc2cc(C(F)(F)F)c3ncc(-c4cccc([C@]5(c6nncn6C)C[C@H](C)C5)c4)c(=O)n3c2)C1]

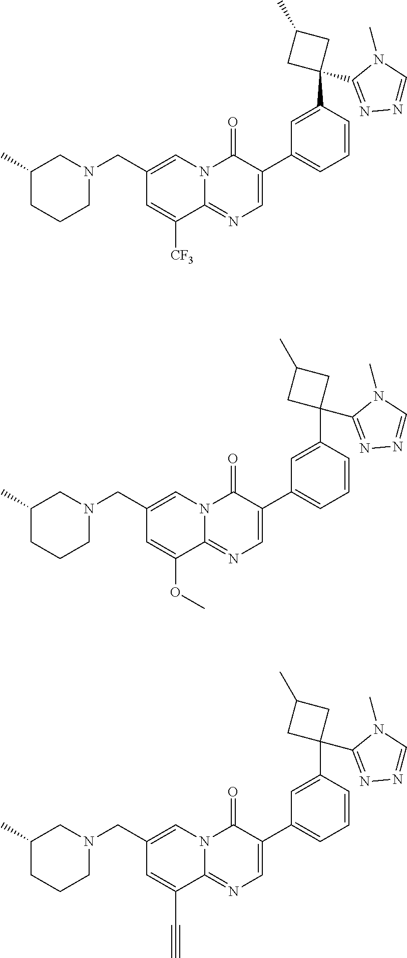 [SMILES: C[C@H]1CCCN(Cc2cc(C(F)(F)F)c3ncc(-c4cccc([C@]5(c6nncn6C)C[C@@H](C)C5)c4)c(=O)n3c2)C1 ; COc1cc(CN2CCC[C@H](C)C2)cn2c(=O)c(-c3cccc(C4(c5nncn5C)CC(C)C4)c3)cnc12 ; C#Cc1cc(CN2CCC[C@H](C)C2)cn2c(=O)c(-c3cccc(C4(c5nncn5C)CC(C)C4)c3)cnc12]

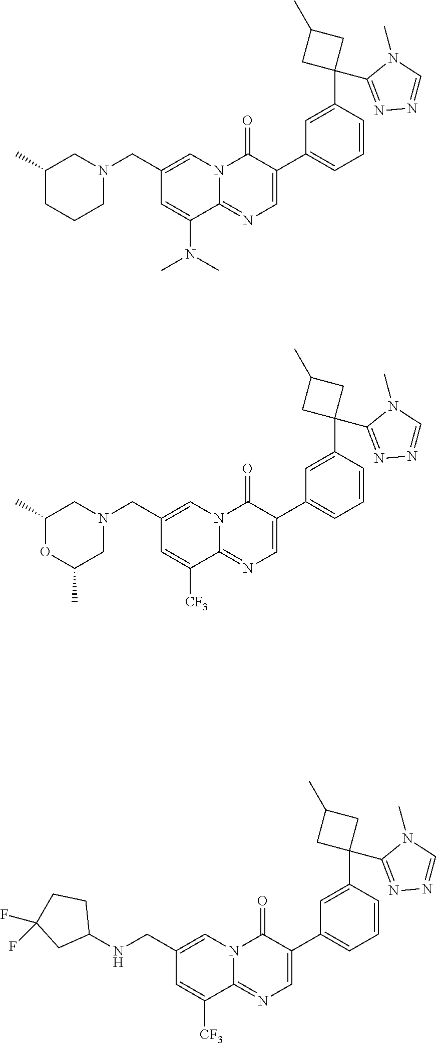 [SMILES: CC1CC(c2cccc(-c3cnc4c(N(C)C)cc(CN5CCC[C@H](C)C5)cn4c3=O)c2)(c2nncn2C)C1 ; CC1CC(c2cccc(-c3cnc4c(C(F)(F)F)cc(CN5C[C@@H](C)O[C@@H](C)C5)cn4c3=O)c2)(c2nncn2C)C1 ; CC1CC(c2cccc(-c3cnc4c(C(F)(F)F)cc(CNC5CCC(F)(F)C5)cn4c3=O)c2)(c2nncn2C)C1]

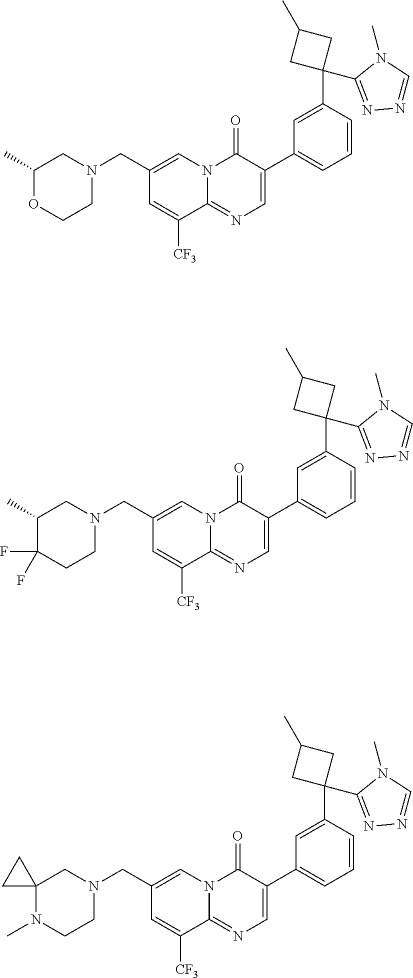 [SMILES: CC1CC(c2cccc(-c3cnc4c(C(F)(F)F)cc(CN5CCO[C@H](C)C5)cn4c3=O)c2)(c2nncn2C)C1 ; CC1CC(c2cccc(-c3cnc4c(C(F)(F)F)cc(CN5CCC(F)(F)[C@H](C)C5)cn4c3=O)c2)(c2nncn2C)C1 ; CC1CC(c2cccc(-c3cnc4c(C(F)(F)F)cc(CN5CCN(C)C6(CC6)C5)cn4c3=O)c2)(c2nncn2C)C1]

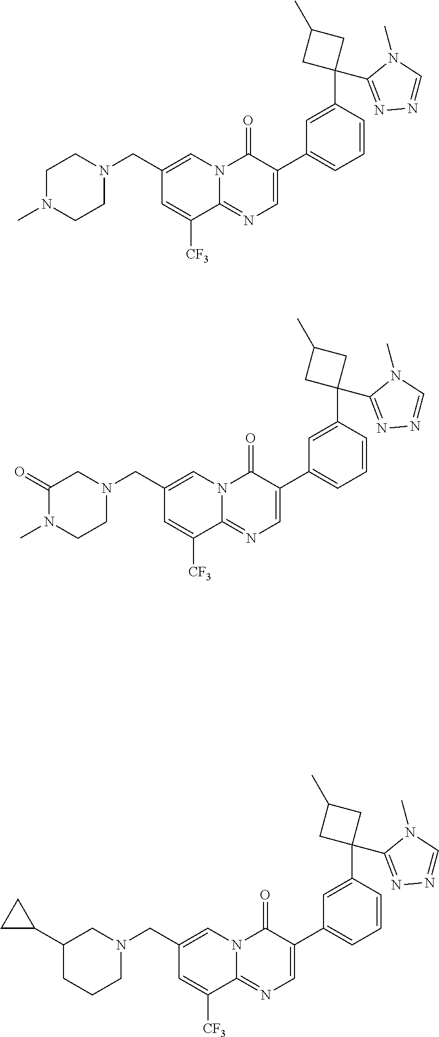 [SMILES: CC1CC(c2cccc(-c3cnc4c(C(F)(F)F)cc(CN5CCN(C)CC5)cn4c3=O)c2)(c2nncn2C)C1 ; CC1CC(c2cccc(-c3cnc4c(C(F)(F)F)cc(CN5CCN(C)C(=O)C5)cn4c3=O)c2)(c2nncn2C)C1 ; CC1CC(c2cccc(-c3cnc4c(C(F)(F)F)cc(CN5CCCC(C6CC6)C5)cn4c3=O)c2)(c2nncn2C)C1]

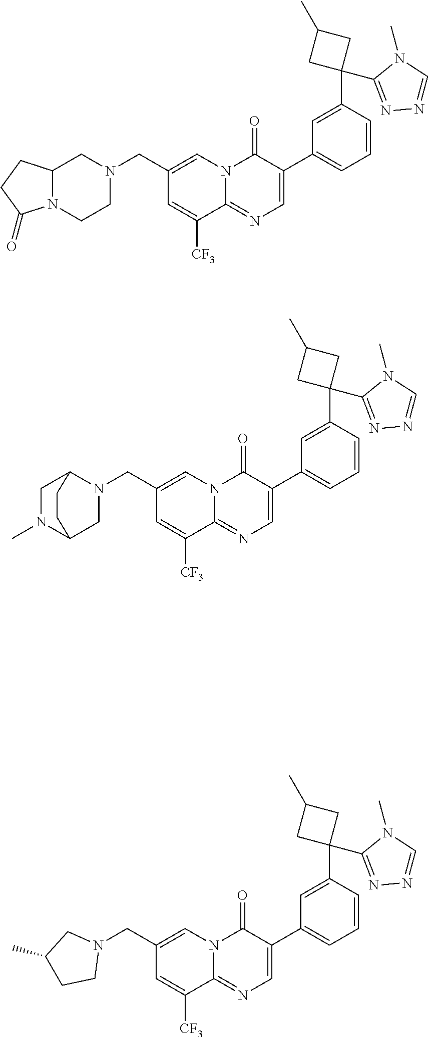 [SMILES: CC1CC(c2cccc(-c3cnc4c(C(F)(F)F)cc(CN5CCN6C(=O)CCC6C5)cn4c3=O)c2)(c2nncn2C)C1 ; CC1CC(c2cccc(-c3cnc4c(C(F)(F)F)cc(CN5CC6CCC5CN6C)cn4c3=O)c2)(c2nncn2C)C1 ; CC1CC(c2cccc(-c3cnc4c(C(F)(F)F)cc(CN5CC[C@H](C)C5)cn4c3=O)c2)(c2nncn2C)C1]

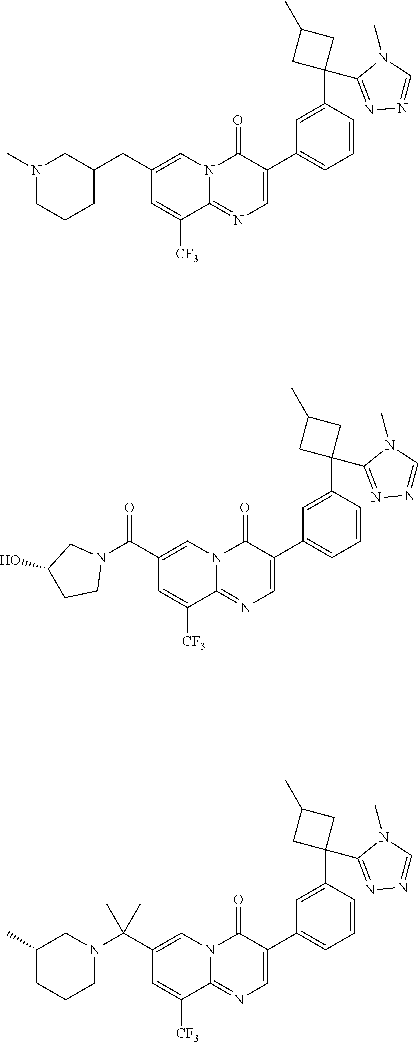 [SMILES: CC1CC(c2cccc(-c3cnc4c(C(F)(F)F)cc(CC5CCCN(C)C5)cn4c3=O)c2)(c2nncn2C)C1 ; CC1CC(c2cccc(-c3cnc4c(C(F)(F)F)cc(C(=O)N5CC[C@H](O)C5)cn4c3=O)c2)(c2nncn2C)C1 ; CC1CC(c2cccc(-c3cnc4c(C(F)(F)F)cc(C(C)(C)N5CCC[C@H](C)C5)cn4c3=O)c2)(c2nncn2C)C1]

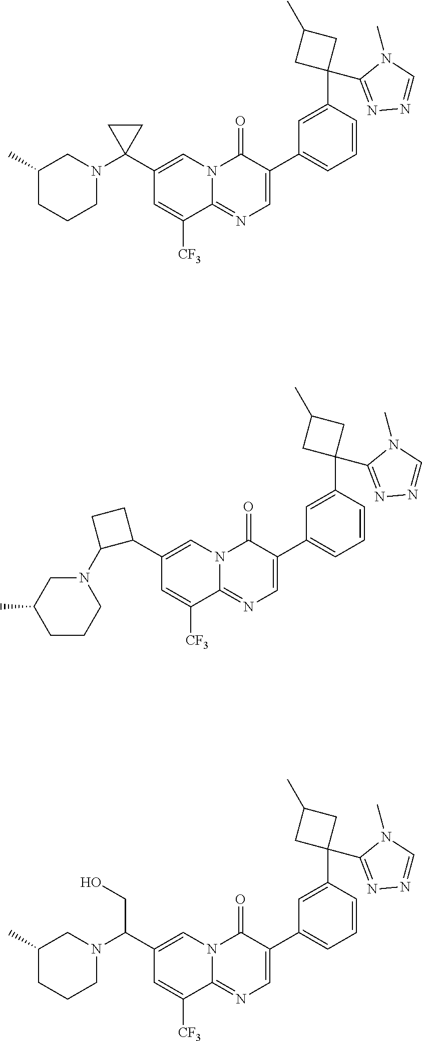 [SMILES: CC1CC(c2cccc(-c3cnc4c(C(F)(F)F)cc(C5(N6CCC[C@H](C)C6)CC5)cn4c3=O)c2)(c2nncn2C)C1 ; CC1CC(c2cccc(-c3cnc4c(C(F)(F)F)cc(C5CCC5N5CCC[C@H](C)C5)cn4c3=O)c2)(c2nncn2C)C1 ; CC1CC(c2cccc(-c3cnc4c(C(F)(F)F)cc(C(CO)N5CCC[C@H](C)C5)cn4c3=O)c2)(c2nncn2C)C1]

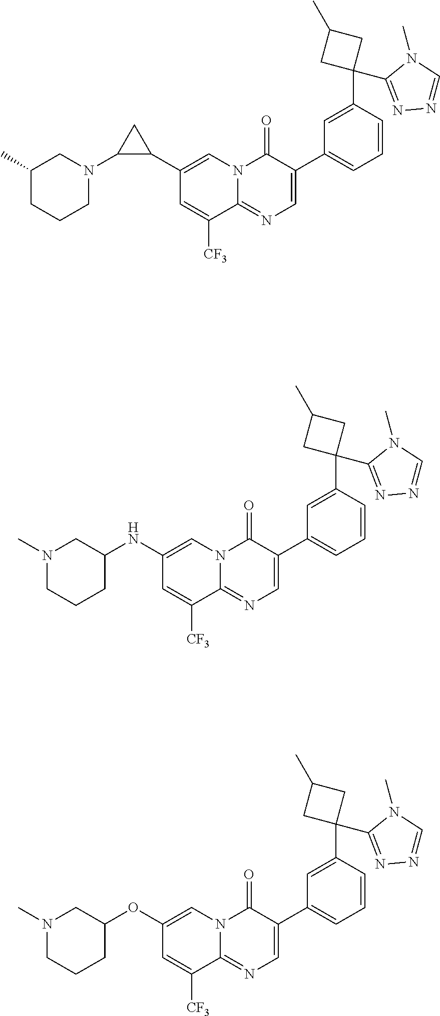 [SMILES: CC1CC(c2cccc(-c3cnc4c(C(F)(F)F)cc(C5CC5N5CCC[C@H](C)C5)cn4c3=O)c2)(c2nncn2C)C1 ; CC1CC(c2cccc(-c3cnc4c(C(F)(F)F)cc(NC5CCCN(C)C5)cn4c3=O)c2)(c2nncn2C)C1 ; CC1CC(c2cccc(-c3cnc4c(C(F)(F)F)cc(OC5CCCN(C)C5)cn4c3=O)c2)(c2nncn2C)C1]

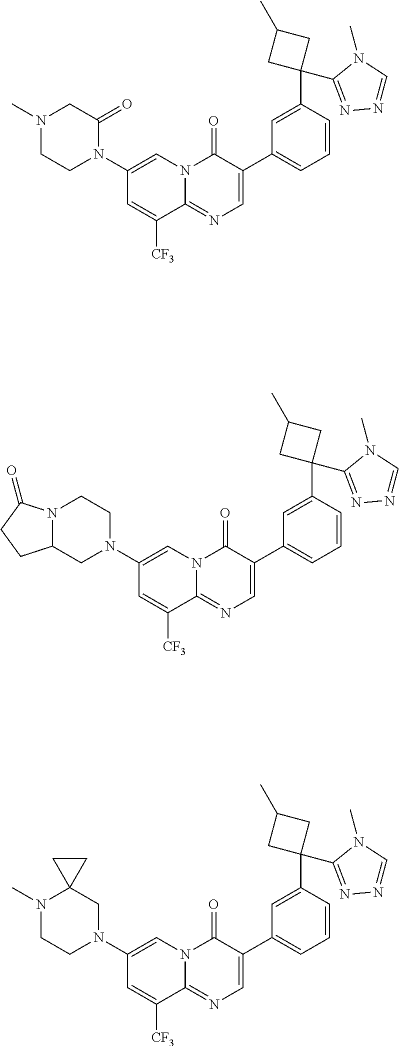 [SMILES: CC1CC(c2cccc(-c3cnc4c(C(F)(F)F)cc(N5CCN(C)CC5=O)cn4c3=O)c2)(c2nncn2C)C1 ; CC1CC(c2cccc(-c3cnc4c(C(F)(F)F)cc(N5CCN6C(=O)CCC6C5)cn4c3=O)c2)(c2nncn2C)C1 ; CC1CC(c2cccc(-c3cnc4c(C(F)(F)F)cc(N5CCN(C)C6(CC6)C5)cn4c3=O)c2)(c2nncn2C)C1]

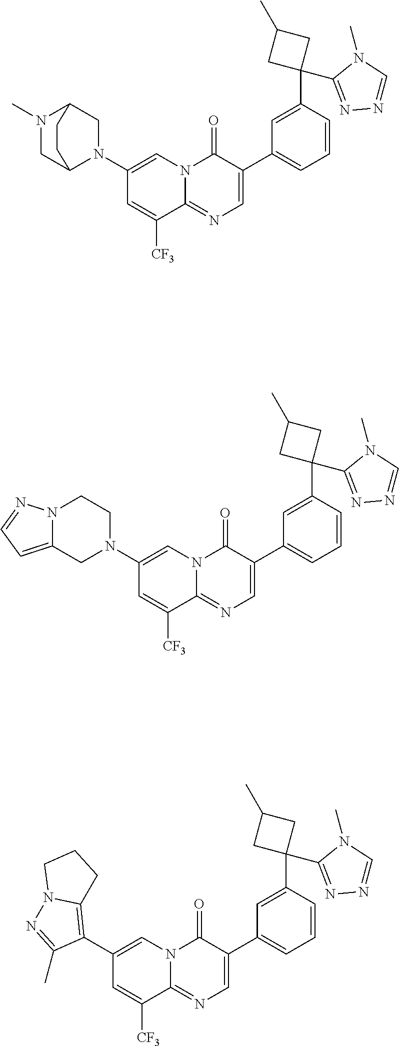 [SMILES: CC1CC(c2cccc(-c3cnc4c(C(F)(F)F)cc(N5CC6CCC5CN6C)cn4c3=O)c2)(c2nncn2C)C1 ; CC1CC(c2cccc(-c3cnc4c(C(F)(F)F)cc(N5CCn6nccc6C5)cn4c3=O)c2)(c2nncn2C)C1 ; Cc1nn2c(c1-c1cc(C(F)(F)F)c3ncc(-c4cccc(C5(c6nncn6C)CC(C)C5)c4)c(=O)n3c1)CCC2]

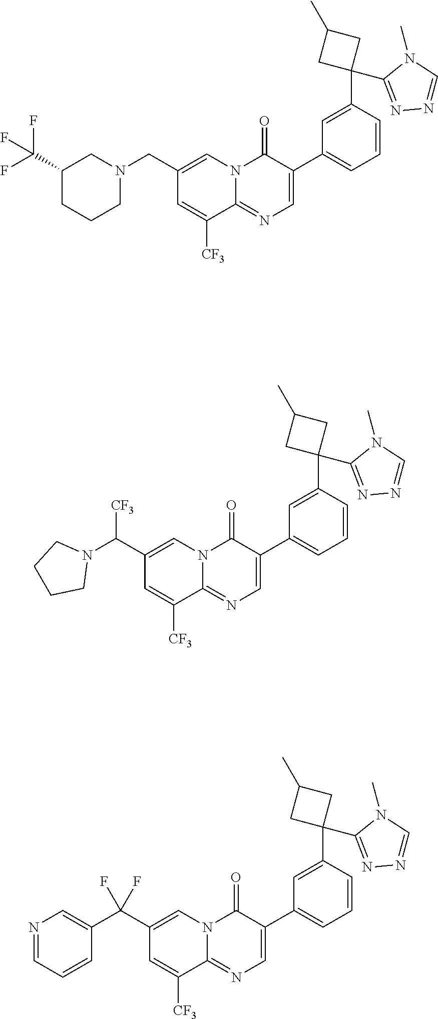 [SMILES: CC1CC(c2cccc(-c3cnc4c(C(F)(F)F)cc(CN5CCC[C@H](C(F)(F)F)C5)cn4c3=O)c2)(c2nncn2C)C1 ; CC1CC(c2cccc(-c3cnc4c(C(F)(F)F)cc(C(N5CCCC5)C(F)(F)F)cn4c3=O)c2)(c2nncn2C)C1 ; CC1CC(c2cccc(-c3cnc4c(C(F)(F)F)cc(C(F)(F)c5cccnc5)cn4c3=O)c2)(c2nncn2C)C1]

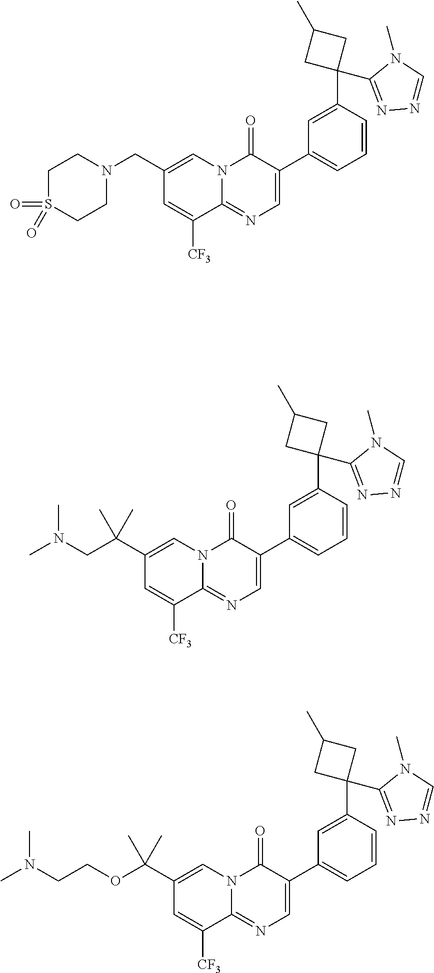 [SMILES: CC1CC(c2cccc(-c3cnc4c(C(F)(F)F)cc(CN5CCS(=O)(=O)CC5)cn4c3=O)c2)(c2nncn2C)C1 ; CC1CC(c2cccc(-c3cnc4c(C(F)(F)F)cc(C(C)(C)CN(C)C)cn4c3=O)c2)(c2nncn2C)C1 ; CC1CC(c2cccc(-c3cnc4c(C(F)(F)F)cc(C(C)(C)OCCN(C)C)cn4c3=O)c2)(c2nncn2C)C1]

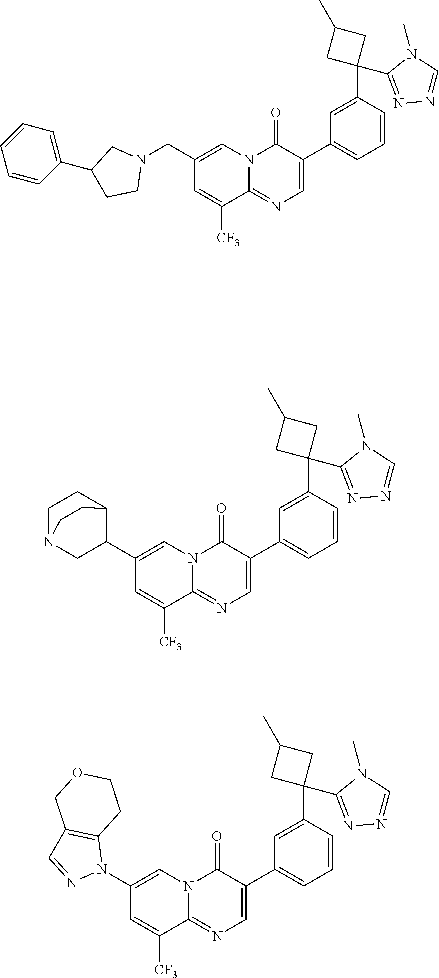 [SMILES: CC1CC(c2cccc(-c3cnc4c(C(F)(F)F)cc(CN5CCC(c6ccccc6)C5)cn4c3=O)c2)(c2nncn2C)C1 ; CC1CC(c2cccc(-c3cnc4c(C(F)(F)F)cc(C5CN6CCC5CC6)cn4c3=O)c2)(c2nncn2C)C1 ; CC1CC(c2cccc(-c3cnc4c(C(F)(F)F)cc(-n5ncc6c5CCOC6)cn4c3=O)c2)(c2nncn2C)C1]

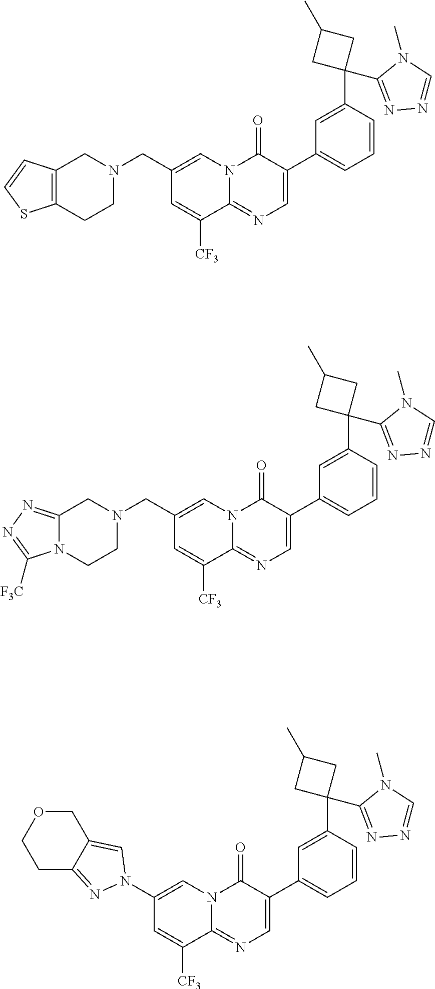 [SMILES: CC1CC(c2cccc(-c3cnc4c(C(F)(F)F)cc(CN5CCc6sccc6C5)cn4c3=O)c2)(c2nncn2C)C1 ; CC1CC(c2cccc(-c3cnc4c(C(F)(F)F)cc(CN5CCn6c(nnc6C(F)(F)F)C5)cn4c3=O)c2)(c2nncn2C)C1 ; CC1CC(c2cccc(-c3cnc4c(C(F)(F)F)cc(-n5cc6c(n5)CCOC6)cn4c3=O)c2)(c2nncn2C)C1]

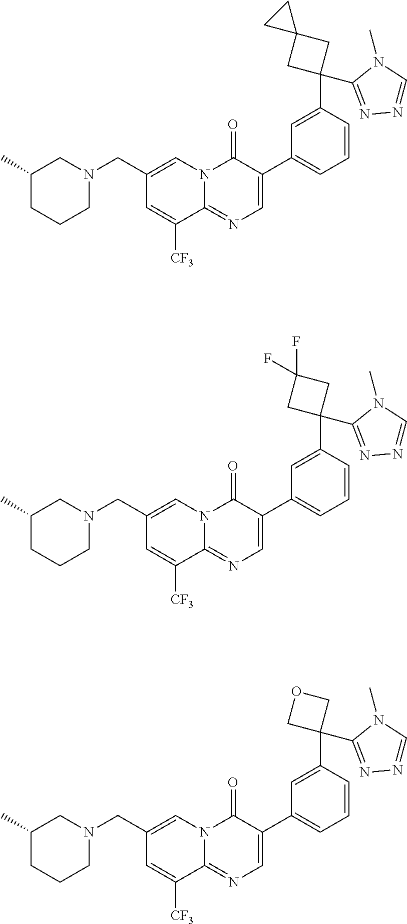 [SMILES: C[C@H]1CCCN(Cc2cc(C(F)(F)F)c3ncc(-c4cccc(C5(c6nncn6C)CC6(CC6)C5)c4)c(=O)n3c2)C1 ; C[C@H]1CCCN(Cc2cc(C(F)(F)F)c3ncc(-c4cccc(C5(c6nncn6C)CC(F)(F)C5)c4)c(=O)n3c2)C1 ; C[C@H]1CCCN(Cc2cc(C(F)(F)F)c3ncc(-c4cccc(C5(c6nncn6C)COC5)c4)c(=O)n3c2)C1]

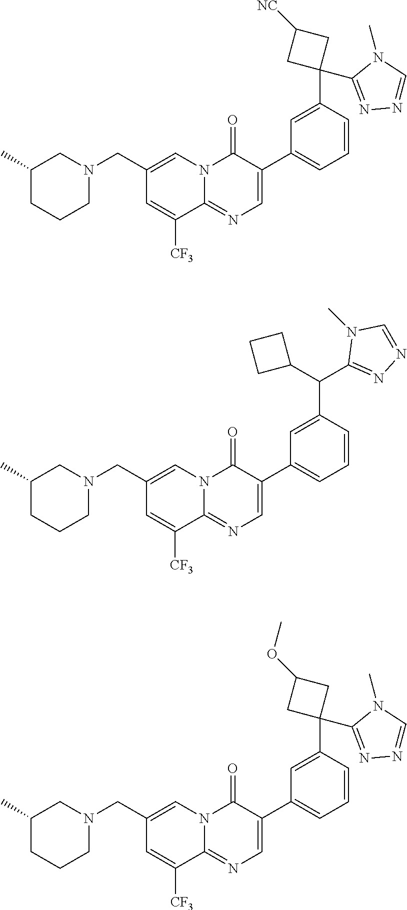 [SMILES: C[C@H]1CCCN(Cc2cc(C(F)(F)F)c3ncc(-c4cccc(C5(c6nncn6C)CC(C#N)C5)c4)c(=O)n3c2)C1 ; C[C@H]1CCCN(Cc2cc(C(F)(F)F)c3ncc(-c4cccc(C(c5nncn5C)C5CCC5)c4)c(=O)n3c2)C1 ; COC1CC(c2cccc(-c3cnc4c(C(F)(F)F)cc(CN5CCC[C@H](C)C5)cn4c3=O)c2)(c2nncn2C)C1]

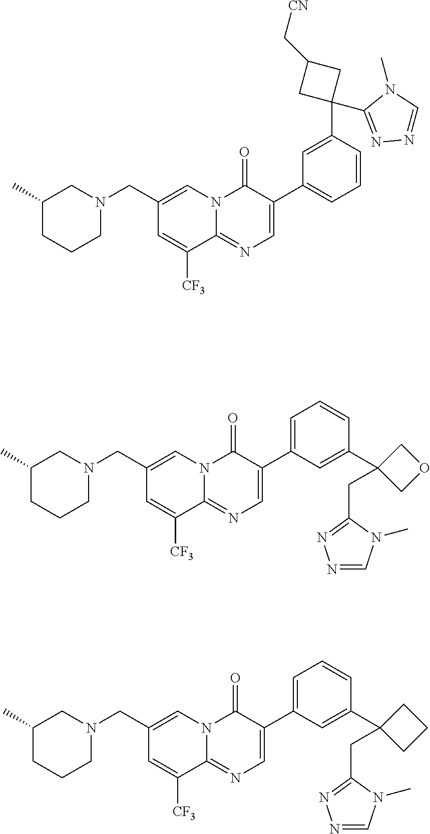 [SMILES: C[C@H]1CCCN(Cc2cc(C(F)(F)F)c3ncc(-c4cccc(C5(c6nncn6C)CC(CC#N)C5)c4)c(=O)n3c2)C1 ; C[C@H]1CCCN(Cc2cc(C(F)(F)F)c3ncc(-c4cccc(C5(Cc6nncn6C)COC5)c4)c(=O)n3c2)C1 ; C[C@H]1CCCN(Cc2cc(C(F)(F)F)c3ncc(-c4cccc(C5(Cc6nncn6C)CCC5)c4)c(=O)n3c2)C1]

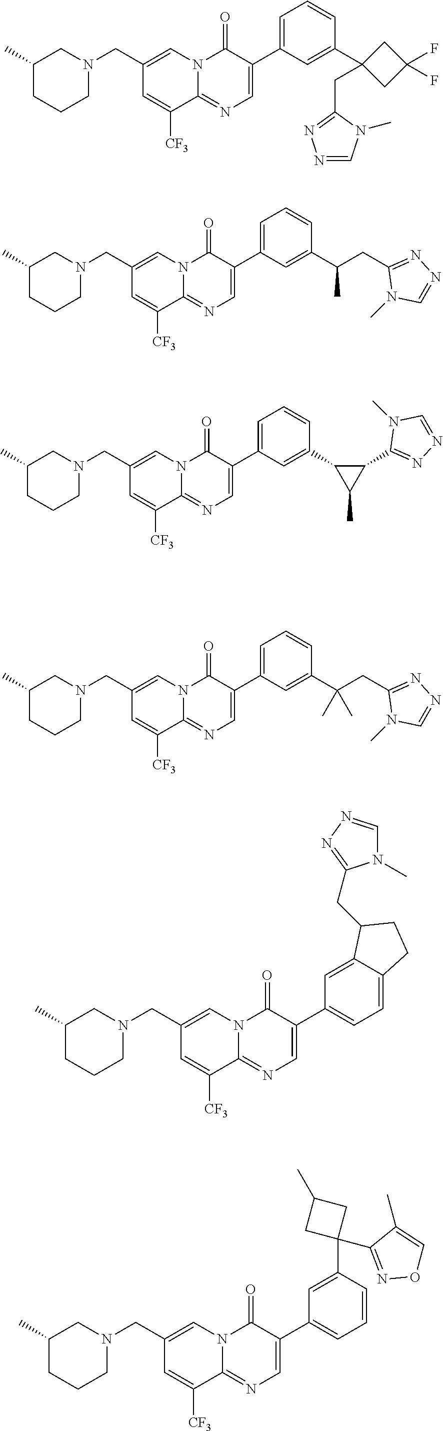 [SMILES: C[C@H]1CCCN(Cc2cc(C(F)(F)F)c3ncc(-c4cccc(C5(Cc6nncn6C)CC(F)(F)C5)c4)c(=O)n3c2)C1 ; C[C@H]1CCCN(Cc2cc(C(F)(F)F)c3ncc(-c4cccc([C@H](C)Cc5nncn5C)c4)c(=O)n3c2)C1 ; C[C@H]1CCCN(Cc2cc(C(F)(F)F)c3ncc(-c4cccc([C@H]5[C@H](C)[C@H]5c5nncn5C)c4)c(=O)n3c2)C1 ; C[C@H]1CCCN(Cc2cc(C(F)(F)F)c3ncc(-c4cccc(C(C)(C)Cc5nncn5C)c4)c(=O)n3c2)C1 ; C[C@H]1CCCN(Cc2cc(C(F)(F)F)c3ncc(-c4ccc5c(c4)C(Cc4nncn4C)CC5)c(=O)n3c2)C1 ; Cc1conc1C1(c2cccc(-c3cnc4c(C(F)(F)F)cc(CN5CCC[C@H](C)C5)cn4c3=O)c2)CC(C)C1]

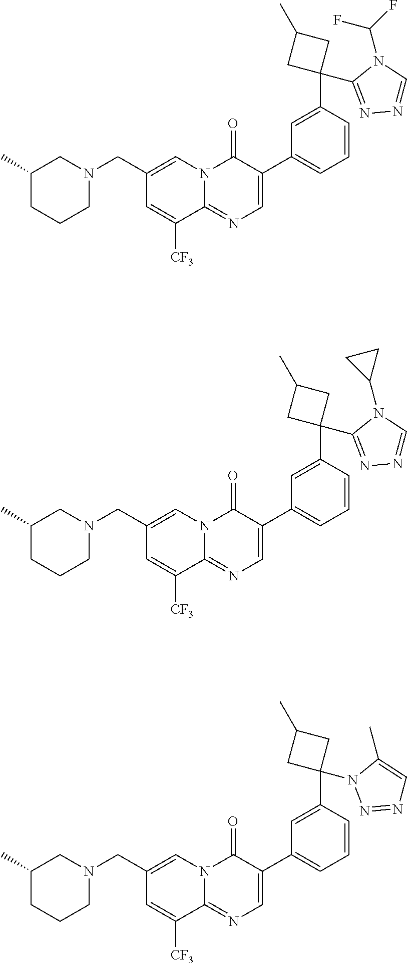 [SMILES: CC1CC(c2cccc(-c3cnc4c(C(F)(F)F)cc(CN5CCC[C@H](C)C5)cn4c3=O)c2)(c2nncn2C(F)F)C1 ; CC1CC(c2cccc(-c3cnc4c(C(F)(F)F)cc(CN5CCC[C@H](C)C5)cn4c3=O)c2)(c2nncn2C2CC2)C1 ; Cc1cnnn1C1(c2cccc(-c3cnc4c(C(F)(F)F)cc(CN5CCC[C@H](C)C5)cn4c3=O)c2)CC(C)C1]

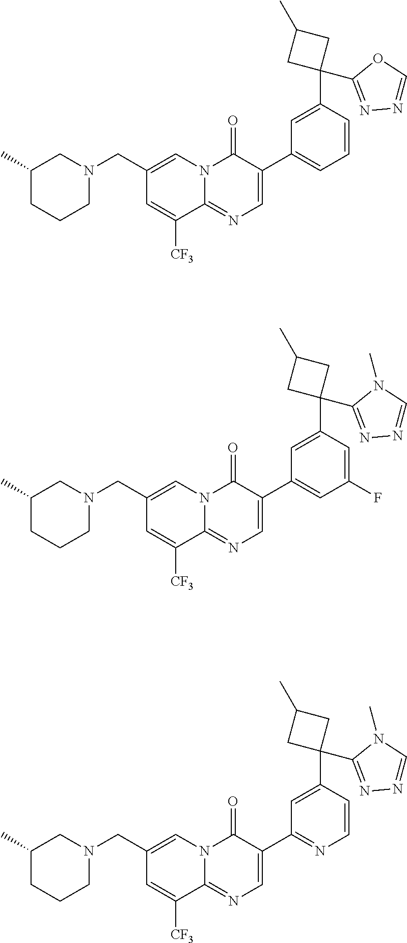 [SMILES: CC1CC(c2cccc(-c3cnc4c(C(F)(F)F)cc(CN5CCC[C@H](C)C5)cn4c3=O)c2)(c2nnco2)C1 ; CC1CC(c2cc(F)cc(-c3cnc4c(C(F)(F)F)cc(CN5CCC[C@H](C)C5)cn4c3=O)c2)(c2nncn2C)C1 ; CC1CC(c2ccnc(-c3cnc4c(C(F)(F)F)cc(CN5CCC[C@H](C)C5)cn4c3=O)c2)(c2nncn2C)C1]

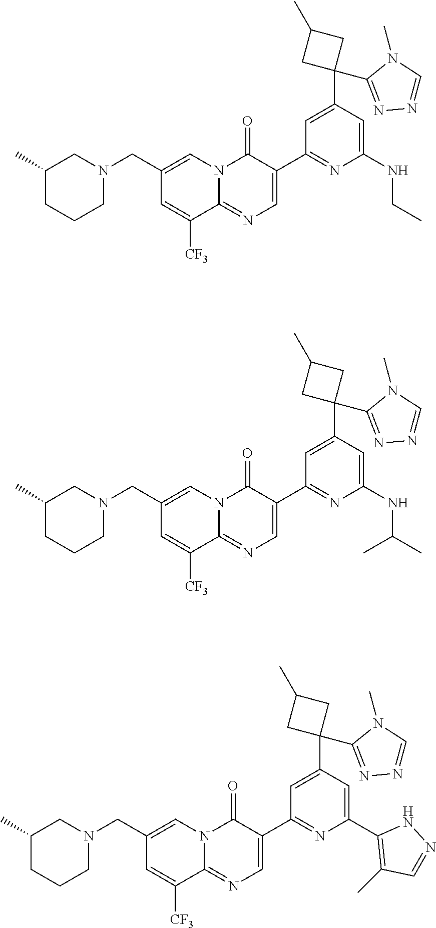

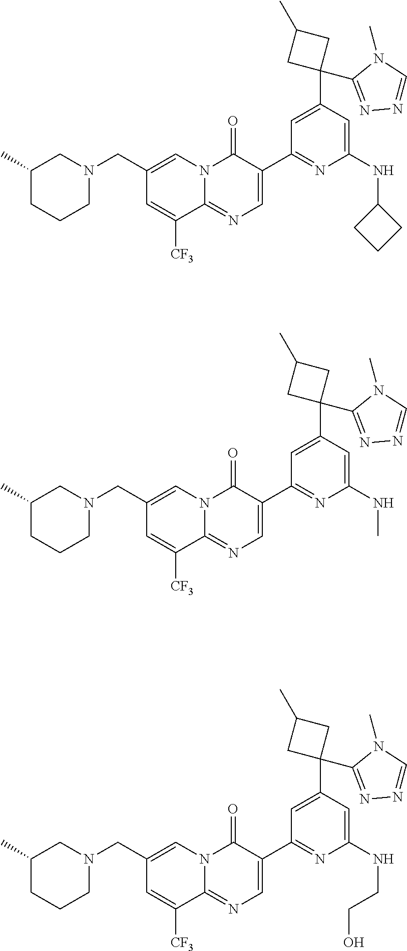 [SMILES: CC1CC(c2cc(NC3CCC3)nc(-c3cnc4c(C(F)(F)F)cc(CN5CCC[C@H](C)C5)cn4c3=O)c2)(c2nncn2C)C1 ; CNc1cc(C2(c3nncn3C)CC(C)C2)cc(-c2cnc3c(C(F)(F)F)cc(CN4CCC[C@H](C)C4)cn3c2=O)n1 ; CC1CC(c2cc(NCCO)nc(-c3cnc4c(C(F)(F)F)cc(CN5CCC[C@H](C)C5)cn4c3=O)c2)(c2nncn2C)C1]

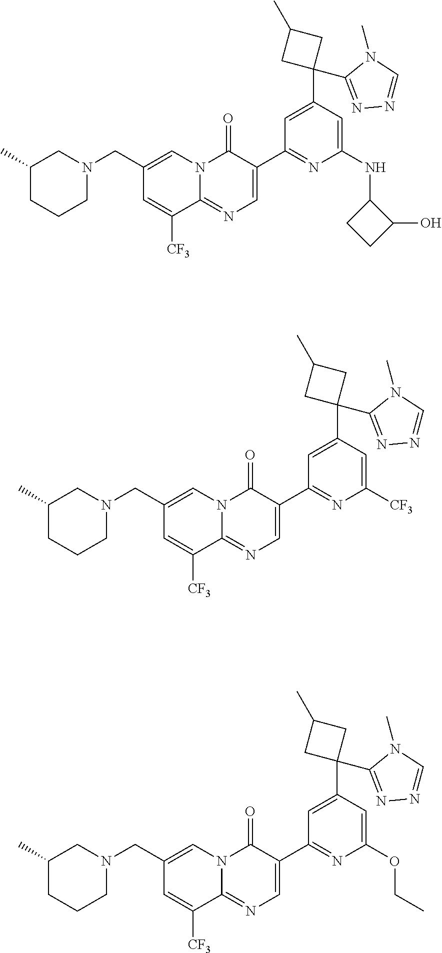 [SMILES: CC1CC(c2cc(NC3CCC3O)nc(-c3cnc4c(C(F)(F)F)cc(CN5CCC[C@H](C)C5)cn4c3=O)c2)(c2nncn2C)C1 ; CC1CC(c2cc(-c3cnc4c(C(F)(F)F)cc(CN5CCC[C@H](C)C5)cn4c3=O)nc(C(F)(F)F)c2)(c2nncn2C)C1 ; CCOc1cc(C2(c3nncn3C)CC(C)C2)cc(-c2cnc3c(C(F)(F)F)cc(CN4CCC[C@H](C)C4)cn3c2=O)n1]

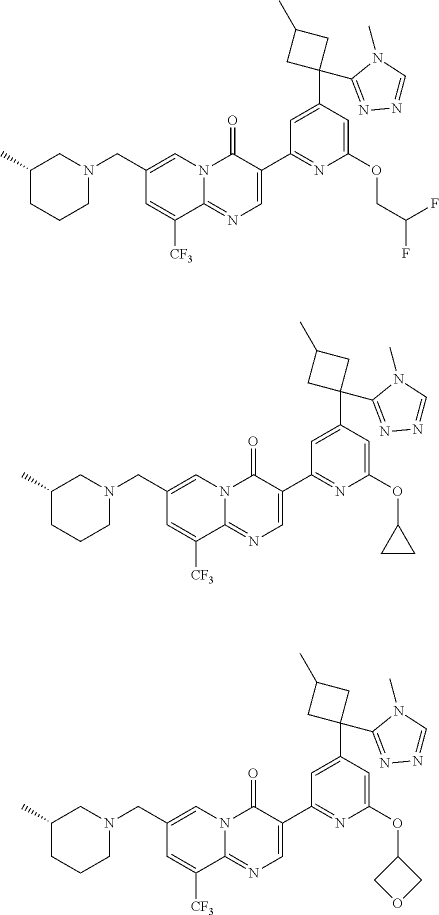 [SMILES: CC1CC(c2cc(OCC(F)F)nc(-c3cnc4c(C(F)(F)F)cc(CN5CCC[C@H](C)C5)cn4c3=O)c2)(c2nncn2C)C1 ; CC1CC(c2cc(OC3CC3)nc(-c3cnc4c(C(F)(F)F)cc(CN5CCC[C@H](C)C5)cn4c3=O)c2)(c2nncn2C)C1 ; CC1CC(c2cc(OC3COC3)nc(-c3cnc4c(C(F)(F)F)cc(CN5CCC[C@H](C)C5)cn4c3=O)c2)(c2nncn2C)C1]

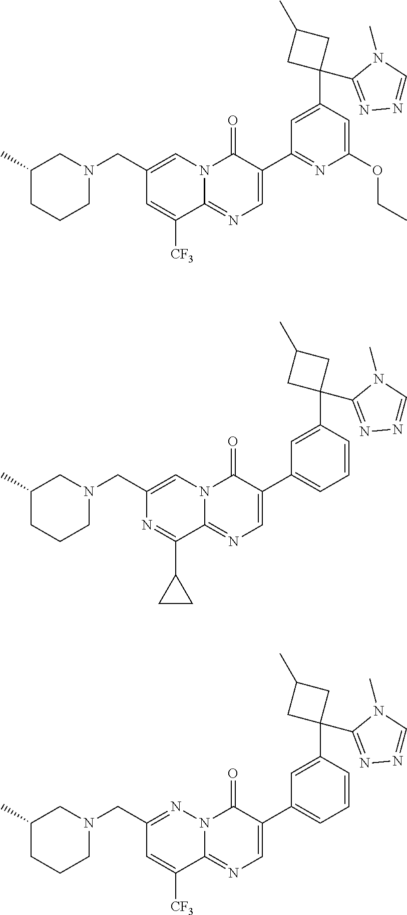

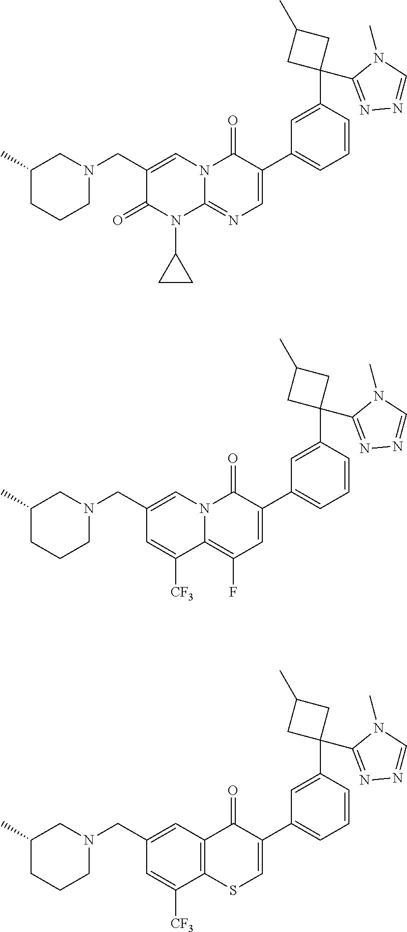 [SMILES: CC1CC(c2cccc(-c3cnc4n(C5CC5)c(=O)c(CN5CCC[C@H](C)C5)cn4c3=O)c2)(c2nncn2C)C1 ; CC1CC(c2cccc(-c3cc(F)c4c(C(F)(F)F)cc(CN5CCC[C@H](C)C5)cn4c3=O)c2)(c2nncn2C)C1 ; CC1CC(c2cccc(-c3csc4c(C(F)(F)F)cc(CN5CCC[C@H](C)C5)cc4c3=O)c2)(c2nncn2C)C1]

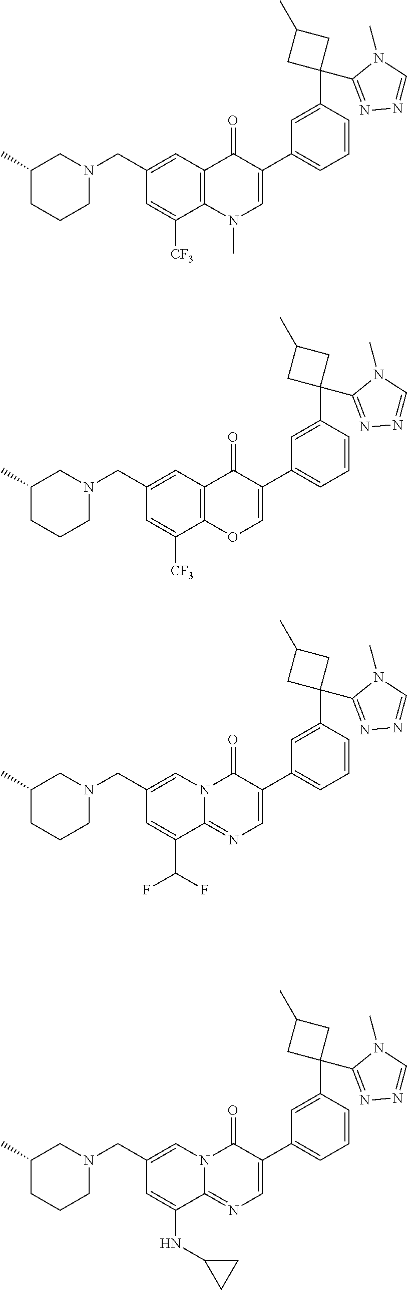 [SMILES: CC1CC(c2cccc(-c3cn(C)c4c(C(F)(F)F)cc(CN5CCC[C@H](C)C5)cc4c3=O)c2)(c2nncn2C)C1 ; CC1CC(c2cccc(-c3coc4c(C(F)(F)F)cc(CN5CCC[C@H](C)C5)cc4c3=O)c2)(c2nncn2C)C1 ; CC1CC(c2cccc(-c3cnc4c(C(F)F)cc(CN5CCC[C@H](C)C5)cn4c3=O)c2)(c2nncn2C)C1 ; CC1CC(c2cccc(-c3cnc4c(NC5CC5)cc(CN5CCC[C@H](C)C5)cn4c3=O)c2)(c2nncn2C)C1]

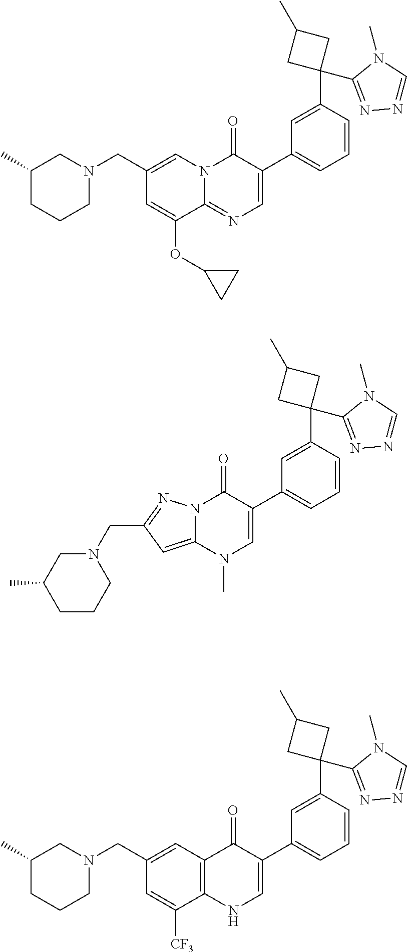 [SMILES: CC1CC(c2cccc(-c3cnc4c(OC5CC5)cc(CN5CCC[C@H](C)C5)cn4c3=O)c2)(c2nncn2C)C1 ; CC1CC(c2cccc(-c3cn(C)c4cc(CN5CCC[C@H](C)C5)nn4c3=O)c2)(c2nncn2C)C1 ; CC1CC(c2cccc(-c3c[nH]c4c(C(F)(F)F)cc(CN5CCC[C@H](C)C5)cc4c3=O)c2)(c2nncn2C)C1]

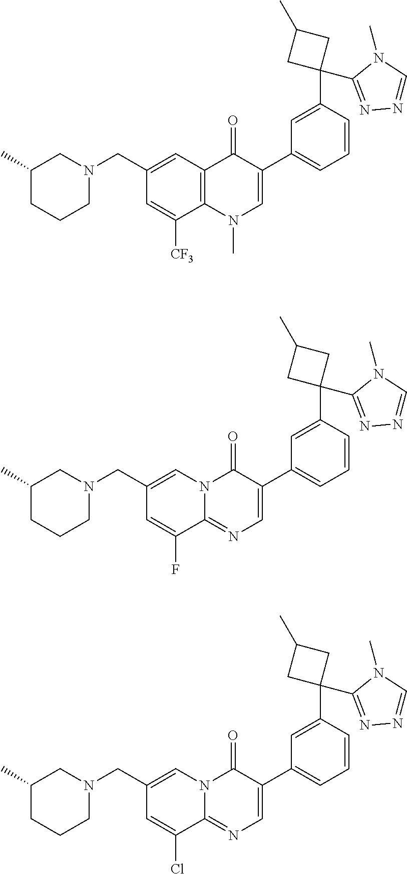 [SMILES: CC1CC(c2cccc(-c3cn(C)c4c(C(F)(F)F)cc(CN5CCC[C@H](C)C5)cc4c3=O)c2)(c2nncn2C)C1 ; CC1CC(c2cccc(-c3cnc4c(F)cc(CN5CCC[C@H](C)C5)cn4c3=O)c2)(c2nncn2C)C1 ; CC1CC(c2cccc(-c3cnc4c(Cl)cc(CN5CCC[C@H](C)C5)cn4c3=O)c2)(c2nncn2C)C1]

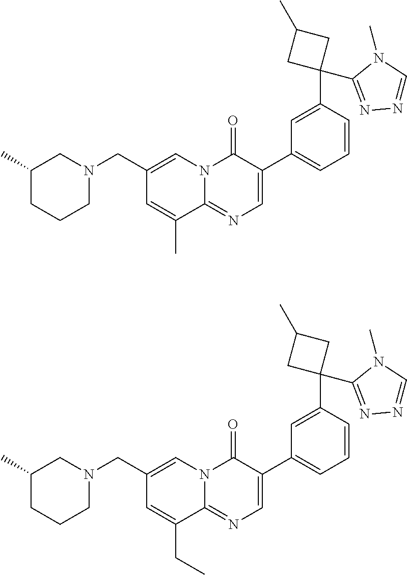 [SMILES: Cc1cc(CN2CCC[C@H](C)C2)cn2c(=O)c(-c3cccc(C4(c5nncn5C)CC(C)C4)c3)cnc12 ; CCc1cc(CN2CCC[C@H](C)C2)cn2c(=O)c(-c3cccc(C4(c5nncn5C)CC(C)C4)c3)cnc12]

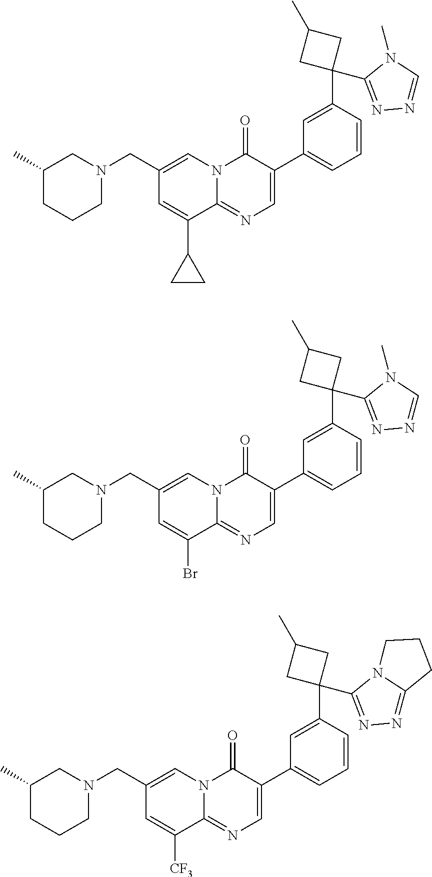 [SMILES: CC1CC(c2cccc(-c3cnc4c(C5CC5)cc(CN5CCC[C@H](C)C5)cn4c3=O)c2)(c2nncn2C)C1 ; CC1CC(c2cccc(-c3cnc4c(Br)cc(CN5CCC[C@H](C)C5)cn4c3=O)c2)(c2nncn2C)C1 ; CC1CC(c2cccc(-c3cnc4c(C(F)(F)F)cc(CN5CCC[C@H](C)C5)cn4c3=O)c2)(c2nnc3n2CCC3)C1]

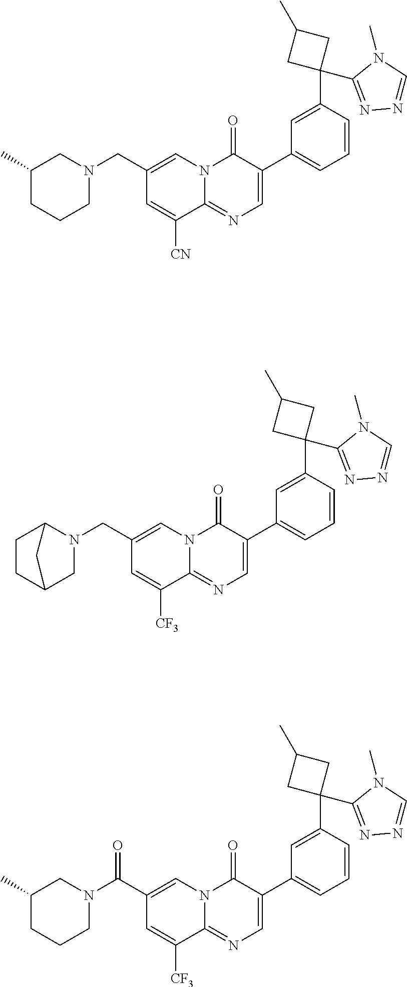 [SMILES: CC1CC(c2cccc(-c3cnc4c(C#N)cc(CN5CCC[C@H](C)C5)cn4c3=O)c2)(c2nncn2C)C1 ; CC1CC(c2cccc(-c3cnc4c(C(F)(F)F)cc(CN5CC6CCC5C6)cn4c3=O)c2)(c2nncn2C)C1 ; CC1CC(c2cccc(-c3cnc4c(C(F)(F)F)cc(C(=O)N5CCC[C@H](C)C5)cn4c3=O)c2)(c2nncn2C)C1]

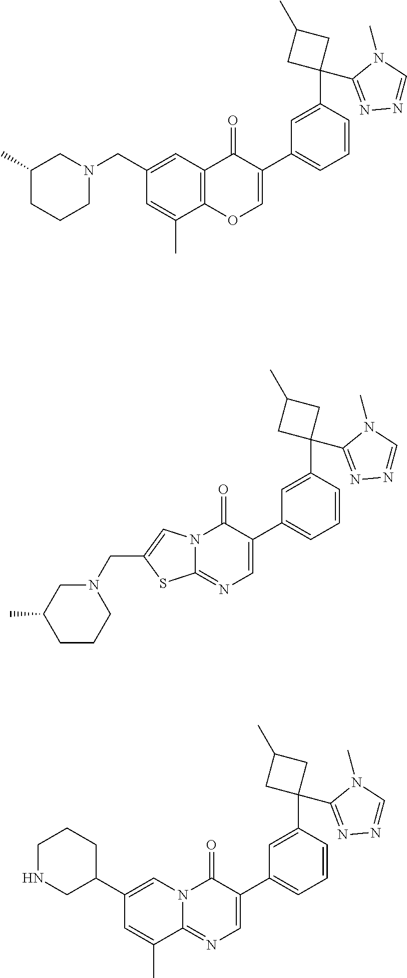 [SMILES: Cc1cc(CN2CCC[C@H](C)C2)cc2c(=O)c(-c3cccc(C4(c5nncn5C)CC(C)C4)c3)coc12 ; CC1CC(c2cccc(-c3cnc4sc(CN5CCC[C@H](C)C5)cn4c3=O)c2)(c2nncn2C)C1 ; Cc1cc(C2CCCNC2)cn2c(=O)c(-c3cccc(C4(c5nncn5C)CC(C)C4)c3)cnc12]

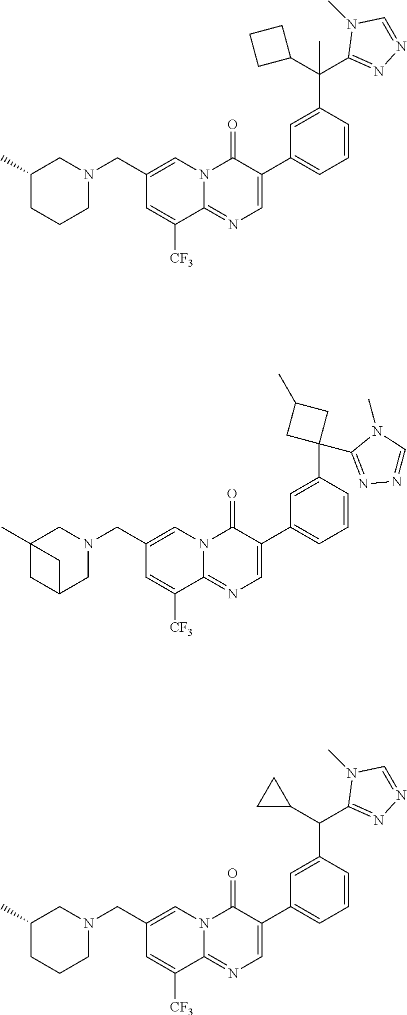 [SMILES: C[C@H]1CCCN(Cc2cc(C(F)(F)F)c3ncc(-c4cccc(C(C)(c5nncn5C)C5CCC5)c4)c(=O)n3c2)C1 ; CC1CC(c2cccc(-c3cnc4c(C(F)(F)F)cc(CN5CC6CC(C)(C6)C5)cn4c3=O)c2)(c2nncn2C)C1 ; C[C@H]1CCCN(Cc2cc(C(F)(F)F)c3ncc(-c4cccc(C(c5nncn5C)C5CC5)c4)c(=O)n3c2)C1]

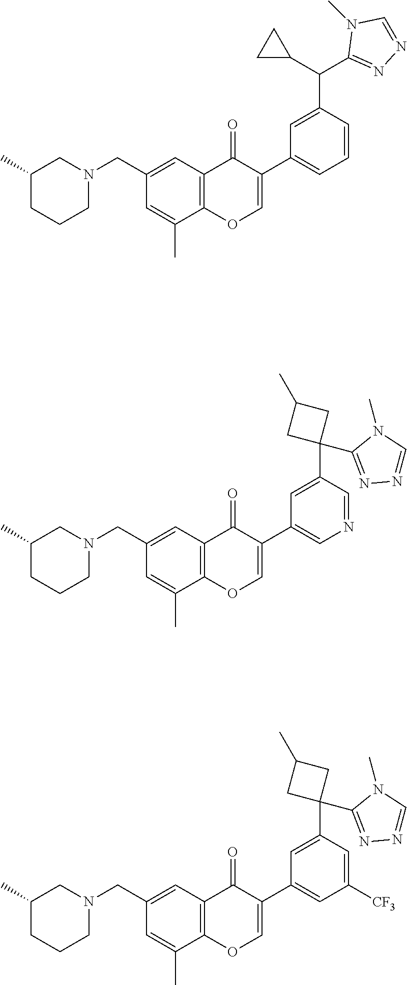 [SMILES: Cc1cc(CN2CCC[C@H](C)C2)cc2c(=O)c(-c3cccc(C(c4nncn4C)C4CC4)c3)coc12 ; Cc1cc(CN2CCC[C@H](C)C2)cc2c(=O)c(-c3cncc(C4(c5nncn5C)CC(C)C4)c3)coc12 ; Cc1cc(CN2CCC[C@H](C)C2)cc2c(=O)c(-c3cc(C(F)(F)F)cc(C4(c5nncn5C)CC(C)C4)c3)coc12]

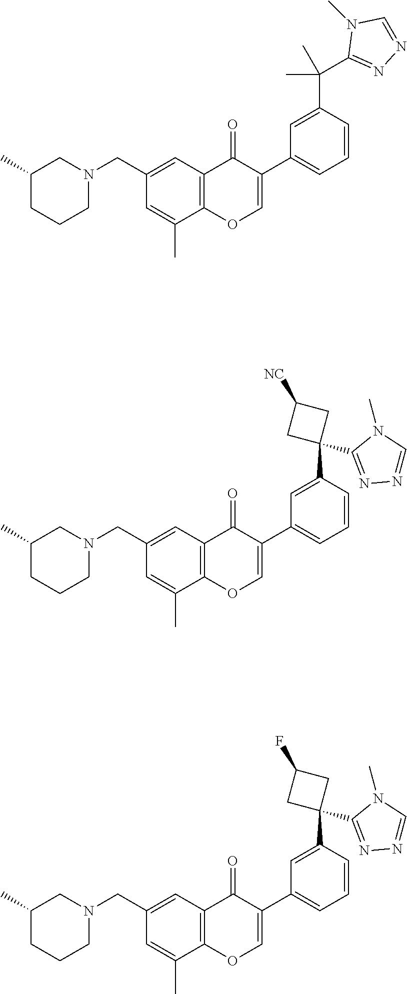 [SMILES: Cc1cc(CN2CCC[C@H](C)C2)cc2c(=O)c(-c3cccc(C(C)(C)c4nncn4C)c3)coc12 ; Cc1cc(CN2CCC[C@H](C)C2)cc2c(=O)c(-c3cccc([C@]4(c5nncn5C)C[C@H](C#N)C4)c3)coc12 ; Cc1cc(CN2CCC[C@H](C)C2)cc2c(=O)c(-c3cccc([C@]4(c5nncn5C)C[C@H](F)C4)c3)coc12]

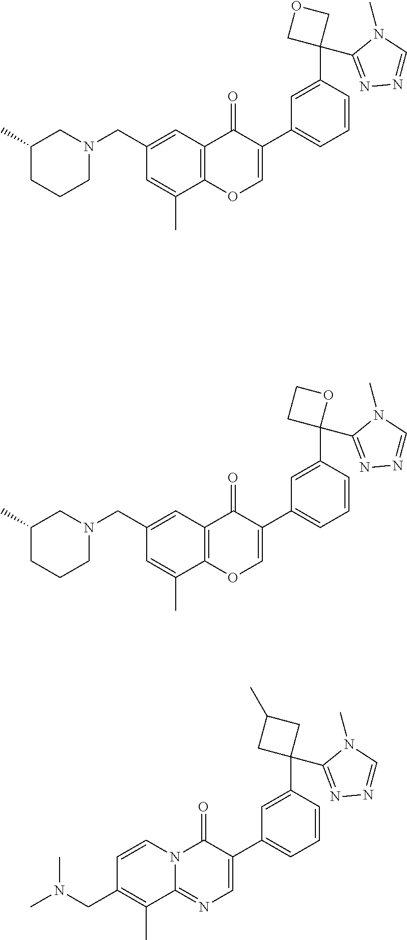 [SMILES: Cc1cc(CN2CCC[C@H](C)C2)cc2c(=O)c(-c3cccc(C4(c5nncn5C)COC4)c3)coc12 ; Cc1cc(CN2CCC[C@H](C)C2)cc2c(=O)c(-c3cccc(C4(c5nncn5C)CCO4)c3)coc12 ; Cc1c(CN(C)C)ccn2c(=O)c(-c3cccc(C4(c5nncn5C)CC(C)C4)c3)cnc12]

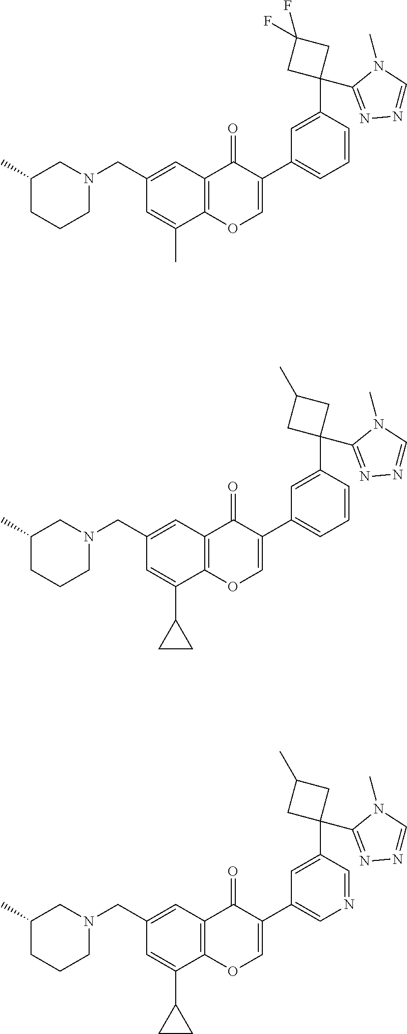 [SMILES: Cc1cc(CN2CCC[C@H](C)C2)cc2c(=O)c(-c3cccc(C4(c5nncn5C)CC(F)(F)C4)c3)coc12 ; CC1CC(c2cccc(-c3coc4c(C5CC5)cc(CN5CCC[C@H](C)C5)cc4c3=O)c2)(c2nncn2C)C1 ; CC1CC(c2cncc(-c3coc4c(C5CC5)cc(CN5CCC[C@H](C)C5)cc4c3=O)c2)(c2nncn2C)C1]

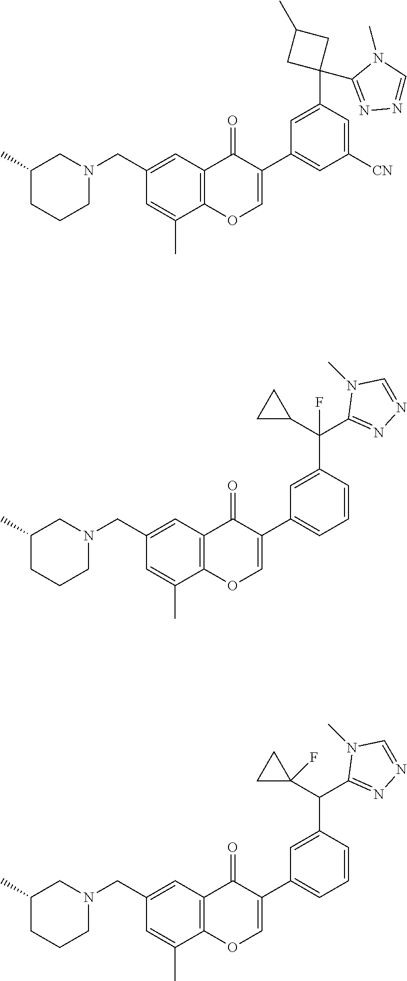 [SMILES: Cc1cc(CN2CCC[C@H](C)C2)cc2c(=O)c(-c3cc(C#N)cc(C4(c5nncn5C)CC(C)C4)c3)coc12 ; Cc1cc(CN2CCC[C@H](C)C2)cc2c(=O)c(-c3cccc(C(F)(c4nncn4C)C4CC4)c3)coc12 ; Cc1cc(CN2CCC[C@H](C)C2)cc2c(=O)c(-c3cccc(C(c4nncn4C)C4(F)CC4)c3)coc12]

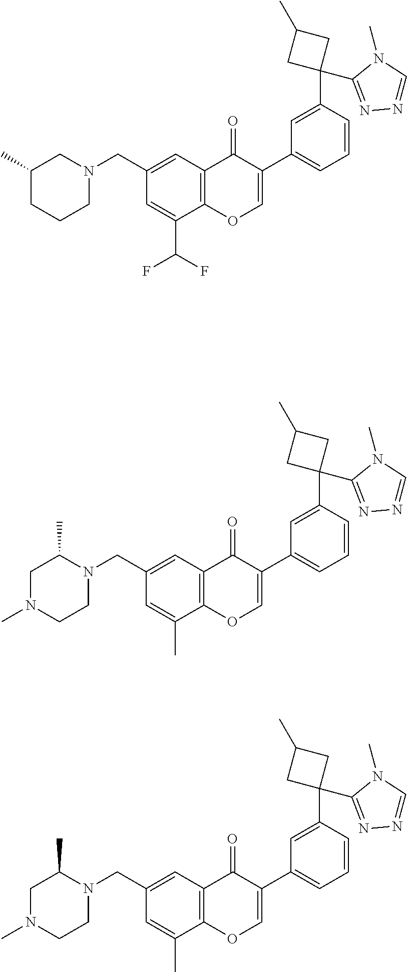 [SMILES: CC1CC(c2cccc(-c3coc4c(C(F)F)cc(CN5CCC[C@H](C)C5)cc4c3=O)c2)(c2nncn2C)C1 ; Cc1cc(CN2CCN(C)C[C@@H]2C)cc2c(=O)c(-c3cccc(C4(c5nncn5C)CC(C)C4)c3)coc12 ; Cc1cc(CN2CCN(C)C[C@H]2C)cc2c(=O)c(-c3cccc(C4(c5nncn5C)CC(C)C4)c3)coc12]

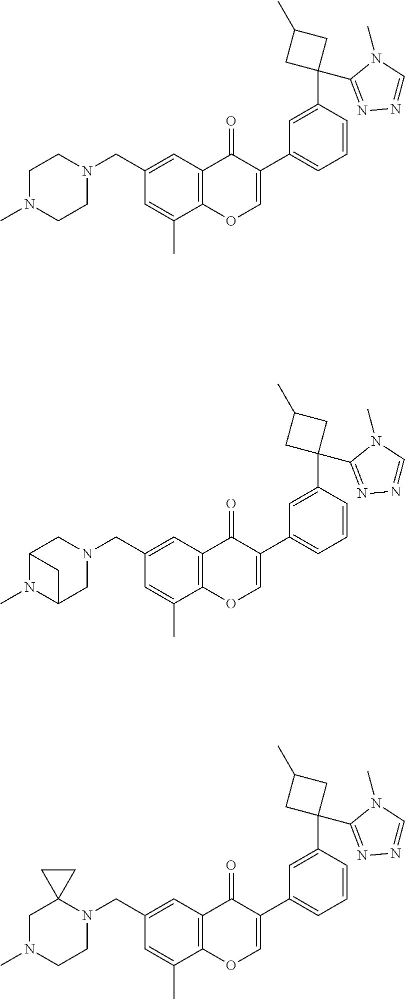 [SMILES: Cc1cc(CN2CCN(C)CC2)cc2c(=O)c(-c3cccc(C4(c5nncn5C)CC(C)C4)c3)coc12 ; Cc1cc(CN2CC3CC(C2)N3C)cc2c(=O)c(-c3cccc(C4(c5nncn5C)CC(C)C4)c3)coc12 ; Cc1cc(CN2CCN(C)CC23CC3)cc2c(=O)c(-c3cccc(C4(c5nncn5C)CC(C)C4)c3)coc12]

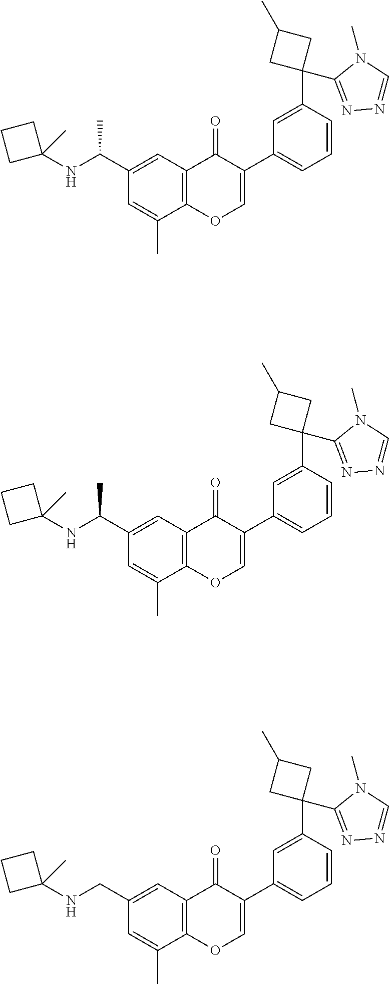 [SMILES: Cc1cc([C@@H](C)NC2(C)CCC2)cc2c(=O)c(-c3cccc(C4(c5nncn5C)CC(C)C4)c3)coc12 ; Cc1cc([C@H](C)NC2(C)CCC2)cc2c(=O)c(-c3cccc(C4(c5nncn5C)CC(C)C4)c3)coc12 ; Cc1cc(CNC2(C)CCC2)cc2c(=O)c(-c3cccc(C4(c5nncn5C)CC(C)C4)c3)coc12]

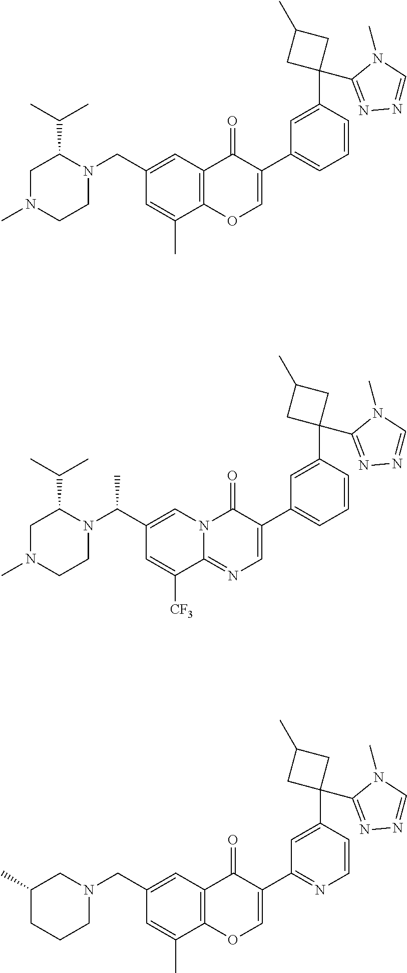 [SMILES: Cc1cc(CN2CCN(C)C[C@@H]2C(C)C)cc2c(=O)c(-c3cccc(C4(c5nncn5C)CC(C)C4)c3)coc12 ; CC1CC(c2cccc(-c3cnc4c(C(F)(F)F)cc([C@@H](C)N5CCN(C)C[C@@H]5C(C)C)cn4c3=O)c2)(c2nncn2C)C1 ; Cc1cc(CN2CCC[C@H](C)C2)cc2c(=O)c(-c3cc(C4(c5nncn5C)CC(C)C4)ccn3)coc12]

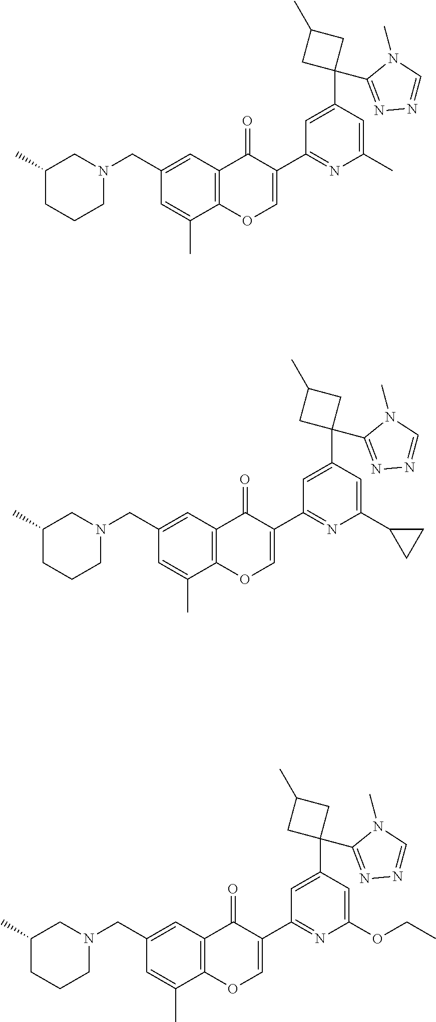 [SMILES: Cc1cc(C2(c3nncn3C)CC(C)C2)cc(-c2coc3c(C)cc(CN4CCC[C@H](C)C4)cc3c2=O)n1 ; Cc1cc(CN2CCC[C@H](C)C2)cc2c(=O)c(-c3cc(C4(c5nncn5C)CC(C)C4)cc(C4CC4)n3)coc12 ; CCOc1cc(C2(c3nncn3C)CC(C)C2)cc(-c2coc3c(C)cc(CN4CCC[C@H](C)C4)cc3c2=O)n1]

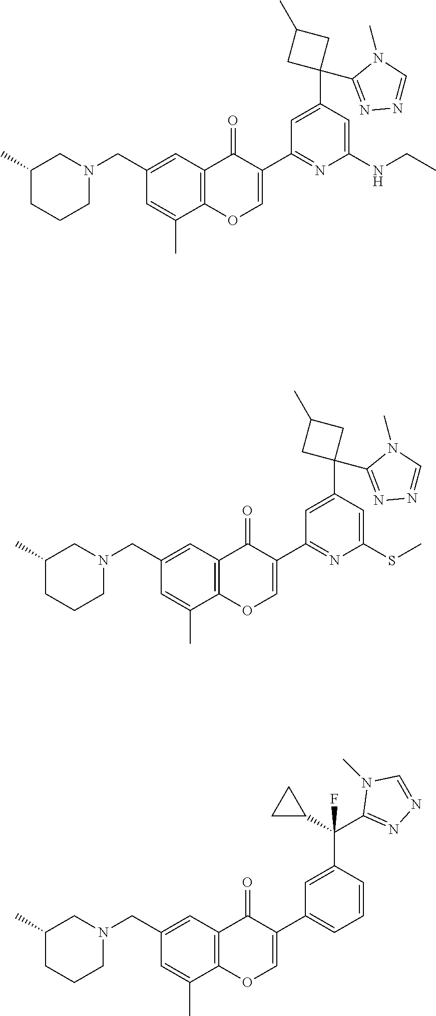

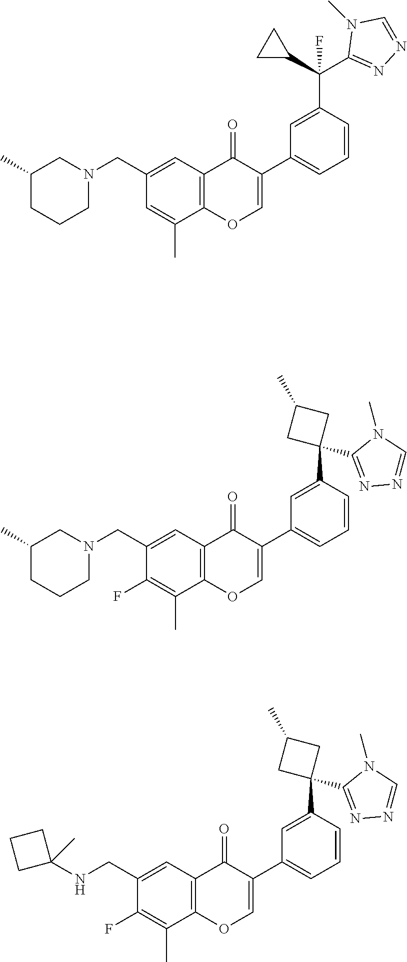 [SMILES: Cc1cc(CN2CCC[C@H](C)C2)cc2c(=O)c(-c3cccc([C@](F)(c4nncn4C)C4CC4)c3)coc12 ; Cc1c(F)c(CN2CCC[C@H](C)C2)cc2c(=O)c(-c3cccc([C@]4(c5nncn5C)C[C@@H](C)C4)c3)coc12 ; Cc1c(F)c(CNC2(C)CCC2)cc2c(=O)c(-c3cccc([C@]4(c5nncn5C)C[C@@H](C)C4)c3)coc12]

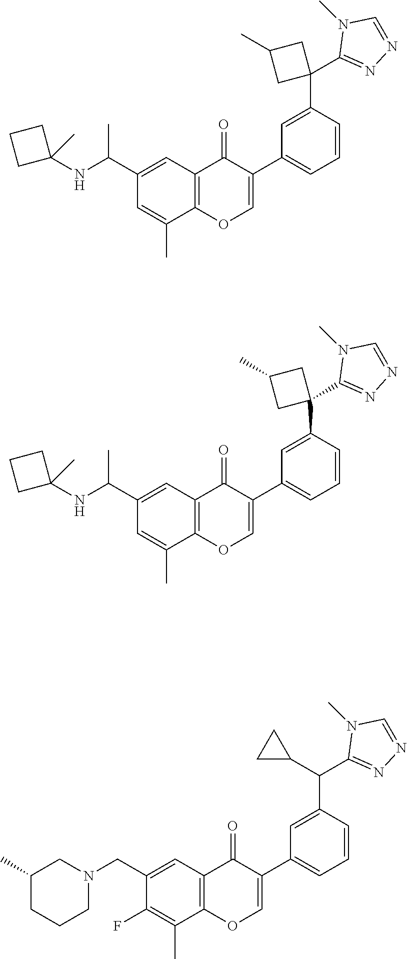

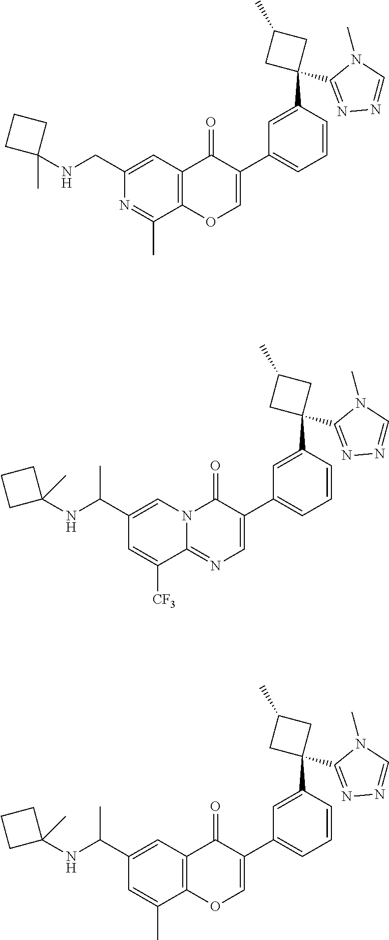 [SMILES: Cc1nc(CNC2(C)CCC2)cc2c(=O)c(-c3cccc([C@]4(c5nncn5C)C[C@@H](C)C4)c3)coc12 ; CC(NC1(C)CCC1)c1cc(C(F)(F)F)c2ncc(-c3cccc([C@]4(c5nncn5C)C[C@@H](C)C4)c3)c(=O)n2c1 ; Cc1cc(C(C)NC2(C)CCC2)cc2c(=O)c(-c3cccc([C@]4(c5nncn5C)C[C@@H](C)C4)c3)coc12]

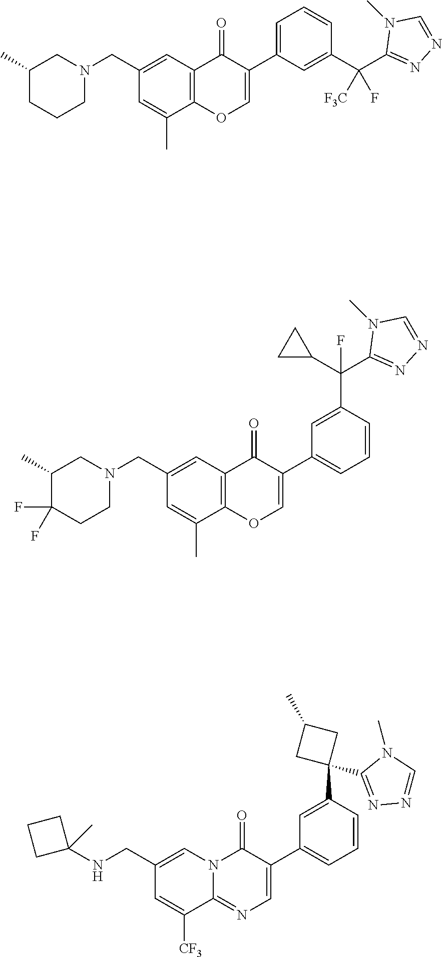 [SMILES: Cc1cc(CN2CCC[C@H](C)C2)cc2c(=O)c(-c3cccc(C(F)(c4nncn4C)C(F)(F)F)c3)coc12 ; Cc1cc(CN2CCC(F)(F)[C@H](C)C2)cc2c(=O)c(-c3cccc(C(F)(c4nncn4C)C4CC4)c3)coc12 ; Cn1cnnc1[C@]1(c2cccc(-c3cnc4c(C(F)(F)F)cc(CNC5(C)CCC5)cn4c3=O)c2)C[C@H](C)C1]

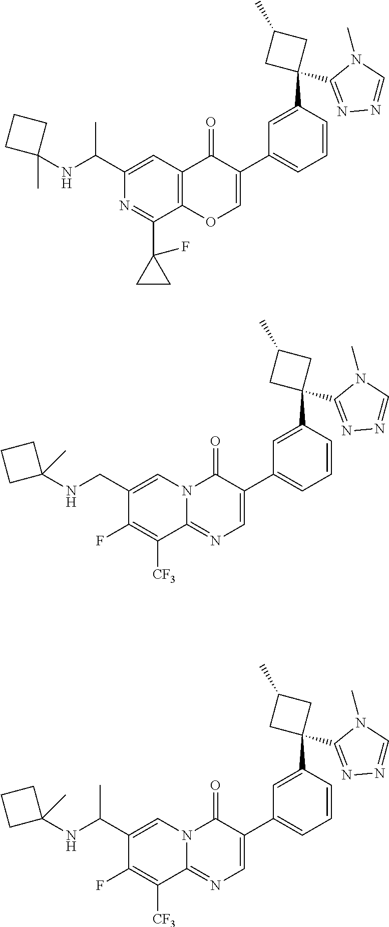 [SMILES: CC(NC1(C)CCC1)c1cc2c(=O)c(-c3cccc([C@]4(c5nncn5C)C[C@@H](C)C4)c3)coc2c(C2(F)CC2)n1 ; Cn1cnnc1[C@]1(c2cccc(-c3cnc4c(C(F)(F)F)c(F)c(CNC5(C)CCC5)cn4c3=O)c2)C[C@H](C)C1 ; CC(NC1(C)CCC1)c1cn2c(=O)c(-c3cccc([C@]4(c5nncn5C)C[C@@H](C)C4)c3)cnc2c(C(F)(F)F)c1F]

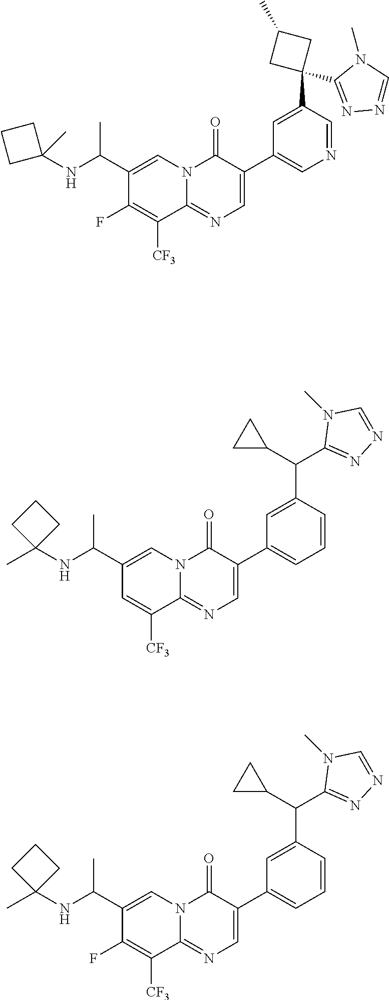 [SMILES: CC(NC1(C)CCC1)c1cn2c(=O)c(-c3cncc([C@]4(c5nncn5C)C[C@@H](C)C4)c3)cnc2c(C(F)(F)F)c1F ; CC(NC1(C)CCC1)c1cc(C(F)(F)F)c2ncc(-c3cccc(C(c4nncn4C)C4CC4)c3)c(=O)n2c1 ; CC(NC1(C)CCC1)c1cn2c(=O)c(-c3cccc(C(c4nncn4C)C4CC4)c3)cnc2c(C(F)(F)F)c1F]

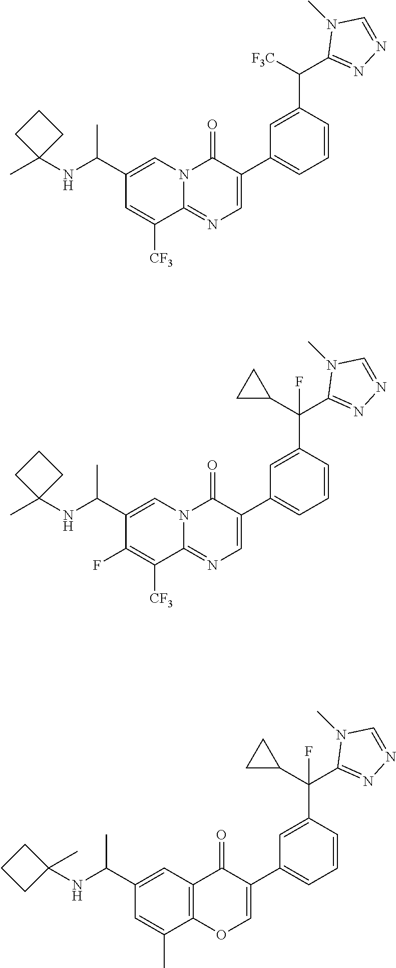 [SMILES: CC(NC1(C)CCC1)c1cc(C(F)(F)F)c2ncc(-c3cccc(C(c4nncn4C)C(F)(F)F)c3)c(=O)n2c1 ; CC(NC1(C)CCC1)c1cn2c(=O)c(-c3cccc(C(F)(c4nncn4C)C4CC4)c3)cnc2c(C(F)(F)F)c1F ; Cc1cc(C(C)NC2(C)CCC2)cc2c(=O)c(-c3cccc(C(F)(c4nncn4C)C4CC4)c3)coc12]

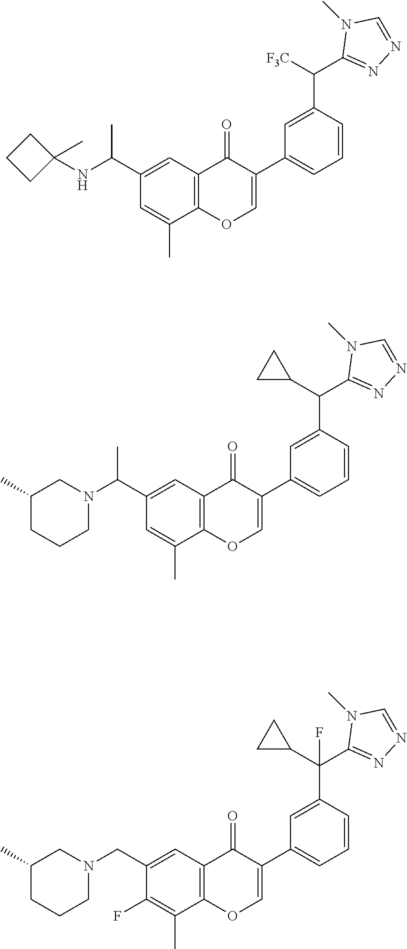 [SMILES: Cc1cc(C(C)NC2(C)CCC2)cc2c(=O)c(-c3cccc(C(c4nncn4C)C(F)(F)F)c3)coc12 ; Cc1cc(C(C)N2CCC[C@H](C)C2)cc2c(=O)c(-c3cccc(C(c4nncn4C)C4CC4)c3)coc12 ; Cc1c(F)c(CN2CCC[C@H](C)C2)cc2c(=O)c(-c3cccc(C(F)(c4nncn4C)C4CC4)c3)coc12]

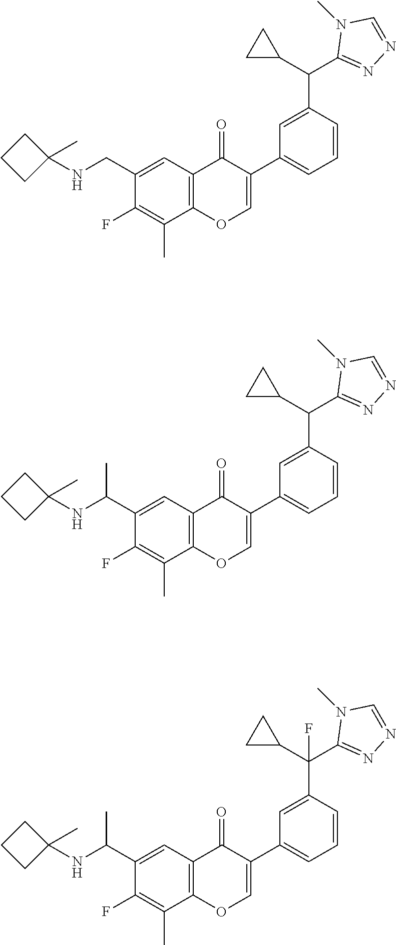 [SMILES: Cc1c(F)c(CNC2(C)CCC2)cc2c(=O)c(-c3cccc(C(c4nncn4C)C4CC4)c3)coc12 ; Cc1c(F)c(C(C)NC2(C)CCC2)cc2c(=O)c(-c3cccc(C(c4nncn4C)C4CC4)c3)coc12 ; Cc1c(F)c(C(C)NC2(C)CCC2)cc2c(=O)c(-c3cccc(C(F)(c4nncn4C)C4CC4)c3)coc12]

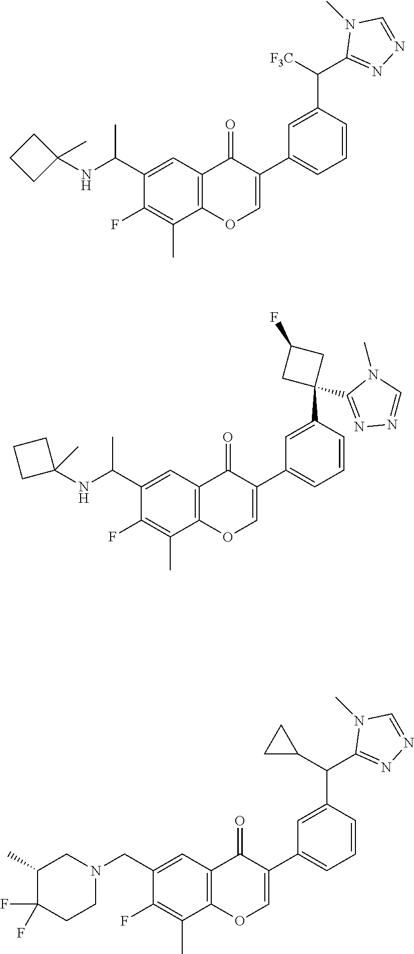 [SMILES: Cc1c(F)c(C(C)NC2(C)CCC2)cc2c(=O)c(-c3cccc(C(c4nncn4C)C(F)(F)F)c3)coc12 ; Cc1c(F)c(C(C)NC2(C)CCC2)cc2c(=O)c(-c3cccc([C@]4(c5nncn5C)C[C@H](F)C4)c3)coc12 ; Cc1c(F)c(CN2CCC(F)(F)[C@H](C)C2)cc2c(=O)c(-c3cccc(C(c4nncn4C)C4CC4)c3)coc12]

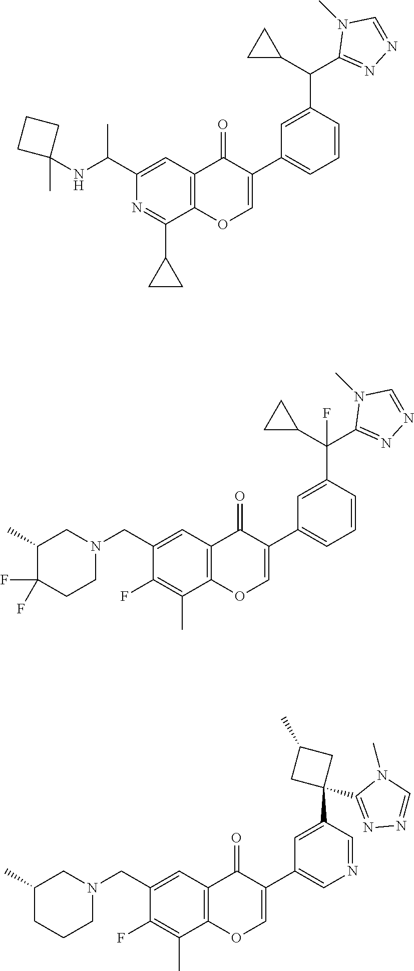 [SMILES: CC(NC1(C)CCC1)c1cc2c(=O)c(-c3cccc(C(c4nncn4C)C4CC4)c3)coc2c(C2CC2)n1 ; Cc1c(F)c(CN2CCC(F)(F)[C@H](C)C2)cc2c(=O)c(-c3cccc(C(F)(c4nncn4C)C4CC4)c3)coc12 ; Cc1c(F)c(CN2CCC[C@H](C)C2)cc2c(=O)c(-c3cncc([C@]4(c5nncn5C)C[C@@H](C)C4)c3)coc12]

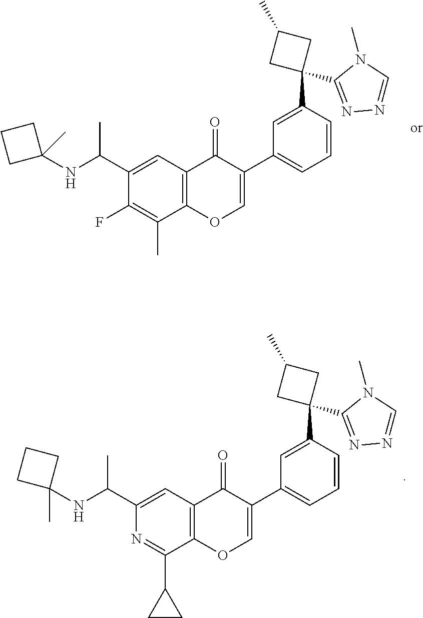 [SMILES: Cc1c(F)c(C(C)NC2(C)CCC2)cc2c(=O)c(-c3cccc([C@]4(c5nncn5C)C[C@@H](C)C4)c3)coc12 ; CC(NC1(C)CCC1)c1cc2c(=O)c(-c3cccc([C@]4(c5nncn5C)C[C@@H](C)C4)c3)coc2c(C2CC2)n1]

24. A pharmaceutical composition, comprising the compound, or the stereoisomer or pharmaceutically acceptable salt thereof according to claim 1, and a pharmaceutically acceptable adjuvant.

25. A method for preventing or treating a disease or condition mediated by Cbl-b, comprising administering to an individual in need thereof the compound, or the stereoisomer or pharmaceutically acceptable salt thereof according to claim 1, wherein the disease or condition mediated by Cbl-b is preferably a tumor or an autoimmune disease.

26. (canceled)
